(19) United States
(12) Patent Application Publication
(10) Pub. No.: US 2014/0120060 A1
(43) Pub. Date: May 1, 2014

(54) TREATMENT OF RHEUMATOID ARTHRITIS AND ASTHMA USING PI3 KINASE INHIBITORS

(71) Applicant: INFINITY PHARMACEUTICALS, INC., Cambridge, MA (US)

(72) Inventors: Vito J. Palombella, Needham, MA (US); Jeffrey L. Kutok, Natick, MA (US)

(73) Assignee: INFINITY PHARMACEUTICALS, INC., Cambridge, MA (US)

(21) Appl. No.: 13/839,912

(22) Filed: Mar. 15, 2013

Related U.S. Application Data
(60) Provisional application No. 61/721,416, filed on Nov. 1, 2012.
(60) Provisional application No. 61/721,422, filed on Nov. 1, 2012.
(60) Provisional application No. 61/767,625, filed on Feb. 21, 2013.

(51) Int. Cl. C07D 473/34; A61K 31/5377; A61K 45/06; A61K 31/52

(52) U.S. Cl. CPC C07D 473/34; A61K 31/52; A61K 31/5377; A61K 45/06
USPC 424/85.7; 514/263.22; 514/234.2; 424/142.1; 424/133.1; 514/64; 514/16.6; 514/167

(57) ABSTRACT

Provided herein are methods, kits, and pharmaceutical compositions that include a PI3 kinase inhibitor for treating rheumatoid arthritis or asthma.

This application claims priority to U.S. Provisional Application Nos. 61/721,416, filed Nov. 1, 2012, 61/721,422, filed Nov. 1, 2012, and 61/767,625, filed Feb. 21, 2013, the entireties of which are incorporated herein by reference.

### BACKGROUND OF THE INVENTION

Rheumatoid arthritis (RA) is an autoimmune disorder characterized by chronic inflammatory polyarthritis. Over time, RA may result in progressive joint destruction, deformity, disability, and premature death. Between 0.5% and 1% of most adult populations worldwide may suffer from this disease, and RA is more common in women than men. Genetic factors play a role in RA, with heritability estimated to be around 60%, predominantly due to human leukocyte antigen (HLA) genes; however, other genes and smoking appear to play a role in the development of RA.

The majority of patients with RA present following several weeks of symmetrical polyarthritis, especially of the small joints of the hands and feet. In many patients, Rheumatoid Factor (RF) and/or anti-citrullinated peptide (ACPA or anti-CCP) antibodies are present. Diffuse, symmetrical swelling and tenderness of the joints (which can initially be asymmetrical) may be accompanied by joint erythema. Morning stiffness is common, and patients may also have fatigue, malaise, fevers, weight loss, palmar erythema, lymphadenopathy, and/or diffuse musculoskeletal pain. Muscle weakness, muscle spasms, reduced range of motion and loss of function may develop as a result of ongoing inflammation and may lead to significant disability. Chronic synovial inflammation in RA leads to destruction of cartilage, subchondral bone, tendons and ligaments; radiographs show joint subluxation and deformities, symmetrical joint space narrowing and erosions of bone. Even nonsynovial joints, most notably the diskovertebral joints in the cervical spine, can also be severely affected by osteochondral destruction and subluxation. Rheumatoid nodules, which are pathognomonic chronic inflammatory lesions in RA that can also be found outside of joints, often subcutaneously but also throughout the body, causing destruction of other tissues. Additional extra-articular manifestations of RA include scleritis and episcleritis, systemic vasculitis, pulmonary disease (including but not limited to interstitial lung disease). Patients with RA are at increased risk of cancer and coronary artery disease.

Present first-line therapy for RA includes anti-inflammatory medications, such as nonsteroidal anti-inflammatory drugs (NSAIDs) and corticosteroids (oral and intra-articular). Once the polyarthritis becomes chronic (typically more than 6 weeks in duration) and persistent despite anti-inflammatory treatments, a disease-modifying anti-rheumatic drug (DMARD) is typically initiated, with methotrexate (MTX) often the first employed. MTX is administered on a weekly schedule, often with daily folate supplementation to reduce adverse effects. In some patients, MTX may lack sufficient efficacy and adequate tolerability. Eventually, many patients with RA will require additional DMARDs or other therapies to adequately control their disease. Other treatments include antimalarial medications, sulfasalazine, a pyrimidine synthesis inhibitor (leflunomide), and more recently, biologic agents such as tumor necrosis factor (TNF)-inhibitors (such as adalimumab, etanercept, infliximab, golimumab, and certolizumab) and other immune modulators (such as abatacept (a co-stimulatory modulator), rituximab (a B-cell-depleting anti-CD20 agent), and the interleukin-6 (IL-6) inhibitor tocilizumab). Although helpful in treating RA, not all patients respond to these agents, responses may be temporary, and many DMARDs have risks associated with their long-term use including infections. In addition, it is difficult to predict which patient will respond to a specific treatment. See, e.g., Plenge et al., Genetic variants that predict response to anti-tumor necrosis factor therapy in rheumatoid arthritis: current challenges and future directions, Curr. Opin. Rheumatol., 2008, 20(2), 145-52. Although there are many treatments available for RA, their administration rarely leads to clinical remission. See, e.g., Felson, Defining remission in rheumatoid arthritis, Ann. Rheum. Dis., 2012, 71(Supplement 2), i86-8. Thus, there remains a significant need for improved therapy for RA.

Asthma is a chronic inflammatory disease of the airways that affects people of all ages. An estimated 300 million people have the disease worldwide, with the prevalence varying by country globally from 1 to 18%. The World Health Organization (WHO) has estimated that asthma is associated with an annual loss of 15 million disability-adjusted life years (DALY), accounting for approximately 1% of the total healthcare burden. Worldwide as many as 250,000 patients die from asthma each year. It is estimated that 5 to 10% of asthma patients have severe and/or refractory asthma that is not well managed with current therapies. See Wenzel S., American Journal of Respiratory and Critical Care Medicine, 2005, 172(2):149-60.

Treatment of severe and/or refractory asthma remains highly problematic, with systemic corticosteroids often used to control symptoms. Id. See also, Holgate S T, Polosa R., Lancet., 2006, 368(9537):780-93. Long-term use of systemic corticosteroids are associated with well-known side effects, including hyperglycemia, increased susceptibility to infections, myopathy, cataracts, and osteoporosis. Resistance or poor responsiveness to corticosteroids is characteristic of many severe/refractory asthmatics, thus control may not be achieved despite long-term use of potentially toxic medication. At this time there remains an unmet clinical need for novel agents for severe/refractory asthma.

PI3K δ and γ have been shown in preclinical studies to modulate inflammatory pathways and cell types believed to be important in asthma and allergic inflammation. Importantly, many of the pathways affected by PI3K δ/γ inhibition are different from those affected by corticosteroids, thus PI3K inhibitor represents a potentially novel anti-inflammatory agent with the ability to impact asthmatic inflammation in ways that are different from currently available therapies.

### SUMMARY OF THE INVENTION

Methods, compositions, and kits for treating or preventing rheumatoid arthritis or asthma are provided herein. The methods, compositions and kits include administering a PI3K inhibitor, alone or in combination with other agents or therapeutic modalities, to a subject, e.g., a mammalian subject, e.g., a human. Disclosed herein is, at least in part, that a PI3 kinase (PI3K) inhibitor, as a single agent or in combination with one or more additional therapies, can ameliorate rheumatoid arthritis or asthma (e.g., by decreasing one or more rheumatoid arthritis or asthma-associated symptoms) in a subject, e.g., a mammalian subject. Symptoms of rheumatoid arthritis or asthma that can be ameliorated include any one or combination of symptoms of rheumatoid arthritis or asthma, as known the art and/or as disclosed herein. Experimental conditions for evaluating the effects of a PI3K inhibitor in ameliorating rheumatoid arthritis or asthma in animal models of rheumatoid arthritis or asthma are disclosed.

In one embodiment, provided herein is a method of reducing a rheumatoid arthritis or asthma associated symptom in a biological sample, comprising contacting the biological sample with a compound provided herein (e.g., a compound of Formula I (e.g., Compound 292), or an enantiomer or a mixture of enantiomers thereof, or a pharmaceutically acceptable salt, solvate, hydrate, co-crystal, clathrate, or polymorph thereof), in an amount sufficient to reduce the rheumatoid arthritis or asthma associated symptom.

In one embodiment, provided herein is a method of treating, preventing, and/or managing rheumatoid arthritis or asthma in a subject, comprising administering an effective amount of a compound provided herein (e.g., a compound of Formula I (e.g., Compound 292), or an enantiomer or a mixture of enantiomers thereof, or a pharmaceutically acceptable salt, solvate, hydrate, co-crystal, clathrate, or polymorph thereof).

In one embodiment of the present disclosure, the compound is a compound of Formula I below, or an enantiomer or a mixture of enantiomers thereof, or a pharmaceutically acceptable salt, solvate, hydrate, co-crystal, clathrate, or polymorph thereof, wherein

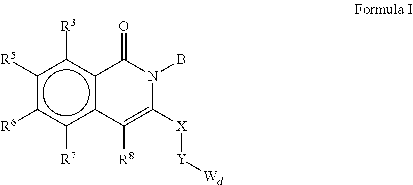

W_{d} is heterocycloalkyl, aryl or heteroaryl;
B is alkyl or a moiety of Formula II;

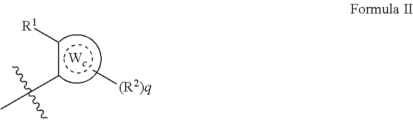

wherein W_{c} is aryl, heteroaryl, heterocycloalkyl, or cycloalkyl, and q is an integer of 0, 1, 2, 3, or 4;
X is absent or —(CH(R⁹))_{z}—, and z is an integer of 1;
Y is absent, or —N(R⁹)—;
R¹ is hydrogen, alkyl, alkenyl, alkynyl, alkoxy, amido, alkoxycarbonyl, sulfonamido, halo, cyano, or nitro;
R² is alkyl, alkenyl, alkynyl, cycloalkyl, heterocycloalkyl, aryl, heteroaryl, heteroarylalkyl, alkoxy, amino, halo, cyano, hydroxy or nitro;
R³ is hydrogen, alkyl, alkenyl, alkynyl, cycloalkyl, heterocycloalkyl, alkoxy, amido, amino, alkoxycarbonyl sulfonamido, halo, cyano, hydroxy or nitro;
R⁵, R⁶, R⁷, and R⁸ are independently hydrogen, alkyl, alkenyl, alkynyl, cycloalkyl, hetercycloalkyl, alkoxy, amido, amino, acyl, acyloxy, sulfonamido, halo, cyano, hydroxy or nitro; and
each instance of R⁹ is independently hydrogen, alkyl, cycloalkyl, or heterocycloalkyl.

In some embodiments of the compounds of Formula I, when both X and Y are present then Y is —NH—.

In some embodiments of the compounds of Formula I, X is absent or is —(CH(R⁹))_{z}—, and z is independently an integer of 1, 2, 3, or 4; and Y is absent, —O—, —S—, —S(═O)—, —S(═O)₂—, —N(R⁹)—, —C(═O)—(CHR⁹)_{z}—, —C(═O)—, —N(R⁹)(C═O)—, —N(R⁹)(C═O)NH—, or —N(R⁹)C(R⁹)₂—.

In some of the embodiments, X is —CH₂—, —CH(CH₂CH₃), or —CH(CH₃)—.

In some embodiments, X—Y is —CH₂—N(CH₃), —CH₂—N(CH₂CH₃), —CH(CH₂CH₃)—NH— or —CH(CH₃)—NH—.

In some embodiments, W_{d} is a pyrazolopyrimidine of Formula III(a), or purine of Formula III(b), Formula III(c) or Formula III(d) below:

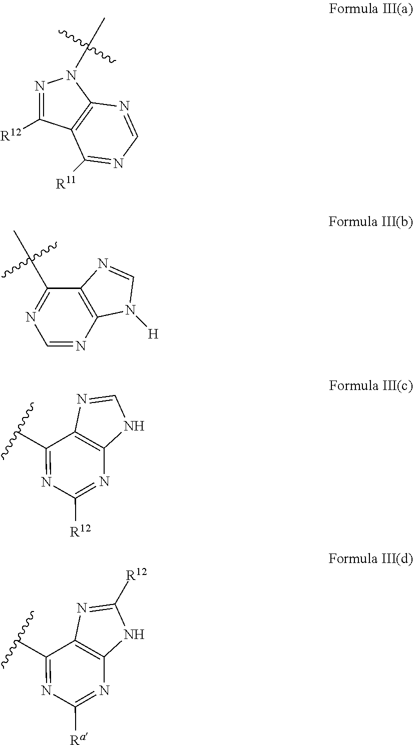

wherein R^{a′} if Formula III(d) is hydrogen, halo, phosphate, urea, a carbonate, amino, alkyl, alkenyl, alkynyl, cycloalkyl, heteroalkyl, or heterocycloalkyl; R¹¹ of Formula III(a) is H, alkyl, halo, amino, amido, hydroxy, or alkoxy, and R¹² of Formula III(a), Formula III(c) or Formula III(d) is H, alkyl, alkynyl, alkenyl, halo, aryl, heteroaryl, heterocycloalkyl, or cycloalkyl. In some embodiments, W_{d} is a pyrazolopyrimidine of Formula III(a), wherein R¹¹ is H, alkyl, halo, amino, amido, hydroxy, or alkoxy, and R¹² is cyano, amino, carboxylic acid, or amido.

In some embodiments, the compound of Formula I has the structure of Formula IV:

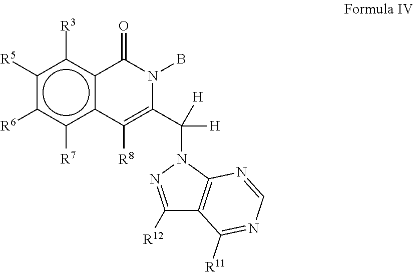

wherein R¹¹ is H, alkyl, halo, amino, amido, hydroxy, or alkoxy, and R¹² is H, alkyl, alkynyl, alkenyl, halo, aryl, heteroaryl, heterocycloalkyl, or cycloalkyl. In some embodiments, the compound of Formula I has the structure of Formula IV wherein R¹¹ is H, alkyl, halo, amino, amido, hydroxy, or alkoxy, and R¹² is cyano, amino, carboxylic acid, or amido.

In some embodiments of the compound of Formula IV, R¹¹ is amino. In some embodiments of the compound of Formula IV, R¹² is alkyl, alkenyl, alkynyl, heteroaryl, aryl, or heterocycloalkyl. In some embodiments of the compound of Formula IV, R¹² is cyano, amino, carboxylic acid, amido, monocyclic heteroaryl, or bicyclic heteroaryl.

In some embodiments of the compound of Formula I, the compound has the structure of Formula V:

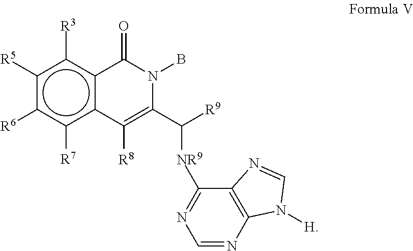

In some of the embodiments of Formula V, NR⁹ is —N(CH₂CH₃)CH₂— or N(CH₃)CH₂—.

In some of the embodiments of Formula I, the compound has a structure of Formula VI:

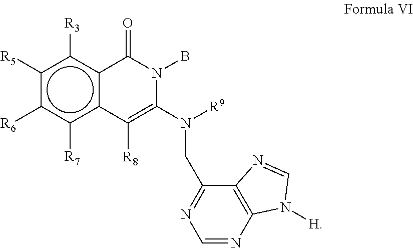

In some of the embodiments of the compound of Formula VI, R³ is —H, —CH₃, —Cl, or —F, and R⁵, R⁶, R⁷, and R⁸ are independently hydrogen.

In some of the embodiments of Formula VI, B is a moiety of Formula II;

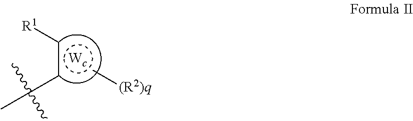

wherein W_{c} is aryl, heteroaryl, heterocycloalkyl, or cycloalkyl, and q is an integer of 0, 1, 2, 3, or 4.

In one embodiment of the present disclosure, the PI3 kinase inhibitor is a compound, or pharmaceutically acceptable salt thereof, having the structure of Formula I-1, wherein:

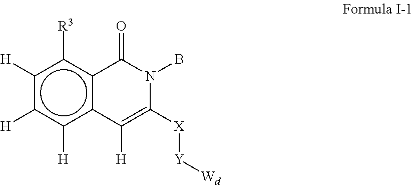

B is a moiety of Formula II;
wherein W_{c} in B is aryl, heteroaryl, heterocycloalkyl, or cycloalkyl, and q is an integer of 0, 1, 2, 3, or 4;
X is absent or —(CH(R⁹))_{z}—, and z is an integer of 1;
Y is absent, or —N(R⁹)—;
when Y is absent, Wd is:

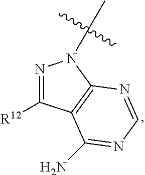

or when Y is present, Wd is:

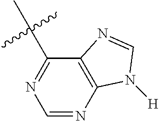 [SMILES: *c1ncnc2[nH]cnc12]

R¹ is hydrogen, alkyl, alkenyl, alkynyl, alkoxy, amido, alkoxycarbonyl, sulfonamido, halo, cyano, or nitro;
R² is alkyl, alkenyl, alkynyl, cycloalkyl, heterocycloalkyl, aryl, heteroaryl, heteroarylalkyl, alkoxy, amino, halo, cyano, hydroxy or nitro;
R³ is hydrogen, alkyl, alkenyl, alkynyl, cycloalkyl, heterocycloalkyl, alkoxy, amido, amino, alkoxycarbonyl sulfonamido, halo, cyano, hydroxy or nitro;
each instance of R⁹ is independently hydrogen, C₁-C₁₀alkyl, cycloalkyl, or hetercyclooalkyl; and R¹² is H, alkyl, alkynyl, alkenyl, halo, aryl, heteroaryl, heterocycloalkyl, or cycloalkyl.

In some embodiments, a compound of Formula I or Formula I-1 has the structure of Formula IV-A:

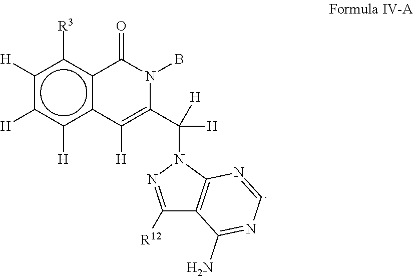

In some embodiments of the compound of Formula IV-A, R¹² is substituted benzoxazole.

In some embodiments, a compound of Formula I or Formula I-1 has the structure of Formula V-A:

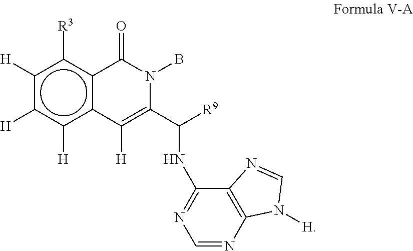

In some embodiments, a compound of Formula I or Formula I-1 has the structure of Formula IV-A or Formula V-A.

In some embodiments, a compound of Formula I or Formula I-1 has the structure of Formula V-B:

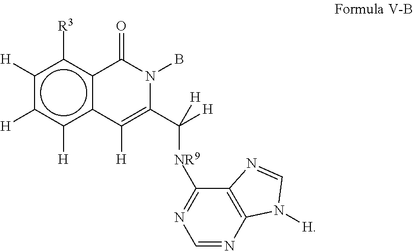

In some embodiments, a compound of Formula I or Formula I-1 has the structure of Formula VI-A:

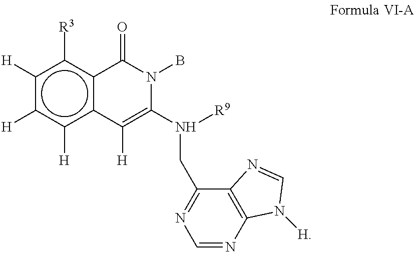

In some embodiments, a compound of Formula I or Formula I-1 is the compound wherein B is a moiety of Formula II;

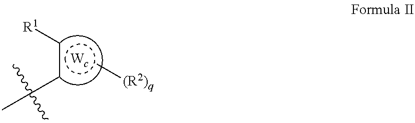

wherein W_{c} is aryl, heteroaryl, heterocycloalkyl, or cycloalkyl; q is an integer of 0 or 1; R¹ is hydrogen, alkyl, or halo; R² is alkyl or halo; and R³ is hydrogen, alkyl, or halo. In some embodiments, when both X and Y are present then Y is —NH—. In other embodiments, R³ is —H, —CH₃, —CH₂CH₃, —CF₃, —Cl or —F. In further embodiments, R³ is methyl or chloro.

In some embodiments of the compound of Formula I or Formula I-1, X is —(CH(R⁹))_{z}—, wherein R⁹ is methyl and z=1; and
Wd is

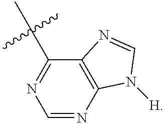

In other embodiments of the compound of Formula I or Formula I-1, the compound is predominately in an (S)-stereochemical configuration.

In further embodiments of the compound of Formula I or Formula I-1, the compound has a structure of Formula V-A2:

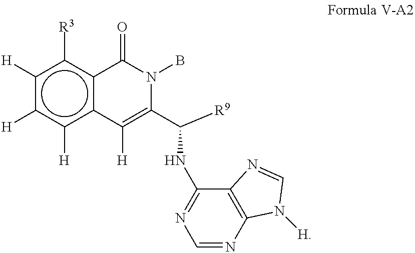

In some other embodiments of the compound of Formula I or Formula I-1, R¹² is a monocyclic heteroaryl, bicyclic heteroaryl, or heterocycloalkyl.

In some other embodiments of the compound of Formula I or Formula I-1, B is a moiety of Formula II:

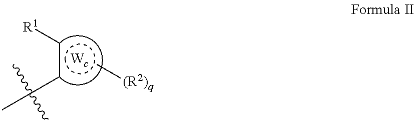

wherein W_{c} is aryl or cycloalkyl.

In some embodiments, the compound of Formula I is a polymorph Form C of Compound 292 as disclosed herein.

In some embodiments, the compound inhibits a class I PI3K. In certain embodiments, the class I PI3K is selected from p110 α, p110 β, p110 γ, and p110 δ.

In some embodiments, the compound inhibits one or more class I PI3K isoforms selected from the group consisting of PI3 kinase-α, PI3 kinase-β, PI3 kinase-γ, and PI3 kinase-δ.

In some embodiments, the compound selectively inhibits a class I PI3 kinase-δ isoform, or selectively inhibits a class I PI3 kinase-δ and a PI3 kinase-γ isoform, as compared with other class I PI3 kinase isoforms.

In some embodiments, a pharmaceutical composition is used, wherein the composition comprises a pharmaceutically acceptable excipient and one or more compounds of any formulae provided herein, including but not limited to Formula I, I-1, IV, IV-A, V, V-A, V-A2, V-B, VI, and VI-A. In some embodiments, the composition is a liquid, solid, semi-solid, gel, or an aerosol form.

In other embodiments, one or more PI3K inhibitors (e.g., one or more PI3K inhibitors described herein) are administered in combination. In one embodiment, the PI3K inhibitors are administered concurrently. In another embodiment the inhibitors are administered sequentially. For example, a combination of e.g., Compound 292 and a second PI3K inhibitor, can be administered concurrently or sequentially. In one embodiment, the second PI3K inhibitor, is administered first, followed, with or without a period of overlap, by administration of Compound 292. In another embodiment, Compound 292 is administered first, followed, with or without a period of overlap, by administration of the second PI3K inhibitor.

In one embodiment, the subject treated is a mammal, e.g., a primate, typically a human (e.g., a patient having, or at risk of having, rheumatoid arthritis, as described herein). In some embodiments, the subject treated is in need of PI3 kinase inhibition (e.g., has been evaluated to show elevated PI3K levels or alterations in another component of the PI3K pathway). In one embodiment, the subject previously received other rheumatoid arthritis treatment (e.g., methotrexate). In one embodiment, the subject treated is a mammal, e.g., a primate, typically a human (e.g., a patient having, or at risk of having, asthma, as described herein). In some embodiments, the subject treated is in need of PI3 kinase inhibition (e.g., has been evaluated to show elevated PI3K levels or alterations in another component of the PI3K pathway). In one embodiment, the subject previously received other asthma treatment.

In some embodiments, the PI3K inhibitor is administered as a pharmaceutical composition comprising the PI3K inhibitor, or a pharmaceutically acceptable salt thereof, and a pharmaceutically acceptable excipient.

In certain embodiments, the PI3K inhibitor is administered or is present in the composition, e.g., the pharmaceutical composition.

The PI3K inhibitors described herein can be administered to the subject systemically (e.g., orally, parenterally, subcutaneously, intravenously, rectally, intramuscularly, intraperitoneally, intranasally, transdermally, or by inhalation or intracavitary installation). Typically, the PI3K inhibitors are administered orally.

In one embodiment, the PI3K inhibitor is Compound 292, as disclosed in Table 4, or a pharmaceutically acceptable salt thereof. Compound 292, or a pharmaceutically acceptable salt thereof, can be administered orally. Other routes of administration are also provided herein.

The methods and compositions of the invention can, optionally, be used in combination with other therapies (e.g., one or more agents, surgical procedures, or radiation procedures). Any combination of one or more PI3K inhibitor(s) and one or more other agents or therapies can be used. The PI3K inhibitor(s) and other therapies can be administered before treatment, concurrently with treatment, post-treatment, or during remission of the disorder. In one embodiment, a second agent is administered simultaneously or sequentially with the PI3K inhibitor.

The methods of the invention can further include the step of monitoring the subject, e.g., for a change (e.g., an increase or decrease) in levels of one or more signs or symptoms or biological concomitants of rheumatoid arthritis or asthma, as disclosed herein. For example, biological concomitants can include immune complexes, elevated levels of cytokines (e.g., interferons (e.g., Type I interferons, e.g., IFN-α and/or IFN-β); interleukins (e.g., IL-6, IL-8, IL-1, and IL-18) and TNF-α), elevated levels of antibodies associated with rheumatoid arthritis or asthma (e.g., antinuclear antibodies (e.g., anti-Smith antibodies, anti-double stranded DNA (dsDNA) antibodies, anti-U1 RNP, SS-a (or anti-Ro), SS-b (or anti-La)), antiphospholipid antibodies, anti-ss DNA antibodies, anti-histone antibodies, or anticardiolipin antibodies), overexpression of IFN-α and/or IFN-β inducible genes, elevated levels of IP-10, elevated levels of sCD40L, reduced levels of C3-derived C3b, reduced peripheral iNKT cell frequencies, defective B cell-mediated stimulation of iNKT cells, altered CD1d expression on B cells, and reduced numbers of natural regulatory T cells (Treg)). In some embodiments, one or more of these biological concomitants correlates with a decrease in one or more clinical symptoms associated with rheumatoid arthritis or asthma.

In some embodiments, a normalization (e.g., a decrease in an elevated level or increase in a diminished level) of a biological concomitant is indicative of treatment efficacy and/or is predictive of improvement in clinical symptoms. For example, in some embodiments, a decrease in IFN-α is indicative of treatment efficacy. In some embodiments, a decrease in IFN-α correlates with a decrease in one or more clinical symptoms associated with rheumatoid arthritis or asthma. In some embodiments, the subject is monitored for a change in urine protein levels (e.g., a decrease in urine protein levels, which can be indicative of treatment efficacy). In some embodiments, the subject is monitored for a change in spleen inflammation (e.g., by monitoring spleen size, wherein a decrease or lack of increase in spleen size can be indicative of treatment efficacy). In some embodiments, the subject is monitored for a change in nephritis. A reduction in nephritis can be indicative of treatment efficacy. In some embodiments, the subject is monitored for a change in formation of immune complexes. A decrease in immune complexes can be indicative of treatment efficacy.

The subject can be monitored in one or more of the following periods: prior to beginning of treatment; during the treatment; or after one or more elements of the treatment have been administered. Monitoring can be used to evaluate the need for further treatment with the same PI3K inhibitor, alone or in combination with, another agent, or for additional treatment with additional agents.

The methods of the invention can further include the step of analyzing a nucleic acid or protein from the subject, e.g., analyzing the genotype of the subject. In one embodiment, a PI3K protein, or a nucleic acid encoding a PI3K protein, and/or an upstream or downstream component(s) of a PI3K signaling pathway is analyzed. The nucleic acid or protein can be detected in any biological sample (e.g., blood, urine, circulating cells, a tissue biopsy or a bone marrow biopsy) using any method disclosed herein or known in the art. For example, the PI3K protein can be detected by systemic administration of a labeled form of an antibody to PI3K followed by imaging.

The analysis can be used, e.g., to evaluate the suitability of, or to choose between alternative treatments, e.g., a particular dosage, mode of delivery, time of delivery, inclusion of adjunctive therapy, e.g., administration in combination with a second agent, or generally to determine the subject's probable drug response phenotype or genotype. The nucleic acid or protein can be analyzed at any stage of treatment, but preferably, prior to administration of the PI3K inhibitor and/or agent, to thereby determine appropriate dosage(s) and treatment regimen(s) of the PI3K inhibitor (e.g., amount per treatment or frequency of treatments) for prophylactic or therapeutic treatment of the subject.

In certain embodiments, the methods of the invention further include the step of detecting an altered PI3K level in the subject, prior to, or after, administering a PI3K inhibitor to the patient. The PI3K level can be assessed in any biological sample, e.g., blood, urine, circulating cells, or a tissue biopsy. In some embodiments, the PI3K level is assessed by systemic administration of a labeled form of an antibody to PI3K followed by imaging.

In another aspect, the invention features a composition (e.g., a pharmaceutical composition) that includes one or more PI3K inhibitors (e.g., a PI3K inhibitor as described herein) and one or more agents (e.g., an agent as disclosed herein). The composition can further include a pharmaceutically-acceptable carrier or excipient.

In another aspect, the invention features a composition for use, or the use, of a PI3K inhibitor, alone or in combination with a second agent or therapeutic modality described herein for the treatment of rheumatoid arthritis or asthma, as described herein.

In another aspect, the invention features therapeutic kits that include the PI3K inhibitor, alone or in combination with one or more additional agents, and instructions for use the treatment of rheumatoid arthritis or asthma, as described herein.

### INCORPORATION BY REFERENCE

All publications, patents, and patent applications mentioned in this specification are herein incorporated by reference in their entirety and to the same extent as if each individual publication, patent, or patent application was specifically and individually indicated to be incorporated by reference.

### BRIEF DESCRIPTION OF THE DRAWINGS

FIG. 1 depicts the effect of Compound 292 on IFN-α production induced by 0.1 μM CPG-A.

FIG. 2 depicts that Compound 292 inhibited CPG-A induced IFN-α production. The results are graphed as the percent inhibition (all samples combined).

FIG. 3 depicts that Compound 292 inhibited CPG-A induced TNF-α production.

FIG. 4 depicts that Compound 292 inhibited CPG-A induced IL-6 production.

FIG. 5 depicts that Compound 292 inhibited CPG-A induced IL-8 production.

FIG. 6 depicts that Compound 292 inhibited PAMCSK induced TNF-α production.

FIG. 7 depicts that Compound 292 inhibited PAMCSK induced IL-6 production.

FIG. 8 depicts that Compound 292 inhibited PAMCSK induced IL-8 production.

FIG. 9 depicts that Compound 292 inhibited PAMCSK induced IL-1 production.

FIG. 10 depicts the dose-dependent effect of Compound 292 in rat collagen induced arthritis (CIA) model.

FIG. 11 depicts the correlation between the AUC of Compound 292 and the reductions in the ankle diameter AUC in rat CIA model.

FIG. 12 depicts that Compound 292 prevented inflammation and protects joint bone and cartilage in rat CIA model.

FIG. 13 depicts the dose-dependent effect of Compound 292 in Freund's complete adjuvant induced rat model of arthritis.

FIG. 14A and FIG. 14B depict the dose-dependent effect of Compound 292 in rat mono-articular PG-PS model.

FIG. 15 depicts that Compound 292 inhibited leukocyte migration into the bronchoalveolar space in the murine ovalbumin induced asthma model.

FIG. 16 depicts that that Compound 292 inhibited leukocyte migration into the bronchoalveolar space in the rat ovalbumin allergic asthma model.

FIG. 17 depicts that that Compound 292 inhibited cytokine production in the rat ovalbumin allergic asthma model.

FIG. 18 depicts that Compound 292 inhibits neutrophil migration into rat air pouches stimulated with IL-8.

FIG. 19 depicts the effects of Compound 292 and a PI3K-δ selective inhibitor on inhibiting neutrophil migration into rat air pouches stimulated with IL-8.

FIG. 20A and FIG. 20B depict the PK/PD relationship following single dose administration and multiple dose administration of Compound 292 in clinical safety studies, respectively.

FIG. 21 depicts the relationship between the pharmacodynamic response and the concentration of Compound 292 in clinical safety studies.

### DETAILED DESCRIPTION

While preferred embodiments of the present invention have been shown and described herein, such embodiments are provided by way of example only. Numerous variations, changes, and substitutions will occur to those skilled in the art without departing from the invention. It should be understood that various alternatives to the embodiments of the invention described herein can be employed in practicing the invention. It is intended that the appended claims define the scope of the invention and that methods and structures within the scope of these claims and their equivalents be covered thereby.

Unless defined otherwise, all technical and scientific terms used herein have the same meaning as is commonly understood by one of skill in the art to which this invention belongs. All patents and publications referred to herein are incorporated by reference.

As used in the specification and claims, the singular form “a”, “an” and “the” includes plural references unless the context clearly dictates otherwise.

As used herein, the term “patient” or “subject” refers to an animal, typically a human (i.e., a male or female of any age group, e.g., a pediatric patient (e.g., infant, child, adolescent) or adult patient (e.g., young adult, middle-aged adult or senior adult) or other mammal, such as a primate (e.g., cynomolgus monkey, rhesus monkey); other mammals such as rodents (mice, rats), cattle, pigs, horses, sheep, goats, cats, dogs; and/or birds, that will be or has been the object of treatment, observation, and/or experiment. When the term is used in conjunction with administration of a compound or drug, then the patient has been the object of treatment, observation, and/or administration of the compound or drug.

“Treating,” “treat,” and “treatment” as used herein, refers to partially or completely inhibiting or reducing the condition from which the subject is suffering. In one embodiment, this term refers to an action that occurs while a patient is suffering from, or is diagnosed with, the condition, which reduces the severity of the condition, or retards or slows the progression of the condition. Treatment need not result in a complete cure of the condition; partial inhibition or reduction of the condition is encompassed by this term. Treatment is intended to encompass prevention or prophylaxis.

“Therapeutically effective amount,” as used herein, refers to a minimal amount or concentration of a PI3K inhibitor that, when administered alone or in combination, is sufficient to provide a therapeutic benefit in the treatment of the condition, or to delay or minimize one or more symptoms associated with the condition. The term “therapeutically effective amount” can encompass an amount that improves overall therapy, reduces or avoids symptoms or causes of the condition, or enhances the therapeutic efficacy of another therapeutic agent. The therapeutic amount need not result in a complete cure of the condition; partial inhibition or reduction of the condition is encompassed by this term. The therapeutically effective amount can also encompass a prophylactically effective amount.

As used herein, unless otherwise specified, the terms “prevent” “preventing” and “prevention” refers to an action that occurs before the subject begins to suffer from the condition, or relapse of the condition. The prevention need not result in a complete prevention of the condition; partial prevention or reduction of the condition or a symptom of the condition, or reduction of the risk of developing the condition, is encompassed by this term.

As used herein, unless otherwise specified, a “prophylactically effective amount” of a PI3K inhibitor that, when administered alone or in combination, prevents or reduces the risk of developing the condition, or one or more symptoms associated with the condition, or prevents its recurrence. The term “prophylactically effective amount” can encompass an amount that improves overall prophylaxis or enhances the prophylactic efficacy of another prophylactic agent. The prophylactic amount need not result in a complete prevention of the condition; partial prevention or reduction of the condition is encompassed by this term.

As used herein, to “decrease”, “ameliorate,” “reduce,” “treat” (or the like) a condition or symptoms associated with the condition includes reducing the severity and/or frequency of symptoms of the condition, as well as preventing the condition and/or symptoms of the condition (e.g., by reducing the severity and/or frequency of flares of symptoms). In some embodiments, the symptom is reduced by at least 10%, at least 20%, at least 30%, at least 40%, at least 50%, at least 60%, at least 70%, at least 80%, at least 90%, or at least 95% relative to a control level. The control level includes any appropriate control as known in the art. For example, the control level can be the pre-treatment level in the sample or subject treated, or it can be the level in a control population (e.g., the level in subjects who do not have the condition or the level in samples derived from subjects who do not have the condition). In some embodiments, the decrease is statistically significant, for example, as assessed using an appropriate parametric or non-parametric statistical comparison.

As used herein, “agent” or “biologically active agent” refers to a biological, pharmaceutical, or chemical compound or other moiety. Non-limiting examples include simple or complex organic or inorganic molecule, a peptide, a protein, an oligonucleotide, an antibody, an antibody derivative, antibody fragment, a vitamin derivative, a carbohydrate, a toxin, or a chemotherapeutic compound. Various compounds can be synthesized, for example, small molecules and oligomers (e.g., oligopeptides and oligonucleotides), and synthetic organic compounds based on various core structures. In addition, various natural sources can provide compounds for screening, such as plant or animal extracts, and the like. A skilled artisan can readily recognize that there is no limit as to the structural nature of the agents of the present invention.

The terms “antagonist” and “inhibitor” are used interchangeably, and they refer to a compound having the ability to inhibit a biological function of a target protein (e.g., a PI3K, e.g., PI3K-6), whether by inhibiting the activity or expression of the target protein. Accordingly, the terms “antagonist” and “inhibitors” are defined in the context of the biological role of the target protein. While antagonists can specifically interact with (e.g., bind to) the target, compounds that inhibit a biological activity of the target protein by interacting with other members of the signal transduction pathway of which the target protein is a member are also specifically included within this definition.

As used herein, a “phosphoinositide 3-kinase (PI3K) inhibitor” or “PI3K inhibitor” refers to an inhibitor of any PI3K. PI3Ks are members of a unique and conserved family of intracellular lipid kinases that phosphorylate the 3′-OH group on phosphatidylinositols or phosphoinositides. The PI3K family includes kinases with distinct substrate specificities, expression patterns, and modes of regulation (see, e.g., Katso et al., 2001, Annu. Rev. Cell Dev. Biol. 17, 615-675; Foster, F. M. et al., 2003, J Cell Sci 116, 3037-3040). The class I PI3Ks (e.g., p110 α, p110 β, p110 γ, and p110 δ) are typically activated by tyrosine kinases or G-protein coupled receptors to generate PIP3, which engages downstream mediators such as those in the Akt/PDK1 pathway, mTOR, the Tec family kinases, and the Rho family GTPases. The class II PI3Ks (e.g., PI3K-C2α, PI3K-C2β, PI3K-C2γ) and III PI3Ks (e.g., Vps34) play a key role in intracellular trafficking through the synthesis of PI(3)P and PI(3,4)P2. Specific exemplary PI3K inhibitors are disclosed herein.

The class I PI3Ks comprise a p110 catalytic subunit and a regulatory adapter subunit. See, e.g., Cantrell, D. A. (2001) Journal of Cell Science 114: 1439-1445. Four isoforms of the p110 subunit (including PI3K-α (alpha), PI3K-β (beta), PI3K-γ (gamma), and PI3K-δ (delta) isoforms) have been implicated in various biological functions. Class I PI3Kα is involved, for example, in insulin signaling, and has been found to be mutated in solid tumors. Class I PI3K-β is involved, for example, in platelet activation and insulin signaling. Class I PI3K-γ plays a role in mast cell activation, innate immune function, and immune cell trafficking (chemokines). Class I PI3K-δ is involved, for example, in B-cell and T-cell activation and function and in Fc receptor signaling in mast cells. In some embodiments provided herein, the PI3K inhibitor is a class I PI3K inhibitor. In some such embodiments, the PI3K inhibitor inhibits a PI3K-α (alpha), PI3K-β (beta), PI3K-γ (gamma), or PI3K-δ (delta) isoform, or a combination thereof.

Downstream mediators of PI3K signal transduction include Akt and mammalian target of rapamycin (mTOR). Akt possesses a pleckstrin homology (PH) domain that binds PIP3, leading to Akt kinase activation. Akt phosphorylates many substrates and is a central downstream effector of PI3K for diverse cellular responses. One important function of Akt is to augment the activity of mTOR, through phosphorylation of TSC2 and other mechanisms. mTOR is a serine-threonine kinase related to the lipid kinases of the PI3K family.

The term “pharmaceutically acceptable salt” refers to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

“Pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions of the invention is contemplated. Supplementary active ingredients can also be incorporated into the compositions.

The term “selective inhibition” or “selectively inhibit” as applied to a biologically active agent refers to the agent's ability to selectively reduce the target signaling activity as compared to off-target signaling activity, via direct or interact interaction with the target.

“Radiation therapy” means exposing a patient, using routine methods and compositions known to the practitioner, to radiation emitters such as alpha-particle emitting radionucleotides (e.g., actinium and thorium radionuclides), low linear energy transfer (LET) radiation emitters (i.e., beta emitters), conversion electron emitters (e.g., strontium-89 and samarium-153-EDTMP, or high-energy radiation, including without limitation x-rays, gamma rays, and neutrons.

“Prodrug” is meant to indicate a compound that can be converted under physiological conditions or by solvolysis to a biologically active compound described herein. Thus, the term “prodrug” refers to a precursor of a biologically active compound that is pharmaceutically acceptable. A prodrug can be inactive when administered to a subject, but is converted in vivo to an active compound, for example, by hydrolysis. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam). A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein. The term “prodrug” is also meant to include any covalently bonded carriers, which release the active compound in vivo when such prodrug is administered to a mammalian subject. Prodrugs of an active compound, as described herein, can be prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a mammalian subject, cleaves to form a free hydroxy, free amino or free mercapto group, respectively. Examples of prodrugs include, but are not limited to, acetate, formate and benzoate derivatives of an alcohol or acetamide, formamide and benzamide derivatives of an amine functional group in the active compound and the like.

The term “in vivo” refers to an event that takes place in a subject's body.

The term “in vitro” refers to an event that takes places outside of a subject's body. For example, an in vitro assay encompasses any assay run outside of a subject assay. In vitro assays encompass cell-based assays in which cells alive or dead are employed. In vitro assays also encompass a cell-free assay in which no intact cells are employed.

Unless otherwise stated, structures depicted herein are also meant to include compounds which differ only in the presence of one or more isotopically enriched atoms. For example, compounds having the present structures wherein hydrogen is replaced by deuterium or tritium, or wherein carbon atom is replaced by ¹³C- or ¹⁴C-enriched carbon, are within the scope of this invention.

The compounds described herein can also contain unnatural proportions of atomic isotopes at one or more of atoms that constitute such compounds. For example, the compounds can be radiolabeled with radioactive isotopes, such as for example tritium (³H), iodine-125 (¹²⁵I) or carbon-14 (¹⁴C). All isotopic variations of the compounds described herein, whether radioactive or not, are encompassed within the scope of the present invention.

When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range can vary from, for example, between 1% and 15% of the stated number or numerical range. The term “comprising” (and related terms such as “comprise” or “comprises” or “having” or “including”) includes those embodiments, for example, an embodiment of any composition of matter, composition, method, or process, or the like, that “consist of” or “consist essentially of” the described features.

The following abbreviations and terms have the indicated meanings throughout: PI3-K=Phosphoinositide 3-kinase; PI=phosphatidylinositol; PDK=Phosphoinositide Dependent Kinase; DNA-PK=Deoxyribose Nucleic Acid Dependent Protein Kinase; PTEN=Phosphatase and Tensin homolog deleted on chromosome Ten; PIKK=Phosphoinositide Kinase Like Kinase; AIDS=Acquired Immuno Deficiency Syndrome; HIV=Human Immunodeficiency Virus; MeI=Methyl Iodide; POCl₃=Phosphorous Oxychloride; KCNS=Potassium IsoThiocyanate; TLC=Thin Layer Chromatography; MeOH=Methanol; and CHCl₃=Chloroform.

Abbreviations used herein have their conventional meaning within the chemical and biological arts.

“Alkyl” refers to a straight or branched hydrocarbon chain radical consisting solely of carbon and hydrogen atoms, containing no unsaturation, having from one to ten carbon atoms (e.g., C₁-C₁₀ alkyl). Whenever it appears herein, a numerical range such as “1 to 10” refers to each integer in the given range; e.g., “1 to 10 carbon atoms” means that the alkyl group can consist of 1 carbon atom, 2 carbon atoms, 3 carbon atoms, etc., up to and including 10 carbon atoms, although the present definition also covers the occurrence of the term “alkyl” where no numerical range is designated. In some embodiments, it is a C₁-C₄ alkyl group. Typical alkyl groups include, but are in no way limited to, methyl, ethyl, propyl, isopropyl, n-butyl, iso-butyl, sec-butyl isobutyl, tertiary butyl, pentyl, isopentyl, neopentyl, hexyl, septyl, octyl, nonyl, decyl, and the like. The alkyl is attached to the rest of the molecule by a single bond, for example, methyl (Me), ethyl (Et), n-propyl, 1-methylethyl (iso-propyl), n-butyl, n-pentyl, 1,1-dimethylethyl (t-butyl), 3-methylhexyl, 2-methylhexyl, and the like. Unless stated otherwise specifically in the specification, an alkyl group is optionally substituted by one or more of substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (wherein t is 1 or 2), —S(O)ₜOR^{a} (where t is 1 or 2), —S(O)ₜN(R^{a})₂ (where t is 1 or 2), or PO₃(R^{a})₂ where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Alkylaryl” refers to an -(alkyl)aryl radical where aryl and alkyl are as disclosed herein and which are optionally substituted by one or more of the substituents described as suitable substituents for aryl and alkyl respectively.

“Alkylhetaryl” refers to an -(alkyl)hetaryl radical where hetaryl and alkyl are as disclosed herein and which are optionally substituted by one or more of the substituents described as suitable substituents for aryl and alkyl respectively.

“Alkylheterocycloalkyl” refers to an -(alkyl) heterocycyl radical where alkyl and heterocycloalkyl are as disclosed herein and which are optionally substituted by one or more of the substituents described as suitable substituents for heterocycloalkyl and alkyl respectively.

An “alkene” moiety refers to a group consisting of at least two carbon atoms and at least one carbon-carbon double bond, and an “alkyne” moiety refers to a group consisting of at least two carbon atoms and at least one carbon-carbon triple bond. The alkyl moiety, whether saturated or unsaturated, can be branched, straight chain, or cyclic.

“Alkenyl” refers to a straight or branched hydrocarbon chain radical group consisting solely of carbon and hydrogen atoms, containing at least one double bond, and having from two to ten carbon atoms (ie. C₂-C₁₀ alkenyl). Whenever it appears herein, a numerical range such as “2 to 10” refers to each integer in the given range; e.g., “2 to 10 carbon atoms” means that the alkenyl group can consist of 2 carbon atoms, 3 carbon atoms, etc., up to and including 10 carbon atoms. In certain embodiments, an alkenyl comprises two to eight carbon atoms. In other embodiments, an alkenyl comprises two to five carbon atoms (e.g., C₂-C₅ alkenyl). The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Alkenyl-cycloalkyl” refers to an -(alkenyl)cycloalkyl radical where alkenyl and cyclo alkyl are as disclosed herein and which are optionally substituted by one or more of the substituents described as suitable substituents for alkenyl and cycloalkyl respectively.

“Alkynyl” refers to a straight or branched hydrocarbon chain radical group consisting solely of carbon and hydrogen atoms, containing at least one triple bond, having from two to ten carbon atoms (ie. C₂-C₁₀ alkynyl). Whenever it appears herein, a numerical range such as “2 to 10” refers to each integer in the given range; e.g., “2 to 10 carbon atoms” means that the alkynyl group can consist of 2 carbon atoms, 3 carbon atoms, etc., up to and including 10 carbon atoms. In certain embodiments, an alkynyl comprises two to eight carbon atoms. In other embodiments, an alkynyl has two to five carbon atoms (e.g., C₂-C₅ alkynyl). The alkynyl is attached to the rest of the molecule by a single bond, for example, ethynyl, propynyl, butynyl, pentynyl, hexynyl, and the like. Unless stated otherwise specifically in the specification, an alkynyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (wherein t is 1 or 2), —S(O)ₜOR^{a} (where t is 1 or 2), —S(O)ₜN(R^{a})₂ (where t is 1 or 2), or PO₃(R^{a})₂, wherein each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Alkynyl-cycloalkyl” refers to an -(alkynyl)cycloalkyl radical where alkynyl and cyclo alkyl are as disclosed herein and which are optionally substituted by one or more of the substituents described as suitable substituents for alkynyl and cycloalkyl respectively.

“Carboxaldehyde” refers to a —(C═O)H radical.

“Carboxyl” refers to a —(C═O)OH radical.

“Cyano” refers to a —CN radical.

“Cycloalkyl” refers to a monocyclic or polycyclic radical that contains only carbon and hydrogen, and can be saturated, or partially unsaturated. Cycloalkyl groups include groups having from 3 to 10 ring atoms (ie. C₂-C₁₀ cycloalkyl). Whenever it appears herein, a numerical range such as “3 to 10” refers to each integer in the given range; e.g., “3 to 10 carbon atoms” means that the cycloalkyl group can consist of 3 carbon atoms, etc., up to and including 10 carbon atoms. In some embodiments, it is a C₃-C₈ cycloalkyl radical. In some embodiments, it is a C₃-C₅ cycloalkyl radical. Illustrative examples of cycloalkyl groups include, but are not limited to the following moieties: cyclopropyl, cyclobutyl, cyclopentyl, cyclopentenyl, cyclohexyl, cyclohexenyl, cycloseptyl, cyclooctyl, cyclononyl, cyclodecyl, norbornyl, and the like. Unless stated otherwise specifically in the specification, a cycloalkyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Cycloalkyl-alkenyl” refers to a -(cycloalkyl)alkenyl radical where cycloalkyl and heterocycloalkyl are as disclosed herein and which are optionally substituted by one or more of the substituents described as suitable substituents for heterocycloalkyl and cycloalkyl respectively.

“Cycloalkyl-heterocycloalkyl” refers to a -(cycloalkyl) heterocycyl radical where cycloalkyl and heterocycloalkyl are as disclosed herein and which are optionally substituted by one or more of the substituents described as suitable substituents for heterocycloalkyl and cycloalkyl respectively.

“Cycloalkyl-heteroaryl” refers to a -(cycloalkyl) heteroaryl radical where cycloalkyl and heterocycloalkyl are as disclosed herein and which are optionally substituted by one or more of the substituents described as suitable substituents for heterocycloalkyl and cycloalkyl respectively.

The term “alkoxy” refers to the group —O-alkyl, including from 1 to 8 carbon atoms of a straight, branched, cyclic configuration and combinations thereof attached to the parent structure through an oxygen. Examples include methoxy, ethoxy, propoxy, isopropoxy, cyclopropyloxy, cyclohexyloxy and the like. “Lower alkoxy” refers to alkoxy groups containing one to six carbons. In some embodiments, C₁-C₄ alkyl, is an alkyl group which encompasses both straight and branched chain alkyls of from 1 to 4 carbon atoms.

The term “substituted alkoxy” refers to alkoxy wherein the alkyl constituent is substituted (i.e., —O-(substituted alkyl)). Unless stated otherwise specifically in the specification, the alkyl moiety of an alkoxy group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The term “alkoxycarbonyl” refers to a group of the formula (alkoxy)(C═O)— attached through the carbonyl carbon wherein the alkoxy group has the indicated number of carbon atoms. Thus a C₁-C₆ alkoxycarbonyl group is an alkoxy group having from 1 to 6 carbon atoms attached through its oxygen to a carbonyl linker. “Lower alkoxycarbonyl” refers to an alkoxycarbonyl group wherein the alkoxy group is a lower alkoxy group. In some embodiments, C₁-C₄ alkoxy, is an alkoxy group which encompasses both straight and branched chain alkoxy groups of from 1 to 4 carbon atoms.

The term “substituted alkoxycarbonyl” refers to the group (substituted alkyl)-O—C(O)— wherein the group is attached to the parent structure through the carbonyl functionality. Unless stated otherwise specifically in the specification, the alkyl moiety of an alkoxycarbonyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Acyl” refers to the groups (alkyl)-C(O)—, (aryl)-C(O)—, (heteroaryl)-C(O)—, (heteroalkyl)-C(O)—, and (heterocycloalkyl)-C(O)—, wherein the group is attached to the parent structure through the carbonyl functionality. In some embodiments, it is a C₁-C₁₀ acyl radical which refers to the total number of chain or ring atoms of the alkyl, aryl, heteroaryl or heterocycloalkyl portion of the acyloxy group plus the carbonyl carbon of acyl, i.e., three other ring or chain atoms plus carbonyl. If the R radical is heteroaryl or heterocycloalkyl, the hetero ring or chain atoms contribute to the total number of chain or ring atoms. Unless stated otherwise specifically in the specification, the “R” of an acyloxy group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Acyloxy” refers to a R(C═O)O— radical wherein “R” is alkyl, aryl, heteroaryl, heteroalkyl, or heterocycloalkyl, which are as described herein. In some embodiments, it is a C₁-C₄ acyloxy radical which refers to the total number of chain or ring atoms of the alkyl, aryl, heteroaryl or heterocycloalkyl portion of the acyloxy group plus the carbonyl carbon of acyl, i.e., three other ring or chain atoms plus carbonyl. If the R radical is heteroaryl or heterocycloalkyl, the hetero ring or chain atoms contribute to the total number of chain or ring atoms. Unless stated otherwise specifically in the specification, the “R” of an acyloxy group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (wherein t is 1 or 2-S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Amino” or “amine” refers to a —N(R^{a})₂ radical group, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl, unless stated otherwise specifically in the specification. When a —N(R^{a})₂ group has two Ra other than hydrogen they can be combined with the nitrogen atom to form a 4-, 5-, 6-, or 7-membered ring. For example, —N(R^{a})₂ is meant to include, but not be limited to, 1-pyrrolidinyl and 4-morpholinyl. Unless stated otherwise specifically in the specification, an amino group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (wherein t is 1 or 2), —S(O)ₜOR^{a} (wherein t is 1 or 2), —S(O)N(R^{a})₂ (where t is 1 or 2), or PO₃(R^{a})₂, wherein each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl and each of these moieties can be optionally substituted as defined herein.

The term “substituted amino” also refers to N-oxides of the groups —NHR^{d}, and NR^{d}R^{d} each as described above. N-oxides can be prepared by treatment of the corresponding amino group with, for example, hydrogen peroxide or m-chloroperoxybenzoic acid. The person skilled in the art is familiar with reaction conditions for carrying out the N-oxidation.

“Amide” or “amido” refers to a chemical moiety with formula —C(O)N(R)₂ or —NHC(O)R, where R is selected from the group consisting of hydrogen, alkyl, cycloalkyl, aryl, heteroaryl (bonded through a ring carbon) and heteroalicyclic (bonded through a ring carbon), each of which moiety can itself be optionally substituted. In some embodiments it is a C₁-C₄ amido or amide radical, which includes the amide carbonyl in the total number of carbons in the radical. The R₂ of —N(R)₂ of the amide can optionally be taken together with the nitrogen to which it is attached to form a 4-, 5-, 6-, or 7-membered ring. Unless stated otherwise specifically in the specification, an amido group is optionally substituted independently by one or more of the substituents as described herein for alkyl, cycloalkyl, aryl, heteroaryl, or heterocycloalkyl. An amide can be an amino acid or a peptide molecule attached to a compound of Formula (I), thereby forming a prodrug. Any amine, hydroxy, or carboxyl side chain on the compounds described herein can be amidified. The procedures and specific groups to make such amides are known to those of skill in the art and can readily be found in reference sources such as Greene and Wuts, Protective Groups in Organic Synthesis, 3.sup.rd Ed., John Wiley & Sons, New York, N.Y., 1999, which is incorporated herein by reference in its entirety.

“Aromatic” or “aryl” refers to an aromatic radical with six to ten ring atoms (e.g., C₆-C₁₀ aromatic or C₆-C₁₀ aryl) which has at least one ring having a conjugated pi electron system which is carbocyclic (e.g., phenyl, fluorenyl, and naphthyl). Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene. Whenever it appears herein, a numerical range such as “6 to 10” refers to each integer in the given range; e.g., “6 to 10 ring atoms” means that the aryl group can consist of 6 ring atoms, 7 ring atoms, etc., up to and including 10 ring atoms. The term includes monocyclic or fused-ring polycyclic (i.e., rings which share adjacent pairs of ring atoms) groups. Unless stated otherwise specifically in the specification, an aryl moiety is optionally substituted by one or more substituents which are independently: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Aralkyl” or “arylalkyl” refers to an (aryl)alkyl-radical where aryl and alkyl are as disclosed herein and which are optionally substituted by one or more of the substituents described as suitable substituents for aryl and alkyl respectively.

“Ester” refers to a chemical radical of formula —COOR, where R is selected from the group consisting of alkyl, cycloalkyl, aryl, heteroaryl (bonded through a ring carbon) and heteroalicyclic (bonded through a ring carbon). Any amine, hydroxy, or carboxyl side chain on the compounds described herein can be esterified. The procedures and specific groups to make such esters are known to those of skill in the art and can readily be found in reference sources such as Greene and Wuts, Protective Groups in Organic Synthesis, 3.sup.rd Ed., John Wiley & Sons, New York, N.Y., 1999, which is incorporated herein by reference in its entirety. Unless stated otherwise specifically in the specification, an ester group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Fluoroalkyl” refers to an alkyl radical, as defined above, that is substituted by one or more fluoro radicals, as defined above, for example, trifluoromethyl, difluoromethyl, 2,2,2-trifluoroethyl, 1-fluoromethyl-2-fluoroethyl, and the like. The alkyl part of the fluoroalkyl radical can be optionally substituted as defined above for an alkyl group.

“Halo”, “halide”, or, alternatively, “halogen” means fluoro, chloro, bromo or iodo. The terms “haloalkyl,” “haloalkenyl,” “haloalkynyl” and “haloalkoxy” include alkyl, alkenyl, alkynyl and alkoxy structures that are substituted with one or more halo groups or with combinations thereof. For example, the terms “fluoroalkyl” and “fluoroalkoxy” include haloalkyl and haloalkoxy groups, respectively, in which the halo is fluorine.

“Heteroalkyl” “heteroalkenyl” and “heteroalkynyl” include optionally substituted alkyl, alkenyl and alkynyl radicals and which have one or more skeletal chain atoms selected from an atom other than carbon, e.g., oxygen, nitrogen, sulfur, phosphorus or combinations thereof. A numerical range can be given, e.g. C₁-C₄ heteroalkyl which refers to the chain length in total, which in this example is 4 atoms long. For example, a —CH₂OCH₂CH₃ radical is referred to as a “C₄” heteroalkyl, which includes the heteroatom center in the atom chain length description. Connection to the rest of the molecule can be through either a heteroatom or a carbon in the heteroalkyl chain. A heteroalkyl group can be substituted with one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Heteroalkylaryl” refers to an -(heteroalkyl)aryl radical where heteroalkyl and aryl are as disclosed herein and which are optionally substituted by one or more of the substituents described as suitable substituents for heteroalkyl and aryl respectively.

“Heteroalkylheteroaryl” refers to an -(heteroalkyl)heteroaryl radical where heteroalkyl and heteroaryl are as disclosed herein and which are optionally substituted by one or more of the substituents described as suitable substituents for heteroalkyl and heteroaryl respectively.

“Heteroalkylheterocycloalkyl” refers to an -(heteroalkyl)heterocycloalkyl radical where heteroalkyl and heteroaryl are as disclosed herein and which are optionally substituted by one or more of the substituents described as suitable substituents for heteroalkyl and heterocycloalkyl respectively

“Heteroalkylcycloalkyl” refers to an -(heteroalkyl)cycloalkyl radical where heteroalkyl and cycloalkyl are as disclosed herein and which are optionally substituted by one or more of the substituents described as suitable substituents for heteroalkyl and cycloalkyl respectively.

“Heteroaryl” or, alternatively, “heteroaromatic” refers to a 5- to 18-membered aromatic radical (e.g., C₅-C₁₃ heteroaryl) that includes one or more ring heteroatoms selected from nitrogen, oxygen and sulfur, and which can be a monocyclic, bicyclic, tricyclic or tetracyclic ring system. Whenever it appears herein, a numerical range such as “5 to 18” refers to each integer in the given range; e.g., “5 to 18 ring atoms” means that the heteroaryl group can consist of 5 ring atoms, 6 ring atoms, etc., up to and including 18 ring atoms. Bivalent radicals derived from univalent heteroaryl radicals whose names end in “-yl” by removal of one hydrogen atom from the atom with the free valence are named by adding “-idene” to the name of the corresponding univalent radical, e.g., a pyridyl group with two points of attachment is a pyridylidene. An N-containing “heteroaromatic” or “heteroaryl” moiety refers to an aromatic group in which at least one of the skeletal atoms of the ring is a nitrogen atom. The polycyclic heteroaryl group can be fused or non-fused. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzoxazolyl, benzopyranyl, benzopyranonyl, benzofuranyl, benzopyranonyl, benzofurazanyl, benzothiazolyl, benzothienyl(benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furazanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyranyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, thiapyranyl, triazolyl, tetrazolyl, triazinyl, thieno[2,3-d]pyrimidinyl, thieno[3,2-d]pyrimidinyl, thieno[2,3-c]pridinyl, and thiophenyl (i.e., thienyl). Unless stated otherwise specifically in the specification, a heteraryl moiety is optionally substituted by one or more substituents which are independently: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2), —S(O)ₜN(R^{a})₂ (wherein t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

Substituted heteroaryl also includes ring systems substituted with one or more oxide (—O—) substituents, such as pyridinyl N-oxides.

“Heteroarylalkyl” refers to a moiety having an aryl moiety, as described herein, connected to an alkylene moiety, as described herein, wherein the connection to the remainder of the molecule is through the alkylene group.

“Heterocycloalkyl” refers to a stable 3- to 18-membered non-aromatic ring radical that comprises two to twelve carbon atoms and from one to six heteroatoms selected from nitrogen, oxygen and sulfur. Whenever it appears herein, a numerical range such as “3 to 18” refers to each integer in the given range; e.g., “3 to 18 ring atoms” means that the heterocycloalkyl group can consist of 3 ring atoms, 4 ring atoms, etc., up to and including 18 ring atoms. In some embodiments, it is a C₅-C₁₀ heterocycloalkyl. In some embodiments, it is a C₄-C₁₀ heterocycloalkyl. In some embodiments, it is a C₃-C₁₀ heterocycloalkyl. Unless stated otherwise specifically in the specification, the heterocycloalkyl radical is a monocyclic, bicyclic, tricyclic or tetracyclic ring system, which can include fused or bridged ring systems. The heteroatoms in the heterocycloalkyl radical can be optionally oxidized. One or more nitrogen atoms, if present, are optionally quaternized. The heterocycloalkyl radical is partially or fully saturated. The heterocycloalkyl can be attached to the rest of the molecule through any atom of the ring(s). Examples of such heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocycloalkyl moiety is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heteroaryl or heteroarylalkyl.

“Heterocycloalkyl” also includes bicyclic ring systems wherein one non-aromatic ring, usually with 3 to 7 ring atoms, contains at least 2 carbon atoms in addition to 1-3 heteroatoms independently selected from oxygen, sulfur, and nitrogen, as well as combinations comprising at least one of the foregoing heteroatoms; and the other ring, usually with 3 to 7 ring atoms, optionally contains 1-3 heteroatoms independently selected from oxygen, sulfur, and nitrogen and is not aromatic.

“Isomers” are different compounds that have the same molecular formula. “Stereoisomers” are isomers that differ only in the way the atoms are arranged in space, i.e., having a different stereochemical configuration. “Enantiomers” are a pair of stereoisomers that are non-superimposable mirror images of each other. A 1:1 mixture of a pair of enantiomers is a “racemic” mixture. The term “(.±.)” is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer the stereochemistry at each chiral carbon can be specified by either R or S. Resolved compounds whose absolute configuration is unknown can be designated (+) or (−) depending on the direction (dextro- or levorotatory) which they rotate plane polarized light at the wavelength of the sodium D line. Certain of the compounds described herein contain one or more asymmetric centers and can thus give rise to enantiomers, diastereomers, and other stereoisomeric forms that can be defined, in terms of absolute stereochemistry, as (R)- or (S)-. The present chemical entities, pharmaceutical compositions and methods are meant to include all such possible isomers, including racemic mixtures, optically pure forms and intermediate mixtures. Optically active (R)- and (S)-isomers can be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers.

“Enantiomeric purity” as used herein refers to the relative amounts, expressed as a percentage, of the presence of a specific enantiomer relative to the other enantiomer. For example, if a compound, which can potentially have an (R)- or an (S)-isomeric configuration, is present as a racemic mixture, the enantiomeric purity is about 50% with respect to either the (R)- or (S)-isomer. If that compound has one isomeric form predominant over the other, for example, 80% (S)- and 20% (R)-, the enantiomeric purity of the compound with respect to the (S)-isomeric form is 80%. The enantiomeric purity of a compound can be determined in a number of ways known in the art, including but not limited to chromatography using a chiral support, polarimetric measurement of the rotation of polarized light, nuclear magnetic resonance spectroscopy using chiral shift reagents which include but are not limited to lanthanide containing chiral complexes or the Pirkle alcohol, or derivatization of a compounds using a chiral compound such as Mosher's acid followed by chromatography or nuclear magnetic resonance spectroscopy.

“Moiety” refers to a specific segment or functional group of a molecule. Chemical moieties are often recognized chemical entities embedded in or appended to a molecule.

“Nitro” refers to the —NO₂ radical.

“Oxa” refers to the —O— radical.

“Oxo” refers to the ═O radical.

“Tautomers” are structurally distinct isomers that interconvert by tautomerization. “Tautomerization” is a form of isomerization and includes prototropic or proton-shift tautomerization, which is considered a subset of acid-base chemistry. “Prototropic tautomerization” or “proton-shift tautomerization” involves the migration of a proton accompanied by changes in bond order, often the interchange of a single bond with an adjacent double bond. Where tautomerization is possible (e.g., in solution), a chemical equilibrium of tautomers can be reached. An example of tautomerization is keto-enol tautomerization. A specific example of keto-enol tautomerization is the interconversion of pentane-2,4-dione and 4-hydroxypent-3-en-2-one tautomers. Another example of tautomerization is phenol-keto tautomerization. A specific example of phenol-keto tautomerization is the interconversion of pyridin-4-ol and pyridin-4(1H)-one tautomers.

The compounds of the present invention can also contain unnatural proportions of atomic isotopes at one or more of atoms that constitute such compounds. For example, the compounds can be radiolabeled with radioactive isotopes, such as for example tritium (³H), iodine-125 (¹²⁵I) or carbon-14 (¹⁴C). All isotopic variations of the compounds of the present invention, whether radioactive or not, are encompassed within the scope of the present invention.

A “leaving group or atom” is any group or atom that will, under the reaction conditions, cleave from the starting material, thus promoting reaction at a specified site. Suitable examples of such groups unless otherwise specified are halogen atoms, mesyloxy, p-nitrobenzensulphonyloxy and tosyloxy groups.

“Protecting group” has the meaning conventionally associated with it in organic synthesis, i.e., a group that selectively blocks one or more reactive sites in a multifunctional compound such that a chemical reaction can be carried out selectively on another unprotected reactive site and such that the group can readily be removed after the selective reaction is complete. A variety of protecting groups are disclosed, for example, in T. H. Greene and P. G. M. Wuts, Protective Groups in Organic Synthesis, Third Edition, John Wiley & Sons, New York (1999). For example, a hydroxy protected form is where at least one of the hydroxy groups present in a compound is protected with a hydroxy protecting group. Likewise, amines and other reactive groups can similarly be protected.

“Solvate” refers to a compound (e.g., a compound selected from Formula I or a pharmaceutically acceptable salt thereof) in physical association with one or more molecules of a pharmaceutically acceptable solvent. It will be understood that “a compound of Formula I” encompass the compound of Formula I and solvates of the compound, as well as mixtures thereof.

“Substituted” means that the referenced group can be substituted with one or more additional group(s) individually and independently selected from acyl, alkyl, alkylaryl, cycloalkyl, aralkyl, aryl, carbohydrate, carbonate, heteroaryl, heterocycloalkyl, hydroxy, alkoxy, aryloxy, mercapto, alkylthio, arylthio, cyano, halo, carbonyl, ester, thiocarbonyl, isocyanato, thiocyanato, isothiocyanato, nitro, oxo, perhaloalkyl, perfluoroalkyl, phosphate, silyl, sulfinyl, sulfonyl, sulfonamidyl, sulfoxyl, sulfonate, urea, and amino, including mono- and di-substituted amino groups, and the protected derivatives thereof. Di-substituted amino groups encompass those which form a ring together with the nitrogen of the amino group, such as for instance, morpholino. The substituents themselves can be substituted, for example, a cycloakyl substituent can have a halide substituted at one or more ring carbons, and the like. The protecting groups that can form the protective derivatives of the above substituents are known to those of skill in the art and can be found in references such as Greene and Wuts, above.

“Sulfanyl” refers to the groups: —S-(optionally substituted alkyl), —S-(optionally substituted aryl), —S-(optionally substituted heteroaryl), and —S-(optionally substituted heterocycloalkyl).

“Sulfinyl” refers to the groups: —S(O)—H, —S(O)-(optionally substituted alkyl), —S(O)-(optionally substituted amino), —S(O)-(optionally substituted aryl), —S(O)-(optionally substituted heteroaryl), and —S(O)-(optionally substituted heterocycloalkyl).

“Sulfonyl” refers to the groups: —S(O₂)—H, —S(O₂)-(optionally substituted alkyl), —S(O₂)-(optionally substituted amino), —S(O₂)-(optionally substituted aryl), —S(O₂)-(optionally substituted heteroaryl), and —S(O₂)-(optionally substituted heterocycloalkyl).

“Sulfonamidyl” or “sulfonamido” refers to a —S(═O)₂—NRR radical, where each R is selected independently from the group consisting of hydrogen, alkyl, cycloalkyl, aryl, heteroaryl (bonded through a ring carbon) and heteroalicyclic (bonded through a ring carbon). The R groups in —NRR of the —S(═O)₂—NRR radical can be taken together with the nitrogen to which it is attached to form a 4-, 5-, 6-, or 7-membered ring. In some embodiments, it is a C₁-C₁₀ sulfonamido, wherein each R in sulfonamido contains 1 carbon, 2 carbons, 3 carbons, or 4 carbons total. A sulfonamido group is optionally substituted by one or more of the substituents described for alkyl, cycloalkyl, aryl, heteroaryl respectively

“Sulfoxyl” refers to a —S(═O)₂OH radical.

“Sulfonate” refers to a —S(═O)₂—OR radical, where R is selected from the group consisting of alkyl, cycloalkyl, aryl, heteroaryl (bonded through a ring carbon) and heteroalicyclic (bonded through a ring carbon). A sulfonate group is optionally substituted on R by one or more of the substituents described for alkyl, cycloalkyl, aryl, and heteroaryl, respectively.

Where substituent groups are specified by their conventional chemical formulae, written from left to right, they equally encompass the chemically identical substituents that would result from writing the structure from right to left, e.g., —CH₂O— is equivalent to —OCH₂—.

Compounds that can be used as described herein also include crystalline and amorphous forms of compounds, including, for example, polymorphs, pseudopolymorphs, solvates, hydrates, unsolvated polymorphs (including anhydrates), conformational polymorphs, and amorphous forms of the compounds, as well as mixtures thereof.

As used herein, and unless otherwise specified, “polymorph” can be used herein to describe a crystalline material, e.g., a crystalline form. In certain embodiments, “polymorph” as used herein are also meant to include all crystalline and amorphous forms of a compound or a salt thereof, including, for example, crystalline forms, polymorphs, pseudopolymorphs, solvates, hydrates, co-crystals, unsolvated polymorphs (including anhydrates), conformational polymorphs, tautomeric forms, disordered crystalline forms, and amorphous forms, as well as mixtures thereof, unless a particular crystalline or amorphous form is referred to. Compounds of the present disclosure include crystalline and amorphous forms of those compounds, including, for example, crystalline forms, polymorphs, pseudopolymorphs, solvates, hydrates, co-crystals, unsolvated polymorphs (including anhydrates), conformational polymorphs, tautomeric forms, disordered crystalline forms, and amorphous forms of the compounds or a salt thereof, as well as mixtures thereof.

Chemical entities include, but are not limited to, compounds of Formula I, I-1, IV, IV-A, V, V-A, V-A2, V-B, VI or VI-A, and all pharmaceutically acceptable forms thereof. Pharmaceutically acceptable forms of the compounds recited herein include pharmaceutically acceptable salts, chelates, non-covalent complexes, prodrugs, and mixtures thereof. In certain embodiments, the compounds described herein are in the form of pharmaceutically acceptable salts. Hence, the terms “chemical entity” and “chemical entities” also encompass pharmaceutically acceptable salts, chelates, non-covalent complexes, prodrugs, and mixtures.

In addition, if the compound of Formula I is obtained as an acid addition salt, the free base can be obtained by basifying a solution of the acid salt. Conversely, if the product is a free base, an addition salt, particularly a pharmaceutically acceptable addition salt, can be produced by dissolving the free base in a suitable organic solvent and treating the solution with an acid, in accordance with conventional procedures for preparing acid addition salts from base compounds. Those skilled in the art will recognize various synthetic methodologies that can be used to prepare non-toxic pharmaceutically acceptable addition salts.

### Compounds

The compounds provided below are exemplary PI3K inhibitors that can be used in the pharmaceutical compositions, methods and kits disclosed herein.

In some aspects, the PI3K inhibitor is a compound of Formula I:

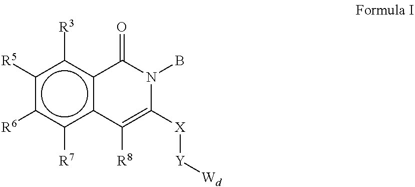

or its pharmaceutically acceptable salt thereof, wherein
W_{d} is heterocycloalkyl, aryl or heteroaryl;
B is alkyl, amino, heteroalkyl, or a moiety of Formula II;

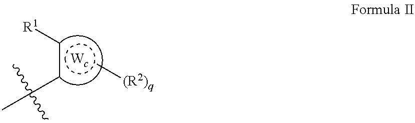

wherein W_{c} is aryl, heteroaryl, heterocycloalkyl, or cycloalkyl, and
q is an integer of 0, 1, 2, 3, or 4;
X is absent or is —(CH(R⁹))_{z} and z is an integer of 1, 2, 3, or 4;
Y is absent, —O—, —S—, —S(═O)—, —S(═O)₂—, —N(R⁹)—, —C(═O)—(CHR⁹)_{z}—, —C(C═O)—, —N(R⁹)—C(C═O)—, or —N(R⁹)—C(═O)NH—, —N(R⁹)C(R⁹)₂—, or —C(C═O)—(CHR⁹)_{z}—;
R¹ is hydrogen, alkyl, heteroalkyl, alkenyl, alkynyl, cycloalkyl, heterocycloalkyl, aryl, arylalkyl, heteroaryl, heteroarylalkyl, alkoxy, amido, amino, acyl, acyloxy, alkoxycarbonyl, sulfonamido, halo, cyano, hydroxy, nitro, phosphate, urea, or carbonate;
R² is alkyl, heteroalkyl, alkenyl, alkynyl, cycloalkyl, heterocycloalkyl, aryl, arylalkyl, heteroaryl, heteroarylalkyl, alkoxy, amido, amino, acyl, acyloxy, alkoxycarbonyl, sulfonamido, halo, cyano, hydroxy, nitro, phosphate, urea, or carbonate;
R³ is hydrogen, alkyl, alkenyl, alkynyl, cycloalkyl, heterocycloalkyl, alkoxy, amido, amino, acyl, acyloxy, alkoxycarbonyl, sulfonamido, halo, cyano, hydroxy, nitro, aryl, or heteroaryl;
R⁵, R⁶, R⁷, and R⁸ are independently hydrogen, C₁-C₄alkyl, C₂-C₅alkenyl, C₂-C₅alkynyl, C₃-C₅cycloalkyl, C₁-C₄heteroalkyl, C₁-C₄alkoxy, C₁-C₄amido, amino, acyl, C₁-C₄acyloxy, C₁-C₄sulfonamido, halo, cyano, hydroxy or nitro; and
each instance of R⁹ is independently hydrogen, C₁-C₁₀alkyl, C₃-C₇cycloalkyl, heterocycloalkyl, or C₂-C₁₀heteroalkyl.

In some embodiments, B is unsubstituted or substituted alkyl, including but not limited to —(CH₂)₂—NR^{a}R^{a}, wherein each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl, or NR^{a}R^{a} are combined together to form a cyclic moiety, which includes but is not limited to piperidinyl, piperazinyl, and morpholinyl. In some embodiments, B is unsubstituted or substituted amino. In some embodiments, B is unsubstituted or substituted heteroalkyl.

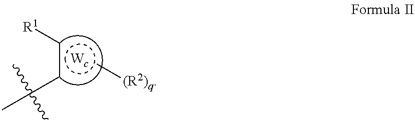

In some embodiments, B is a moiety of Formula II and wherein W_{c} is a member selected from the group consisting of unsubstituted or substituted aryl, substituted phenyl, unsubstituted or substituted heteroaryl including but not limited to pyridin-2-yl, pyridin-3-yl, pyridin-4-yl, pyrimidin-4-yl, pyrimidin-2-yl, pyrimidin-5-yl, or pyrazin-2-yl, unsubstituted or substituted monocyclic heteroaryl, unsubstituted or substituted bicyclic heteroaryl, a heteroaryl comprising two heteroatoms as ring atoms, unsubstituted or substituted heteroaryl comprising a nitrogen ring atom, heteroaryl comprising two nitrogen ring atoms, heteroaryl comprising a nitrogen and a sulfur as ring atoms, unsubstituted or substituted heterocycloalkyl including but not limited to morpholinyl, tetrahydropyranyl, piperazinyl, and piperidinyl, unsubstituted or substituted cycloalkyl including but not limited to cyclopentyl and cyclohexyl.

In some embodiments, B is one of the following moieties:

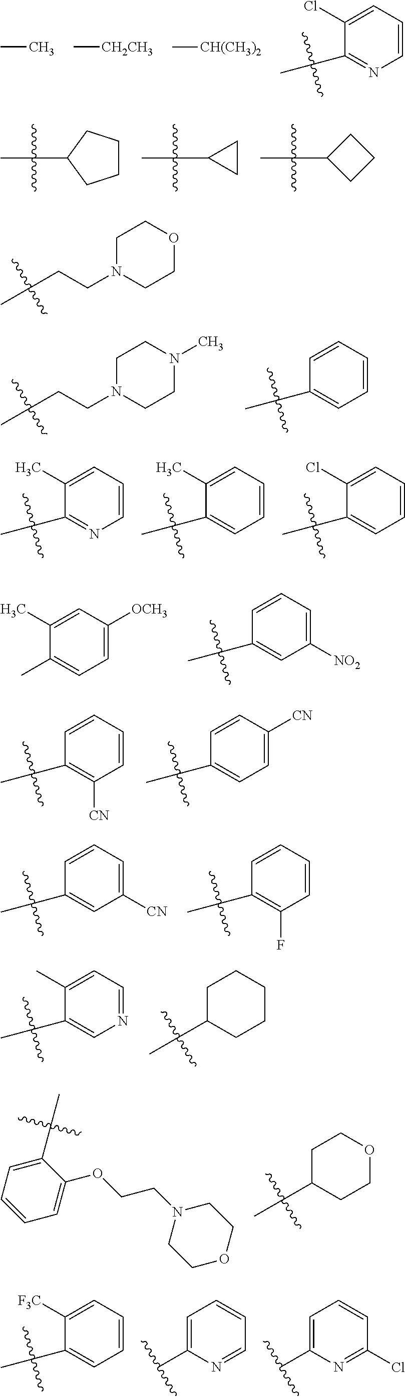

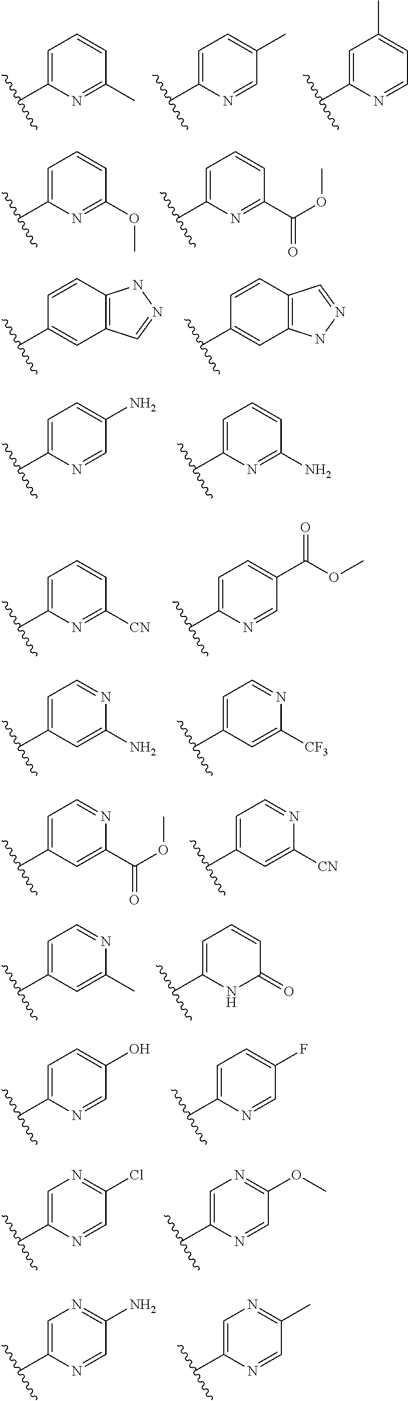

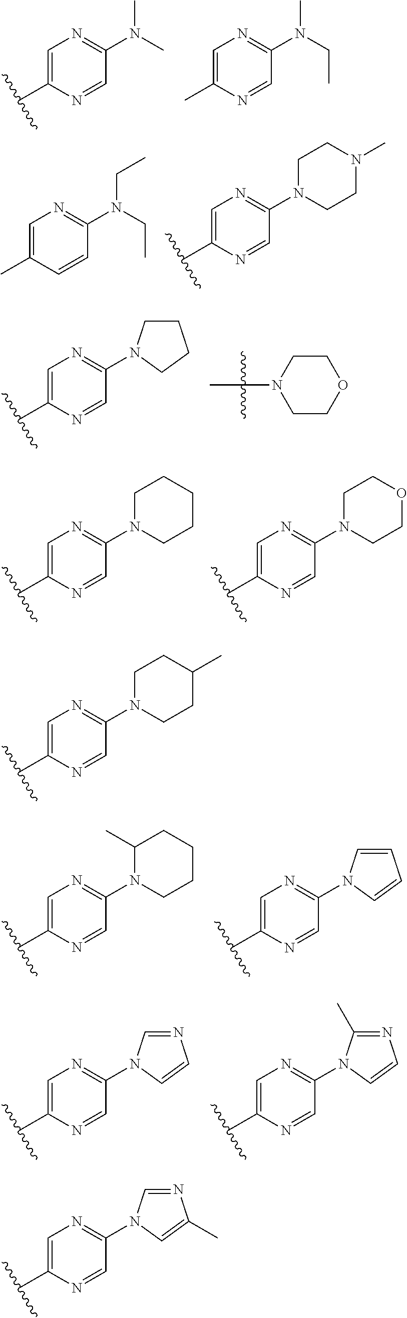

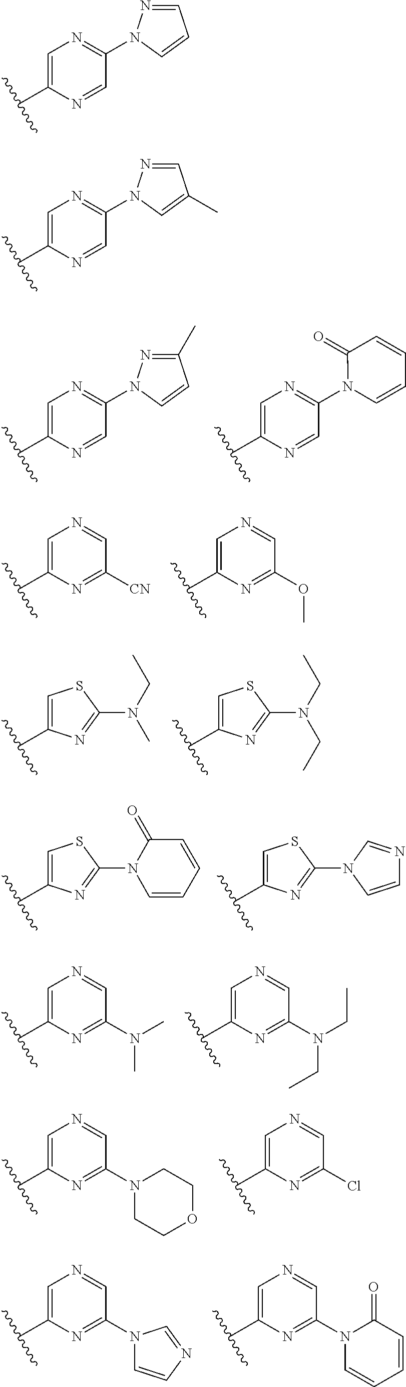

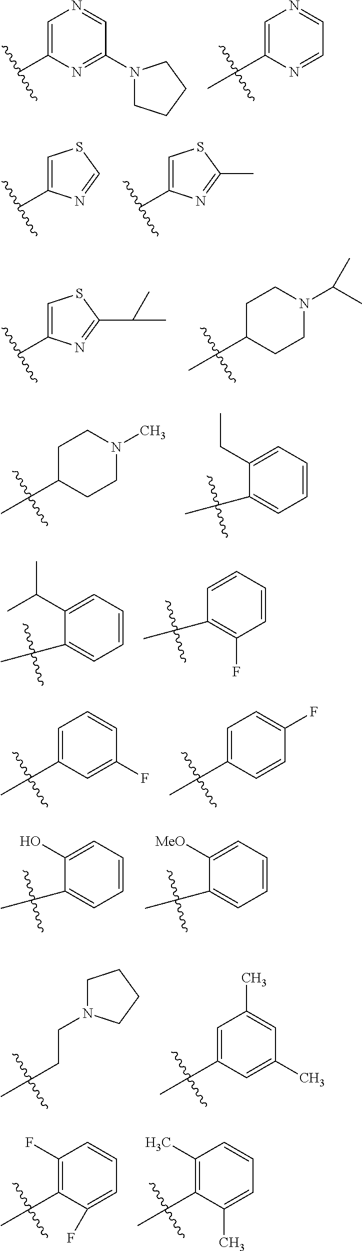

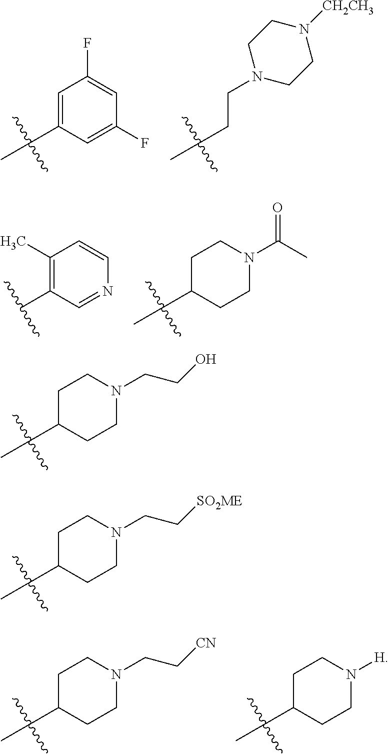

In some embodiments, B is substituted by one or more of alkyl, heteroalkyl, alkenyl, alkynyl, cycloalkyl, heterocycloalkyl, aryl, heteroaryl alkoxy, amido, amino, acyl, acyloxy, alkoxycarbonyl, sulfonamido, halo, cyano, hydroxy or nitro, each of which alkyl, heteroalkyl, alkenyl, alkynyl, cycloalkyl, heterocycloalkyl, aryl, heteroaryl, alkoxy, amido, amino, acyl, acyloxy, or sulfonamido, can itself be substituted.

In some embodiments, R¹ is a member selected from the group consisting of hydrogen, unsubstituted or substituted alkyl, unsubstituted or substituted heteroalkyl, unsubstituted or substituted alkenyl, unsubstituted or substituted alkynyl, unsubstituted or substituted cycloalkyl, or unsubstituted or substituted heterocycloalkyl. In some embodiments, R¹ is unsubstituted or substituted aryl, unsubstituted or substituted arylalkyl, unsubstituted or substituted heteroaryl, or unsubstituted or substituted heteroarylalkyl. In some embodiments, R¹ is unsubstituted or substituted alkoxy, unsubstituted or substituted amido, unsubstituted or substituted amino. In some embodiments, R¹ is unsubstituted or substituted acyl, unsubstituted or substituted acyloxy, unsubstituted or substituted alkoxycarbonyl, or unsubstituted or substituted sulfonamido. In some embodiments, R¹ is halo which includes —Cl, —F, —I, and —Br. In some embodiments, R¹ is selected from the group consisting of cyano, hydroxy, nitro, unsubstituted or substituted phosphate, unsubstituted or substituted urea, and carbonate.

In some embodiments, when R¹ is alkyl, R¹ is methyl, ethyl, propyl, isopropyl, n-butyl, tert-butyl, sec-butyl, pentyl, hexyl or heptyl.

In some embodiments, when R¹ is alkyl, heteroalkyl, alkenyl, alkynyl, cycloalkyl, heterocycloalkyl, aryl, arylalkyl, heteroaryl, heteroarylalkyl, alkoxy, amido, amino, acyl, acyloxy, alkoxycarbonyl, sulfonamido, or hydroxy, R¹ is substituted by phosphate, or unsubstituted urea, or substituted urea, or carbonic acid, or carbonate.

In some embodiments, when R¹ is alkyl, heteroalkyl, alkenyl, alkynyl, cycloalkyl, heterocycloalkyl, aryl, arylalkyl, heteroaryl, heteroarylalkyl, alkoxy, amido, amino, acyl, acyloxy, alkoxycarbonyl, or sulfonamido, R¹ is substituted by one or more of alkyl, heteroalkyl, alkenyl, alkynyl, cycloalkyl, heterocycloalkyl, aryl, arylalkyl, heteroaryl, heteroarylalkyl, alkoxy, amido, amino, acyl, acyloxy, alkoxycarbonyl, sulfonamido, halo, cyano, hydroxy or nitro, each of which alkyl, heteroalkyl, alkenyl, alkynyl, cycloalkyl, heterocycloalkyl, aryl, arylalkyl, heteroaryl, heteroarylalkyl, alkoxy, amido, amino, acyl, acyloxy, alkoxycarbonyl, or sulfonamido can itself be substituted.

In some embodiments, R² is a member selected from the group consisting of unsubstituted or substituted alkyl, unsubstituted or substituted heteroalkyl, unsubstituted or substituted alkenyl, unsubstituted or substituted alkynyl, unsubstituted or substituted cycloalkyl, and unsubstituted or substituted heterocycloalkyl. In some embodiments, R² is unsubstituted or substituted aryl, unsubstituted or substituted arylalkyl, unsubstituted or substituted heteroaryl, or unsubstituted or substituted heteroarylalkyl. In some embodiments, R² is unsubstituted or substituted alkoxy, unsubstituted or substituted amido, unsubstituted or substituted amino. In some embodiments, R² is unsubstituted or substituted acyl, unsubstituted or substituted acyloxy, unsubstituted or substituted alkoxycarbonyl, or unsubstituted or substituted sulfonamido. In some embodiments, R² is halo, which is —I, —F, —Cl, or —Br. In some embodiments, R² is selected from the group consisting of cyano, hydroxy, nitro, a carbonic acid, and a carbonate. In some embodiments, R² is unsubstituted or substituted phosphate. In some embodiments, R² is unsubstituted or substituted urea. In some embodiments, when R² is alkyl, R² is methyl, ethyl, propyl, isopropyl, n-butyl, tert-butyl, sec-butyl, pentyl, hexyl or heptyl.

In some embodiments, when R² is alkyl, heteroalkyl, alkenyl, alkynyl, cycloalkyl, heterocycloalkyl, aryl, arylalkyl, heteroaryl, heteroarylalkyl, alkoxy, amido, amino, acyl, acyloxy, alkoxycarbonyl, sulfonamido, or hydroxy, it is substituted by phosphate, substituted by urea, or substituted by carbonate.

In some embodiments, when R² is alkyl, heteroalkyl, alkenyl, alkynyl, cycloalkyl, heterocycloalkyl, aryl, arylalkyl, heteroaryl, heteroarylalkyl, alkoxy, amido, amino, acyl, acyloxy, alkoxycarbonyl, or sulfonamido, it is substituted by one or more of alkyl, heteroalkyl, alkenyl, alkynyl, cycloalkyl, heterocycloalkyl, aryl, heteroaryl, alkoxy, amido, amino, acyl, acyloxy, alkoxycarbonyl, sulfonamido, halo, cyano, hydroxy or nitro, each of which alkyl, heteroalkyl, alkenyl, alkynyl, cycloalkyl, heterocycloalkyl, aryl, heteroaryl, alkoxy, amido, amino, acyl, acyloxy, alkoxycarbonyl, or sulfonamido can itself be substituted.

In some embodiments, q is an integer of 0. In some embodiments, q is an integer of 1. In some embodiments, q is an integer of 2. In some embodiments, q is an integer of 3. In some embodiments, q is an integer of 4.

In some embodiments of the compound of Formula I, R³ is a member selected from the group consisting of hydrogen, unsubstituted or substituted alkyl, unsubstituted or substituted alkenyl, and unsubstituted or substituted alkynyl. In some embodiments, R³ is unsubstituted or substituted aryl, unsubstituted or substituted heteroaryl, unsubstituted or substituted cycloalkyl, or unsubstituted or substituted heterocycloalkyl. In some embodiments, R³ is unsubstituted or substituted alkoxy, unsubstituted or substituted amido, unsubstituted or substituted amino. In some embodiments, R³ is unsubstituted or substituted acyl, unsubstituted or substituted acyloxy, unsubstituted or substituted alkoxycarbonyl, or unsubstituted or substituted sulfonamido. In some embodiments, R³ is halo, which is is —I, —F, —Cl, or —Br.

In some embodiments, R³ is selected from the group consisting of cyano, hydroxy, and nitro. In some embodiments, when R³ is alkyl, R³ is methyl, ethyl, propyl, isopropyl, n-butyl, tert-butyl, sec-butyl, pentyl, hexyl or heptyl. In some embodiments, R³ is —CF₃.

In some embodiments, when R³ is alkyl, alkenyl, alkynyl, aryl, heteroaryl, cycloalkyl, heterocycloalkyl, alkoxy, amido, amino, acyl, acyloxy, alkoxycarbonyl, or sulfonamido, it is substituted with one or more of alkyl, heteroalkyl, alkenyl, alkynyl, cycloalkyl, heterocycloalkyl, aryl, heteroaryl, alkoxy, amido, amino, acyl, acyloxy, alkoxycarbonyl, sulfonamido, halo, cyano, hydroxy or nitro, each of which alkyl, heteroalkyl, alkenyl, alkynyl, cycloalkyl, heterocycloalkyl, aryl, heteroaryl, alkoxy, amido, amino, acyl, acyloxy, alkoxycarbonyl, or sulfonamido can itself be substituted.

In some embodiments of the compound of Formula I, R⁵ is hydrogen, unsubstituted or substituted alkyl (including but not limited to unsubstituted or substituted C₁-C₄alkyl). In some embodiments, R⁵ is unsubstituted or substituted alkenyl including but not limited to unsubstituted or substituted C₂-C₅alkenyl. In some embodiments, R⁵ is unsubstituted or substituted alkynyl including but not limited to unsubstituted or substituted C₂-C₅alkynyl. In some embodiments, R⁵ is unsubstituted or substituted cycloalkyl including but not limited to unsubstituted or substituted C₃-C₅cycloalkyl. In some embodiments, R⁵ is unsubstituted or substituted heterocycloalkyl. In some embodiments, R⁵ is unsubstituted or substituted heteroalkyl including but not limited to unsubstituted or substituted C₁-C₄heteroalkyl. In some embodiments, R⁵ is unsubstituted or substituted alkoxy including but not limited to unsubstituted or substituted C₁-C₄alkoxy. In some embodiments, R⁵ is unsubstituted or substituted amido including but not limited to unsubstituted or substituted C₁-C₄amido. In some embodiments, R⁵ is unsubstituted or substituted amino. In some embodiments, R⁵ is unsubstituted or substituted acyl, unsubstituted or substituted acyloxy, unsubstituted or substituted C₁-C₄acyloxy, unsubstituted or substituted alkoxycarbonyl, unsubstituted or substituted sulfonamido, or unsubstituted or substituted C₁-C₄sulfonamido. In some embodiments, R⁵ is halo, which is —I, —F, —Cl, or —Br. In some embodiments, R⁵ is selected from the group consisting of cyano, hydroxy, and nitro. In some other embodiments, R⁵ is —CH₃, —CH₂CH₃, n-propyl, isopropyl, —OCH₃, —OCH₂CH₃, or —CF₃.

In some embodiments, when R⁵ is alkyl, alkenyl, alkynyl, cycloalkyl, heteroalkyl, acyl, alkoxy, amido, amino, acyloxy, alkoxycarbonyl, or sulfonamido, R⁵ is optionally substituted with one or more of alkyl, heteroalkyl, alkenyl, alkynyl, cycloalkyl, heterocycloalkyl, aryl, heteroaryl, alkoxy, amido, amino, acyl, acyloxy, alkoxycarbonyl, sulfonamido, halo, cyano, hydroxy or nitro, each of which alkyl, heteroalkyl, alkenyl, alkynyl, cycloalkyl, heterocycloalkyl, aryl, heteroaryl, alkoxy, amido, amino, acyl, acyloxy, alkoxycarbonyl, or sulfonamido can itself be substituted.

In some embodiments of the compound of Formula I, R⁶ is hydrogen, unsubstituted or substituted alkyl (including but not limited to unsubstituted or substituted C₁-C₄alkyl). In some embodiments, R⁶ is unsubstituted or substituted alkenyl including but not limited to unsubstituted or substituted C₂-C₅alkenyl. In some embodiments, R⁶ is unsubstituted or substituted alkynyl including but not limited to unsubstituted or substituted C₂-C₅alkynyl. In some embodiments, R⁶ is unsubstituted or substituted cycloalkyl including but not limited to unsubstituted or substituted C₃-C₅cycloalkyl. In some embodiments, R⁶ is unsubstituted or substituted heterocycloalkyl. In some embodiments, R⁶ is unsubstituted or substituted heteroalkyl including but not limited to unsubstituted or substituted C₁-C₄heteroalkyl. In some embodiments, R⁶ is unsubstituted or substituted alkoxy including but not limited to unsubstituted or substituted C₁-C₄alkoxy. In some embodiments, R⁶ is unsubstituted or substituted amido including but not limited to unsubstituted or substituted C₁-C₄amido. In some embodiments, R⁶ is unsubstituted or substituted amino. In some embodiments, R⁶ is unsubstituted or substituted acyl, unsubstituted or substituted acyloxy, unsubstituted or substituted C₁-C₄acyloxy, unsubstituted or substituted alkoxycarbonyl, unsubstituted or substituted sulfonamido, or unsubstituted or substituted C₁-C₄sulfonamido. In some embodiments, R⁶ is halo, which is —I, —F, —Cl, or —Br. In some embodiments, R⁶ is selected from the group consisting of cyano, hydroxy, and nitro. In some other embodiments, R⁶ is —CH₃, —CH₂CH₃, n-propyl, isopropyl, —OCH₃, —OCH₂CH₃, or —CF₃.

In some embodiments, when R⁶ is alkyl, alkenyl, alkynyl, cycloalkyl, heteroalkyl, acyl, alkoxy, amido, amino, acyloxy, alkoxycarbonyl, or sulfonamido, R⁶ is optionally substituted with one or more of alkyl, heteroalkyl, alkenyl, alkynyl, cycloalkyl, heterocycloalkyl, aryl, heteroaryl, alkoxy, amido, amino, acyl, acyloxy, alkoxycarbonyl, sulfonamido, halo, cyano, hydroxy or nitro, each of which alkyl, heteroalkyl, alkenyl, alkynyl, cycloalkyl, heterocycloalkyl, aryl, heteroaryl, alkoxy, amido, amino, acyl, acyloxy, alkoxycarbonyl, or sulfonamido can itself be substituted.

In some embodiments of the compound of Formula I, R⁷ is hydrogen, unsubstituted or substituted alkyl (including but not limited to unsubstituted or substituted C₁-C₄alkyl). In some embodiments, R⁷ is unsubstituted or substituted alkenyl including but not limited to unsubstituted or substituted C₂-C₅alkenyl. In some embodiments, R⁷ is unsubstituted or substituted alkynyl including but not limited to unsubstituted or substituted C₂-C₅alkynyl. In some embodiments, R⁷ is unsubstituted or substituted cycloalkyl including but not limited to unsubstituted or substituted C₃-C₅cycloalkyl. In some embodiments, R⁷ is unsubstituted or substituted heterocycloalkyl. In some embodiments, R⁷ is unsubstituted or substituted heteroalkyl including but not limited to unsubstituted or substituted C₁-C₄heteroalkyl. In some embodiments, R⁷ is unsubstituted or substituted alkoxy including but not limited to unsubstituted or substituted C₁-C₄alkoxy. In some embodiments, R⁷ is unsubstituted or substituted amido including but not limited to unsubstituted or substituted C₁-C₄amido. In some embodiments, R⁷ is unsubstituted or substituted amino. In some embodiments, R⁷ is unsubstituted or substituted acyl, unsubstituted or substituted acyloxy, unsubstituted or substituted C₁-C₄acyloxy, unsubstituted or substituted alkoxycarbonyl, unsubstituted or substituted sulfonamido, or unsubstituted or substituted C₁-C₄sulfonamido. In some embodiments, R⁷ is halo, which is —I, —F, —Cl, or —Br. In some embodiments, R⁷ is selected from the group consisting of cyano, hydroxy, and nitro. In some other embodiments, R⁷ is —CH₃, —CH₂CH₃, n-propyl, isopropyl, —OCH₃, —OCH₂CH₃, or —CF₃.

In some embodiments, when R⁷ is alkyl, alkenyl, alkynyl, cycloalkyl, heteroalkyl, acyl, alkoxy, amido, amino, acyloxy, alkoxycarbonyl, or sulfonamido, R⁷ is optionally substituted with one or more of alkyl, heteroalkyl, alkenyl, alkynyl, cycloalkyl, heterocycloalkyl, aryl, heteroaryl, alkoxy, amido, amino, acyl, acyloxy, alkoxycarbonyl, sulfonamido, halo, cyano, hydroxy or nitro, each of which alkyl, heteroalkyl, alkenyl, alkynyl, cycloalkyl, heterocycloalkyl, aryl, heteroaryl, alkoxy, amido, amino, acyl, acyloxy, alkoxycarbonyl, or sulfonamido can itself be substituted.

In some embodiments of the compound of Formula I, R⁸ is hydrogen, unsubstituted or substituted alkyl (including but not limited to unsubstituted or substituted C₁-C₄alkyl). In some embodiments, R⁸ is unsubstituted or substituted alkenyl including but not limited to unsubstituted or substituted C₂-C₅alkenyl. In some embodiments, R⁸ is unsubstituted or substituted alkynyl including but not limited to unsubstituted or substituted C₂-C₅alkynyl. In some embodiments, R⁸ is unsubstituted or substituted cycloalkyl including but not limited to unsubstituted or substituted C₃-C₅cycloalkyl. In some embodiments, R⁸ is unsubstituted or substituted heterocycloalkyl. In some embodiments, R⁸ is unsubstituted or substituted heteroalkyl including but not limited to unsubstituted or substituted C₁-C₄heteroalkyl. In some embodiments, R⁸ is unsubstituted or substituted alkoxy including but not limited to unsubstituted or substituted C₁-C₄alkoxy. In some embodiments, R⁸ is unsubstituted or substituted amido including but not limited to unsubstituted or substituted C₁-C₄amido. In some embodiments, R⁸ is unsubstituted or substituted amino. In some embodiments, R⁸ is unsubstituted or substituted acyl, unsubstituted or substituted acyloxy, unsubstituted or substituted C₁-C₄acyloxy, unsubstituted or substituted alkoxycarbonyl, unsubstituted or substituted sulfonamido, or unsubstituted or substituted C₁-C₄sulfonamido. In some embodiments, R⁸ is halo, which is —I, —F, —Cl, or —Br. In some embodiments, R⁸ is selected from the group consisting of cyano, hydroxy, and nitro. In some other embodiments, R⁸ is —CH₃, —CH₂CH₃, n-propyl, isopropyl, —OCH₃, —OCH₂CH₃, or —CF₃.

In some embodiments, when R⁸ is alkyl, alkenyl, alkynyl, cycloalkyl, heteroalkyl, acyl, alkoxy, amido, amino, acyloxy, alkoxycarbonyl, or sulfonamido, R⁸ is optionally substituted with one or more of alkyl, heteroalkyl, alkenyl, alkynyl, cycloalkyl, heterocycloalkyl, aryl, heteroaryl, alkoxy, amido, amino, acyl, acyloxy, alkoxycarbonyl, sulfonamido, halo, cyano, hydroxy or nitro, each of which alkyl, heteroalkyl, alkenyl, alkynyl, cycloalkyl, heterocycloalkyl, aryl, heteroaryl, alkoxy, amido, amino, acyl, acyloxy, alkoxycarbonyl, or sulfonamido can itself be substituted.

In some embodiments of the compound of Formula I, R⁵, R⁶, R⁷, and R⁸ are H and the compound has a structure of Formula I-1:

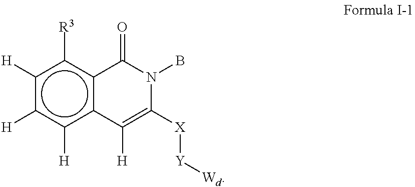

In some embodiments of the compound of Formula I, X is absent. In some embodiments, X is —(CH(R⁹))_{z}, and z is an integer of 1, 2, 3 or 4.

In some embodiments, R⁹ is unsubstituted or substituted alkyl including but not limited to unsubstituted or substituted C₁-C₁₀alkyl. In some embodiments, R⁹ is unsubstituted or substituted cycloalkyl including but not limited to unsubstituted or substituted C₃-C₇cycloalkyl. In some embodiments, R⁹ is ethyl, methyl or hydrogen. In some embodiments, R⁹ is unsubstituted or substituted heterocycloalkyl including but not limited to unsubstituted or substituted C₂-C₁₀heteroalkyl. In some embodiments, R⁹ is unsubstituted or substituted heteroalkyl including but not limited to unsubstituted or substituted C₂-C₁₀heteroalkyl.

The invention also provides a compound of Formula I wherein R⁹ is hydrogen, and X is —CH₂—, —CH₂CH₂—, —CH₂CH₂CH₂—, —CH(CH₃)—, or —CH(CH₂CH₃)—. In other embodiments, X is —(CH(R⁹))_{z}, R⁹ is not hydrogen, and z is an integer of 1. When X is —CH(R⁹)— and R⁹ is not hydrogen, then the compound can adopt either an (S)- or (R)-stereochemical configuration with respect to carbon X. In some embodiments, the compound is a racemic mixture of (S)- and (R) isomers with respect to carbon X. In other embodiments, the present invention provides a mixture of compounds of Formula I wherein individual compounds of the mixture exist predominately in an (S)- or (R)-isomeric configuration. For example, the compound mixture has an (S)-enantiomeric purity of greater than about 55%, about 60%, about 65%, about 70%, about 75%, about 80%, about 85%, about 90%, about 95%, about 96%, about 97%, about 98%, about 99%, about 99.5%, or more at the X carbon. In other embodiments, the compound mixture has an (S)-enantiomeric purity of greater than about 55% to about 99.5%, greater than about 60% to about 99.5%, greater than about 65% to about 99.5%, greater than about 70% to about 99.5%, greater than about 75% to about 99.5%, greater than about 80% to about 99.5%, greater than about 85% to about 99.5%, greater than about 90% to about 99.5%, greater than about 95% to about 99.5%, greater than about 96% to about 99.5%, greater than about 97% to about 99.5%, greater than about 98% to greater than about 99.5%, greater than about 99% to about 99.5%, or more.

In other embodiments, the compound mixture has an (R)-enantiomeric purity of greater than about 55%, about 60%, about 65%, about 70%, about 75%, about 80%, about 85%, about 90%, about 95%, about 96%, about 97%, about 98%, about 99%, about 99.5%, or more at the X carbon. In some other embodiments, the compound mixture has an (R)-enantiomeric purity of greater than about 55% to about 99.5%, greater than about 60% to about 99.5%, greater than about 65% to about 99.5%, greater than about 70% to about 99.5%, greater than about 75% to about 99.5%, greater than about 80% to about 99.5%, greater than about 85% to about 99.5%, greater than about 90% to about 99.5%, greater than about 95% to about 99.5%, greater than about 96% to about 99.5%, greater than about 97% to about 99.5%, greater than about 98% to greater than about 99.5%, greater than about 99% to about 99.5%, or more.

In other embodiments, the compound mixture contains identical chemical entities except for their stereochemical orientations, namely (S)- or (R)-isomers. For instance, in the compounds of Formula I, when X is —CH(R⁹)—, and R⁹ is not hydrogen, then the —CH(R⁹)— is in an (S)- or (R)-sterochemical orientation for each of the identical chemical entities. In some embodiments, the mixture of identical chemical entities of Formula I is a racemic mixture of (S)- and (R)-isomers at the carbon represented by X. In another embodiment, the mixture of the identical chemical entities (except for their stereochemical orientations) contain predominately (S)-isomers or predominately (R)-isomers. For example, the (S)-isomers in the mixture of identical chemical entities are present at about 55%, about 60%, about 65%, about 70%, about 75%, about 80%, about 85%, about 90%, about 95%, about 96%, about 97%, about 98%, about 99%, about 99.5%, or more, relative to the (R)-isomers. In some embodiments, the (S)-isomers in the mixture of identical chemical entities are present at an (S)-enantiomeric purity of greater than about 55% to about 99.5%, greater than about 60% to about 99.5%, greater than about 65% to about 99.5%, greater than about 70% to about 99.5%, greater than about 75% to about 99.5%, greater than about 80% to about 99.5%, greater than about 85% to about 99.5%, greater than about 90% to about 99.5%, greater than about 95% to about 99.5%, greater than about 96% to about 99.5%, greater than about 97% to about 99.5%, greater than about 98% to greater than about 99.5%, greater than about 99% to about 99.5%, or more.

In another embodiment, the (R)-isomers in the mixture of identical chemical entities (except for their stereochemical orientations), are present at about 55%, about 60%, about 65%, about 70%, about 75%, about 80%, about 85%, about 90%, about 95%, about 96%, about 97%, about 98%, about 99%, about 99.5%, or more, relative to the (S)-isomers. In some embodiments, the (R)-isomers in the mixture of identical chemical entities (except for their stereochemical orientations), are present at a (R)-enantiomeric purity greater than about 55% to about 99.5%, greater than about 60% to about 99.5%, greater than about 65% to about 99.5%, greater than about 70% to about 99.5%, greater than about 75% to about 99.5%, greater than about 80% to about 99.5%, greater than about 85% to about 99.5%, greater than about 90% to about 99.5%, greater than about 95% to about 99.5%, greater than about 96% to about 99.5%, greater than about 97% to about 99.5%, greater than about 98% to greater than about 99.5%, greater than about 99% to about 99.5%, or more.

In some embodiments, the compound of Formula I, X is —CH(R⁹)—, R⁹ is methyl or ethyl, and the compound is the (S)-isomer.

In some embodiments of the compound of Formula I, Y is absent. In some embodiments, Y is —O—, —S—, —S(═O)—, —S(═O)₂—, —C(C═O)—, —N(R⁹)(C═O)—, —N(R⁹)(C═O)NH—, —N(R⁹)C(R⁹)₂— (such as —N(R⁹)CH₂—, specifically —N(CH₃)CH₂—, N(CH(CH₃)₂)CH₂— or N(CH₂CH₃)CH₂—), —N(R⁹)—, —N(CH₃)—, —N(CH₂CH₃)—, or —N(CH(CH₃)₂)—. In some embodiments, Y is —C(═O)—(CHR⁹)_{z}— and z is an integer of 1, 2, 3, or 4.

In some embodiments, at least one of X and Y is present. In some embodiments of the compound of Formula I, —XY— is —CH₂—, —CH₂—N(CH₃), —CH₂—N(CH₂CH₃), —CH(CH₃)—NH—, (S)—CH(CH₃)—NH—, or (R)—CH(CH₃)—NH—. In other embodiments, X—Y is —N(CH₃)—CH₂—, N(CH₂CH₃) CH₂—, —N(CH(CH₃)₂)CH₂—, or —NHCH₂—. The invention provides other compounds of Formula I wherein when X—Y is X is —(CH(R⁹))_{z}N(R⁹)—, z is an integer of 1, 2, 3 or 4, and —N(R⁹)— is not —NH—, then —XY— is not connected to purinyl.

In some embodiments, W_{d} in a formula disclosed herein (including but not limited to I, I-1, IV, IV-A, V, V-A, V-A2, V-B, VI and VI-A), is a member selected from the group consisting of unsubstituted or substituted heterocycloalkyl, unsubstituted or substituted aryl, and unsubstituted or substituted heteroaryl.

In various embodiments, W_{d} is unsubstituted or substituted monocyclic heteroaryl (including but not limited to pyrimidinyl, pyrrolyl, pyrazinyl, triazinyl, or pyridazinyl) or unsubstituted or substituted bicyclic heteroaryl.

In some embodiments, W_{d} is a monocyclic heteroaryl of the following formula:

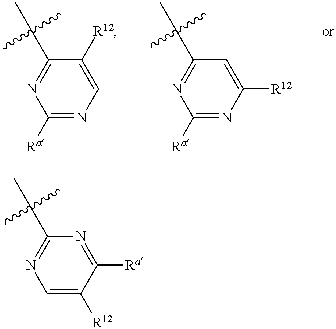

wherein R^{a′} is hydrogen, halo, phosphate, urea, a carbonate, unsubstituted or substituted amino, unsubstituted or substituted alkyl, unsubstituted or substituted alkenyl, unsubstituted or substituted alkynyl, unsubstituted or substituted cycloalkyl, unsubstituted or substituted heteroalkyl, or unsubstituted or substituted heterocycloalkyl; and R¹² is H, unsubstituted or substituted alkyl, unsubstituted or substituted cyano, unsubstituted or substituted alkynyl, unsubstituted or substituted alkenyl, halo, unsubstituted or substituted aryl, unsubstituted or substituted heteroaryl, unsubstituted or substituted heterocycloalkyl, unsubstituted or substituted cycloalkyl, unsubstituted or substituted amino, carboxylic acid, unsubstituted or substituted alkoxycarbonyl, unsubstituted or substituted amido, unsubstituted or substituted acyl, or unsubstituted or substituted sulfonamido.

Also included herein are compounds having monocyclic heteroaryl W_{d} including but not limited to one of the following formulae:

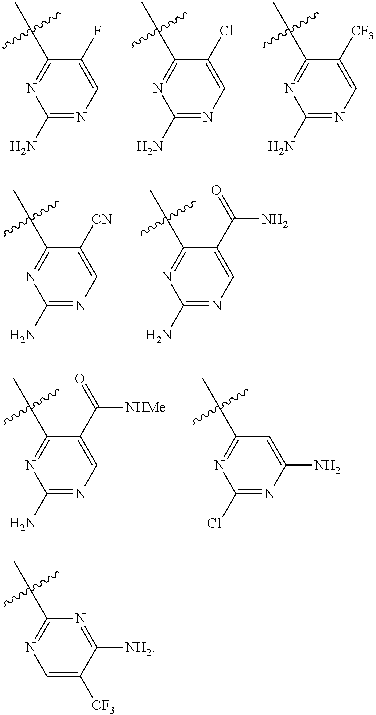

In some embodiments, W_{d} in a formula disclosed herein (including but not limited to I, I-1, IV, IV-A, V, V-A, V-A2, V-B, VI and VI-A), is a bicyclic heteroaryl having at least one heteroatom, e.g., a bicyclic heteroaryl having at least one nitrogen ring atom. In some embodiments, W_{d} is a bicyclic heteroaryl having at least two heteroatoms, e.g., a bicyclic heteroaryl having at least two nitrogen ring atoms. In some embodiments, W_{d} is a bicyclic heteroaryl having two heteroatoms in the ring which is connected to XY. In some embodiments, W_{d} is a bicyclic heteroaryl having two nitrogen ring atoms in the ring to which XY is connected. In some embodiments, W_{d} is a bicyclic heteroaryl having four heteroatoms, e.g., a bicyclic heteroaryl having four nitrogen ring atoms. In some embodiments, W_{d} is unsubstituted or substituted 4-amino-1H-pyrazolo[3,4-d]pyrimidin-1-yl, unsubstituted or substituted 7-amino-2-methyl-2H-pyrazolo[4,3-d]pyrimidin-3-yl. unsubstituted or substituted 6-methylenyl-9H-purin-6-yl, or unsubstituted or substituted 6-amino-9H-purin-9-yl.

In some embodiments W_{d} is one of the following:

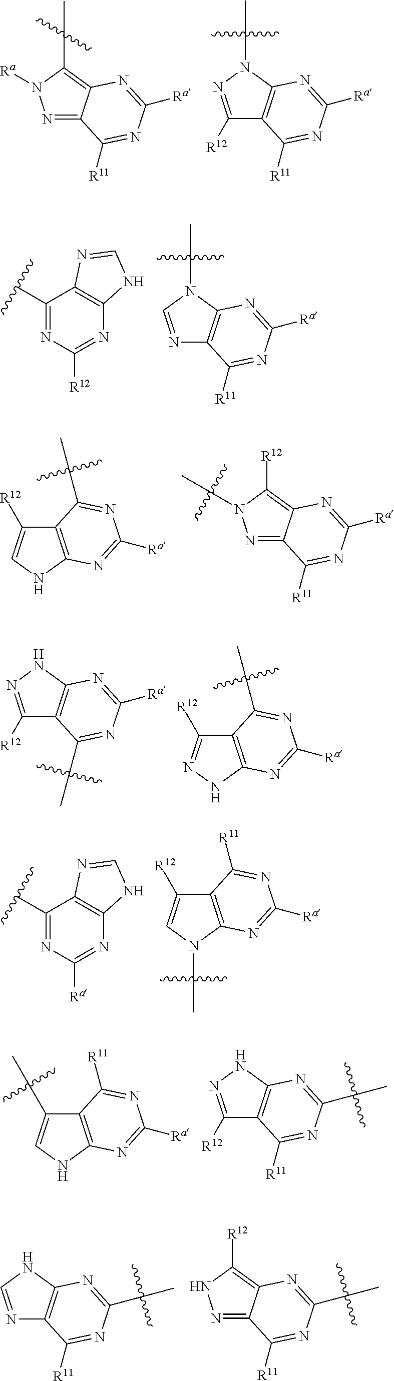

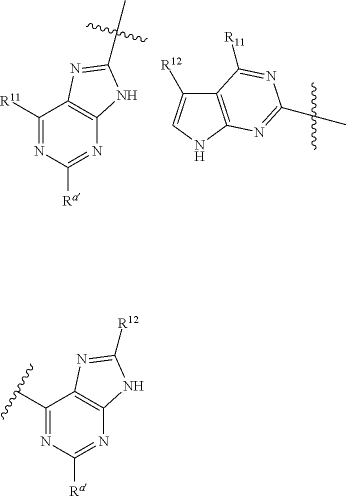

wherein R^{a′} is hydrogen, halo, phosphate, urea, a carbonate, unsubstituted or substituted amino, unsubstituted or substituted alkyl, unsubstituted or substituted alkenyl, unsubstituted or substituted alkynyl, unsubstituted or substituted cycloalkyl, unsubstituted or substituted heteroalkyl, or unsubstituted or substituted heterocycloalkyl;
R¹¹ is hydrogen, unsubstituted or substituted alkyl, halo (which includes —I, —F, —Cl, or —Br), unsubstituted or substituted amino, unsubstituted or substituted amido, hydroxy, or unsubstituted or substituted alkoxy, phosphate, unsubstituted or substituted urea, or carbonate; and
R¹² is H, unsubstituted or substituted alkyl, unsubstituted or substituted cyano, unsubstituted or substituted alkynyl, unsubstituted or substituted alkenyl, halo, unsubstituted or substituted aryl, unsubstituted or substituted heteroaryl, unsubstituted or substituted heterocycloalkyl, unsubstituted or substituted cycloalkyl, unsubstituted or substituted amino, carboxylic acid, unsubstituted or substituted alkoxycarbonyl, unsubstituted or substituted amido, unsubstituted or substituted acyl, or unsubstituted or substituted sulfonamido.

In some embodiments of W_{d} of the compounds of Formula I, when R^{a′} is alkyl, alkynyl, cycloalkyl, heteroalkyl, or heterocycloalkyl, it is substituted by phosphate, urea, or carbonate.

In some embodiments of W_{d} of the compounds of Formula I, when R¹¹ is alkyl, amino, amido, hydroxy, or alkoxy, it is substituted by phosphate, urea, or carbonate.

In some embodiments of the compound of Formula I, —X—Y—W_{d} is one of the following moieties:

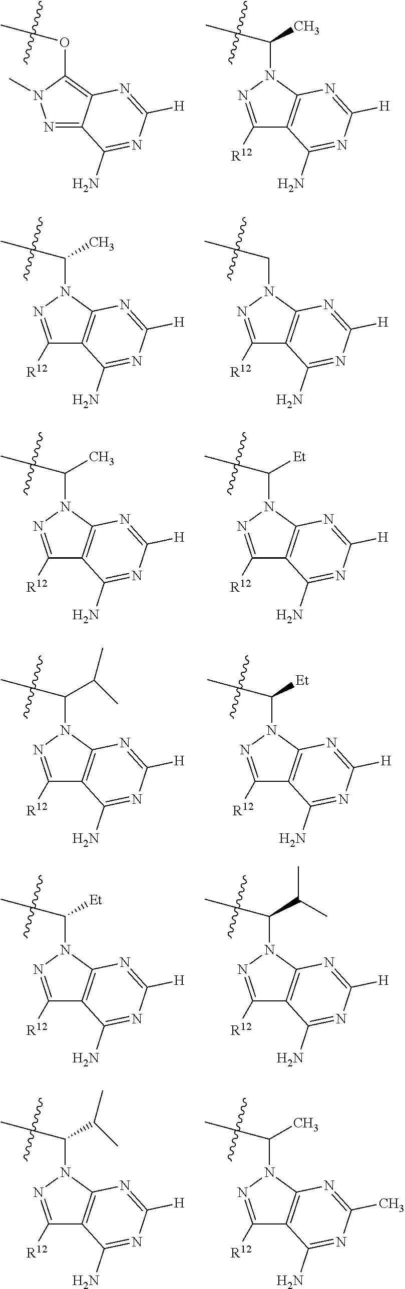

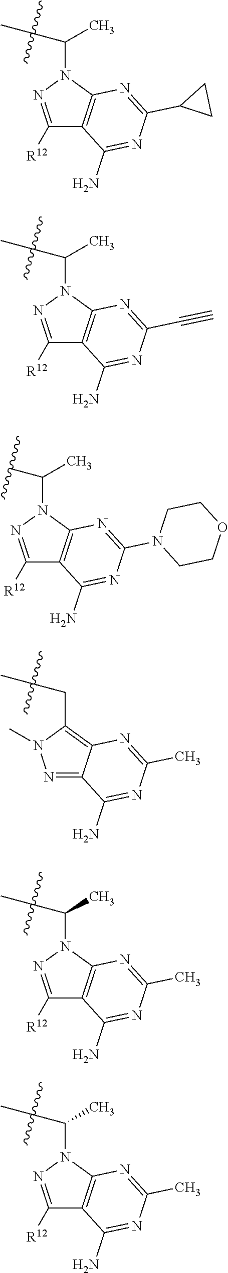

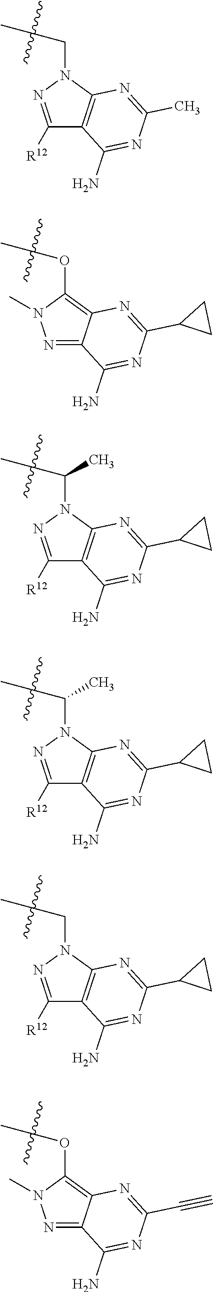

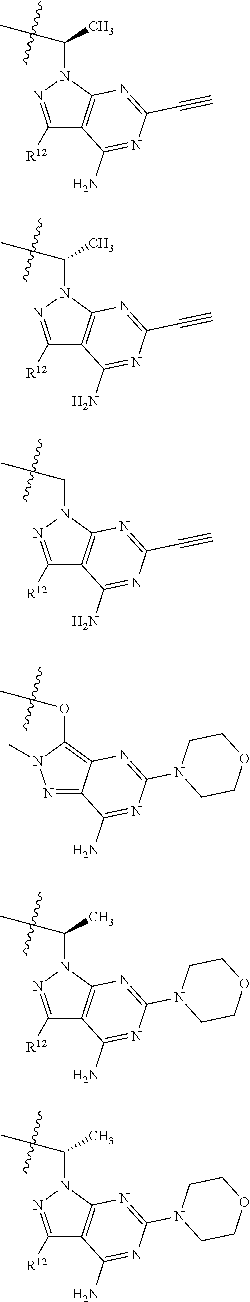

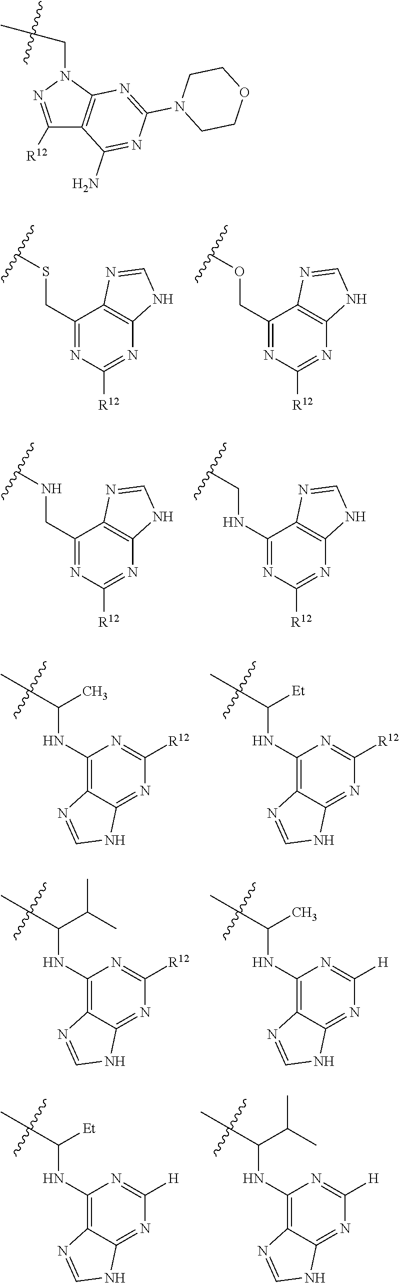

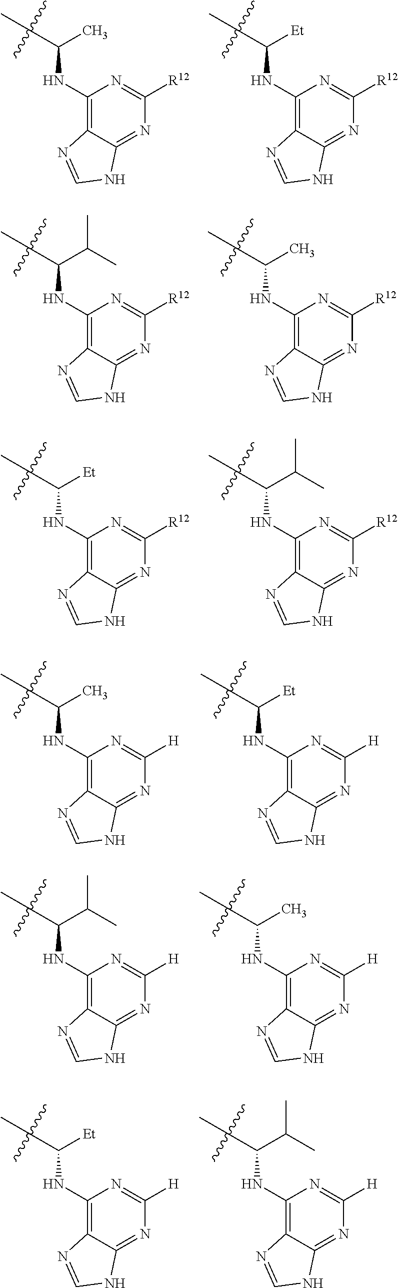

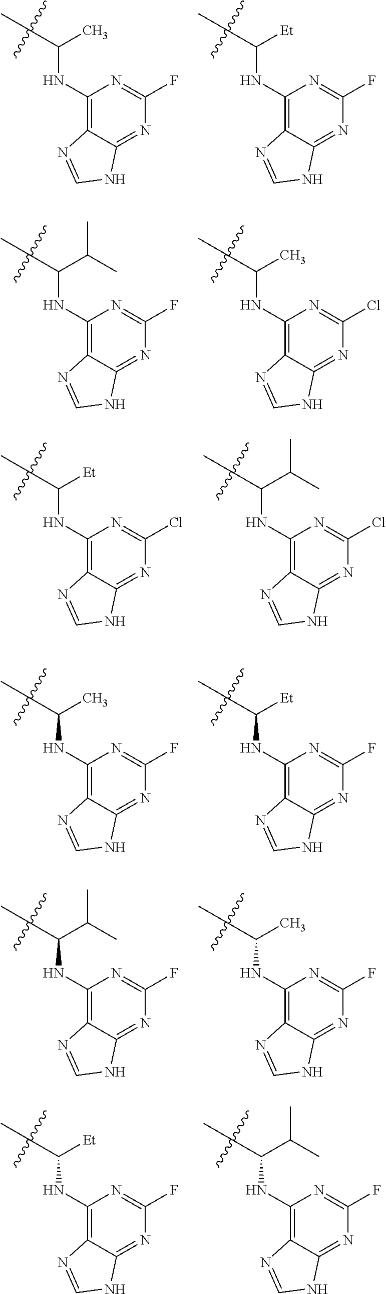

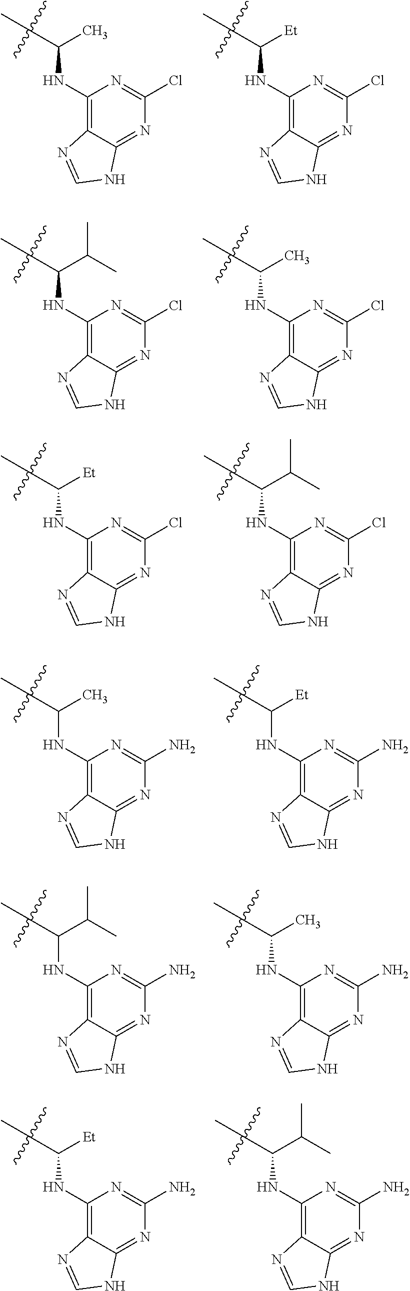

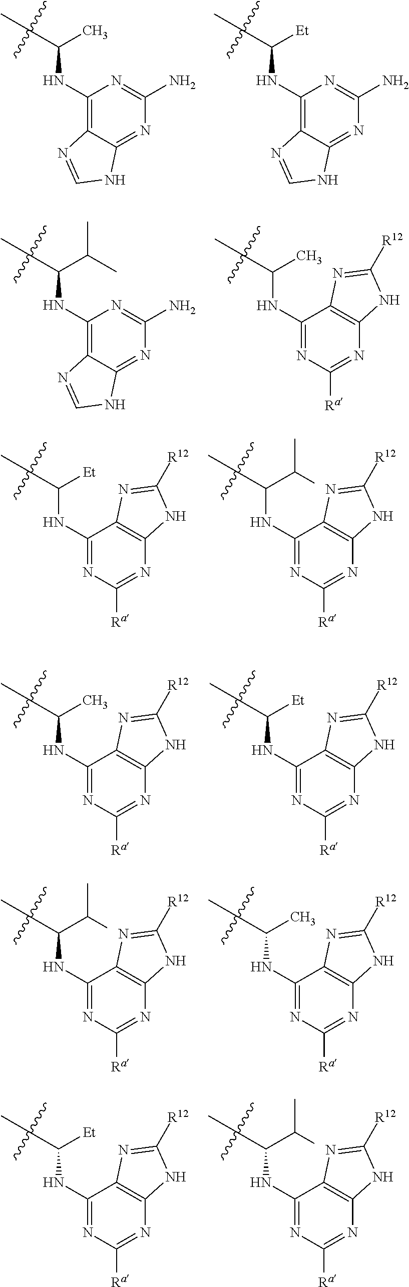

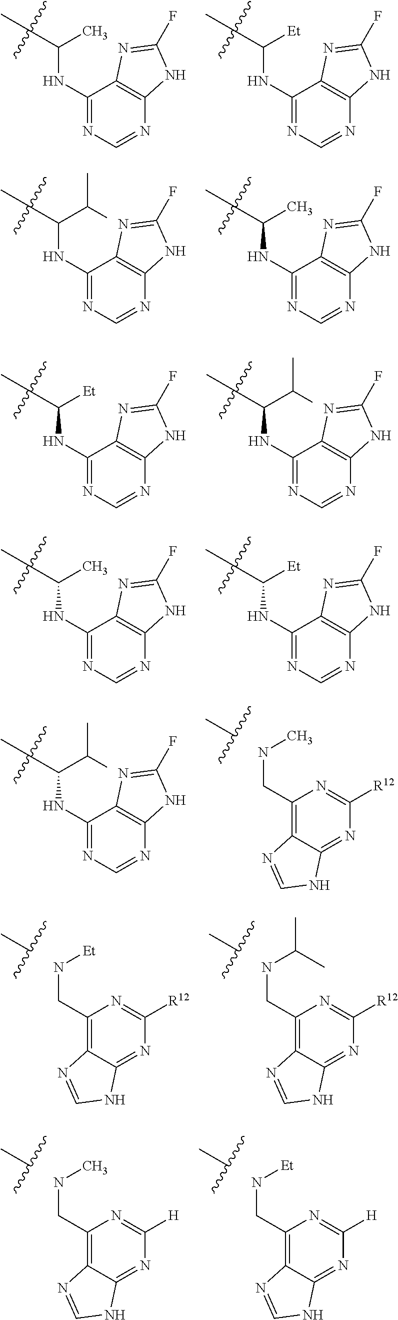

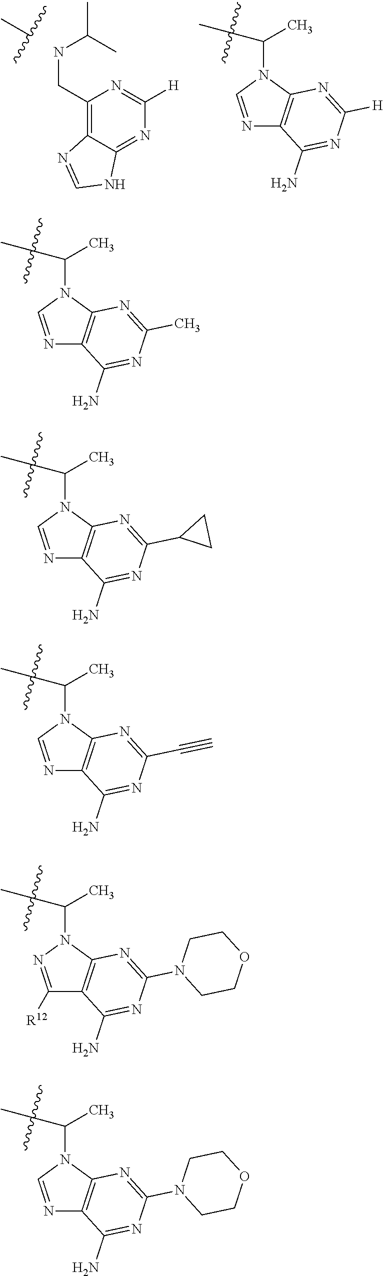

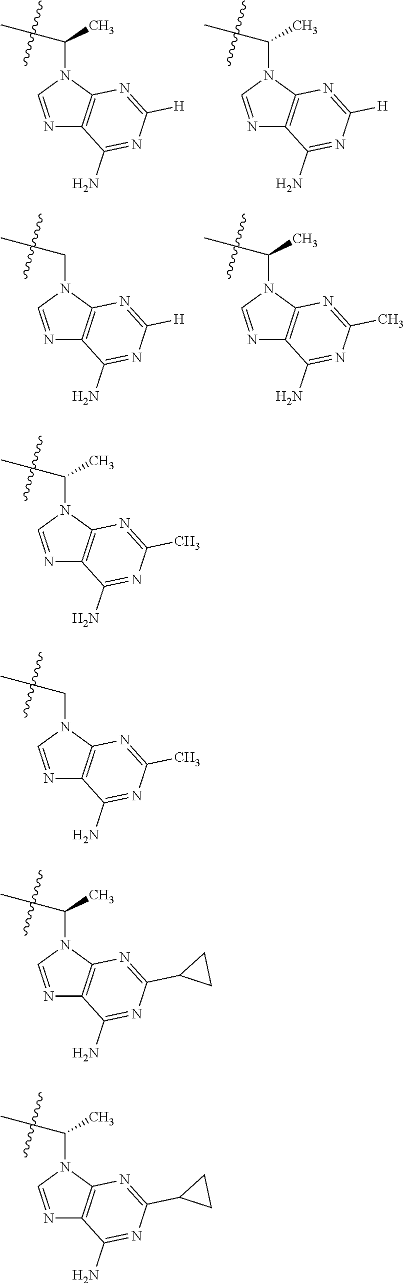

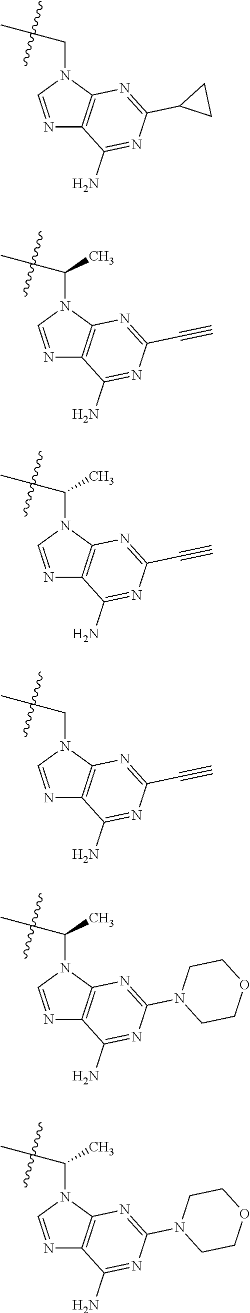 [SMILES: *Cn1cnc2c(N)nc(C3CC3)nc21 ; *[C@@H](C)n1cnc2c(N)nc(C#C)nc21 ; *[C@H](C)n1cnc2c(N)nc(C#C)nc21 ; *Cn1cnc2c(N)nc(C#C)nc21 ; *[C@@H](C)n1cnc2c(N)nc(N3CCOCC3)nc21 ; *[C@H](C)n1cnc2c(N)nc(N3CCOCC3)nc21]

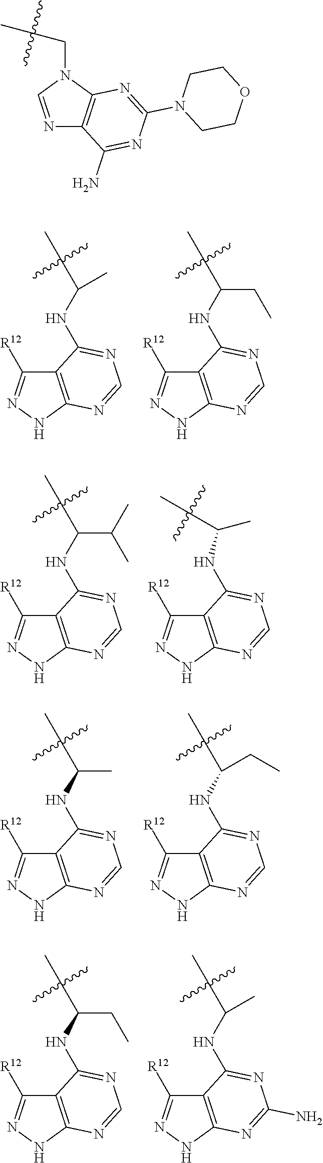

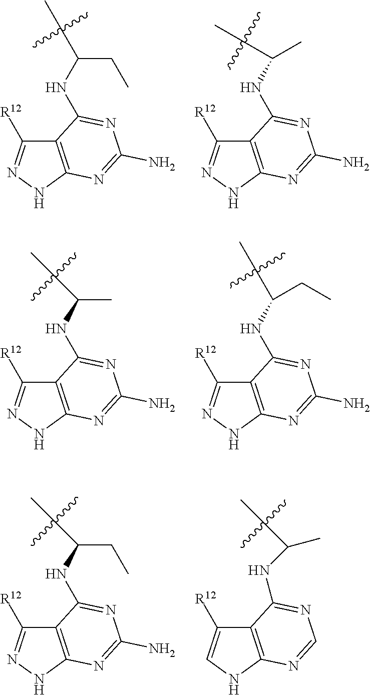

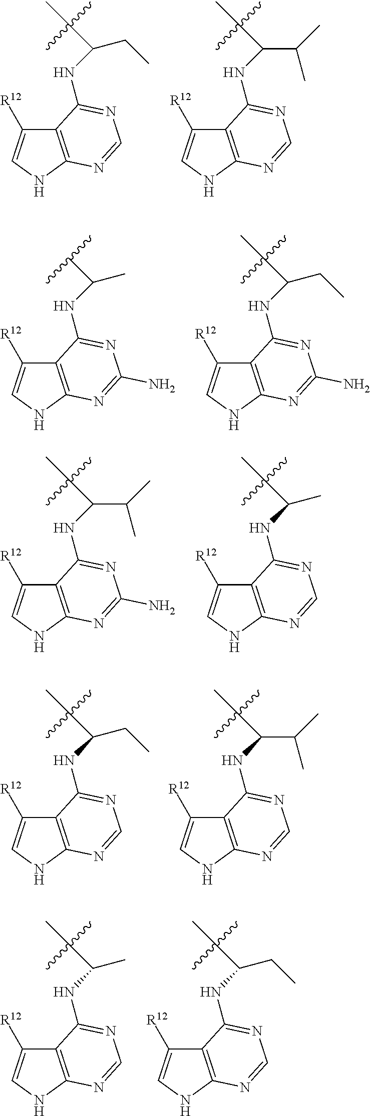

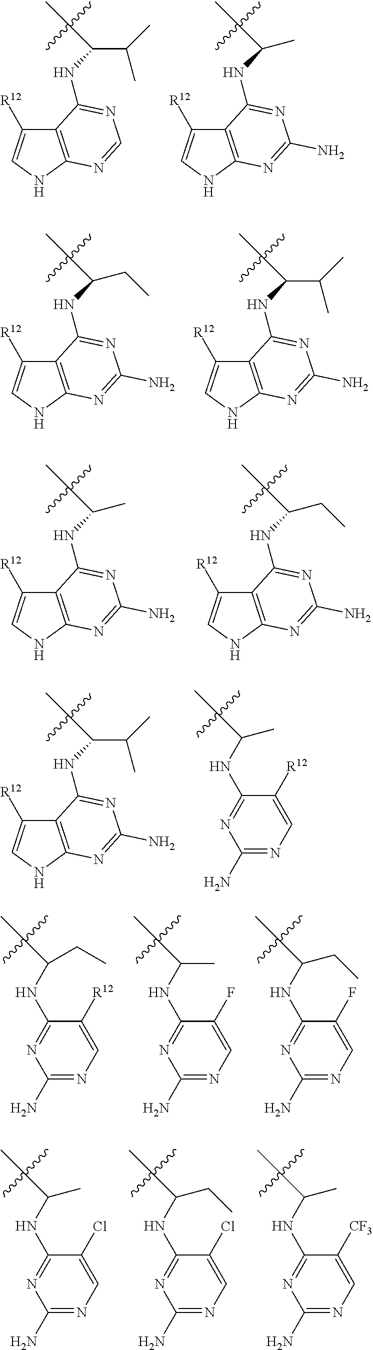

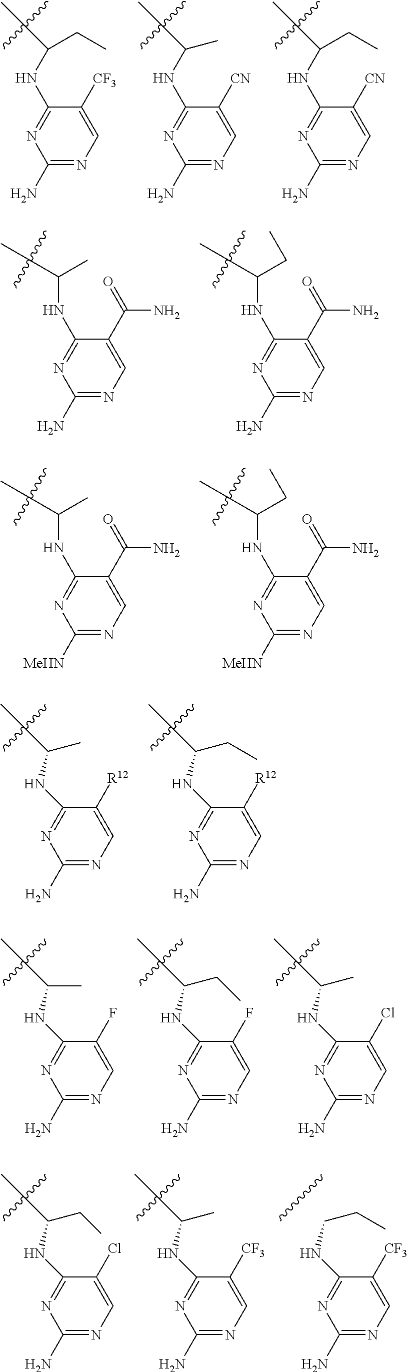

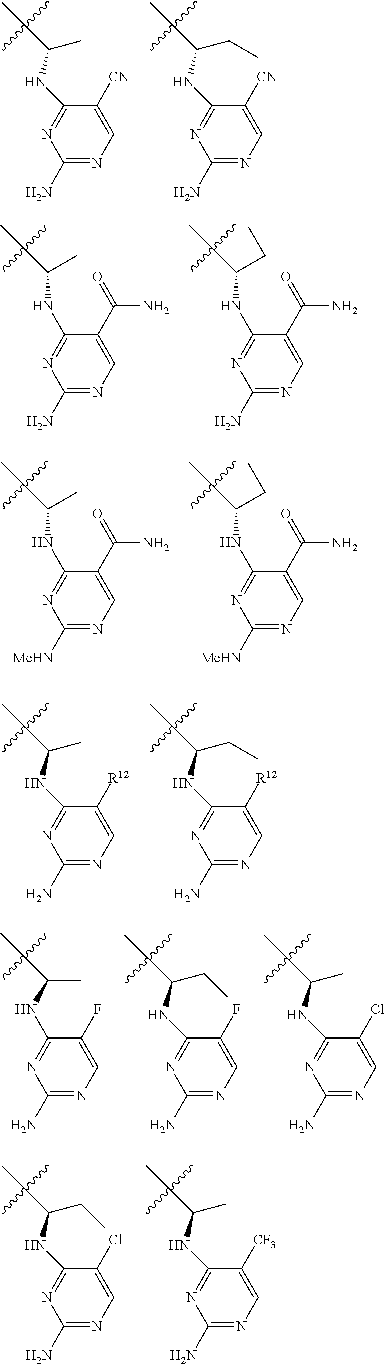

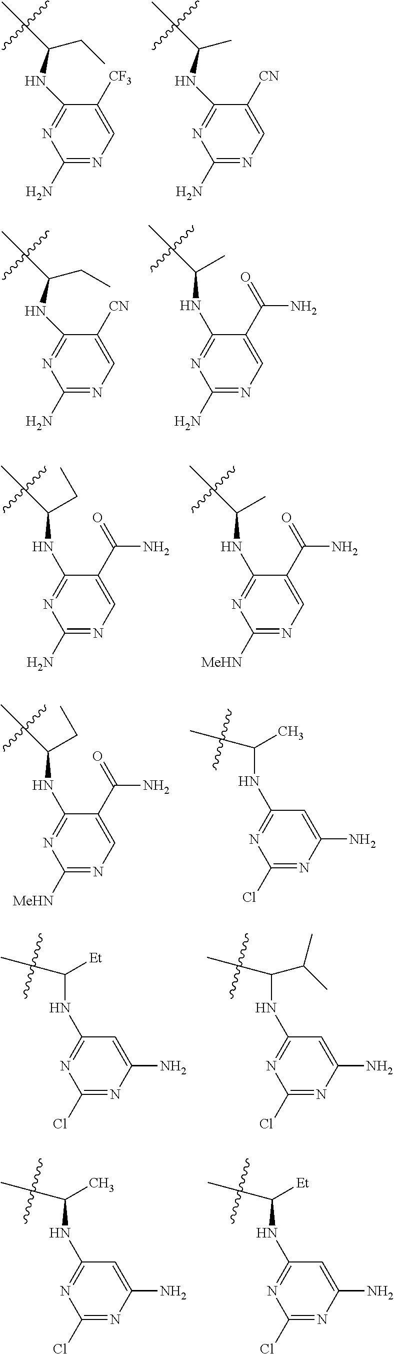

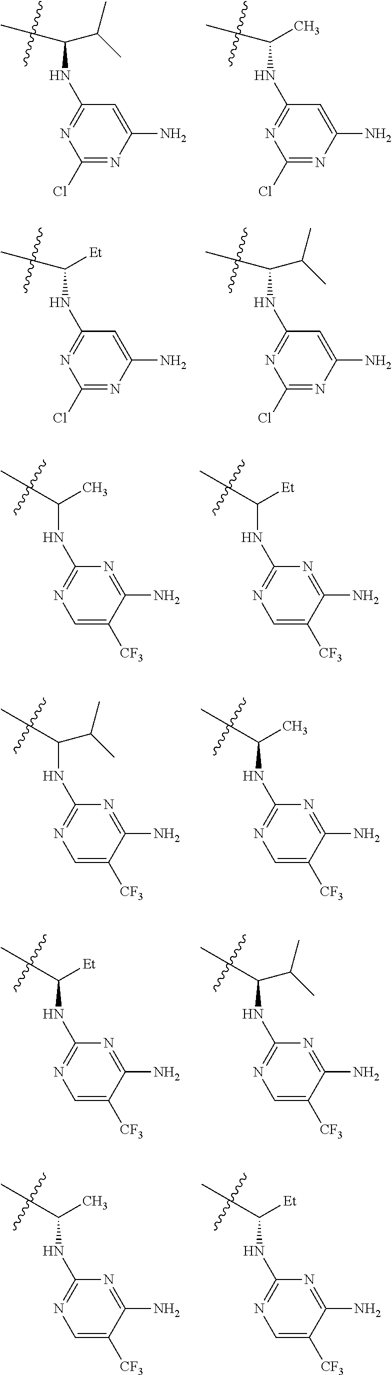

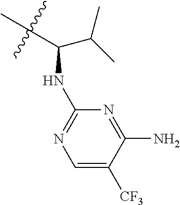 [SMILES: *[C@H](Nc1ncc(C(F)(F)F)c(N)n1)C(C)C]

In some embodiments of the compound of Formula I, R¹² is a member of the group consisting of hydrogen, cyano, halo, unsubstituted or substituted alkyl, unsubstituted or substituted alkynyl, and unsubstituted or substituted alkenyl. In some embodiments, R¹² is unsubstituted or substituted aryl. In some embodiments, R¹² is unsubstituted or substituted heteroaryl, which includes but is not limited to heteroaryl having a 5 membered ring, heteroaryl having a six membered ring, heteroaryl with at least one nitrogen ring atom, heteroaryl with two nitrogen ring atoms, monocylic heteroaryl, and bicylic heteroaryl. In some embodiments, R¹² is unsubstituted or substituted heterocycloalkyl, which includes but is not limited to heterocycloalkyl with one nitrogen ring atom, heterocycloalkyl with one oxygen ring atom, R¹² is heterocycloalkyl with one sulfur ring atom, 5 membered heterocycloalkyl, 6 membered heterocycloalkyl, saturated heterocycloalkyl, unsaturated heterocycloalkyl, heterocycloalkyl having an unsaturated moiety connected to the heterocycloalkyl ring, heterocycloalkyl substituted by oxo, and heterocycloalkyl substituted by two oxo. In some embodiments, R¹² is unsubstituted or substituted cycloalkyl, including but not limited to cyclopropyl, cyclobutyl, cyclopentyl, cyclohexyl, cycloalkyl substituted by one oxo, cycloalkyl having an unsaturated moiety connected to the cycloalkyl ring. In some embodiments, R¹² is unsubstituted or substituted amido, carboxylic acid, unsubstituted or substituted acyloxy, unsubstituted or substituted alkoxycarbonyl, unsubstituted or substituted acyl, or unsubstituted or substituted sulfonamido.

In some embodiments, when R¹² is alkyl, alkynyl, alkenyl, aryl, heteroaryl, heterocycloalkyl, or cycloalkyl, it is substituted with phosphate. In some embodiments, when R¹² is alkyl, alkynyl, alkenyl, aryl, heteroaryl, heterocycloalkyl, or cycloalkyl, it is substituted with urea. In some embodiments, when R¹² is alkyl, alkynyl, alkenyl, aryl, heteroaryl, heterocycloalkyl, or cycloalkyl, it is substituted with carbonate.

In some embodiments, when R¹² is alkyl, alkynyl, alkenyl, aryl, heteroaryl, heterocycloalkyl, cycloalkyl, alkoxycarbonyl, amido, acyloxy, acyl, or sulfonamido, it is substituted with one or more of alkyl, heteroalkyl, alkenyl, alkynyl, cycloalkyl, heterocycloalkyl, aryl, heteroaryl, alkoxy, amido, amino, acyl, acyloxy, alkoxycarbonyl, sulfonamido, halo, cyano, hydroxy or nitro, each of which alkyl, heteroalkyl, alkenyl, alkynyl, cycloalkyl, heterocycloalkyl, aryl, heteroaryl, alkoxy, amido, amino, acyl, acyloxy, alkoxycarbonyl, or sulfonamido can itself be substituted.

In some embodiments of the compound of Formula I, R¹² of W_{d} is one of the following moieties:

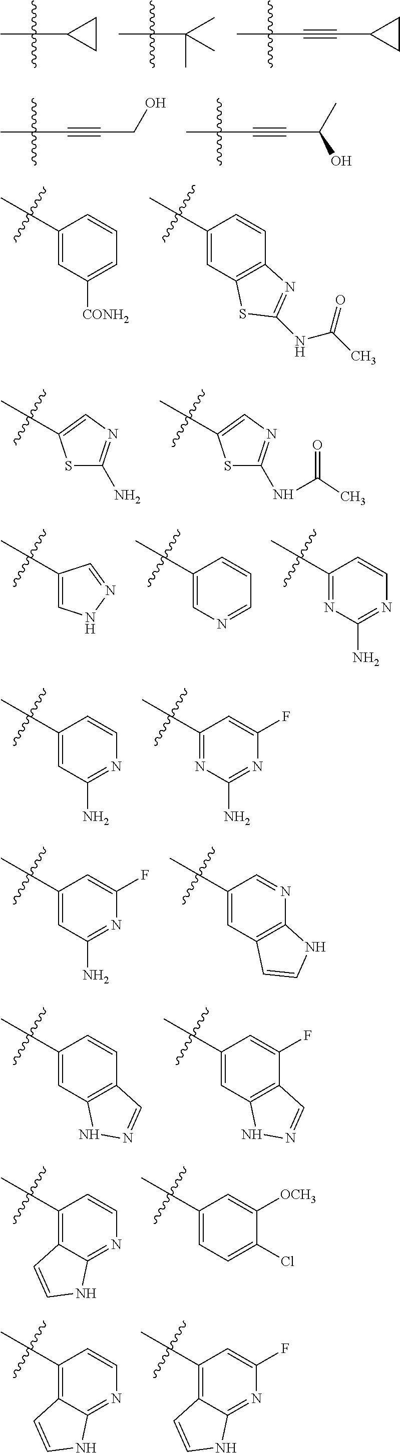

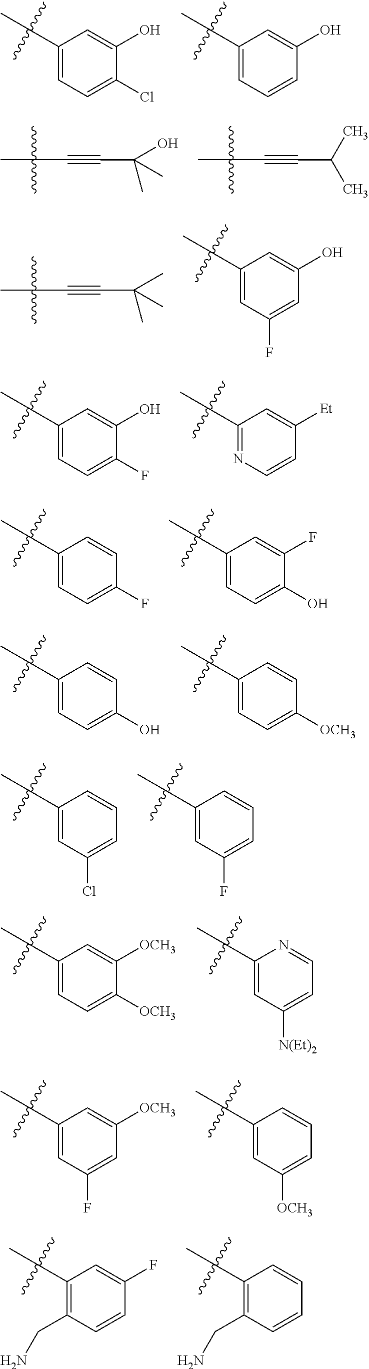

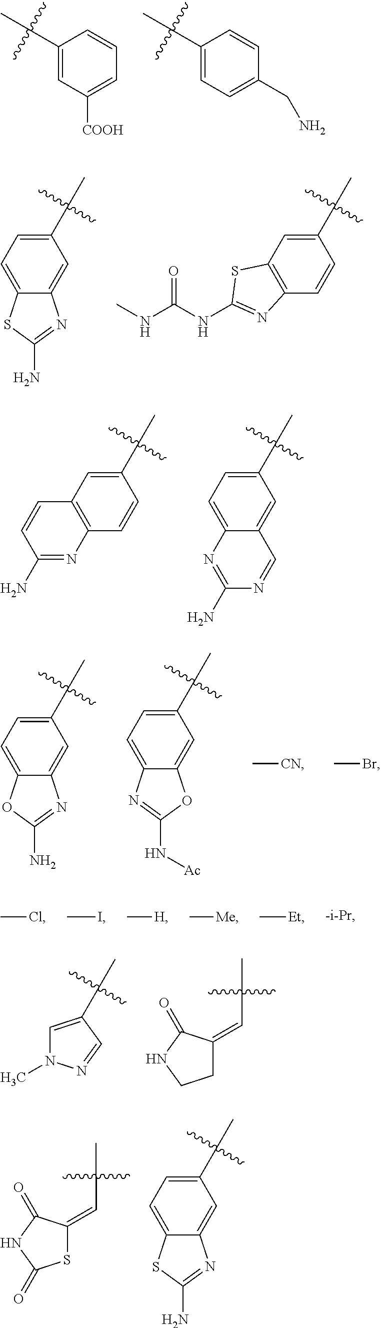

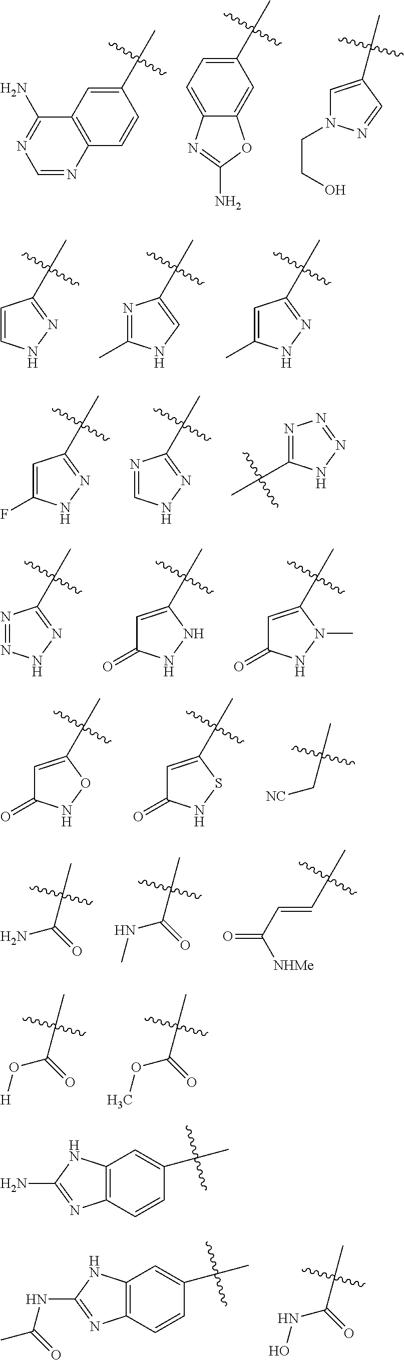

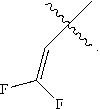 [SMILES: *C=C(F)F]

In some embodiments of the compound of Formula I, W_{d} is a pyrazolopyrimidine of Formula III:

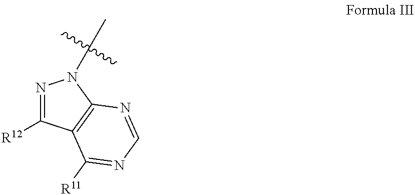

wherein R¹¹ is H, alkyl, halo, amino, amido, hydroxy, or alkoxy, and R¹² is H, alkyl, alkynyl, alkenyl, halo, aryl, heteroaryl, heterocycloalkyl, or cycloalkyl. In some embodiments, R¹¹ is amino and R¹² is H, alkyl, alkynyl, alkenyl, halo, aryl, heteroaryl, heterocycloalkyl, or cycloalkyl. In some embodiments, R¹¹ is amino and R¹² is alkyl, halo, aryl, heteroaryl, heterocycloalkyl, or cycloalkyl. In some embodiments, R¹¹ is amino and R¹² is monocyclic heteroaryl. In some embodiments, R¹¹ is amino and R¹² is bicyclic heteroaryl. In some embodiments, R¹¹ is amino and R¹² is cyano, amino, carboxylic acid, acyloxy, alkoxycarbonyl, or amido.

In some embodiments of the invention, the compound of Formula I is a compound having a structure of Formula IV:

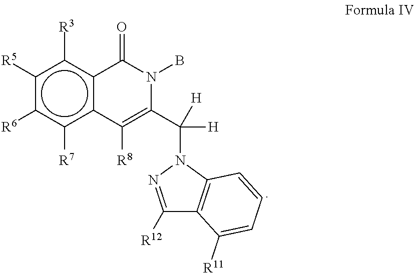

In some embodiments of the compound of Formula IV, R¹¹ is H, alkyl, halo, amino, amido, hydroxy, or alkoxy, and R¹² is H, alkyl, alkynyl, alkenyl, halo, aryl, heteroaryl, heterocycloalkyl, or cycloalkyl. In another embodiment, R¹¹ is amino and R¹² is alkyl, alkenyl, heteroaryl, aryl, or heterocycloalkyl. In some embodiments, R¹¹ is amino and R¹² is cyano, amino, carboxylic acid, alkoxycarbonyl, or amido.

In some embodiments, the compound of Formula IV is a compound of Formula IV-A:

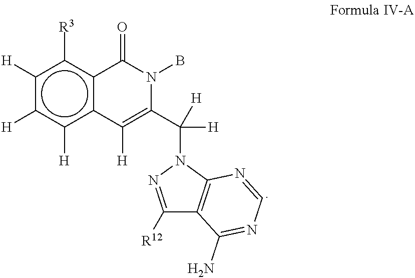

The invention also provides compounds of Formula I having a structure of any of Formulae V, V-A1, V-A2, V-B, VI, VI-A, VII-A1, VII-A2, VIII-A1, VIII-A2, IX-A1, IX-A2, X-A1, X-A2, XI-A1, XI-A2, XII-A, XII-A1, XII-A2, XIII-A, XIII-A1, XIII-A2, XIV-A, XIV-A1, XIV-A2, XV-A, XV-A1, XV-A2, XVI-A, XVI-A1, XVI-A2, XVII-A, XVII-A1, XVII-A2, XVIII-A, XVIII-A1, or XVIII-A2:

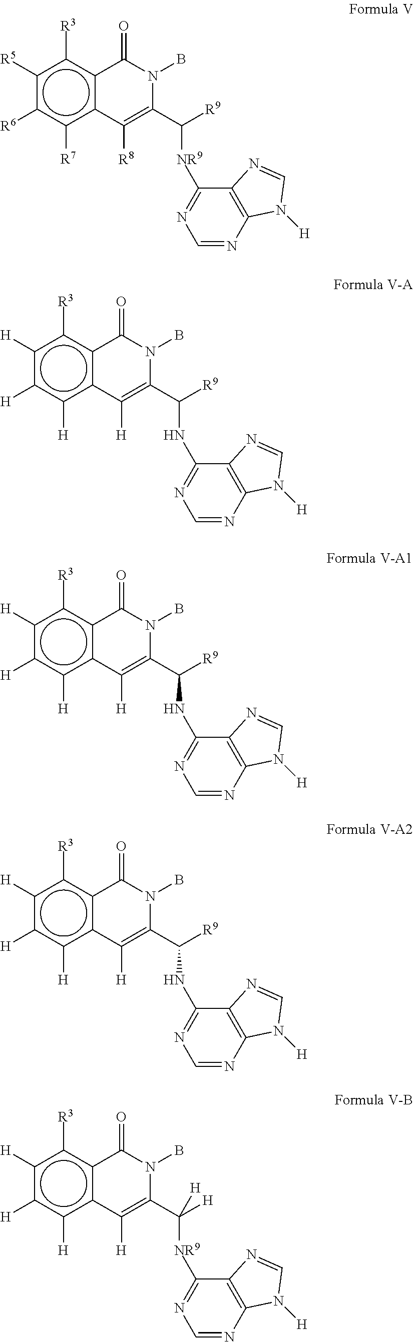

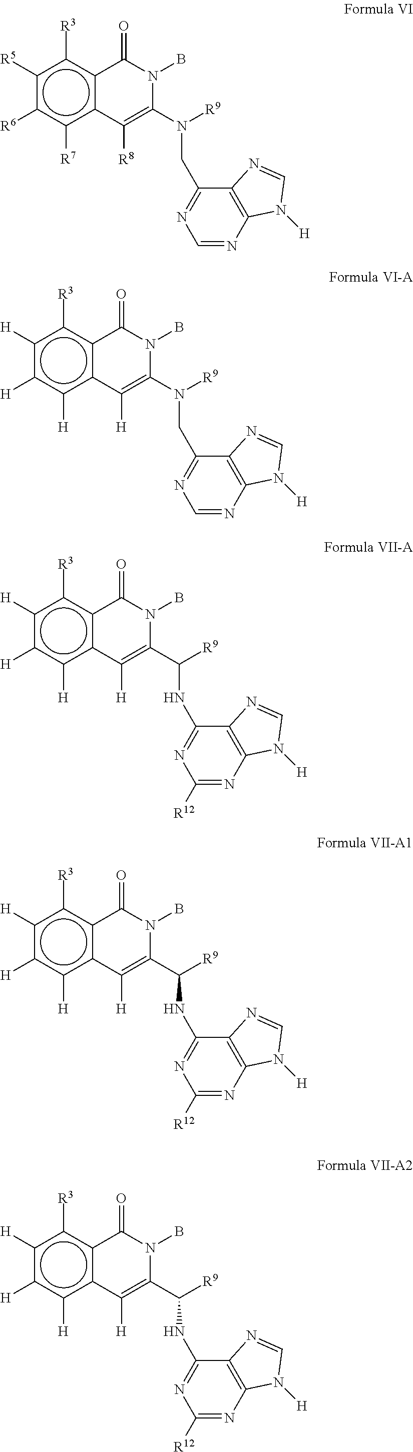

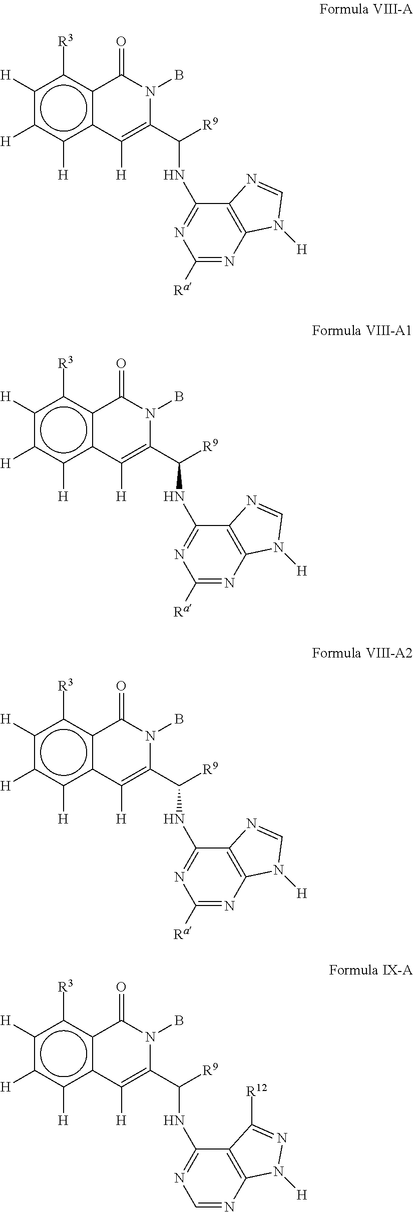

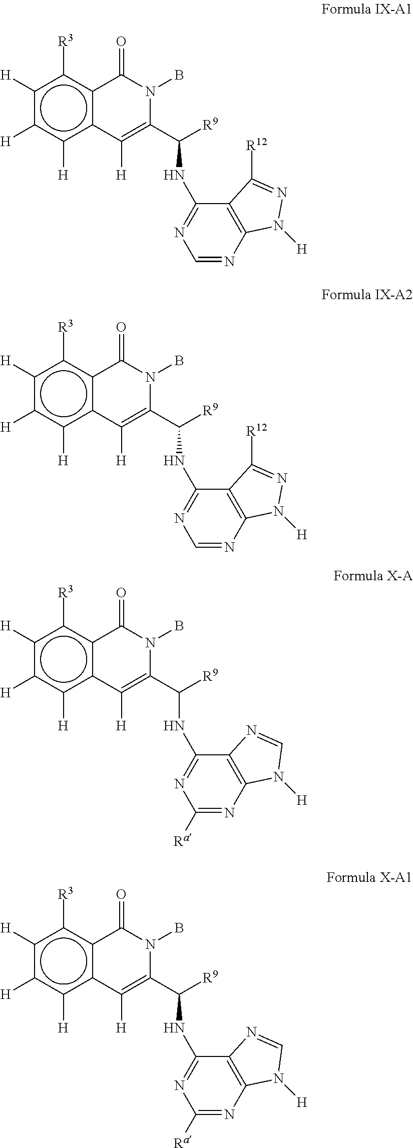

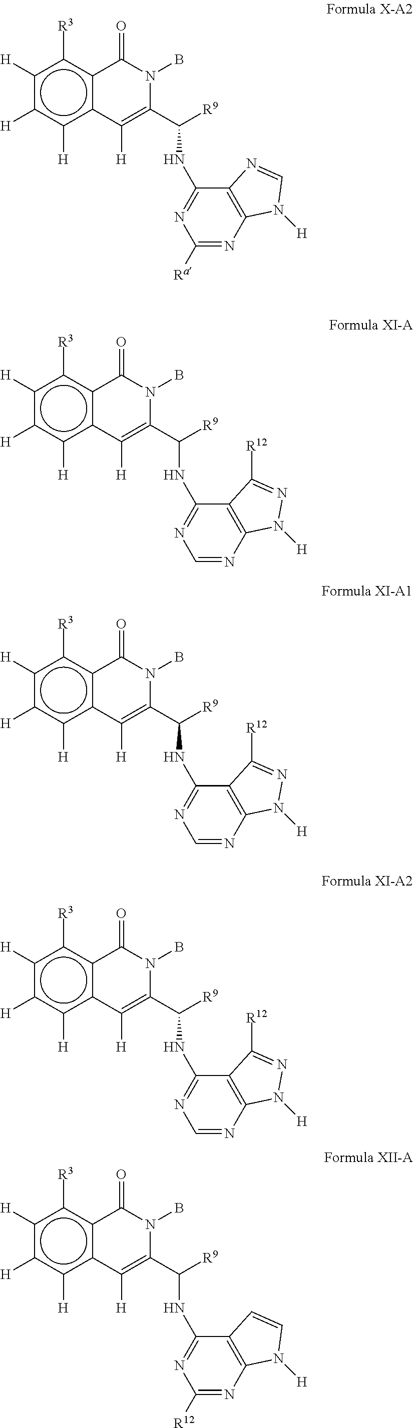

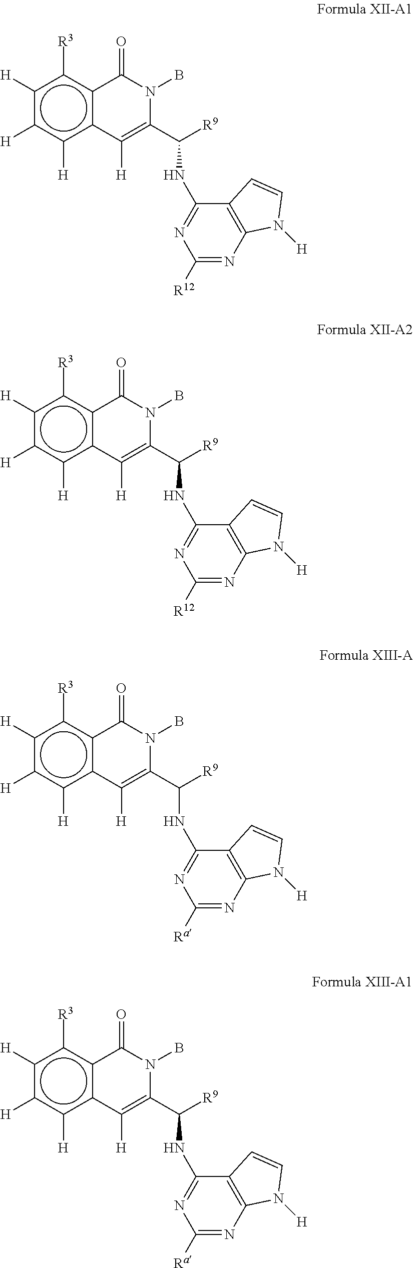

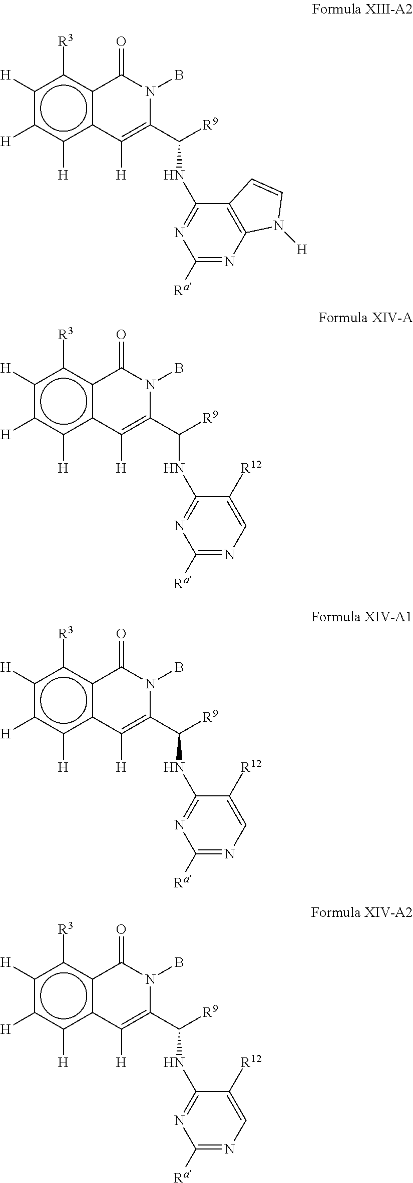

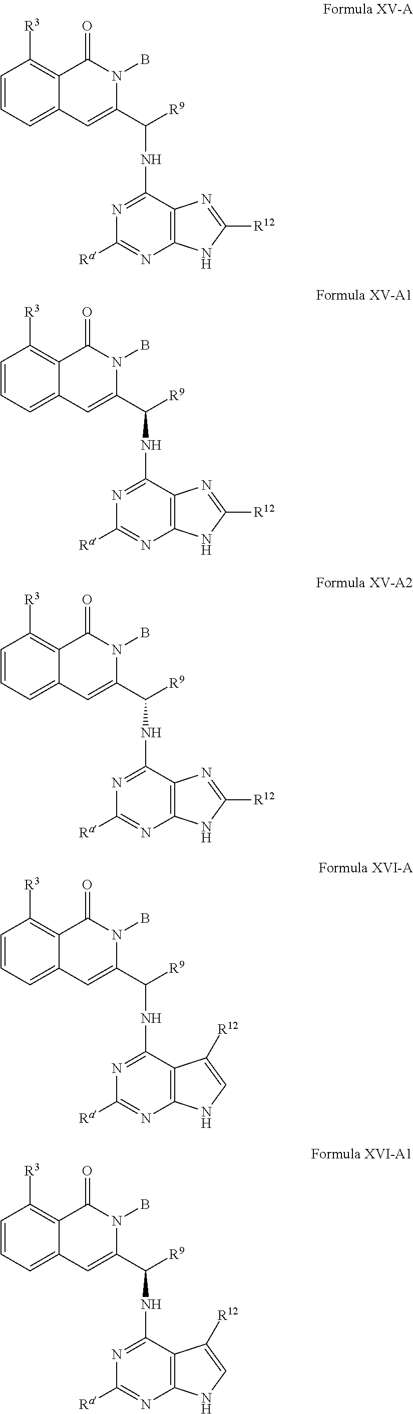

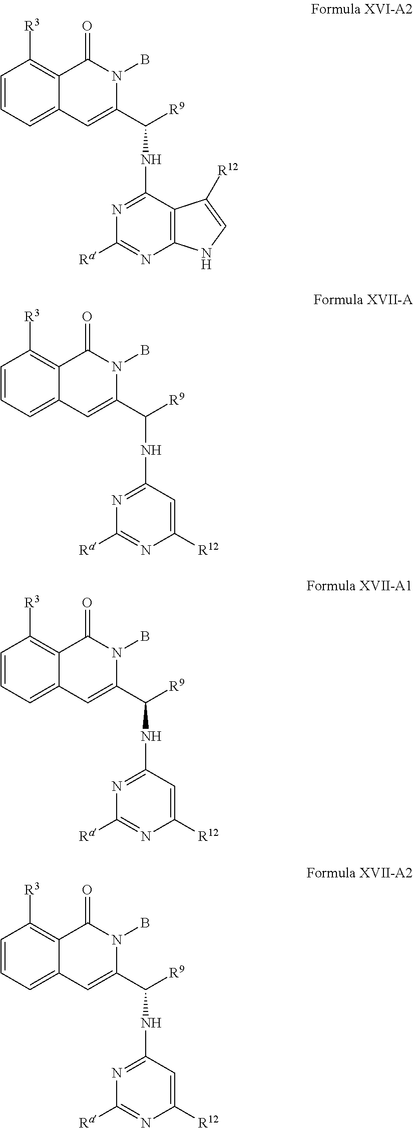

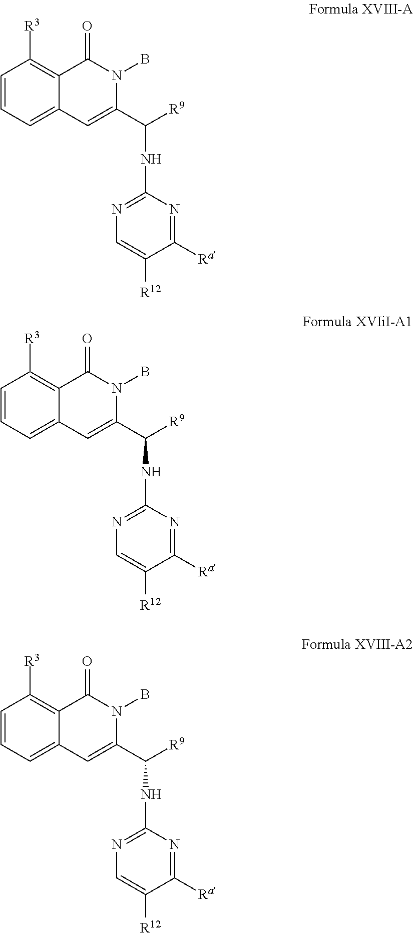

Any of the disclosed elements and their substituents for the compounds of Formula I can be used in any combination.

In one aspect, for the compounds of Formula I, R₃ is H, CH₃, CF₃, Cl, or F; and B is a moiety of Formula II:

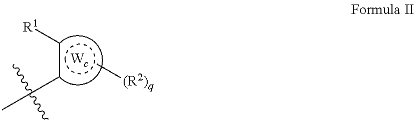

wherein W_{c} is aryl, heteroaryl, heterocycloalkyl, or cycloalkyl; R¹ is H, —F, —Cl, —CN, —CH₃, isopropyl, —CF₃, —OCH₃, nitro, or phosphate; R² is halo, hydroxy, cyano, or nitro; q is an integer of 0, 1, 2, 3, or 4; R⁵, R⁶, R⁷, and R⁸ are H; X is absent or (CH₂)_{z}; z is 1; Y is absent or —N(R⁹)—; R⁹ is hydrogen, C₁-C₁₀alkyl, C₃-C₇cycloalkyl, or C₂-C₁₀heteroalkyl; at least one of X and Y is present; and W_{d} is pyrazolopyrimidine or purine. In some embodiments, when X and Y are present and W_{d} is purine, then —N(R⁹)— is —NH—.

In another aspect, for the compounds of Formula I, R₃ is H, CH₃, CF₃, Cl, or F; B is a moiety of Formula II which is aryl, heteroaryl, heterocycloalkyl, or cycloalkyl, R′ is H, —F, —Cl, —CN, —CH₃, isopropyl, —CF₃, —OCH₃, nitro, or phosphate; R² is halo, hydroxy, cyano, or nitro; q is 0, 1 or 2; R⁵, R⁶, R⁷, and R⁸ are H; X is absent or (CH₂)_{z}; z is 1; Y is absent or —N(R⁹)—; R⁹ is hydrogen, methyl, or ethyl; at least one of X and Y is present; W_{d} is:

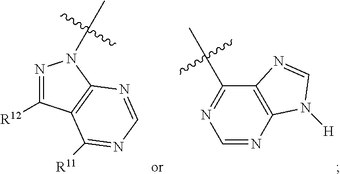

R¹¹ is amino; and R¹² is H, alkyl, alkynyl, alkenyl, halo, aryl, heteroaryl, heterocycloalkyl, or cycloalkyl. In some embodiments, when X and Y are present and W_{d} is purine, then —N(R⁹)— is —NH—.

In another aspect, for the compounds of Formula I, R₃ is H, CH₃, CF₃, Cl, or F; B is a moiety of Formula II, which is aryl, heteroaryl, heterocycloalkyl, or cycloalkyl, R′ is H, —F, —Cl, —CN, —CH₃, isopropyl, —CF₃, —OCH₃, nitro, or phosphate; R² is halo, hydroxy, cyano, or nitro; q is 0, 1 or 2; X is (CH₂)_{z}; z is 1; R⁵, R⁶, R⁷, and R⁸ are H; Y is absent and W_{d} is:

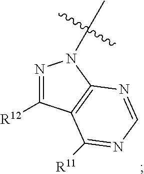

R¹¹ is amino; and R¹² is H, alkyl, alkynyl, alkenyl, halo, aryl, heteroaryl, heterocycloalkyl, or cycloalkyl.

In another aspect, R₃ is H, CH₃, CF₃, Cl, or F; B is aryl, heteroaryl, heterocycloalkyl, or cycloalkyl, R¹ is H, —F, —Cl, —CN, —CH₃, isopropyl, —CF₃, —OCH₃, nitro, or phosphate; R² is halo, hydroxy, cyano, or nitro; q is 0, 1 or 2; R⁵, R⁶, R⁷, and R⁸ are H; X is (CH₂)_{z}; z is 1; X is (CH₂)_{z}; z is 1; Y is —N(R⁹)—; R⁹ is hydrogen, methyl, or ethyl; and W_{d} is

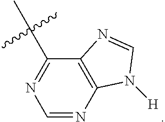 [SMILES: *c1ncnc2[nH]cnc12]

In some embodiments, Y is —NH—.

In another aspect, for the compounds of Formula I R₃ is aryl, heteroaryl, H, CH₃, CF₃, Cl, or F; B is alkyl or a moiety of Formula II;

wherein W_{c} is aryl, heteroaryl, heterocycloalkyl, or cycloalkyl, and q is an integer of 0, 1, 2, 3, or 4; R¹ is H, —F, —Cl, —CN, —CH₃, isopropyl, —CF₃, —OCH₃, nitro, or phosphate; R² is halo, hydroxy, cyano, nitro, or phosphate; q is 0, 1 or 2; R⁵, R⁶, R⁷, and R⁸ are H; X is absent or (CH(R⁹))_{z}; z is an integer of 1, 2, 3, or 4; Y is absent, —N(R⁹)—, or —N(R⁹) CH(R⁹)—; R⁹ is hydrogen, alkyl, cycloalkyl, or heteroalkyl; at least one of X and Y is present; and W_{d} is pyrazolopyrimidine or purine. In some embodiments, when X is present, Y is —N(R⁹)—, and W_{d} is purine, then Y is —NH—.

In another aspect, for the compounds of Formula I, R₃ is aryl, heteroaryl, H, CH₃, CF₃, Cl, or F; B is alkyl or a moiety of Formula II which is aryl, heteroaryl, heterocycloalkyl, or cycloalkyl, R′ is H, —F, —Cl, —CN, —CH₃, isopropyl, —CF₃, —OCH₃, nitro, or phosphate; R² is halo, hydroxy, cyano, nitro, or phosphate; q is 0, 1 or 2; R⁵, R⁶, R⁷, and R⁸ are H; X is absent or (CH(R⁹))_{z}; z is an integer of 1, 2, 3, or 4; Y is absent, —N(R⁹)—, or —N(R⁹) CH(R⁹)—; R⁹ is hydrogen, methyl, or ethyl; at least one of X and Y is present; W_{d} is:

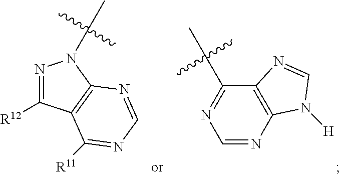

R¹¹ is amino; and R¹² is H, alkyl, alkynyl, alkenyl, halo, aryl, heteroaryl, heterocycloalkyl, cycloalkyl, cyano, amino, carboxylic acid, alkoxycarbonyl, or amido. In some embodiments, when X is present, Y is —N(R⁹)—, and W_{d} is purine, then Y is —NH—.

In another aspect, for the compounds of Formula I, R₃ is H, CH₃, CF₃, Cl, or F; B is alkyl or a moiety of Formula II which is aryl, heteroaryl, heterocycloalkyl, or cycloalkyl, R¹ is H, —F, —Cl, —CN, —CH₃, isopropyl, —CF₃, —OCH₃, nitro, or phosphate; R² is halo, hydroxy, cyano, nitro, or phosphate; q is 0, 1 or 2; R⁵, R⁶, R⁷, and R⁸ are H; X is (CH(R⁹))_{z}; z is an integer of 1; Y is absent-; R⁹ is hydrogen, methyl, or ethyl; W_{d} is:

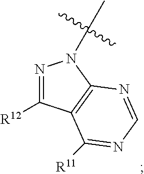

R¹¹ is amino; and R¹² is H, alkyl, alkynyl, alkenyl, halo, aryl, heteroaryl, heterocycloalkyl, cycloalkyl, cyano, amino, carboxylic acid, alkoxycarbonyl, or amido.

In another aspect, for the compounds of Formula I, R₃ is aryl, heteroaryl, H, CH₃, CF₃, Cl, or F; B is a moiety of Formula II which is aryl, heteroaryl, heterocycloalkyl, or cycloalkyl, R′ is H, —F, —Cl, —CN, —CH₃, isopropyl, —CF₃, —OCH₃, nitro, or phosphate; R² is halo, hydroxy, cyano, nitro, or phosphate; q is 0, 1 or 2; R⁵, R⁶, R⁷, and R⁸ are H; X is absent or (CH(R⁹))_{z}; z is an integer of 1; Y is absent, —N(R⁹)—, or —N(R⁹) CH(R⁹)—; R⁹ is hydrogen, methyl, or ethyl; at least one of X and Y is present, and W_{d} is:

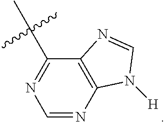 [SMILES: *c1ncnc2[nH]cnc12]

In some embodiments, when X is present, Y is —N(R⁹)—, and W_{d} is purine, then Y is —NH—.

In another aspect, for the compounds of Formula I, R₃ is aryl, heteroaryl, H, CH₃, CF₃, Cl, or F; B is a moiety of Formula II which is aryl, heteroaryl, heterocycloalkyl, or cycloalkyl, R′ is H, —F, —Cl, —CN, —CH₃, isopropyl, —CF₃, —OCH₃, nitro, or phosphate; R² is halo, hydroxy, cyano, nitro, or phosphate; q is 0, 1 or 2; R⁵, R⁶, R⁷, and R⁸ are H; X is absent; Y is —N(R⁹) CH(R⁹)—; R⁹ is hydrogen, methyl, or ethyl; and W_{d} is:

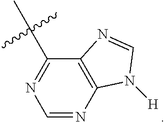 [SMILES: *c1ncnc2[nH]cnc12]

In another aspect, for the compounds of Formula I, R₃ is aryl, heteroaryl, H, CH₃, CF₃, Cl, or F; B is alkyl or a moiety of Formula II which is aryl, heteroaryl, heterocycloalkyl, or cycloalkyl, R¹ is H, —F, —Cl, —CN, —CH₃, isopropyl, —CF₃, —OCH₃, nitro, or phosphate; R² is halo, hydroxy, cyano, nitro, or phosphate; q is 0, 1 or 2; R⁵, R⁶, R⁷, and R⁸ are H; X is absent or (CH(R⁹))_{z}; z is an integer of 1, 2, 3, or 4; Y is absent, —N(R⁹)—, or —N(R⁹) CH(R⁹)—; R⁹ is hydrogen, methyl, or ethyl; at least one of X and Y is present; W_{d} is:

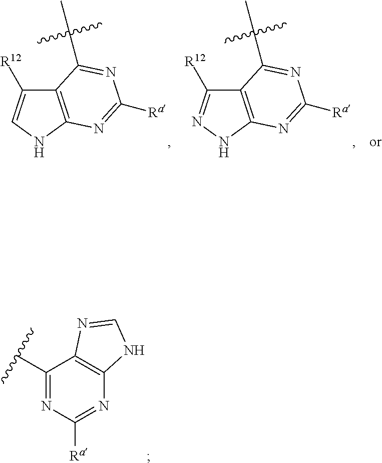

R^{a′} is hydrogen, halo, or amino; and R¹² is H, alkyl, alkynyl, alkenyl, halo, aryl, heteroaryl, heterocycloalkyl, cycloalkyl, cyano, amino, carboxylic acid, alkoxycarbonyl, or amido. In some embodiments, when X is present, Y is —N(R⁹)—, and W_{d} is purine, then Y is —NH—.

Additional exemplary compounds of the present invention are disclosed having a sub-structure of Formula IV-A.

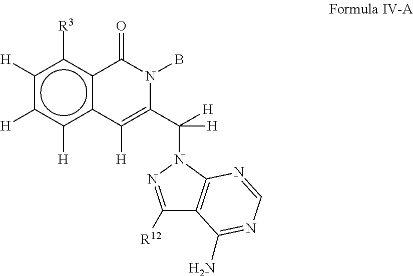

Some illustrative compounds of the present invention having a structure of Formula IV-A include those in which R³ is —H, —Cl, —F, or —CH₃ in combination with any B moiety described in Table 1, and any R¹² as described in Table 2. A compound of Formula IV-A includes any combination of R³, B, and R¹². Additional exemplary compounds of Formula IV-A are illustrated in Table 4.

TABLE 1
Illustrative B moieties of the compounds of Formula I.

| Sub- | |
|---|---|
| class # | B |
| B-1 | 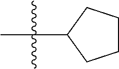 [SMILES: *C1CCCC1] |
| B-2 | 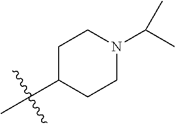 [SMILES: *C1CCN(C(C)C)CC1] |
| B-3 | —CH(CH₃)2 |
| B-4 | 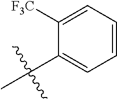 [SMILES: *c1ccccc1C(F)(F)F] |
| B-5 | 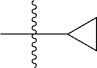 [SMILES: *C1CC1] |
| B-6 | 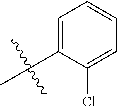 [SMILES: *c1ccccc1Cl] |
| B-7 | 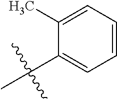 [SMILES: *c1ccccc1C] |
| B-8 | 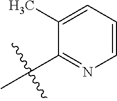 [SMILES: *c1ncccc1C] |
| B-9 | 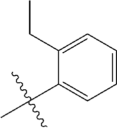 [SMILES: *c1ccccc1CC] |
| B-10 | 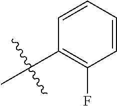 [SMILES: *c1ccccc1F] |
| B-11 | 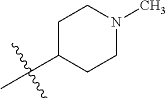 [SMILES: *C1CCN(C)CC1] |
| B-12 | 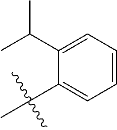 [SMILES: *c1ccccc1C(C)C] |
| B-13 | 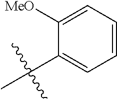 [SMILES: *c1ccccc1OC] |
| B-14 | 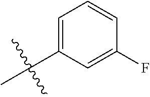 [SMILES: *c1cccc(F)c1] |
| B-15 | 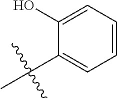 [SMILES: *c1ccccc1O] |
| B-16 | 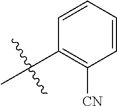 [SMILES: *c1ccccc1C#N] |
| B-17 | 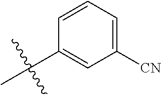 [SMILES: *c1cccc(C#N)c1] |
| B-18 | 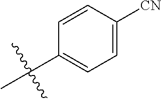 [SMILES: *c1ccc(C#N)cc1] |
| B-19 | 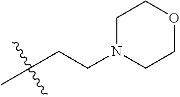 [SMILES: *CCN1CCOCC1] |
| B-20 | 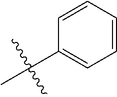 [SMILES: *c1ccccc1] |
| B-21 | 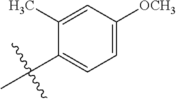 [SMILES: *c1ccc(OC)cc1C] |
| B-22 | 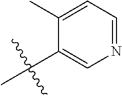 [SMILES: *c1cnccc1C] |
| B-23 | 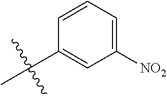 [SMILES: *c1cccc([N+](=O)[O-])c1] |
| B-24 | 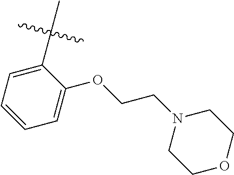 [SMILES: *c1ccccc1OCCN1CCOCC1] |
| B-25 | 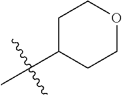 [SMILES: *C1CCOCC1] |
| B-26 | 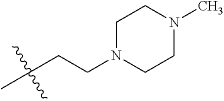 [SMILES: *CCN1CCN(C)CC1] |
| B-27 | 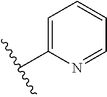 [SMILES: *c1ccccn1] |
| B-28 | 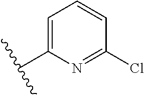 [SMILES: *c1cccc(Cl)n1] |
| B-29 | 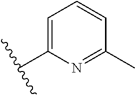 [SMILES: *c1cccc(C)n1] |
| B-30 | 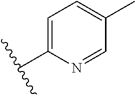 [SMILES: *c1ccc(C)cn1] |
| B-31 | 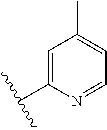 [SMILES: *c1cc(C)ccn1] |
| B-32 | 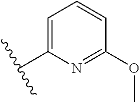 [SMILES: *c1cccc(OC)n1] |
| B-33 | 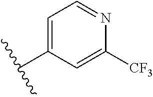 [SMILES: *c1ccnc(C(F)(F)F)c1] |
| B-34 | 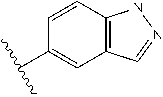 [SMILES: *c1ccc2[nH]ncc2c1] |
| B-35 | 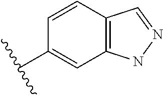 [SMILES: *c1ccc2cn[nH]c2c1] |
| B-36 | 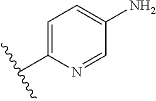 [SMILES: *c1ccc(N)cn1] |
| B-37 | 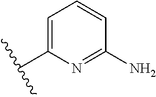 [SMILES: *c1cccc(N)n1] |
| B-38 | 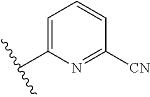 [SMILES: *c1cccc(C#N)n1] |
| B-39 | 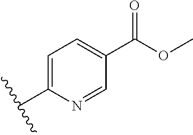 [SMILES: *c1ccc(C(=O)OC)cn1] |
| B-40 | 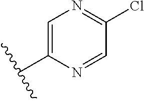 [SMILES: *c1cnc(Cl)cn1] |
| B-41 | 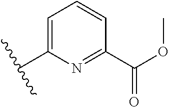 [SMILES: *c1cccc(C(=O)OC)n1] |
| B-42 | 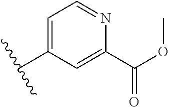 [SMILES: *c1ccnc(C(=O)OC)c1] |
| B-43 | 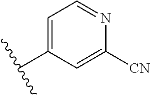 [SMILES: *c1ccnc(C#N)c1] |
| B-44 | 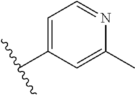 [SMILES: *c1ccnc(C)c1] |
| B-45 | 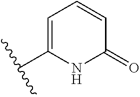 [SMILES: *c1cccc(=O)[nH]1] |
| B-46 | 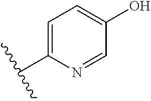 [SMILES: *c1ccc(O)cn1] |
| B-47 | 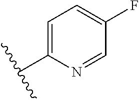 [SMILES: *c1ccc(F)cn1] |
| B-48 | 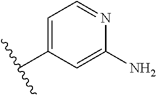 [SMILES: *c1ccnc(N)c1] |
| B-49 | 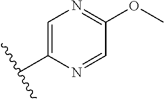 [SMILES: *c1cnc(OC)cn1] |
| B-50 | 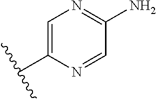 [SMILES: *c1cnc(N)cn1] |
| B-51 | 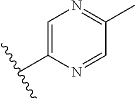 [SMILES: *c1cnc(C)cn1] |
| B-52 | 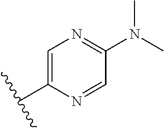 [SMILES: *c1cnc(N(C)C)cn1] |
| B-53 | 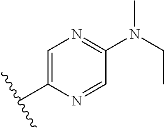 [SMILES: *c1cnc(N(C)CC)cn1] |
| B-54 | 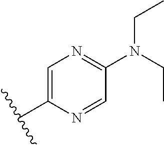 [SMILES: *c1cnc(N(CC)CC)cn1] |
| B-55 | 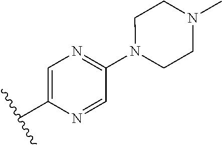 [SMILES: *c1cnc(N2CCN(C)CC2)cn1] |
| B-56 | 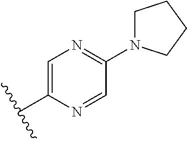 [SMILES: *c1cnc(N2CCCC2)cn1] |
| B-57 | 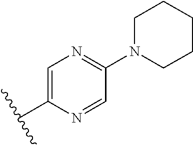 [SMILES: *c1cnc(N2CCCCC2)cn1] |
| B-58 | 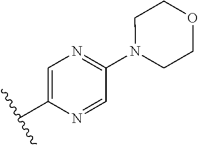 [SMILES: *c1cnc(N2CCOCC2)cn1] |
| B-59 | 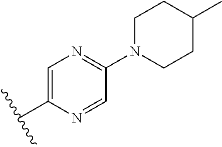 [SMILES: *c1cnc(N2CCC(C)CC2)cn1] |
| B-60 | 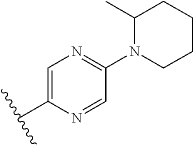 [SMILES: *c1cnc(N2CCCCC2C)cn1] |
| B-61 | 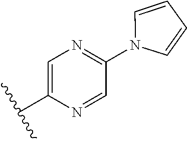 [SMILES: *c1cnc(-n2cccc2)cn1] |
| B-62 | 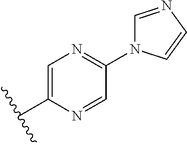 [SMILES: *c1cnc(-n2ccnc2)cn1] |
| B-63 | 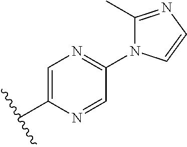 [SMILES: *c1cnc(-n2ccnc2C)cn1] |
| B-64 | 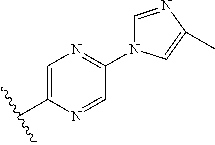 [SMILES: *c1cnc(-n2cnc(C)c2)cn1] |
| B-65 | 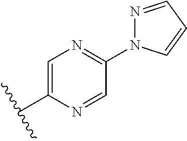 [SMILES: *c1cnc(-n2cccn2)cn1] |
| B-66 | 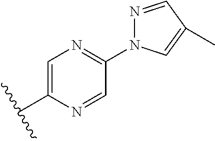 [SMILES: *c1cnc(-n2cc(C)cn2)cn1] |
| B-67 | 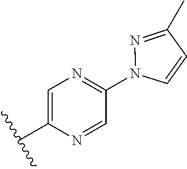 [SMILES: *c1cnc(-n2ccc(C)n2)cn1] |
| B-68 | 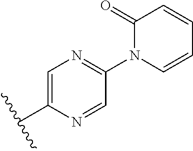 [SMILES: *c1cnc(-n2ccccc2=O)cn1] |
| B-69 | 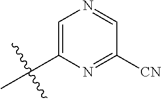 [SMILES: *c1cncc(C#N)n1] |
| B-70 | 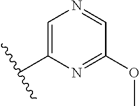 [SMILES: *c1cncc(OC)n1] |
| B-71 | 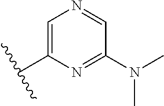 [SMILES: *c1cncc(N(C)C)n1] |
| B-72 | 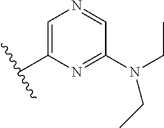 [SMILES: *c1cncc(N(CC)CC)n1] |
| B-73 | 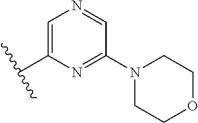 [SMILES: *c1cncc(N2CCOCC2)n1] |
| B-74 | 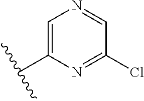 [SMILES: *c1cncc(Cl)n1] |
| B-75 | 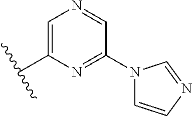 [SMILES: *c1cncc(-n2ccnc2)n1] |
| B-76 | 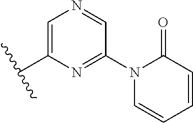 [SMILES: *c1cncc(-n2ccccc2=O)n1] |
| B-77 | 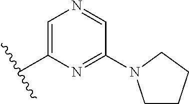 [SMILES: *c1cncc(N2CCCC2)n1] |
| B-78 | 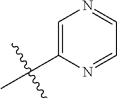 [SMILES: *c1cnccn1] |
| B-79 | 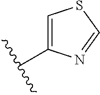 [SMILES: *c1cscn1] |
| B-80 | 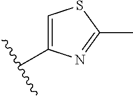 [SMILES: *c1csc(C)n1] |
| B-81 | 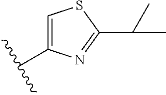 [SMILES: *c1csc(C(C)C)n1] |
| B-82 | 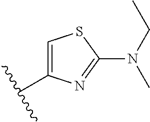 [SMILES: *c1csc(N(C)CC)n1] |
| B-83 | 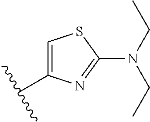 [SMILES: *c1csc(N(CC)CC)n1] |
| B-84 | 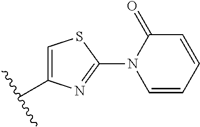 [SMILES: *c1csc(-n2ccccc2=O)n1] |
| B-85 | 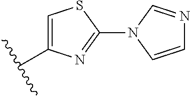 [SMILES: *c1csc(-n2ccnc2)n1] |
| B-86 | 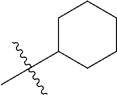 [SMILES: *C1CCCCC1] |
| B-87 | —CH₃ |
| B-88 | —CH₂CH₃ |
| B-89 | 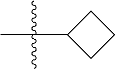 [SMILES: *C1CCC1] |
| B-90 | 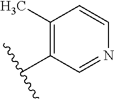 [SMILES: *c1cnccc1C] |
| B-91 | 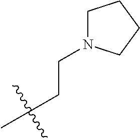 [SMILES: *CCN1CCCC1] |
| B-92 | 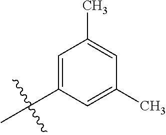 [SMILES: *c1cc(C)cc(C)c1] |
| B-93 | 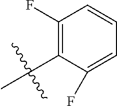 [SMILES: *c1c(F)cccc1F] |
| B-94 | 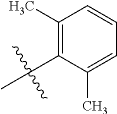 [SMILES: *c1c(C)cccc1C] |
| B-95 | 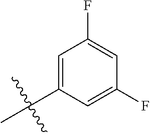 [SMILES: *c1cc(F)cc(F)c1] |
| B-96 | 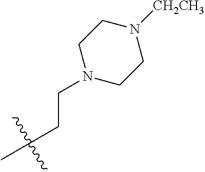 [SMILES: *CCN1CCN(CC)CC1] |
| B-97 | 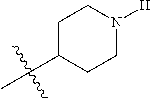 [SMILES: *C1CCNCC1] |
| B-98 | 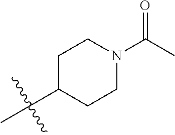 [SMILES: *C1CCN(C(C)=O)CC1] |
| B-99 | 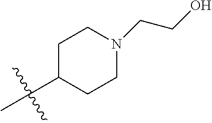 [SMILES: *C1CCN(CCO)CC1] |
| B-100 | 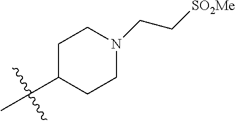 [SMILES: *C1CCN(CCS(C)(=O)=O)CC1] |
| B-101 | 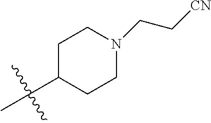 [SMILES: *C1CCN(CCC#N)CC1] |
| B-102 | 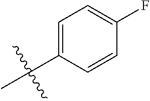 [SMILES: *c1ccc(F)cc1] |

TABLE 2
Illustrative R¹² of compounds of Formula I.

| Sub- | |
|---|---|
| class # | R¹² |
| 12-1 | —CN |
| 12-2 | —Br |
| 12-3 | —Cl |
| 12-4 | —CH₂CH₃ |
| 12-5 | —CH₃ |
| 12-6 | —CH(CH₃)₂ |
| 12-7 | 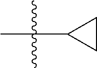 [SMILES: *C1CC1] |
| 12-8 | 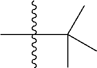 [SMILES: *C(C)(C)C] |
| 12-9 | 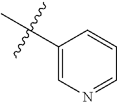 [SMILES: *c1cccnc1] |
| 12-10 | 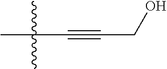 [SMILES: *C#CCO] |
| 12-11 | 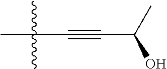 [SMILES: *C#C[C@@H](C)O] |
| 12-12 | 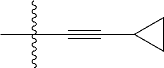 [SMILES: *C#CC1CC1] |
| 12-13 | 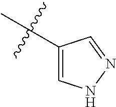 [SMILES: *c1cn[nH]c1] |
| 12-14 | 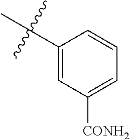 [SMILES: *c1cccc(C(N)=O)c1] |
| 12-15 | 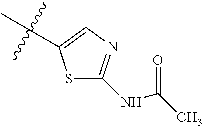 [SMILES: *c1cnc(NC(C)=O)s1] |
| 12-16 | 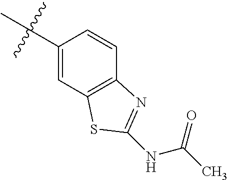 [SMILES: *c1ccc2nc(NC(C)=O)sc2c1] |
| 12-17 | 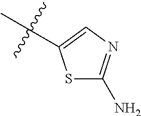 [SMILES: *c1cnc(N)s1] |
| 12-18 | 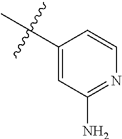 [SMILES: *c1ccnc(N)c1] |
| 12-19 | 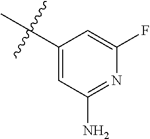 [SMILES: *c1cc(N)nc(F)c1] |
| 12-20 | 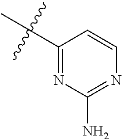 [SMILES: *c1ccnc(N)n1] |
| 12-21 | 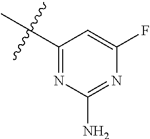 [SMILES: *c1cc(F)nc(N)n1] |
| 12-22 | 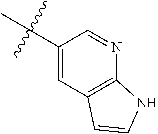 [SMILES: *c1cnc2[nH]ccc2c1] |
| 12-23 | 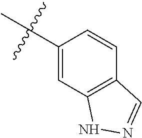 [SMILES: *c1ccc2cn[nH]c2c1] |
| 12-24 | 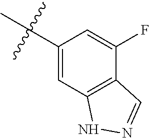 [SMILES: *c1cc(F)c2cn[nH]c2c1] |
| 12-25 | 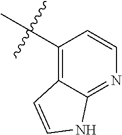 [SMILES: *c1ccnc2[nH]ccc12] |
| 12-26 | 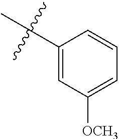 [SMILES: *c1cccc(OC)c1] |
| 12-27 | 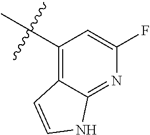 [SMILES: *c1cc(F)nc2[nH]ccc12] |
| 12-28 | 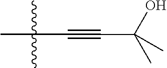 [SMILES: *C#CC(C)(C)O] |
| 12-29 | 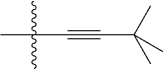 [SMILES: *C#CC(C)(C)C] |
| 12-30 | 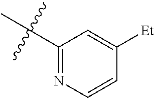 [SMILES: *c1cc(CC)ccn1] |
| 12-31 | 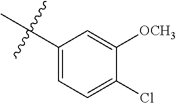 [SMILES: *c1ccc(Cl)c(OC)c1] |
| 12-32 | 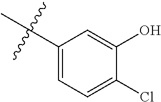 [SMILES: *c1ccc(Cl)c(O)c1] |
| 12-33 | 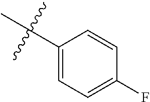 [SMILES: *c1ccc(F)cc1] |
| 12-34 | 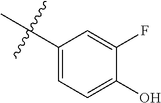 [SMILES: *c1ccc(O)c(F)c1] |
| 12-35 | —H |
| 12-36 | 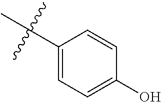 [SMILES: *c1ccc(O)cc1] |
| 12-37 | 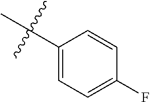 [SMILES: *c1ccc(F)cc1] |
| 12-38 | 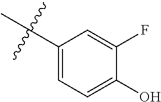 [SMILES: *c1ccc(O)c(F)c1] |
| 12-39 | 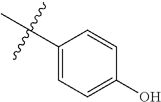 [SMILES: *c1ccc(O)cc1] |
| 12-40 | 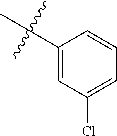 [SMILES: *c1cccc(Cl)c1] |
| 12-41 | 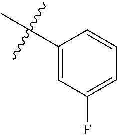 [SMILES: *c1cccc(F)c1] |
| 12-42 | 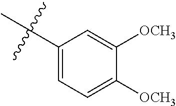 [SMILES: *c1ccc(OC)c(OC)c1] |
| 12-43 | 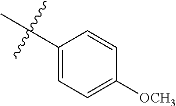 [SMILES: *c1ccc(OC)cc1] |
| 12-44 | 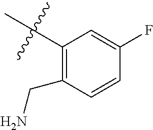 [SMILES: *c1cc(F)ccc1CN] |
| 12-45 | 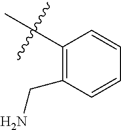 [SMILES: *c1ccccc1CN] |
| 12-46 | 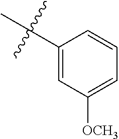 [SMILES: *c1cccc(OC)c1] |
| 12-47 | 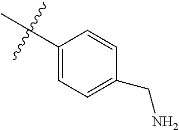 [SMILES: *c1ccc(CN)cc1] |
| 12-48 | 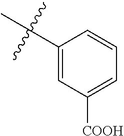 [SMILES: *c1cccc(C(=O)O)c1] |
| 12-49 | 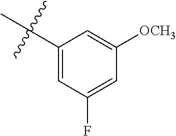 [SMILES: *c1cc(F)cc(OC)c1] |
| 12-50 | 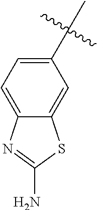 [SMILES: *c1ccc2nc(N)sc2c1] |
| 12-51 | 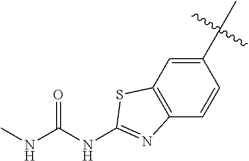 [SMILES: *c1ccc2nc(NC(=O)NC)sc2c1] |
| 12-52 | 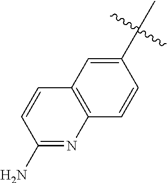 [SMILES: *c1ccc2nc(N)ccc2c1] |
| 12-53 | 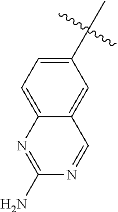 [SMILES: *c1ccc2nc(N)ncc2c1] |
| 12-54 | 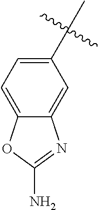 [SMILES: *c1ccc2oc(N)nc2c1] |
| 12-55 | 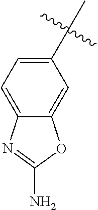 [SMILES: *c1ccc2nc(N)oc2c1] |
| 12-56 | 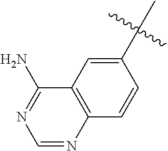 [SMILES: *c1ccc2ncnc(N)c2c1] |
| 12-57 | 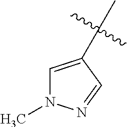 [SMILES: *c1cnn(C)c1] |
| 12-58 | 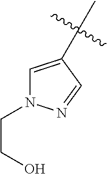 [SMILES: *c1cnn(CCO)c1] |
| 12-59 | 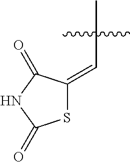 [SMILES: */C=C1/SC(=O)NC1=O] |
| 12-60 | 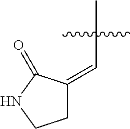 [SMILES: */C=C1/CCNC1=O] |
| 12-61 | —I |
| 12-62 | 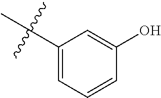 [SMILES: *c1cccc(O)c1] |
| 12-63 | 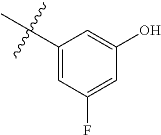 [SMILES: *c1cc(O)cc(F)c1] |
| 12-64 | 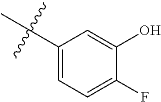 [SMILES: *c1ccc(F)c(O)c1] |
| 12-65 | 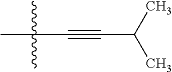 [SMILES: *C#CC(C)C] |
| 12-66 | 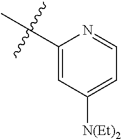 |
| 12-67 | 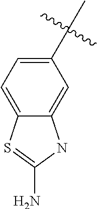 |
| 12-68 | 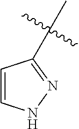 [SMILES: *c1cc[nH]n1] |
| 12-69 | 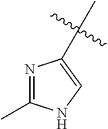 [SMILES: *c1c[nH]c(C)n1] |
| 12-70 | 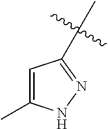 [SMILES: *c1cc(C)[nH]n1] |
| 12-71 | 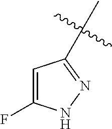 [SMILES: *c1cc(F)[nH]n1] |
| 12-72 | 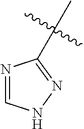 [SMILES: *c1nc[nH]n1] |
| 12-73 | 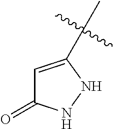 [SMILES: *c1cc(=O)[nH][nH]1] |
| 12-74 | 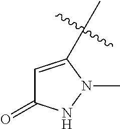 [SMILES: *c1cc(=O)[nH]n1C] |
| 12-75 | 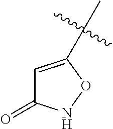 [SMILES: *c1cc(=O)[nH]o1] |
| 12-76 | 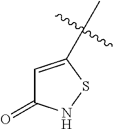 [SMILES: *c1cc(=O)[nH]s1] |
| 12-77 | 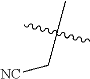 [SMILES: *CC#N] |
| 12-78 | 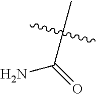 [SMILES: *C(N)=O] |
| 12-79 | 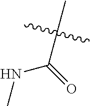 [SMILES: *C(=O)NC] |
| 12-80 | 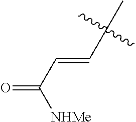 [SMILES: */C=C/C(=O)NC] |
| 12-81 | 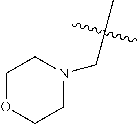 [SMILES: *CN1CCOCC1] |
| 12-82 | 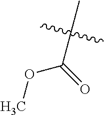 [SMILES: *C(=O)OC] |
| 12-83 | 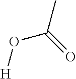 |
| 12-84 | 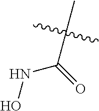 [SMILES: *C(=O)NO] |
| 12-85 | 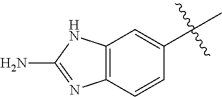 [SMILES: *c1ccc2nc(N)[nH]c2c1] |
| 12-86 | 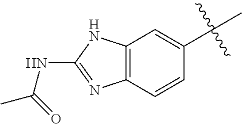 [SMILES: *c1ccc2nc(NC(C)=O)[nH]c2c1] |
| 12-87 | 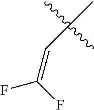 [SMILES: *C=C(F)F] |
| 12-88 | 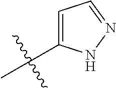 [SMILES: *c1ccn[nH]1] |
| 12-89 | 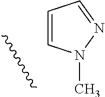 |
| 12-90 | 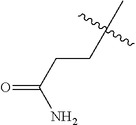 [SMILES: *CCC(N)=O] |
| 12-91 | 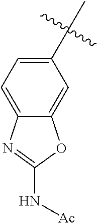 [SMILES: *c1ccc2nc(NC(C)=O)oc2c1] |
| 12-92 | 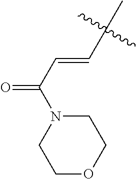 [SMILES: */C=C/C(=O)N1CCOCC1] |
| 12-93 | 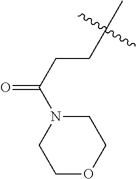 [SMILES: *CCC(=O)N1CCOCC1] |
| 12-94 | 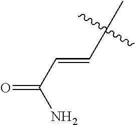 [SMILES: */C=C/C(N)=O] |
| 12-95 | 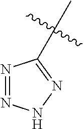 [SMILES: *c1nn[nH]n1] |
| 12-96 | 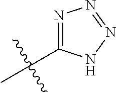 [SMILES: *c1nnn[nH]1] |
| 12-97 | —F |
| 12-98 | 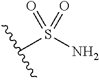 [SMILES: *S(N)(=O)=O] |
| 12-99 | 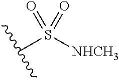 [SMILES: *S(=O)(=O)NC] |
| 12-100 | 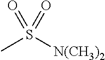 |
| 12-101 | 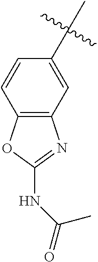 [SMILES: *c1ccc2oc(NC(C)=O)nc2c1] |
| 12-102 | 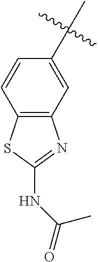 [SMILES: *c1ccc2sc(NC(C)=O)nc2c1] |

Other illustrative compounds of the present invention have a structure of Formula V-A, V-A1, or V-A2, wherein B is a moiety described in Table 1, in combination with R³, which is —H, —Cl, —F, or CH₃, and R⁹, which is —H, —CH₃, or —CH₂CH₃. A compound of Formula V-A, V-A1, or V-A2 includes any combination of R³, B, and R⁹.

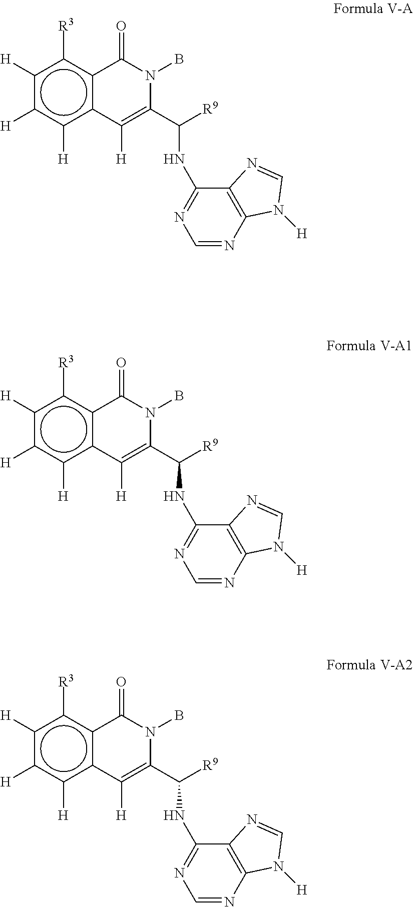

Yet other illustrative compounds of the present invention have a structure of Formula V-B, wherein B is a moiety described in Table 1, in combination with R³, which is —H, —Cl, —F, or CH₃, and R⁹, which is —H, —CH₃, or —CH₂CH₃. A compound of Formula V-B includes any combination of R³, B, and R⁹.

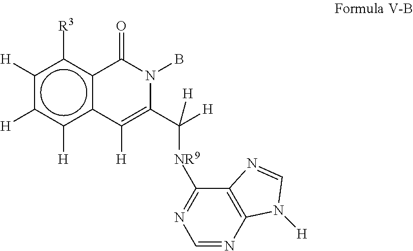

Some other illustrative compounds of the present invention have a structure of Formula VI-A, wherein B is a moiety described in Table 1, in combination with R³, which is —H, —Cl, —F, or CH₃, and R⁹, which is —H, —CH₃, or —CH₂CH₃. A compound of Formula VI-A includes any combination of R³, B, and R⁹.

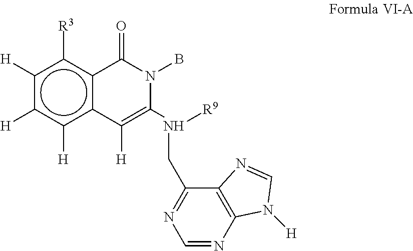

Further illustrative compounds that can be employed as described herein have a structure of one of Formulae VII-A1, VII-A2, VIII-A1, VIII-A2, IX-A1, IX-A2, X-A1, X-A2, XI-A1, XI-A2, XII-A, XII-A1, XII-A2, XIII-A, XIII-A1, XIII-A2, XIV-A, XIV-A1, or XIV-A2: wherein B is a moiety described in Table 1, any R¹² as described in Table 2, in combination with R³, which is —H, —Cl, —F, or CH₃, R⁹ which is —H, —CH₃, or —CH₂CH₃, and R^{a′} which is —H, —Cl, —F, or —NH₂. A compound of Formulae VII-A1, VII-A2, VIII-A1, VIII-A2, IX-A1, IX-A2, X-A1, X-A2, XI-A1, XI-A2, XII-A, XII-A1, XII-A2, XIII-A, XIII-A1, XIII-A2, XIV-A, XIV-A1, or XIV-A2: includes any combination of R^{a}, R³, B, R⁹ and R¹².

Additional exemplary compounds include but are not limited to the following:

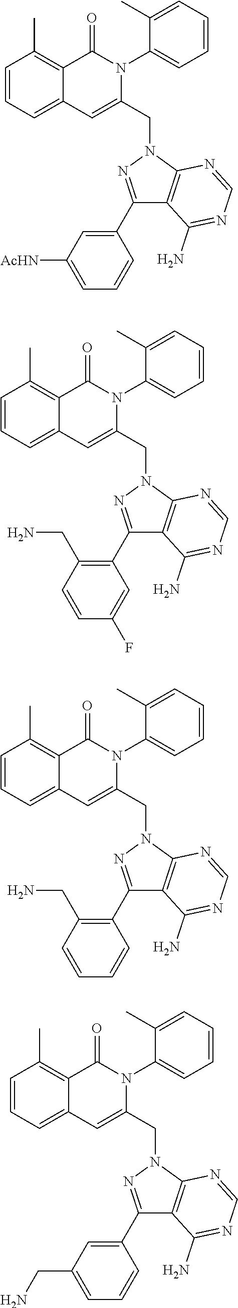

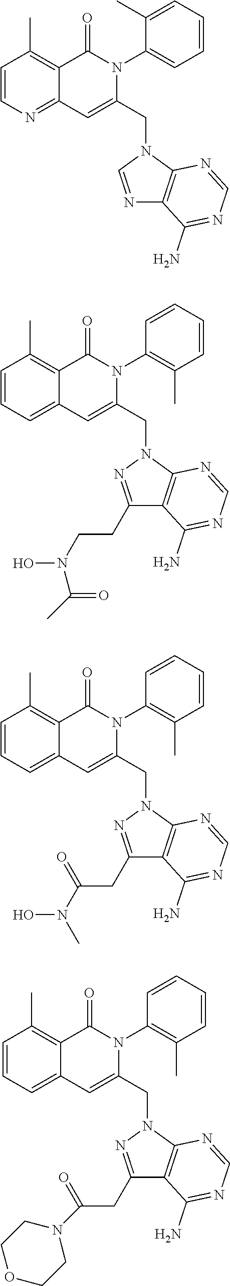

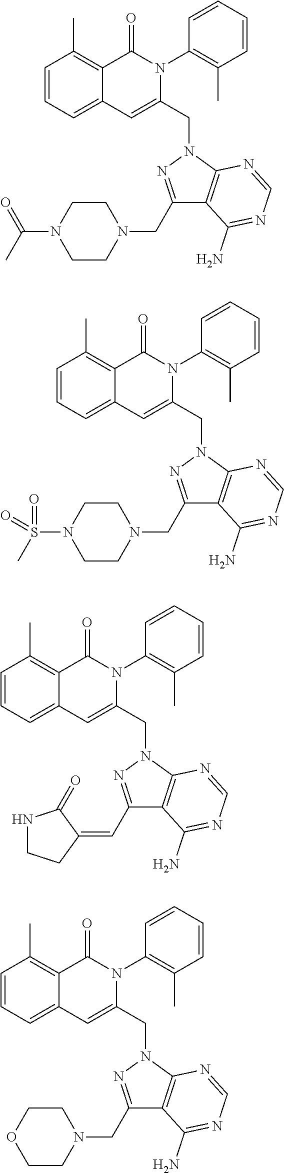

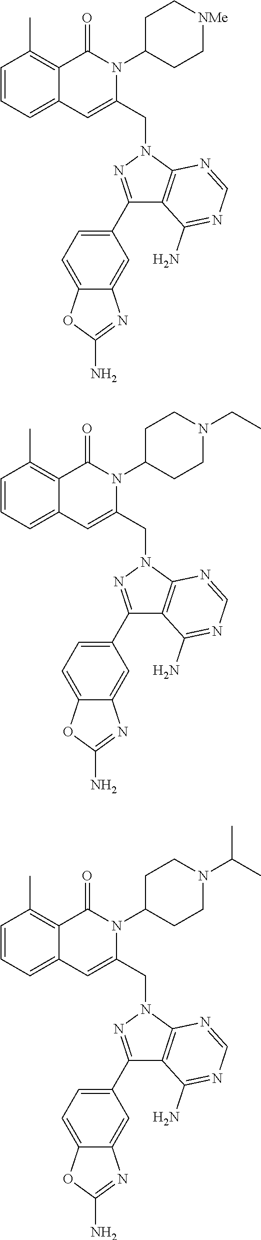 [SMILES: Cc1cccc2cc(Cn3nc(-c4ccc5oc(N)nc5c4)c4c(N)ncnc43)n(C3CCN(C)CC3)c(=O)c12 ; CCN1CCC(n2c(Cn3nc(-c4ccc5oc(N)nc5c4)c4c(N)ncnc43)cc3cccc(C)c3c2=O)CC1 ; Cc1cccc2cc(Cn3nc(-c4ccc5oc(N)nc5c4)c4c(N)ncnc43)n(C3CCN(C(C)C)CC3)c(=O)c12]

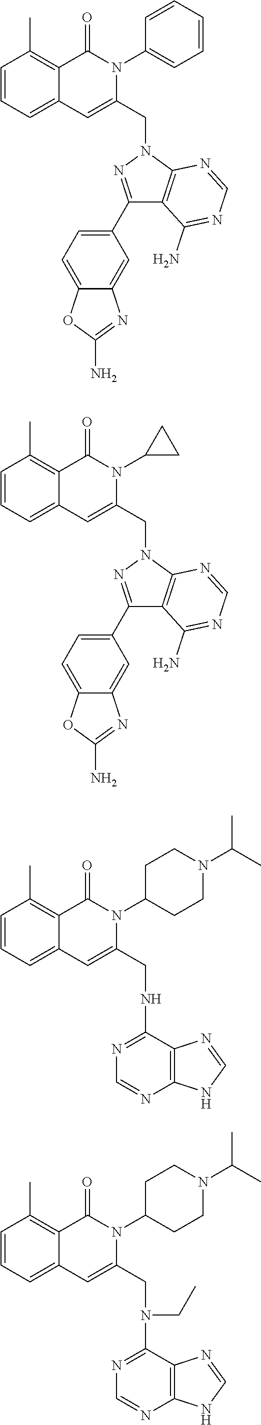 [SMILES: Cc1cccc2cc(Cn3nc(-c4ccc5oc(N)nc5c4)c4c(N)ncnc43)n(-c3ccccc3)c(=O)c12 ; Cc1cccc2cc(Cn3nc(-c4ccc5oc(N)nc5c4)c4c(N)ncnc43)n(C3CC3)c(=O)c12 ; Cc1cccc2cc(CNc3ncnc4[nH]cnc34)n(C3CCN(C(C)C)CC3)c(=O)c12 ; CCN(Cc1cc2cccc(C)c2c(=O)n1C1CCN(C(C)C)CC1)c1ncnc2[nH]cnc12]

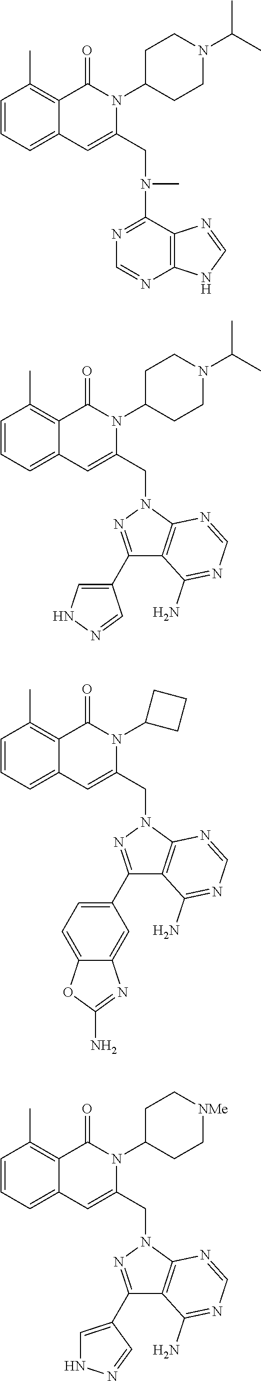 [SMILES: Cc1cccc2cc(CN(C)c3ncnc4[nH]cnc34)n(C3CCN(C(C)C)CC3)c(=O)c12 ; Cc1cccc2cc(Cn3nc(-c4cn[nH]c4)c4c(N)ncnc43)n(C3CCN(C(C)C)CC3)c(=O)c12 ; Cc1cccc2cc(Cn3nc(-c4ccc5oc(N)nc5c4)c4c(N)ncnc43)n(C3CCC3)c(=O)c12 ; Cc1cccc2cc(Cn3nc(-c4cn[nH]c4)c4c(N)ncnc43)n(C3CCN(C)CC3)c(=O)c12]

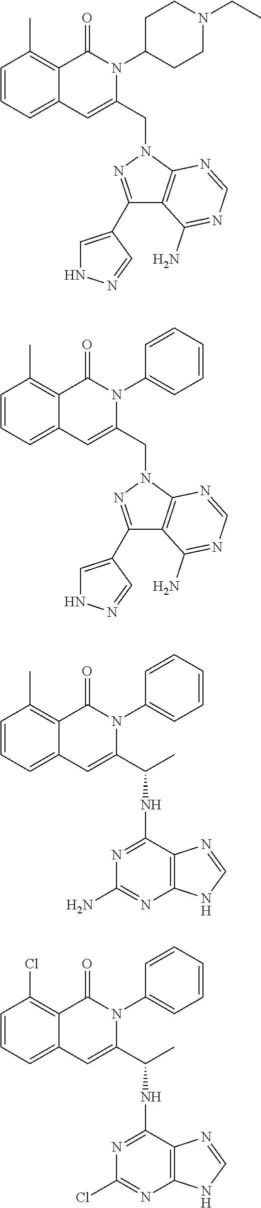 [SMILES: CCN1CCC(n2c(Cn3nc(-c4cn[nH]c4)c4c(N)ncnc43)cc3cccc(C)c3c2=O)CC1 ; Cc1cccc2cc(Cn3nc(-c4cn[nH]c4)c4c(N)ncnc43)n(-c3ccccc3)c(=O)c12 ; Cc1cccc2cc([C@H](C)Nc3nc(N)nc4[nH]cnc34)n(-c3ccccc3)c(=O)c12 ; C[C@H](Nc1nc(Cl)nc2[nH]cnc12)c1cc2cccc(Cl)c2c(=O)n1-c1ccccc1]

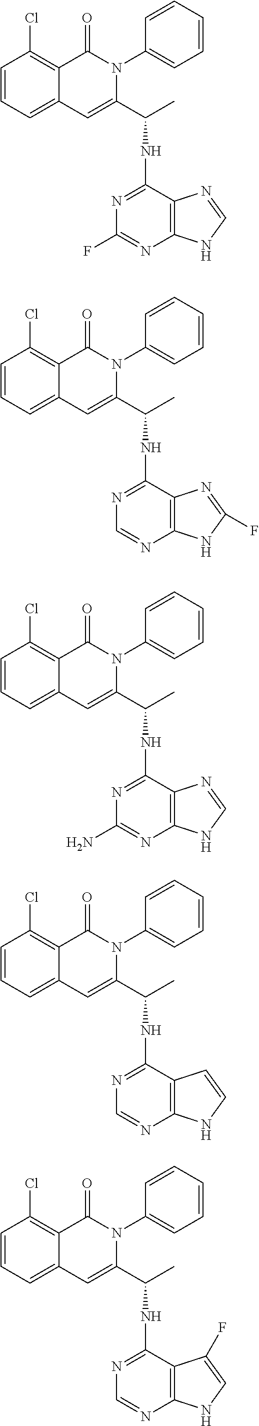

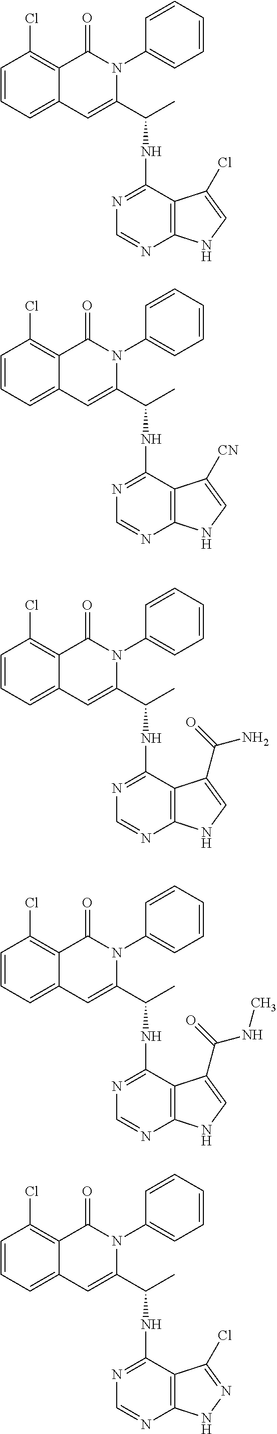 [SMILES: C[C@H](Nc1ncnc2[nH]cc(Cl)c12)c1cc2cccc(Cl)c2c(=O)n1-c1ccccc1 ; C[C@H](Nc1ncnc2[nH]cc(C#N)c12)c1cc2cccc(Cl)c2c(=O)n1-c1ccccc1 ; C[C@H](Nc1ncnc2[nH]cc(C(N)=O)c12)c1cc2cccc(Cl)c2c(=O)n1-c1ccccc1 ; CNC(=O)c1c[nH]c2ncnc(N[C@@H](C)c3cc4cccc(Cl)c4c(=O)n3-c3ccccc3)c12 ; C[C@H](Nc1ncnc2[nH]nc(Cl)c12)c1cc2cccc(Cl)c2c(=O)n1-c1ccccc1]

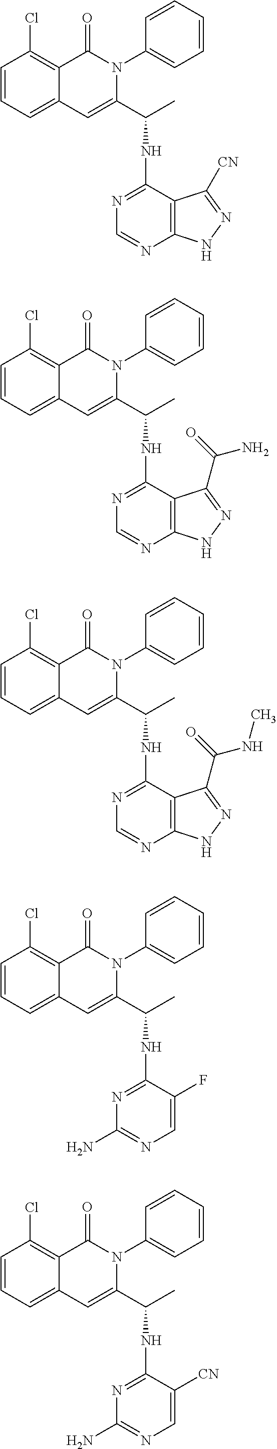 [SMILES: C[C@H](Nc1ncnc2[nH]nc(C#N)c12)c1cc2cccc(Cl)c2c(=O)n1-c1ccccc1 ; C[C@H](Nc1ncnc2[nH]nc(C(N)=O)c12)c1cc2cccc(Cl)c2c(=O)n1-c1ccccc1 ; CNC(=O)c1n[nH]c2ncnc(N[C@@H](C)c3cc4cccc(Cl)c4c(=O)n3-c3ccccc3)c12 ; C[C@H](Nc1nc(N)ncc1F)c1cc2cccc(Cl)c2c(=O)n1-c1ccccc1 ; C[C@H](Nc1nc(N)ncc1C#N)c1cc2cccc(Cl)c2c(=O)n1-c1ccccc1]

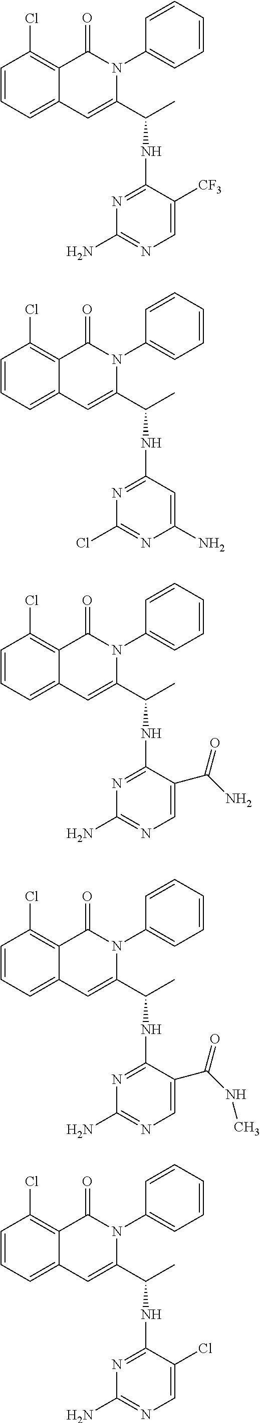

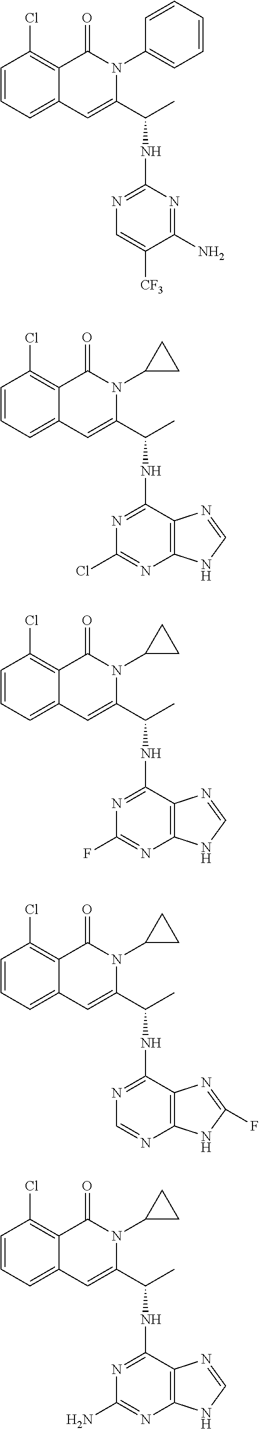 [SMILES: C[C@H](Nc1ncc(C(F)(F)F)c(N)n1)c1cc2cccc(Cl)c2c(=O)n1-c1ccccc1 ; C[C@H](Nc1nc(Cl)nc2[nH]cnc12)c1cc2cccc(Cl)c2c(=O)n1C1CC1 ; C[C@H](Nc1nc(F)nc2[nH]cnc12)c1cc2cccc(Cl)c2c(=O)n1C1CC1 ; C[C@H](Nc1ncnc2[nH]c(F)nc12)c1cc2cccc(Cl)c2c(=O)n1C1CC1 ; C[C@H](Nc1nc(N)nc2[nH]cnc12)c1cc2cccc(Cl)c2c(=O)n1C1CC1]

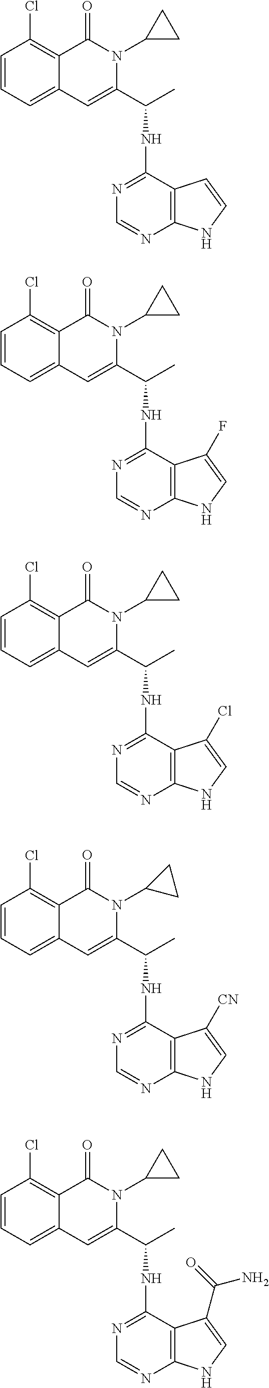 [SMILES: C[C@H](Nc1ncnc2[nH]ccc12)c1cc2cccc(Cl)c2c(=O)n1C1CC1 ; C[C@H](Nc1ncnc2[nH]cc(F)c12)c1cc2cccc(Cl)c2c(=O)n1C1CC1 ; C[C@H](Nc1ncnc2[nH]cc(Cl)c12)c1cc2cccc(Cl)c2c(=O)n1C1CC1 ; C[C@H](Nc1ncnc2[nH]cc(C#N)c12)c1cc2cccc(Cl)c2c(=O)n1C1CC1 ; C[C@H](Nc1ncnc2[nH]cc(C(N)=O)c12)c1cc2cccc(Cl)c2c(=O)n1C1CC1]

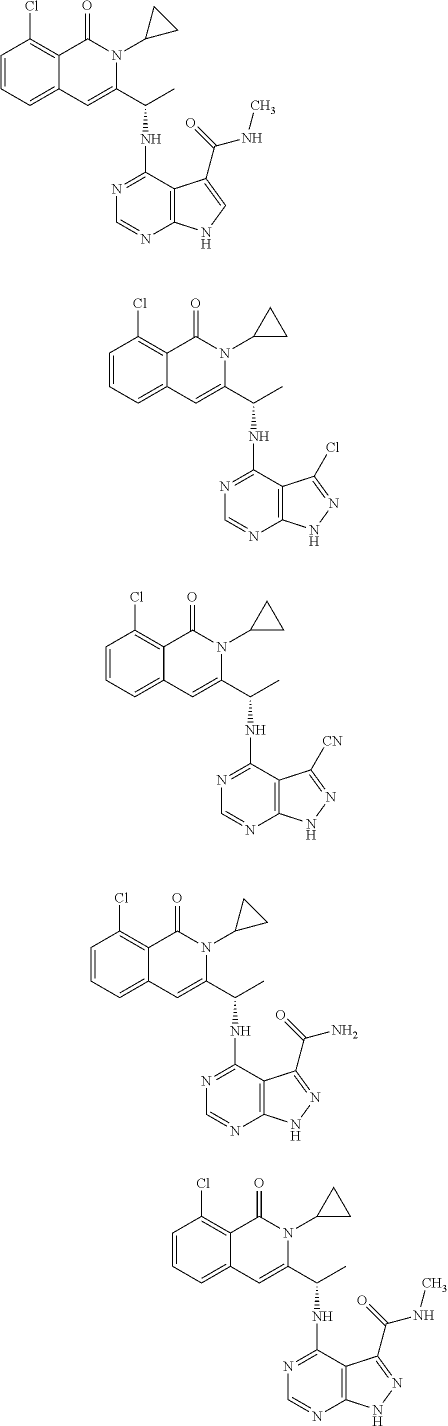 [SMILES: CNC(=O)c1c[nH]c2ncnc(N[C@@H](C)c3cc4cccc(Cl)c4c(=O)n3C3CC3)c12 ; C[C@H](Nc1ncnc2[nH]nc(Cl)c12)c1cc2cccc(Cl)c2c(=O)n1C1CC1 ; C[C@H](Nc1ncnc2[nH]nc(C#N)c12)c1cc2cccc(Cl)c2c(=O)n1C1CC1 ; C[C@H](Nc1ncnc2[nH]nc(C(N)=O)c12)c1cc2cccc(Cl)c2c(=O)n1C1CC1 ; CNC(=O)c1n[nH]c2ncnc(N[C@@H](C)c3cc4cccc(Cl)c4c(=O)n3C3CC3)c12]

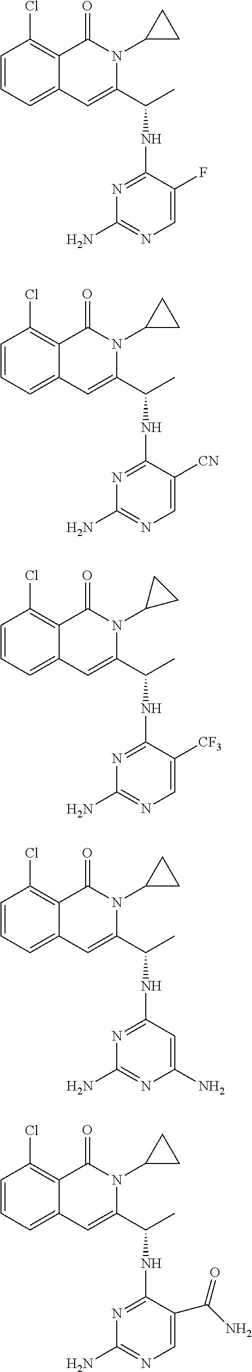

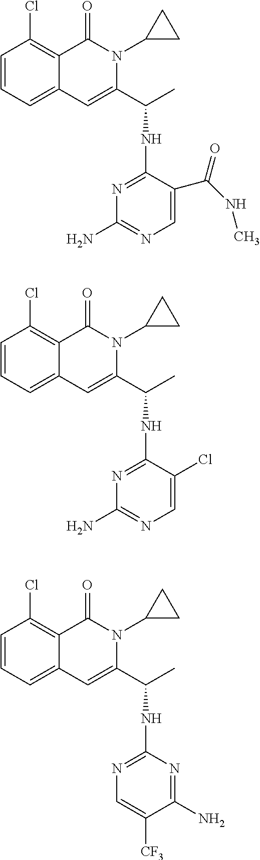 [SMILES: CNC(=O)c1cnc(N)nc1N[C@@H](C)c1cc2cccc(Cl)c2c(=O)n1C1CC1 ; C[C@H](Nc1nc(N)ncc1Cl)c1cc2cccc(Cl)c2c(=O)n1C1CC1 ; C[C@H](Nc1ncc(C(F)(F)F)c(N)n1)c1cc2cccc(Cl)c2c(=O)n1C1CC1]

In some embodiments, the PI3K inhibitor is a compound of Formula I-1:

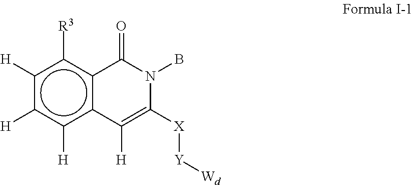

or its pharmaceutically acceptable salt thereof, wherein
B is a moiety of Formula II:

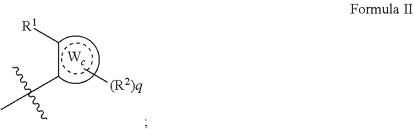

wherein W_{c} is aryl, heteroaryl, heterocycloalkyl, or cycloalkyl, and
q is an integer of 0, 1, 2, 3, or 4;
X is a bond or —(CH(R⁹))_{z}—, and z is an integer of 1;

### Y is —N(R⁹)—;

### W_{d} is:

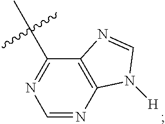 [SMILES: *c1ncnc2[nH]cnc12]

R¹ is hydrogen, alkyl, alkenyl, alkynyl, alkoxy, amido, alkoxycarbonyl, sulfonamido, halo, cyano, or nitro;
R² is alkyl, alkenyl, alkynyl, cycloalkyl, heterocycloalkyl, aryl, heteroaryl, heteroarylalkyl, alkoxy, amino, halo, cyano, hydroxy or nitro;
R³ is hydrogen, alkyl, alkenyl, alkynyl, cycloalkyl, heterocycloalkyl, alkoxy, amido, amino, alkoxycarbonyl sulfonamido, halo, cyano, hydroxy or nitro; and each instance of R⁹ is independently hydrogen, alkyl, or heterocycloalkyl.

In some embodiments, the compound is predominately in an (S)-stereochemical configuration

In some embodiments, X is —(CH(R⁹))_{z}—, and Y is —NH—.

In some embodiments, R³ is —H, —CH₃, —CH₂CH₃, —CF₃, —Cl or —F.

In some embodiments, B is a moiety of Formula II:

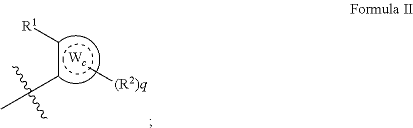

wherein W_{c} is aryl, heteroaryl, heterocycloalkyl, or cycloalkyl;
q is an integer of 0 or 1;
R¹ is hydrogen, alkyl, or halo;
R² is alkyl or halo;
R³ is hydrogen, alkyl, or halo; and, optionally wherein the compound has one or more of the following features:

(i) X is —(CH(R⁹))_{z}—, wherein R⁹ is methyl and z=1; and W_{d} is

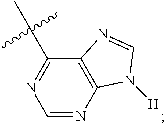 [SMILES: *c1ncnc2[nH]cnc12]

and/or

(ii) R³ is methyl or chloro.

In some embodiments, the compound has a structure of Formula V-A2:

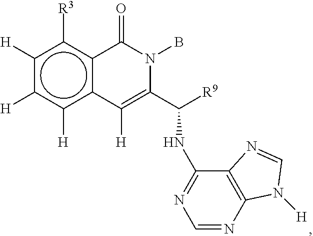

optionally wherein
(i) B is a moiety of Formula II:

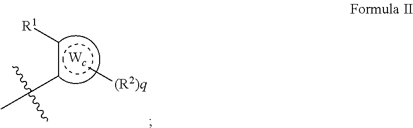

and W_{c} is aryl or cycloalkyl, and/or
(ii) R³ is methyl or chloro and further, optionally wherein one or more of the following also applies: (a) R⁹ is methyl or ethyl, (b) B is substituted or unsubstituted phenyl, (c) B is substituted or unsubstituted cycloalkyl. In some embodiments where B is substituted phenyl, B is substituted with fluoro. In some embodiments, B is phenyl that is substituted with one fluoro in the ortho or meta position of the phenyl ring.

In some embodiments, a compound used as described herein is selected from

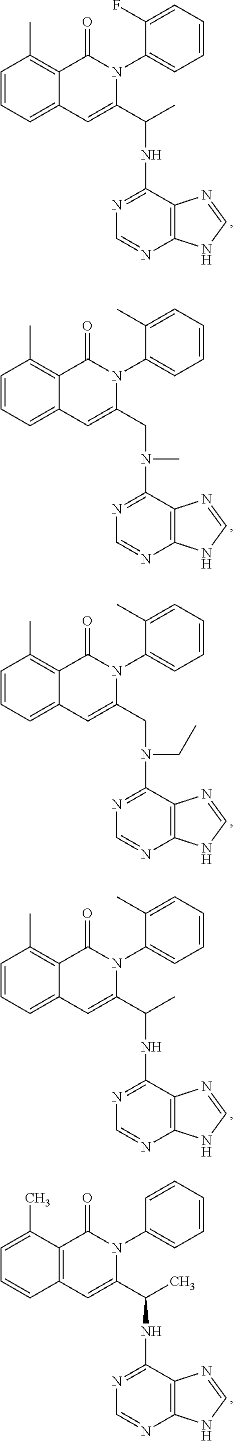

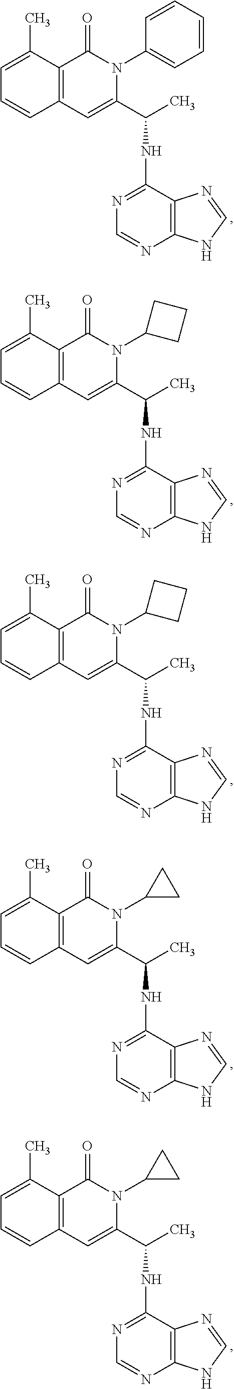 [SMILES: Cc1cccc2cc([C@H](C)Nc3ncnc4[nH]cnc34)n(-c3ccccc3)c(=O)c12 ; Cc1cccc2cc([C@@H](C)Nc3ncnc4[nH]cnc34)n(C3CCC3)c(=O)c12 ; Cc1cccc2cc([C@H](C)Nc3ncnc4[nH]cnc34)n(C3CCC3)c(=O)c12 ; Cc1cccc2cc([C@@H](C)Nc3ncnc4[nH]cnc34)n(C3CC3)c(=O)c12 ; Cc1cccc2cc([C@H](C)Nc3ncnc4[nH]cnc34)n(C3CC3)c(=O)c12]

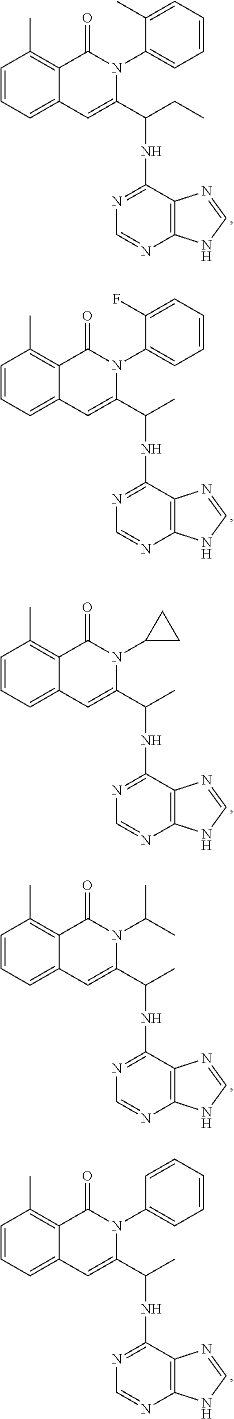

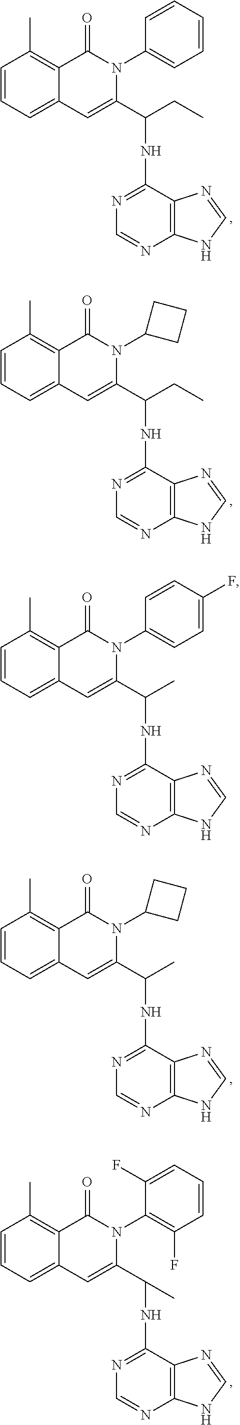 [SMILES: CCC(Nc1ncnc2[nH]cnc12)c1cc2cccc(C)c2c(=O)n1-c1ccccc1 ; CCC(Nc1ncnc2[nH]cnc12)c1cc2cccc(C)c2c(=O)n1C1CCC1 ; Cc1cccc2cc(C(C)Nc3ncnc4[nH]cnc34)n(-c3ccc(F)cc3)c(=O)c12 ; Cc1cccc2cc(C(C)Nc3ncnc4[nH]cnc34)n(C3CCC3)c(=O)c12 ; Cc1cccc2cc(C(C)Nc3ncnc4[nH]cnc34)n(-c3c(F)cccc3F)c(=O)c12]

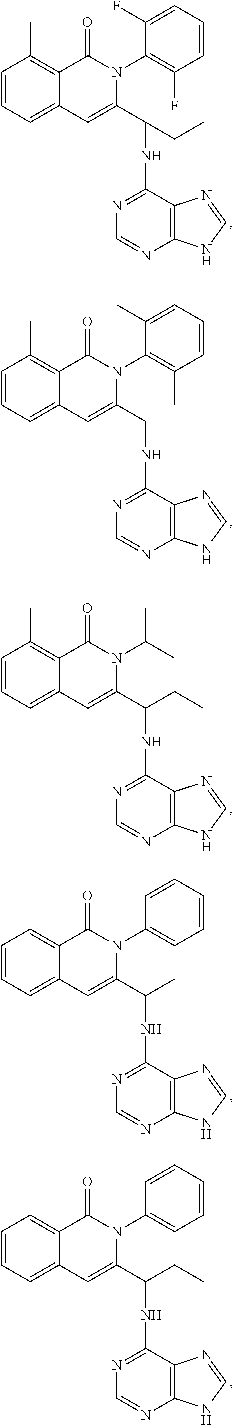

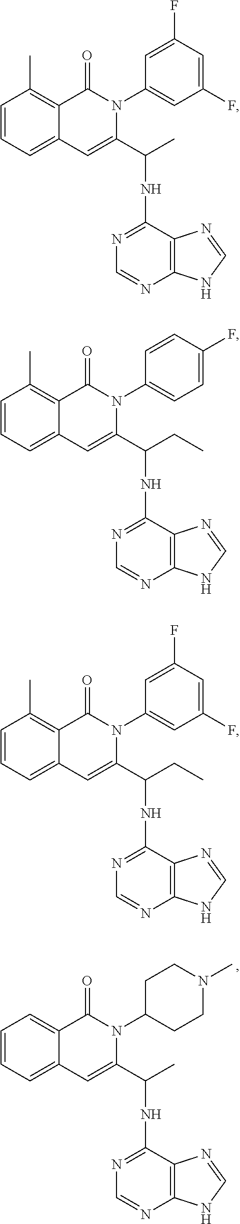 [SMILES: Cc1cccc2cc(C(C)Nc3ncnc4[nH]cnc34)n(-c3cc(F)cc(F)c3)c(=O)c12 ; CCC(Nc1ncnc2[nH]cnc12)c1cc2cccc(C)c2c(=O)n1-c1ccc(F)cc1 ; CCC(Nc1ncnc2[nH]cnc12)c1cc2cccc(C)c2c(=O)n1-c1cc(F)cc(F)c1 ; CC(Nc1ncnc2[nH]cnc12)c1cc2ccccc2c(=O)n1C1CCN(C)CC1]

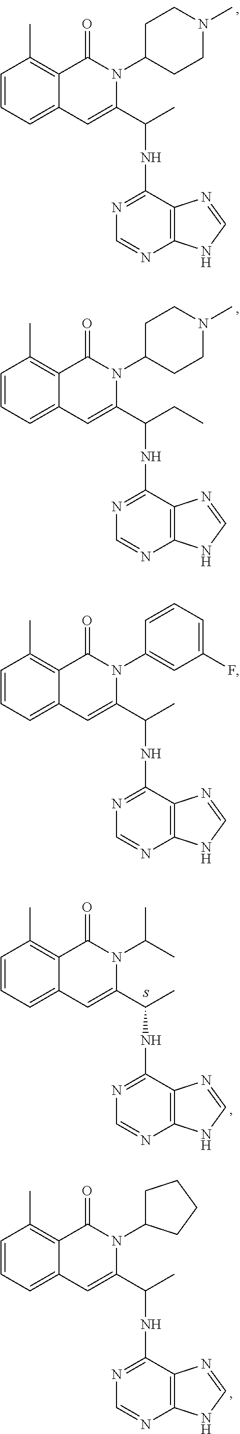

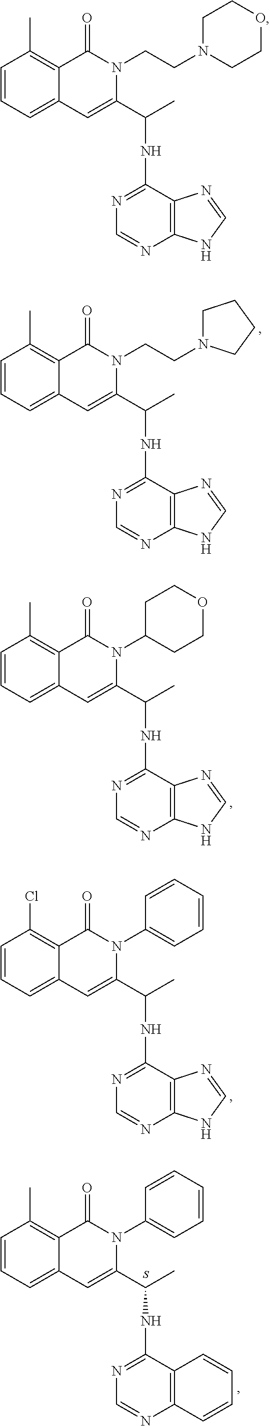

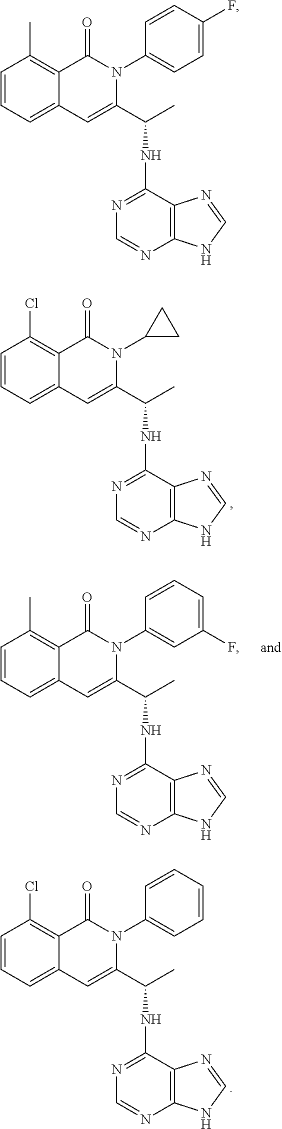 [SMILES: Cc1cccc2cc([C@H](C)Nc3ncnc4[nH]cnc34)n(-c3ccc(F)cc3)c(=O)c12 ; C[C@H](Nc1ncnc2[nH]cnc12)c1cc2cccc(Cl)c2c(=O)n1C1CC1 ; Cc1cccc2cc([C@H](C)Nc3ncnc4[nH]cnc34)n(-c3cccc(F)c3)c(=O)c12 ; C[C@H](Nc1ncnc2[nH]cnc12)c1cc2cccc(Cl)c2c(=O)n1-c1ccccc1]

In some embodiments, the compound is selected from

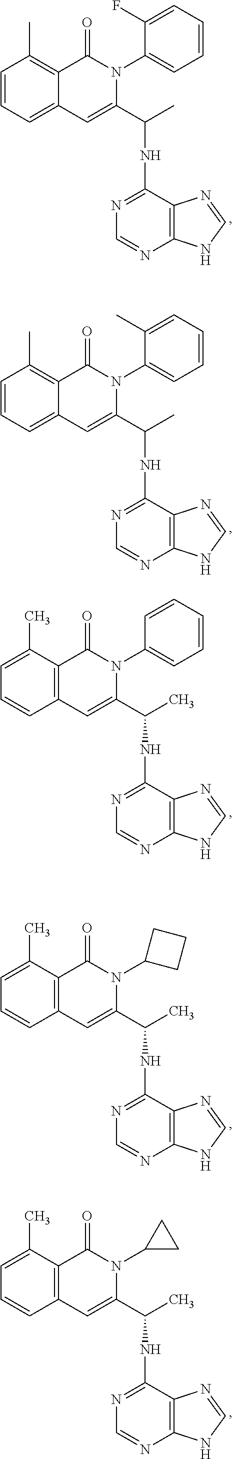

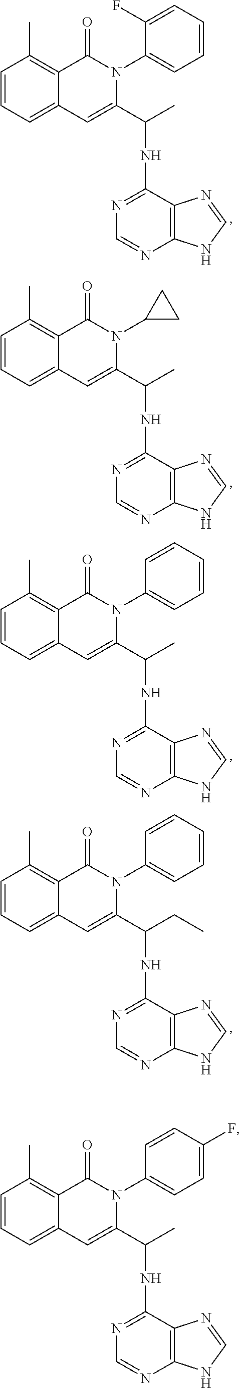

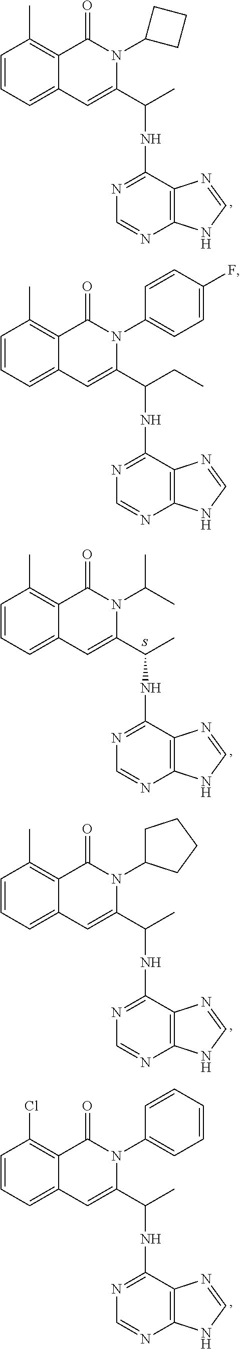

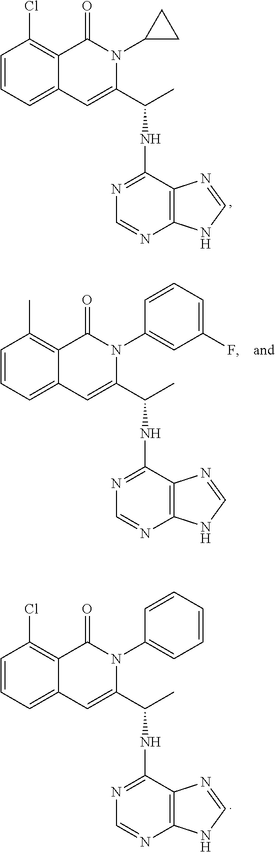 [SMILES: C[C@H](Nc1ncnc2[nH]cnc12)c1cc2cccc(Cl)c2c(=O)n1C1CC1 ; Cc1cccc2cc([C@H](C)Nc3ncnc4[nH]cnc34)n(-c3cccc(F)c3)c(=O)c12 ; C[C@H](Nc1ncnc2[nH]cnc12)c1cc2cccc(Cl)c2c(=O)n1-c1ccccc1]

In some embodiments, the compound is selected from

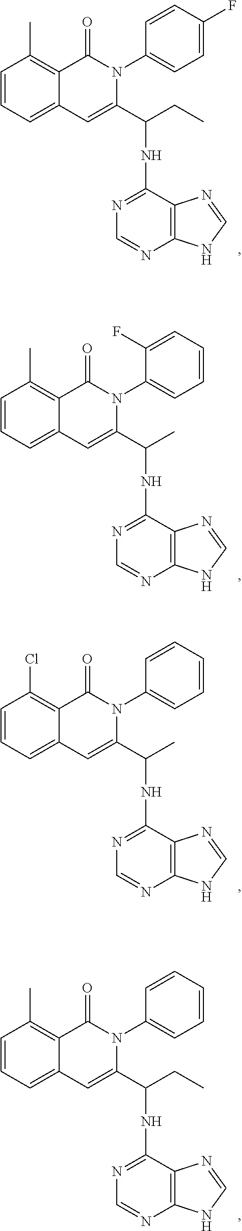 [SMILES: CCC(Nc1ncnc2[nH]cnc12)c1cc2cccc(C)c2c(=O)n1-c1ccc(F)cc1 ; Cc1cccc2cc(C(C)Nc3ncnc4[nH]cnc34)n(-c3ccccc3F)c(=O)c12 ; CC(Nc1ncnc2[nH]cnc12)c1cc2cccc(Cl)c2c(=O)n1-c1ccccc1 ; CCC(Nc1ncnc2[nH]cnc12)c1cc2cccc(C)c2c(=O)n1-c1ccccc1]

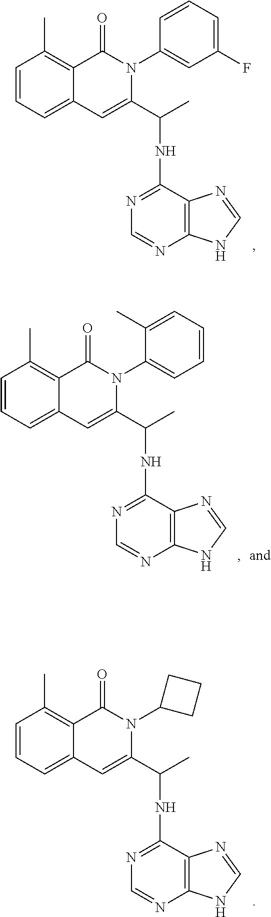 [SMILES: Cc1cccc2cc(C(C)Nc3ncnc4[nH]cnc34)n(-c3cccc(F)c3)c(=O)c12 ; Cc1ccccc1-n1c(C(C)Nc2ncnc3[nH]cnc23)cc2cccc(C)c2c1=O ; Cc1cccc2cc(C(C)Nc3ncnc4[nH]cnc34)n(C3CCC3)c(=O)c12]

In some embodiments, the PI3K inhibitor has a formula selected from the group consisting of:

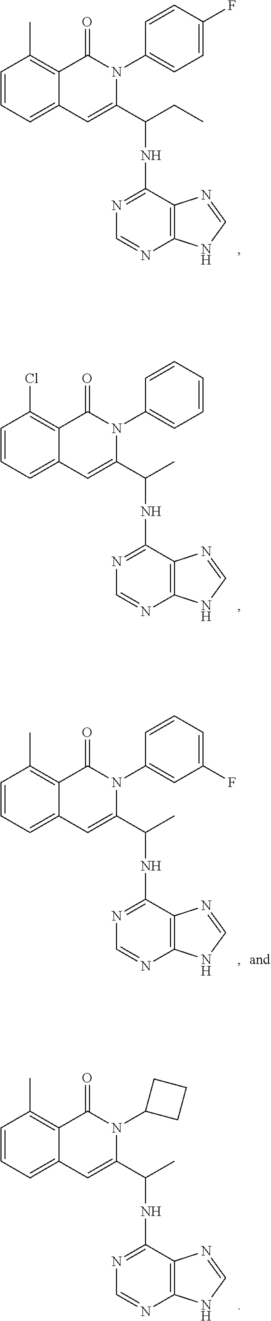 [SMILES: CCC(Nc1ncnc2[nH]cnc12)c1cc2cccc(C)c2c(=O)n1-c1ccc(F)cc1 ; CC(Nc1ncnc2[nH]cnc12)c1cc2cccc(Cl)c2c(=O)n1-c1ccccc1 ; Cc1cccc2cc(C(C)Nc3ncnc4[nH]cnc34)n(-c3cccc(F)c3)c(=O)c12 ; Cc1cccc2cc(C(C)Nc3ncnc4[nH]cnc34)n(C3CCC3)c(=O)c12]

In some embodiments, the compound is the S-enantiomer having an enantiomeric purity selected from greater than about 55%, greater than about 80%, greater than about 90%, and greater than about 95%.

In some such embodiments, the compound is selected from:

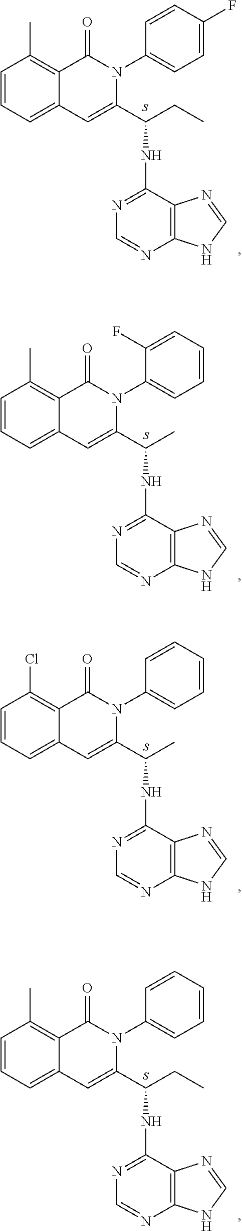

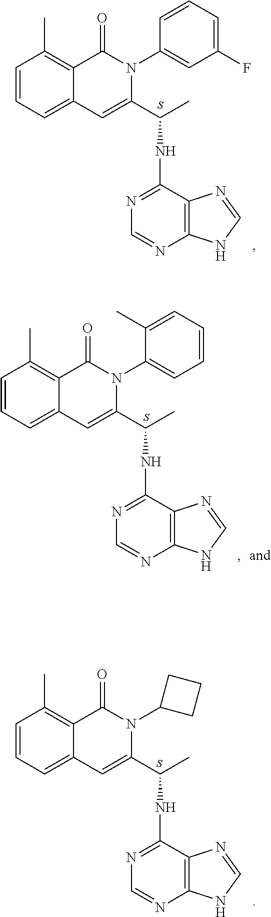

In some embodiments, the PI3K inhibitor has a formula selected from the group consisting of:

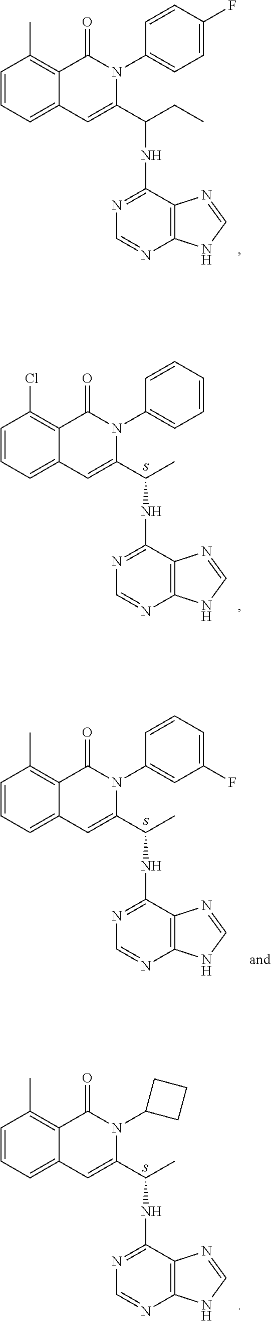

In certain such embodiments, the compound is

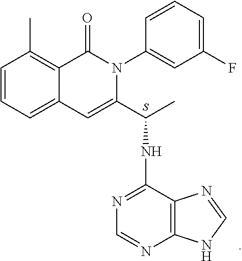

In other such embodiments, the compound is

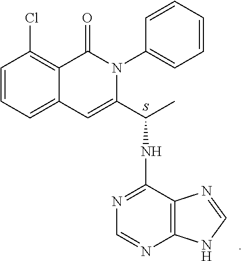

In yet other such embodiments, the compound is

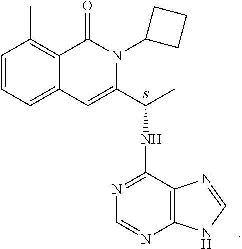

In some embodiments, the compound has the following structure:

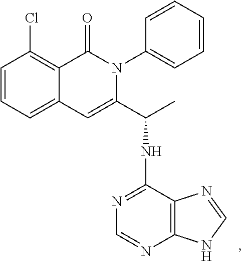 [SMILES: C[C@H](Nc1ncnc2[nH]cnc12)c1cc2cccc(Cl)c2c(=O)n1-c1ccccc1]

which is also referred to herein as Compound 292.

In some embodiments, a polymorph of a compound disclosed herein is used. Exemplary polymorphs are disclosed in U.S. Patent Publication No. 2012-0184568 (“the '568 publication”), which is hereby incorporated by reference in its entirety.

In one embodiment, the compound is Form A of Compound 292, as described in the '568 publication. In another embodiment, the compound is Form B of Compound 292, as described in the '568 publication. In yet another embodiment, the compound is Form C of Compound 292, as described in the '568 publication. In yet another embodiment, the compound is Form D of Compound 292, as described in the '568 publication. In yet another embodiment, the compound is Form E of Compound 292, as described in the '568 publication. In yet another embodiment, the compound is Form F of Compound 292, as described in the '568 publication. In yet another embodiment, the compound is Form G of Compound 292, as described in the '568 publication. In yet another embodiment, the compound is Form H of Compound 292, as described in the '568 publication. In yet another embodiment, the compound is Form I of Compound 292, as described in the '568 publication. In yet another embodiment, the compound is Form J of Compound 292, as described in the '568 publication.

Any of the compounds (PI3K inhibitors) disclosed herein can be in the form of pharmaceutically acceptable salts, hydrates, solvates, chelates, non-covalent complexes, isomers, prodrugs, isotopically labeled derivatives, or mixtures thereof.

Chemical entities described herein can be synthesized according to exemplary methods disclosed in U.S. Patent Publication No. US 2009/0312319, International Patent Publication No. WO 2011/008302A1, and U.S. Patent Publication No. 2012/0184568, each of which is hereby incorporated by reference in its entirety, and/or according to methods known in the art.

### Pharmaceutical Compositions

The compounds disclosed herein can be formulated as pharmaceutical compositions.

In some embodiments, the pharmaceutical compositions comprise a compound disclosed herein, or a pharmaceutically acceptable salt thereof. In some embodiments, the pharmaceutical composition comprises two, three, four, or more compounds disclosed herein, or pharmaceutically acceptable salts thereof, as described herein. In some embodiments, the composition comprises a pharmaceutically acceptable excipient. In some embodiments, the composition comprises a plurality of pharmaceutically acceptable excipients.

The subject pharmaceutical compositions are typically formulated to provide a therapeutically effective amount of a compound of the present invention as the active ingredient, or a pharmaceutically acceptable salt, ester, prodrug, solvate, hydrate or derivative thereof. Where desired, the pharmaceutical compositions contain pharmaceutically acceptable salt and/or coordination complex thereof, and one or more pharmaceutically acceptable excipients, carriers, including inert solid diluents and fillers, diluents, including sterile aqueous solution and various organic solvents, permeation enhancers, solubilizers and adjuvants.

The subject pharmaceutical compositions can be administered alone or in combination with one or more other additional therapies (e.g., one or more additional agents, which are also typically administered in the form of pharmaceutical compositions). Where desired, the subject compounds and other agent(s) can be mixed into a preparation or both components can be formulated into separate preparations to use them in combination separately or at the same time.

In some embodiments, the concentration of one or more of the compounds provided herein in the pharmaceutical compositions provided herein is less than 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v or v/v.

In some embodiments, the concentration of one or more of the compounds provided herein in the pharmaceutical compositions provided herein is greater than 90%, 80%, 70%, 60%, 50%, 40%, 30%, 20%, 19.75%, 19.50%, 19.25%, 19%, 18.75%, 18.50%, 18.25%, 18%, 17.75%, 17.50%, 17.25%, 17%, 16.75%, 16.50%, 16.25%, 16%, 15.75%, 15.50%, 15.25%, 15%, 14.75%, 14.50%, 14.25%, 14%, 13.75%, 13.50%, 13.25%, 13%, 12.75%, 12.50%, 12.25%, 12%, 11.75%, 11.50%, 11.25%, 11%, 10.75%, 10.50%, 10.25%, 10%, 9.75%, 9.50%, 9.25%, 9%, 8.75%, 8.50%, 8.25%, 8%, 7.75%, 7.50%, 7.25%, 7%, 6.75%, 6.50%, 6.25%, 6%, 5.75%, 5.50%, 5.25%, 5%, 4.75%, 4.50%, 4.25%, 4%, 3.75%, 3.50%, 3.25%, 3%, 2.75%, 2.50%, 2.25%, 2%, 1.75%, 1.50%, 1.25%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v, or v/v.

In some embodiments, the concentration of one or more of the compounds provided herein in the pharmaceutical compositions provided herein is in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, approximately 1% to approximately 10% w/w, w/v or v/v. v/v.

In some embodiments, the concentration of one or more of the compounds provided herein in the pharmaceutical compositions provided herein is in the range from approximately 0.001% to approximately 10%, approximately 0.01% to approximately 5%, approximately 0.02% to approximately 4.5%, approximately 0.03% to approximately 4%, approximately 0.04% to approximately 3.5%, approximately 0.05% to approximately 3%, approximately 0.06% to approximately 2.5%, approximately 0.07% to approximately 2%, approximately 0.08% to approximately 1.5%, approximately 0.09% to approximately 1%, approximately 0.1% to approximately 0.9% w/w, w/v or v/v.

In some embodiments, the amount of one or more of the compounds provided herein in the pharmaceutical compositions provided herein is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of one or more of the compounds provided herein in the pharmaceutical compositions provided herein is equal to or more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g.

In some embodiments, the amount of one or more of the compounds provided herein in the pharmaceutical compositions provided herein is in the range of 0.0001-10 g, 0.0005-9 g, 0.001-8 g, 0.005-7 g, 0.01-6 g, 0.05-5 g, 0.1-4 g, 0.5-4 g, or 1-3 g.

Described below are non-limiting exemplary pharmaceutical compositions and methods for preparing the same.

### Pharmaceutical Compositions for Oral Administration

In some embodiments, a pharmaceutical composition for oral administration is used, wherein the composition comprises a compound of the present invention, and a pharmaceutical excipient suitable for oral administration.

In some embodiments, a solid pharmaceutical composition for oral administration is used, wherein the composition comprises (i) an effective amount of a compound of the present invention; and optionally, (ii) an effective amount of a second agent; and (iii) a pharmaceutical excipient suitable for oral administration. In some embodiments, the composition further contains: (iv) an effective amount of a third agent.

In some embodiments, the pharmaceutical composition can be a liquid pharmaceutical composition suitable for oral consumption. Pharmaceutical compositions of the invention suitable for oral administration can be presented as discrete dosage forms, such as capsules, cachets, or tablets, or liquids or aerosol sprays each containing a predetermined amount of an active ingredient as a powder or in granules, a solution, or a suspension in an aqueous or non-aqueous liquid, an oil-in-water emulsion, or a water-in-oil liquid emulsion. Such dosage forms can be prepared by any of the methods of pharmacy, but all methods include the step of bringing the active ingredient into association with the carrier, which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

The pharmaceutical compositions can be anhydrous pharmaceutical compositions and dosage forms comprising an active ingredient, since water can facilitate the degradation of some compounds. For example, water can be added (e.g., 5%) in the pharmaceutical arts as a means of simulating long-term storage in order to determine characteristics such as shelf-life or the stability of formulations over time. Anhydrous pharmaceutical compositions and dosage forms of the invention can be prepared using anhydrous or low moisture containing ingredients and low moisture or low humidity conditions. Pharmaceutical compositions and dosage forms of the invention which contain lactose can be made anhydrous if substantial contact with moisture and/or humidity during manufacturing, packaging, and/or storage is expected. An anhydrous pharmaceutical composition can be prepared and stored such that its anhydrous nature is maintained. Accordingly, anhydrous compositions can be packaged using materials known to prevent exposure to water such that they can be included in suitable formulary kits. Examples of suitable packaging include, but are not limited to, hermetically sealed foils, plastic or the like, unit dose containers, blister packs, and strip packs.

An active ingredient can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants can be used in the compositions to provide tablets that disintegrate when exposed to an aqueous environment. Too much of a disintegrant can produce tablets which can disintegrate in the bottle. Too little can be insufficient for disintegration to occur and can thus alter the rate and extent of release of the active ingredient(s) from the dosage form. Thus, a sufficient amount of disintegrant that is neither too little nor too much to detrimentally alter the release of the active ingredient(s) can be used to form the dosage forms of the compounds disclosed herein. The amount of disintegrant used can vary based upon the type of formulation and mode of administration, and can be readily discernible to those of ordinary skill in the art. About 0.5 to about 15 weight percent of disintegrant, or about 1 to about 5 weight percent of disintegrant, can be used in the pharmaceutical composition. Disintegrants that can be used to form pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethylaureate, agar, or mixtures thereof. Additional lubricants include, for example, a syloid silica gel, a coagulated aerosol of synthetic silica, or mixtures thereof. A lubricant can optionally be added, in an amount of less than about 1 weight percent of the pharmaceutical composition.

When aqueous suspensions and/or elixirs are desired for oral administration, the essential active ingredient therein can be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactant which can be used to form pharmaceutical compositions and dosage forms of the invention include, but are not limited to, hydrophilic surfactants, lipophilic surfactants, and mixtures thereof. That is, a mixture of hydrophilic surfactants can be employed, a mixture of lipophilic surfactants can be employed, or a mixture of at least one hydrophilic surfactant and at least one lipophilic surfactant can be employed.

A suitable hydrophilic surfactant can generally have an HLB value of at least 10, while suitable lipophilic surfactants can generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions. Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants can be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Ionic surfactants can be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants can include, but not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof; polyoxyethylated vitamins and derivatives thereof; polyoxyethylene-polyoxypropylene block copolymers; and mixtures thereof; polyethylene glycol sorbitan fatty acid esters and hydrophilic transesterification products of a polyol with at least one member of the group consisting of triglycerides, vegetable oils, and hydrogenated vegetable oils. The polyol can be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10 oleate, Tween 40, Tween 60, sucrose monostearate, sucrose monolaurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In one embodiment, the composition can include a solubilizer to ensure good solubilization and/or dissolution of the compound of the present invention and to minimize precipitation of the compound of the present invention. This can be especially important for compositions for non-oral use, e.g., compositions for injection. A solubilizer can also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, .epsilon.-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, ε-caprolactone and isomers thereof, 6-valerolactone and isomers thereof, β-butyrolactone and isomers thereof; and other solubilizers known in the art, such as dimethyl acetamide, dimethyl isosorbide, N-methyl pyrrolidones, monooctanoin, diethylene glycol monoethyl ether, and water.

Mixtures of solubilizers can also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer can be limited to a bioacceptable amount, which can be readily determined by one of skill in the art. In some circumstances, it can be advantageous to include amounts of solubilizers far in excess of bioacceptable amounts, for example to maximize the concentration of the drug, with excess solubilizer removed prior to providing the composition to a patient using conventional techniques, such as distillation or evaporation. Thus, if present, the solubilizer can be in a weight ratio of 10%, 25%, 50%, 100%, or up to about 200% by weight, based on the combined weight of the drug, and other excipients. If desired, very small amounts of solubilizer can also be used, such as 5%, 2%, 1% or even less. Typically, the solubilizer can be present in an amount of about 1% to about 100%, more typically about 5% to about 25% by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base can be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals, alkaline earth metals, and the like. Examples can include, but are not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid and the like.

### Pharmaceutical Compositions for Injection

In some embodiments, the pharmaceutical composition is a composition for injection containing a compound as disclosed herein and a pharmaceutical excipient suitable for injection. Components and amounts of agents in the compositions are as described herein.

The forms in which the compositions can be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol, liquid polyethylene glycol, and the like (and suitable mixtures thereof), cyclodextrin derivatives, and vegetable oils can also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating the compound in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

### Pharmaceutical Compositions for Topical (e.g., Transdermal) Delivery

In some embodiments, the pharmaceutical composition is a composition for transdermal delivery containing a compound of the present invention and a pharmaceutical excipient suitable for transdermal delivery.

Compositions can be formulated into preparations in solid, semi-solid, or liquid forms suitable for local or topical administration, such as gels, water soluble jellies, creams, lotions, suspensions, foams, powders, slurries, ointments, solutions, oils, pastes, suppositories, sprays, emulsions, saline solutions, dimethylsulfoxide (DMSO)-based solutions. In general, carriers with higher densities are capable of providing an area with a prolonged exposure to the active ingredients. In contrast, a solution formulation can provide more immediate exposure of the active ingredient to the chosen area.

The pharmaceutical compositions also can comprise suitable solid or gel phase carriers or excipients, which are compounds that allow increased penetration of, or assist in the delivery of, therapeutic molecules across the stratum corneum permeability barrier of the skin. There are many of these penetration-enhancing molecules known to those trained in the art of topical formulation. Examples of such carriers and excipients include, but are not limited to, humectants (e.g., urea), glycols (e.g., propylene glycol), alcohols (e.g., ethanol), fatty acids (e.g., oleic acid), surfactants (e.g., isopropyl myristate and sodium lauryl sulfate), pyrrolidones, glycerol monolaurate, sulfoxides, terpenes (e.g., menthol), amines, amides, alkanes, alkanols, water, calcium carbonate, calcium phosphate, various sugars, starches, cellulose derivatives, gelatin, and polymers such as polyethylene glycols.

Another exemplary formulation for use in the methods of the present invention employs transdermal delivery devices (“patches”). Such patches can be used to provide continuous or discontinuous infusion of a compound in controlled amounts, either with or without one or more additional agents.

The construction and use of transdermal patches for the delivery of pharmaceutical agents is well known in the art. See, e.g., U.S. Pat. Nos. 5,023,252, 4,992,445 and 5,001,139. Such patches can be constructed for continuous, pulsatile, or on demand delivery of pharmaceutical agents.

### Pharmaceutical Compositions for Inhalation

Compositions for inhalation or insufflation include solutions and suspensions in pharmaceutically acceptable, aqueous or organic solvents, or mixtures thereof, and powders. The liquid or solid compositions can contain suitable pharmaceutically acceptable excipients as described supra. Preferably the compositions are administered by the oral or nasal respiratory route for local or systemic effect. Compositions in preferably pharmaceutically acceptable solvents can be nebulized by use of inert gases. Nebulized solutions can be inhaled directly from the nebulizing device or the nebulizing device can be attached to a face mask tert, or intermittent positive pressure breathing machine. Solution, suspension, or powder compositions can be administered, preferably orally or nasally, from devices that deliver the formulation in an appropriate manner.

### Other Pharmaceutical Compositions

Pharmaceutical compositions can also be prepared from compositions described herein and one or more pharmaceutically acceptable excipients suitable for sublingual, buccal, rectal, intraosseous, intraocular, intranasal, epidural, or intraspinal administration. Preparations for such pharmaceutical compositions are well-known in the art. See, e.g., Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, N.Y., 1990; Katzung, ed., Basic and Clinical Pharmacology, Ninth Edition, McGraw Hill, 20037ybg; Goodman and Gilman, eds., The Pharmacological Basis of Therapeutics, Tenth Edition, McGraw Hill, 2001; Remingtons Pharmaceutical Sciences, 20th Ed., Lippincott Williams & Wilkins., 2000; Martindale, The Extra Pharmacopoeia, Thirty-Second Edition (The Pharmaceutical Press, London, 1999); all of which are incorporated by reference herein in their entirety.

Administration of the compounds or pharmaceutical composition of the present invention can be effected by any method that enables delivery of the compounds to the site of action. These methods include oral routes, intraduodenal routes, parenteral injection (including intravenous, intraarterial, subcutaneous, intramuscular, intravascular, intraperitoneal or infusion), topical (e.g., transdermal application), rectal administration, via local delivery by catheter or stent or through inhalation. Compounds can also be administered intraadiposally or intrathecally.

The amount of the compound administered will be dependent on the mammal being treated, the severity of the disorder or condition, the rate of administration, the disposition of the compound and the discretion of the prescribing physician. However, an effective dosage is in the range of about 0.001 to about 100 mg per kg body weight per day, preferably about 1 to about 35 mg/kg/day, in single or divided doses. For a 70 kg human, this would amount to about 0.05 to 7 g/day, preferably about 0.05 to about 2.5 g/day. In some instances, dosage levels below the lower limit of the aforesaid range can be more than adequate, while in other cases still larger doses can be employed without causing any harmful side effect, e.g., by dividing such larger doses into several small doses for administration throughout the day.

In some embodiments, a compound of the invention is administered in a single dose. Typically, such administration will be by injection, e.g., intravenous injection, in order to introduce the agent quickly. However, other routes can be used as appropriate. A single dose of a compound of the invention can also be used for treatment of an acute condition.

In some embodiments, a compound is administered in multiple doses. Dosing can be about once, twice, three times, four times, five times, six times, or more than six times per day. Dosing can be about once a month, once every two weeks, once a week, or once every other day. In another embodiment a compound of the invention and another agent are administered together about once per day to about 6 times per day. In another embodiment the administration of a compound of the invention and an agent continues for less than about 7 days. In yet another embodiment the administration continues for more than about 6, 10, 14, 28 days, two months, six months, or one year. In some cases, continuous dosing is achieved and maintained as long as necessary.

Administration of the compound can continue as long as necessary. In some embodiments, the compound is administered for more than 1, 2, 3, 4, 5, 6, 7, 14, or 28 days. In some embodiments, the compound is administered for less than 28, 14, 7, 6, 5, 4, 3, 2, or 1 day. In some embodiments, the compound is administered chronically on an ongoing basis, e.g., for the treatment of chronic effects.

An effective amount of a compound can be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, or as an inhalant.

The compositions can also be delivered via an impregnated or coated device such as a stent, for example, or an artery-inserted cylindrical polymer. Such a method of administration can, for example, aid in the prevention or amelioration of restenosis following procedures such as balloon angioplasty. Without being bound by theory, compounds of the invention can slow or inhibit the migration and proliferation of smooth muscle cells in the arterial wall which contribute to restenosis. A compound can be administered, for example, by local delivery from the struts of a stent, from a stent graft, from grafts, or from the cover or sheath of a stent. In some embodiments, a compound of the invention is admixed with a matrix. Such a matrix can be a polymeric matrix, and can serve to bond the compound to the stent. Polymeric matrices suitable for such use, include, for example, lactone-based polyesters or copolyesters such as polylactide, polycaprolactonglycolide, polyorthoesters, polyanhydrides, polyaminoacids, polysaccharides, polyphosphazenes, poly(ether-ester) copolymers (e.g., PEO-PLLA); polydimethylsiloxane, poly(ethylene-vinylacetate), acrylate-based polymers or copolymers (e.g., polyhydroxyethyl methylmethacrylate, polyvinyl pyrrolidinone), fluorinated polymers such as polytetrafluoroethylene and cellulose esters. Suitable matrices can be nondegrading or can degrade with time, releasing the compound or compounds. A compound can be applied to the surface of the stent by various methods such as dip/spin coating, spray coating, dip-coating, and/or brush-coating. A compound can be applied in a solvent and the solvent can be allowed to evaporate, thus forming a layer of compound onto the stent. Alternatively, a compound can be located in the body of the stent or graft, for example in microchannels or micropores. When implanted, the compound diffuses out of the body of the stent to contact the arterial wall. Such stents can be prepared by dipping a stent manufactured to contain such micropores or microchannels into a solution of the compound of the invention in a suitable solvent, followed by evaporation of the solvent. Excess drug on the surface of the stent can be removed via an additional brief solvent wash. In yet other embodiments, a compound can be covalently linked to a stent or graft. A covalent linker can be used which degrades in vivo, leading to the release of the compound. Any bio-labile linkage can be used for such a purpose, such as ester, amide or anhydride linkages. A Compound can additionally be administered intravascularly from a balloon used during angioplasty. Extravascular administration of a compounds via the pericard or via advential application of a formulation described herein can also be performed.

A variety of stent devices which can be used as described are disclosed, for example, in the following references, all of which are hereby incorporated by reference: U.S. Pat. No. 5,451,233; U.S. Pat. No. 5,040,548; U.S. Pat. No. 5,061,273; U.S. Pat. No. 5,496,346; U.S. Pat. No. 5,292,331; U.S. Pat. No. 5,674,278; U.S. Pat. No. 3,657,744; U.S. Pat. No. 4,739,762; U.S. Pat. No. 5,195,984; U.S. Pat. No. 5,292,331; U.S. Pat. No. 5,674,278; U.S. Pat. No. 5,879,382; U.S. Pat. No. 6,344,053.

The compounds of the invention can be administered in dosages. It is known in the art that due to intersubject variability in compound pharmacokinetics, individualization of dosing regimen is necessary for optimal therapy. Dosing for a compound of the invention can be found by routine experimentation in light of the instant disclosure.

When a compound of the invention, is administered in a composition that comprises one or more agents, and the agent has a shorter half-life than the compound of the invention unit dose forms of the agent and the compound of the invention can be adjusted accordingly.

The subject pharmaceutical composition can, for example, be in a form suitable for oral administration as a tablet, capsule, pill, powder, sustained release formulations, solution, suspension, for parenteral injection as a sterile solution, suspension or emulsion, for topical administration as an ointment or cream or for rectal administration as a suppository. The pharmaceutical composition can be in unit dosage forms suitable for single administration of precise dosages. The pharmaceutical composition will include a conventional pharmaceutical carrier or excipient and a compound according to the invention as an active ingredient. In addition, it can include other medicinal or pharmaceutical agents, carriers, adjuvants, etc.

Exemplary parenteral administration forms include solutions or suspensions of active compound in sterile aqueous solutions, for example, aqueous propylene glycol or dextrose solutions. Such dosage forms can be suitably buffered, if desired.

The activity of the compounds of the present invention may be determined using any method known in the art, or any method described herein. For example, the activity of the kinase may be assessed, e.g., by measuring the incorporation of γ-³³P-phosphate from γ-³³P-ATP onto N-terminal His tagged substrate, which is expressed in E. coli and is purified by conventional methods, in the presence of the kinase. The assay may be carried out in 96-well polypropylene plate. The incubation mixture (100, μL) may comprise 25 mM Hepes, pH 7.4, 10 mM MgCl₂, 5 mM β-glycerolphosphate, 100 μM Na-orthovanadate, 5 mM DTT, 5 nM kinase, and 1 μM substrate. Inhibitors may be suspended in DMSO, and all reactions, including controls may be performed at a final concentration of 1% DMSO. Reactions may be initiated by the addition of 10 μM ATP (with 0.5 μCi γ-³³P-ATP/well) and incubated at ambient temperature for a suitable time, e.g., for 45 minutes. Equal volume of 25% TCA may be added to stop the reaction and precipitate the proteins. Precipitated proteins may be trapped onto glass fiber B filterplates, and excess labeled ATP washed off using a Tomtec MACH III harvestor. Plates may be allowed to air-dry prior to adding 30 μL/well of Packard Microscint 20, and plates may be counted using a Packard TopCount®.

### Methods

### Treatments for Rheumatoid Arthritis

In one embodiment, provided herein is a method of reducing a rheumatoid arthritis associated symptom in a biological sample, comprising contacting the biological sample with a compound provided herein (e.g., a compound of Formula I (e.g., Compound 292), or an enantiomer or a mixture of enantiomers thereof, or a pharmaceutically acceptable salt, solvate, hydrate, co-crystal, clathrate, or polymorph thereof), in an amount sufficient to reduce the rheumatoid arthritis associated symptom. In one embodiment, the method is carried out in vivo, for example, in a mammalian subject, e.g., an animal model or as part of therapeutic protocol. In one embodiment, the compound is used as a single agent or in combination with another agent or therapeutic modality.

As used herein, and unless otherwise specified, “contacting” can be direct (e.g., by direct application of the compound provided herein to a biological sample, e.g., in vitro) or indirect (e.g., by administering the compound provided herein to a subject (e.g., by any known administration route, e.g., orally), such that the compound provided herein reaches an affected biological sample within the body.

As used herein, and unless otherwise specified, a “biological sample” includes, for example, a cell or group of cells (e.g., PBMCs, or plasmacytoid dendritic cell(s)), a tissue, or a fluid (e.g., whole blood or serum) that comes into contact with the PI3K inhibitor, thereby resulting in a decrease or inhibition of rheumatoid arthritis or rheumatoid arthritis associated symptoms. In some embodiments, the biological sample is present within or derived from a subject who has rheumatoid arthritis, or from a subject at risk for developing rheumatoid arthritis. In some embodiments, the biological sample can be contacted with the compound provided herein outside the body and then introduced into the body of a subject (e.g., into the body of the subject from whom the biological sample was derived or into the body of a different subject). In some embodiments, the biological sample includes cells that express Toll-like receptor 7 (TLR7) and/or Toll-like receptor 9 (TLR9).

In one embodiment, provided herein is a method of treating, preventing, and/or managing rheumatoid arthritis in a subject, comprising administering an effective amount of a compound provided herein (e.g., a compound of Formula I (e.g., Compound 292), or an enantiomer or a mixture of enantiomers thereof, or a pharmaceutically acceptable salt, solvate, hydrate, co-crystal, clathrate, or polymorph thereof) to a subject in need thereof. In one embodiment, the compound is administered as a single agent. In another embodiment, the compound is administered in combination with another agent or therapeutic modality.

As used herein, and unless otherwise specified, “rheumatoid arthritis” or a “symptom” associated with rheumatoid arthritis encompasses all types of manifestation of rheumatoid arthritis as disclosed herein or as known in the art. Examples include, but are not limited to, insidious onset rheumatoid arthritis, acute or immediate onset rheumatoid arthritis, moderate to severe rheumatoid arthritis, severe rheumatoid arthritis, early rheumatoid arthritis, seronegative rheumatoid arthritis, seropositive rheumatoid arthritis, and rheumatoid arthritis unresponsive or inadequately responsive to other disease-modifying anti-rheumatic drugs. Examples also include, but are not limited to, joint pain, which progresses into joint deformation, joint tenderness, joint swelling, morning stiffness in and around joints, arthritis of hand joints, symmetric arthritis, rheumatoid nodules (e.g., subcutaneous nodules, over bony prominences, or extensor surfaces, or in juxtaarticular regions), radiographic changes in or adjacent to joints (e.g., erosions or unequivocal bony decalcification), and joint pathology (e.g., bone resorption, cartilage damage, pannus, and/or inflammation). Joints commonly involved with rheumatoid arthritis include, but are not limited to, right or left proximal interphalangeal (PIP), metacarpophalangeal (MCP), wrist, elbow, knee, ankle, and metatarsophalangeal (MTP) joints.

As used herein, and unless otherwise specified, “rheumatoid arthritis” or a “symptom” associated with rheumatoid arthritis also encompasses all classification of rheumatoid arthritis under classification of global functional status in rheumatoid arthritis. A subject with class I rheumatoid arthritis is completely able to perform usual activities of daily living (self-care, vocational, and avocational). A subject with class II rheumatoid arthritis is able to perform usual self-care and vocational activities, but limited in avocational activities. A subject with class III rheumatoid arthritis is able to perform usual self-care activities, but limited in vocational and avocational activities. A subject with class IV rheumatoid arthritis is limited in ability to perform usual self-care, vocational, and avocational activities.

As used herein, and unless otherwise specified, “rheumatoid arthritis” or a “symptom” associated with rheumatoid arthritis also encompasses biological concomitants of rheumatoid arthritis as disclosed herein or as known in the art. Examples include, but are not limited to, immune complexes, elevated levels of cytokines (e.g., interferons (e.g., Type I interferons, e.g., IFN-α and/or IFN-β); interleukins (e.g., IL-6, IL-8, IL-1, and IL-18) and TNF-α), elevated levels of anti-dsDNA autoantibodies, overexpression of IFN-α and/or IFN-β inducible genes, elevated levels of IP-10, elevated levels of sCD40L, reduced levels of C3-derived C3b, reduced peripheral iNKT cell frequencies, defective B cell-mediated stimulation of iNKT cells, altered CD1d expression on B cells, reduced numbers of natural regulatory T cells (Treg), altered level of C-reactive protein, overexpression of mRNA for IL-4, overexpression of mRNA for IL-21, elevated serium anti-collagen level, germline SNPs that have been previously linked to autoimmune disease susceptibility (e.g., PTPN22) or to pathways of drug metabolism or transport (e.g., CYP3A family and/or other drug metabolizing enzymes that have been associated with metabolism of a compound provided herein), and abnormal absolute counts or percentages of mature human T lymphocytes (CD3+), natural killer cells (CD56+), and B lymphocytes (CD19+), suppressor/cytotoxic (CD3+CD8+) T-lymphocyte subsets, and helper/inducer (CD3+CD4+) T-lymphocyte subsets.

Symptoms can be assessed using assays and scales disclosed and/or exemplified herein and/or as known in the art. Examples include, but are not limited to, the Health Assessment Questionnaire (HAQ)-Disability Index (DI), Visual Analogue Scale (VAS), the Disease Activity Score using 28 joint counts (DAS28), FACIT-fatigue, which measures fatigue while performing activities of daily living during the previous week, SF-36, which is a 36-item questionnaire evaluating 8 domains (Role-Physical (RP), Bodily Pain (BP), Vitality (VT), Social Functioning (SF), Role-Emotional (RE), Mental Health (MH), Physical Functioning (PF), and General Health (GH)), and histopathology scores.

In some embodiments, the symptom is joint tenderness, joint swelling, or joint pain. In one embodiment, the symptom is joint tenderness. In one embodiment, the symptom is joint swelling. In one embodiment, the symptom is joint pain. In some embodiments, the symptom is ankle inflammation or knee inflammation.

In some embodiments, the symptom is overexpression of IFN-α, TNF-α, IL-6, IL-8, or IL-1. In one embodiment, the symptom is overexpression of IFN-α. In one embodiment, the symptom is overexpression of IL-6. In some embodiments, the symptom is overexpression of mRNA for IL-4 or overexpression of mRNA for IL-21. In some embodiments, the symptom is elevated serium anti-collagen level. In some embodiments, the symptom is elevated ankle and/or knee histopathology scores.

As used herein, and unless otherwise specified, to “decrease,” “ameliorate,” “reduce,” “inhibit,” “treat” (or the like) rheumatoid arthritis or a symptom associated with rheumatoid arthritis includes reducing (or preventing an increase in) the severity and/or frequency of one or more symptoms of rheumatoid arthritis, as well as preventing rheumatoid arthritis and/or one or more symptoms of rheumatoid arthritis (e.g., by reducing (or preventing an increase in) the severity and/or frequency of flares of symptoms). In the context of biological molecules, to “decrease”, “ameliorate,” “reduce,” “inhibit,” or the like, includes decreasing the level (e.g., the level, e.g., of mRNA or protein, that can be measured in a biological sample) or the activity (e.g., the function) of the molecule.

In some embodiments, the symptom is reduced by at least about 2%, at least about 5%, at least about 10%, at least about 15%, at least about 20%, at least about 25%, at least about 30%, at least about 40%, at least about 50%, at least about 60%, at least about 70%, at least about 80%, at least about 90%, or at least about 95% relative to a control level. The control level includes any appropriate control as known in the art. For example, the control level can be the pre-treatment level in the sample or subject treated, or it can be the level in a control population (e.g., the level in subjects who do not have rheumatoid arthritis or the level in samples derived from subjects who do not have rheumatoid arthritis). In some embodiments, the decrease is statistically significant, for example, as assessed using an appropriate parametric or non-parametric statistical comparison.

In some embodiments, the subject is a mammal. In some embodiments, the subject is a human.

In certain embodiments, the subject is an animal model of rheumatoid arthritis, a human with rheumatoid arthritis, or a subject (e.g., a human) at risk for developing rheumatoid arthritis. In some embodiments, the subject is a human who has a family history of rheumatoid arthritis, who carries a gene associated with rheumatoid arthritis, who is positive for a biomarker associated with rheumatoid arthritis, or a combination thereof. In some embodiments, the subject has been diagnosed with rheumatoid arthritis. In some embodiments, the subject has one or more signs or symptoms associated with rheumatoid arthritis. In some embodiments, the subject is at risk for developing rheumatoid arthritis (e.g., the subject carries a gene that, individually, or in combination with other genes or environmental factors, is associated with development of rheumatoid arthritis).

In one embodiment, the subject meets the American College of Rheumatology Criteria for rheumatoid arthritis. For example, a patient shall be said to have rheumatoid arthritis if he/she has satisfied at least 4 of the 7 following criteria: 1) morning stiffness in and around the joints, lasting at least 1 hour before maximal improvement; 2) soft tissue swelling (arthritis) of 3 or more joint areas observed by a physician; 3) swelling (arthritis) of the proximal interphalangeal, metacarpophalangeal, or wrist joints; 4) symmetric swelling (arthritis); 5) rheumatoid nodules; 6) the presence of rheumatoid factor; and 7) radiographic (e.g., by ultrasound, MRI, or X-ray imaging) erosions and/or periarticular osteopenia in hand and/or wrist joints. Criteria 1 through 4 must have been present for at least 6 weeks.

In one embodiment, the subject has class I, class II, class III, or class IV rheumatoid arthritis under classification of global functional status in rheumatoid arthritis.

In one embodiment, the subject has one or more swollen or tender joints. In one embodiment, the subject has at least 5 swollen joints or at least 5 tender joints. In one embodiment, the subject has at least 5 swollen joints and at least 5 tender joints.

In one embodiment, the subject exhibits an elevated level of C-reactive protein. In one embodiment, the subject exhibits an elevated level of C-reactive protein of at least 1.0 mg/L. In one embodiment, the subject exhibits an elevated level of C-reactive protein of at least 7 mg/L. In one embodiment, the subject exhibits an altered (e.g., elevated) level of Rheumatoid Factor (RF) and/or anti-citrullinated peptide (ACPA or anti-CCP) antibodies. In another embodiment, the subject exhibits an altered (e.g., elevated) level of Vetrix DA, 14-3-3 protein, or DAMPS.

In some embodiments, the subject exhibits elevated levels of antinuclear antibodies (e.g., anti-Smith antibodies, anti-double stranded DNA (dsDNA) antibodies, anti-U1 RNP, SS-a (or anti-Ro), SS-b (or anti-La)), antiphospholipid antibodies, anti-ss DNA antibodies, anti-histone antibodies, or anticardiolipin antibodies. In some embodiments, the subject exhibits elevated levels of anti-dsDNA antibodies. In some embodiments, the subject exhibits elevated levels of anti-Sm antibodies.

In some embodiments, the subject exhibits autoantibodies against one or more antigens that are known to be associated with rheumatoid arthritis or with rheumatoid arthritis subtypes. In some embodiments, the subject exhibits autoantibodies against Sm/anti-RNP or Ro/La autoantigens.

The levels of antibodies associated with rheumatoid arthritis can be assessed using methods known in the art, e.g., indirect immunofluorescence. In some embodiments, the methods disclosed herein reduce or prevent an increase in the levels of one or more of the foregoing antibodies.

In some embodiments, the subject exhibits elevated levels of IFN-α, TNF-α, IL-6, IL-8, or IL-1. In one embodiment, the subject exhibits an elevated level of IFN-α. In another embodiment, the subject exhibits an elevated level of IL-6. In another embodiment, the subject exhibits an elevated level of mRNA for IL-4 or IL-21.

In some embodiments, the subject has a mutation (e.g., an SNP) in a gene associated with rheumatoid arthritis. In one embodiment, the gene is selected from STAT4, IRF5, BANK1, ITGAM, PD1, FAM167A-BLK, IRF5-TNP03, KIAA1542, TNFAIP3, XKR6, 1q25.1, PXK, ATG5, ICA1, XKR6, LYN and SCUB2 or a combination thereof. In some embodiments, the subject carries the DR3 and DQ2 variants, or the DR2 and DQ6 variants of HLA class II genes. In some embodiments, the subject has a deficiency in one or more complement proteins, e.g., a deficiency of a complement protein coded by the C4A or C2 genes on chromosome 6, or the C1r and C1s genes on chromosome 12.

In some embodiments, the subject exhibits excessive PI3K activity or abnormal activity (e.g., excessive or reduced activity) of one or more components of the PI3K signaling pathway (e.g., Akt (PKB), mTOR, a Tec kinase (e.g., Btk, Itk, Tec), phospholipase C, PDK1, PKCs, NFκB, Rac GEF (e.g., Vav-1), or Rac).

In some embodiments, the subject is an animal model of rheumatoid arthritis provided herein or known in the art. Examples include, but are not limited to, the collagen-induced arthritis model, and Freund's complete adjuvant induced arthritis model.

In some embodiments, the subject has been previously treated for rheumatoid arthritis. In some embodiments, the subject has been previously treated for rheumatoid arthritis but are non-responsive to standard therapies. Thus, in one embodiment, provided herein is a method of treating, preventing, and/or managing rheumatoid arthritis in a subject, comprising administering an effective amount of a compound provided herein (e.g., a compound of Formula I (e.g., Compound 292), or an enantiomer or a mixture of enantiomers thereof, or a pharmaceutically acceptable salt, solvate, hydrate, co-crystal, clathrate, or polymorph thereof) to a subject in need thereof, wherein the subject has been previously administered a therapy for rheumatoid arthritis. In one embodiment, the previous treatment comprises administering methotrexate to the subject.

In one embodiment, the subject has been previously administered a therapy for rheumatoid arthritis (e.g., methotrexate) at least 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks before a compound provided herein (e.g., a compound of Formula I (e.g., Compound 292), or an enantiomer or a mixture of enantiomers thereof, or a pharmaceutically acceptable salt, solvate, hydrate, co-crystal, clathrate, or polymorph thereof) is administered. In one embodiment, the subject has been previously administered a therapy for rheumatoid arthritis (e.g., methotrexate) at least 1 week, 2 weeks, 1 month, 2 months, 3 months, or 4 months before a compound provided herein (e.g., a compound of Formula I (e.g., Compound 292), or an enantiomer or a mixture of enantiomers thereof, or a pharmaceutically acceptable salt, solvate, hydrate, co-crystal, clathrate, or polymorph thereof) is administered. In one embodiment, the subject has been previously administered a therapy for rheumatoid arthritis (e.g., methotrexate) at least 1 month before a compound provided herein (e.g., a compound of Formula I (e.g., Compound 292), or an enantiomer or a mixture of enantiomers thereof, or a pharmaceutically acceptable salt, solvate, hydrate, co-crystal, clathrate, or polymorph thereof) is administered. In another embodiment, the subject has been previously administered a therapy for rheumatoid arthritis (e.g., methotrexate) at least 3 months before a compound provided herein (e.g., a compound of Formula I (e.g., Compound 292), or an enantiomer or a mixture of enantiomers thereof, or a pharmaceutically acceptable salt, solvate, hydrate, co-crystal, clathrate, or polymorph thereof) is administered.

In one embodiment, the subject has been administered a stable dose of a therapy for rheumatoid arthritis (e.g., methotrexate) before a compound provided herein (e.g., a compound of Formula I (e.g., Compound 292), or an enantiomer or a mixture of enantiomers thereof, or a pharmaceutically acceptable salt, solvate, hydrate, co-crystal, clathrate, or polymorph thereof) is administered. In one embodiment, the subject has been administered a stable dose of a therapy for rheumatoid arthritis (e.g., methotrexate) for at least 24 hours, 48 hours, 72 hours, 96 hours, 1 week, 2 weeks, 3 weeks, 4 weeks, 5 weeks, 6 weeks, 8 weeks, 12 weeks, or 16 weeks before a compound provided herein (e.g., a compound of Formula I (e.g., Compound 292), or an enantiomer or a mixture of enantiomers thereof, or a pharmaceutically acceptable salt, solvate, hydrate, co-crystal, clathrate, or polymorph thereof) is administered. In one embodiment, the subject has been administered a stable dose of a therapy for rheumatoid arthritis (e.g., methotrexate) for at least 1 week, 2 weeks, 3 weeks, 4 weeks, 5 weeks, 6 weeks, 8 weeks, 12 weeks, or 16 weeks before a compound provided herein (e.g., a compound of Formula I (e.g., Compound 292), or an enantiomer or a mixture of enantiomers thereof, or a pharmaceutically acceptable salt, solvate, hydrate, co-crystal, clathrate, or polymorph thereof) is administered. In one embodiment, the subject has been administered a stable dose of a therapy for rheumatoid arthritis (e.g., sulfasalazine, chloroquine, or hydroxychloroquine) for at least 4 weeks before a compound provided herein (e.g., a compound of Formula I (e.g., Compound 292), or an enantiomer or a mixture of enantiomers thereof, or a pharmaceutically acceptable salt, solvate, hydrate, co-crystal, clathrate, or polymorph thereof) is administered. In one embodiment, the subject has been administered a stable dose of a therapy for rheumatoid arthritis (e.g., methotrexate) for at least 6 weeks before a compound provided herein (e.g., a compound of Formula I (e.g., Compound 292), or an enantiomer or a mixture of enantiomers thereof, or a pharmaceutically acceptable salt, solvate, hydrate, co-crystal, clathrate, or polymorph thereof) is administered.

In one embodiment, the subject has been previously administered a therapy for rheumatoid arthritis (e.g., methotrexate) at least 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks before, and the subject has been administered a stable dose of the same therapy for rheumatoid arthritis (e.g., methotrexate) for at least 24 hours, 48 hours, 72 hours, 96 hours, 1 week, 2 weeks, 3 weeks, 4 weeks, 5 weeks, 6 weeks, 8 weeks, 12 weeks, or 16 weeks before, a compound provided herein (e.g., a compound of Formula I (e.g., Compound 292), or an enantiomer or a mixture of enantiomers thereof, or a pharmaceutically acceptable salt, solvate, hydrate, co-crystal, clathrate, or polymorph thereof) is administered. In one embodiment, the subject has been previously administered a therapy for rheumatoid arthritis (e.g., methotrexate) at least 3 months before, and the subject has been administered a stable dose of the same therapy for rheumatoid arthritis (e.g., methotrexate) for at least 6 weeks before, a compound provided herein (e.g., a compound of Formula I (e.g., Compound 292), or an enantiomer or a mixture of enantiomers thereof, or a pharmaceutically acceptable salt, solvate, hydrate, co-crystal, clathrate, or polymorph thereof) is administered.

In one embodiment, the stable dose of the previously administered therapy (e.g., methotrexate) is from about 0.005 to about 1,000 mg per week, from about 0.01 to about 500 mg per week, from about 0.1 to about 250 mg per week, from about 1 to about 100 mg per week, from about 2 to about 75 mg per week, from about 3 to about 50 mg per week, from about 5 to about 50 mg per week, from about 7.5 to about 25 mg per week, from about 10 to about 25 mg per week, from about 12.5 to about 25 mg per week, from about 15 to about 25 mg per week, or from about 15 to about 20 mg per week. In one embodiment, the stable dose of the previously administered therapy (e.g., methotrexate) is from about 7.5 to about 25 mg per week. In one embodiment, the stable dose of the previously administered therapy (e.g., methotrexate) is from at least 15 mg per week to about 25 mg per week. The total dose per week may be administered once or administered among split doses.

In some embodiments, the subject has not been previously treated for rheumatoid arthritis.

In certain embodiments, a therapeutically or prophylactically effective amount of a compound provided herein (e.g., a compound of Formula I (e.g., Compound 292), or an enantiomer or a mixture of enantiomers thereof, or a pharmaceutically acceptable salt, solvate, hydrate, co-crystal, clathrate, or polymorph thereof) is from about 0.005 to about 1,000 mg per day, from about 0.01 to about 500 mg per day, from about 0.01 to about 250 mg per day, from about 0.01 to about 100 mg per day, from about 0.1 to about 100 mg per day, from about 0.5 to about 100 mg per day, from about 1 to about 100 mg per day, from about 0.01 to about 50 mg per day, from about 0.1 to about 50 mg per day, from about 0.5 to about 50 mg per day, from about 1 to about 50 mg per day, from about 2 to about 25 mg per day, or from about 5 to about 10 mg per day.

In certain embodiments, the therapeutically or prophylactically effective amount is about 0.1, about 0.2, about 0.5, about 1, about 2, about 5, about 10, about 15, about 20, about 25, about 30, about 40, about 45, about 50, about 60, about 70, about 80, about 90, about 100, or about 150 mg per day.

In one embodiment, the recommended daily dose range of a compound of Formula I (e.g., Compound 292), or an enantiomer or a mixture of enantiomers thereof, or a pharmaceutically acceptable salt, solvate, hydrate, co-crystal, clathrate, or polymorph thereof, for the conditions described herein lie within the range of from about 0.5 mg to about 50 mg per day, preferably given as a single once-a-day dose, or in divided doses throughout a day. In some embodiments, the dosage ranges from about 1 mg to about 50 mg per day. In other embodiments, the dosage ranges from about 0.5 to about 5 mg per day. Specific doses per day include 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 mg per day.

In a specific embodiment, the recommended starting dosage may be 0.5, 1, 2, 3, 4, 5, 10, 15, 20, 25 or 50 mg per day. In another embodiment, the recommended starting dosage may be 0.5, 1, 2, 3, 4, or 5 mg per day. The dose may be escalated to 15, 20, 25, 30, 35, 40, 45 and 50 mg/day.

In certain embodiments, the therapeutically or prophylactically effective amount is from about 0.001 to about 100 mg/kg/day, from about 0.01 to about 50 mg/kg/day, from about 0.01 to about 25 mg/kg/day, from about 0.01 to about 10 mg/kg/day, from about 0.01 to about 9 mg/kg/day, 0.01 to about 8 mg/kg/day, from about 0.01 to about 7 mg/kg/day, from about 0.01 to about 6 mg/kg/day, from about 0.01 to about 5 mg/kg/day, from about 0.01 to about 4 mg/kg/day, from about 0.01 to about 3 mg/kg/day, from about 0.01 to about 2 mg/kg/day, or from about 0.01 to about 1 mg/kg/day.

The administered dose can also be expressed in units other than mg/kg/day. For example, doses for parenteral administration can be expressed as mg/m²/day. One of ordinary skill in the art would readily know how to convert doses from mg/kg/day to mg/m²/day to given either the height or weight of a subject or both (see, www.fda.gov/cder/cancer/animalframe.htm). For example, a dose of 1 mg/kg/day for a 65 kg human is approximately equal to 38 mg/m²/day.

In one embodiment, the amount of the compound administered is sufficient to provide a plasma concentration of the compound at steady state, ranging from about 0.005 to about 100 μM, from about 0.005 to about 10 μM, from about 0.01 to about 10 μM, from about 0.01 to about 5 μM, from about 0.005 to about 1 μM, from about 0.005 to about 0.5 μM, from about 0.005 to about 0.5 μM, from about 0.01 to about 0.2 μM, or from about 0.01 to about 0.1 μM. In one embodiment, the amount of the compound administered is sufficient to provide a plasma concentration at steady state, of about 0.005 to about 100 μM. In another embodiment, the amount of the compound administered is sufficient to provide a plasma concentration at steady state, of about 0.005 to about 10 μM. In yet another embodiment, the amount of the compound administered is sufficient to provide a plasma concentration at steady state, of about 0.01 to about 10 μM. In yet another embodiment, the amount of the compound administered is sufficient to provide a plasma concentration at steady state, of about 0.01 to about 5 μM. In yet another embodiment, the amount of the compound administered is sufficient to provide a plasma concentration at steady state, of about 0.005 to about 1 μM. In yet another embodiment, the amount of the compound administered is sufficient to provide a plasma concentration at steady state, of about 0.005 to about 0.5 μM. In yet another embodiment, the amount of the compound administered is sufficient to provide a plasma concentration of the compound at steady state, of about 0.01 to about 0.2 μM. In still another embodiment, the amount of the compound administered is sufficient to provide a plasma concentration of the compound at steady state, of about 0.01 to about 0.1 μM. As used herein, the term “plasma concentration at steady state” is the concentration reached after a period of administration of a compound. Once steady state is reached, there are minor peaks and troughs on the time dependent curve of the plasma concentration of the compound.

In one embodiment, the amount administered is sufficient to provide a maximum plasma concentration (peak concentration) of the compound, ranging from about 0.005 to about 100 μM, from about 0.005 to about 10 μM, from about 0.01 to about 10 μM, from about 0.01 to about 5 μM, from about 0.005 to about 1 μM, from about 0.005 to about 0.5 μM, from about 0.01 to about 0.2 μM, or from about 0.01 to about 0.1 μM. In one embodiment, the amount of the compound administered is sufficient to provide a maximum plasma concentration of the compound of about 0.005 to about 100 μM. In another embodiment, the amount of the compound administered is sufficient to provide a maximum plasma concentration of the compound of about 0.005 to about 10 μM. In yet another embodiment, the amount of the compound administered is sufficient to provide a maximum plasma concentration of the compound of about 0.01 to about 10 μM. In yet another embodiment, the amount of the compound administered is sufficient to provide a maximum plasma concentration of the compound of about 0.01 to about 5 μM. In yet another embodiment, the amount of the compound administered is sufficient to provide a maximum plasma concentration of the compound of about 0.005 to about 1 μM. In yet another embodiment, the amount of the compound administered is sufficient to provide a maximum plasma concentration of the compound of about 0.005 to about 0.5 μM. In yet another embodiment, the amount of the compound administered is sufficient to provide a maximum plasma concentration of the compound of about 0.01 to about 0.2 μM. In still another embodiment, the amount of the compound administered is sufficient to provide a maximum plasma concentration of the compound of about 0.01 to about 0.1 μM.

In one embodiment, the amount administered is sufficient to provide a minimum plasma concentration (trough concentration) of the compound, ranging from about 0.005 to about 100 μM, from about 0.005 to about 10 μM, from about 0.01 to about 10 μM, from about 0.01 to about 5 μM, from about 0.005 to about 1 μM, about 0.005 to about 0.5 μM, from about 0.01 to about 0.2 μM, or from about 0.01 to about 0.1 μM, when more than one doses are administered. In one embodiment, the amount of the compound administered is sufficient to provide a minimum plasma concentration of the compound of about 0.005 to about 100 μM. In another embodiment, the amount of the compound administered is sufficient to provide a minimum plasma concentration of the compound of about 0.005 to about 10 μM. In yet another embodiment, the amount of the compound administered is sufficient to provide a minimum plasma concentration of the compound of about 0.01 to about 10 μM. In yet another embodiment, the amount of the compound administered is sufficient to provide a minimum plasma concentration of the compound of about 0.01 to about 5 μM. In yet another embodiment, the amount of the compound administered is sufficient to provide a minimum plasma concentration of the compound of about 0.005 to about 1 μM. In yet another embodiment, the amount of the compound administered is sufficient to provide a minimum plasma concentration of the compound of about 0.005 to about 0.5 μM. In yet another embodiment, the amount of the compound administered is sufficient to provide a minimum plasma concentration of the compound of about 0.01 to about 0.2 μM. In still another embodiment, the amount of the compound administered is sufficient to provide a minimum plasma concentration of the compound of about 0.01 to about 0.1 μM.

In one embodiment, the amount administered is sufficient to provide an area under the curve (AUC) of the compound, ranging from about 50 to about 10,000 ng*hr/mL, about 100 to about 50,000 ng*hr/mL, from about 100 to 25,000 ng*hr/mL, or from about 10,000 to 25,000 ng*hr/mL.

The compound provided herein (e.g., a compound of Formula I (e.g., Compound 292), or an enantiomer or a mixture of enantiomers thereof, or a pharmaceutically acceptable salt, solvate, hydrate, co-crystal, clathrate, or polymorph thereof) may be administered by oral, parenteral (e.g., intramuscular, intraperitoneal, intravenous, CIV, intracistemal injection or infusion, subcutaneous injection, or implant), inhalation, nasal, vaginal, rectal, sublingual, or topical (e.g., transdermal or local) routes of administration. In one embodiment, the compound is administered orally. In another embodiment, the compound is administered parenterally. In yet another embodiment, the compound is administered intravenously.

A compound provided herein (e.g., a compound of Formula I (e.g., Compound 292), or an enantiomer or a mixture of enantiomers thereof, or a pharmaceutically acceptable salt, solvate, hydrate, co-crystal, clathrate, or polymorph thereof) can be administered once daily (QD), or divided into multiple daily doses such as twice daily (BID), three times daily (TID), and four times daily (QID). In addition, the administration can be continuous (i.e., daily for consecutive days or every day), intermittent, e.g., in cycles (i.e., including days, weeks, or months of rest without drug). As used herein, the term “daily” is intended to mean that a therapeutic compound, such as a compound of Formula I, is administered once or more than once each day, for example, for a period of time. The term “continuous” is intended to mean that a therapeutic compound, such as a compound of Formula I, is administered daily for an uninterrupted period of at least 10 days to 52 weeks. The term “intermittent” or “intermittently” as used herein is intended to mean stopping and starting at either regular or irregular intervals. For example, intermittent administration of a compound of Formula I is administration for one to six days per week, administration in cycles (e.g., daily administration for two to eight consecutive weeks, then a rest period with no administration for up to one week), or administration on alternate days. The term “cycling” as used herein is intended to mean that a therapeutic compound, such as a compound of Formula I, is administered daily or continuously but with a rest period.

In some embodiments, the frequency of administration is in the range of about a daily dose to about a monthly dose. In certain embodiments, administration is once a day, twice a day, three times a day, four times a day, once every other day, twice a week, once every week, once every two weeks, once every three weeks, or once every four weeks. In one embodiment, the compound provided herein is administered once a day. In another embodiment, the compound provided herein is administered twice a day. In yet another embodiment, the compound provided herein is administered three times a day. In still another embodiment, the compound provided herein is administered four times a day.

In one embodiment, a compound provided herein (e.g., a compound of Formula I (e.g., Compound 292), or an enantiomer or a mixture of enantiomers thereof, or a pharmaceutically acceptable salt, solvate, hydrate, co-crystal, clathrate, or polymorph thereof) is administered about 0.1, 0.2, 0.25, 0.5, 1, 2, 2.5, 5, 10, 15, 20, 25, or 50 mg BID. In one embodiment, a compound provided herein (e.g., a compound of Formula I (e.g., Compound 292), or an enantiomer or a mixture of enantiomers thereof, or a pharmaceutically acceptable salt, solvate, hydrate, co-crystal, clathrate, or polymorph thereof) is administered about 0.5 mg BID. In another embodiment, a compound provided herein (e.g., a compound of Formula I (e.g., Compound 292), or an enantiomer or a mixture of enantiomers thereof, or a pharmaceutically acceptable salt, solvate, hydrate, co-crystal, clathrate, or polymorph thereof) is administered about 1 mg BID. In another embodiment, a compound provided herein (e.g., a compound of Formula I (e.g., Compound 292), or an enantiomer or a mixture of enantiomers thereof, or a pharmaceutically acceptable salt, solvate, hydrate, co-crystal, clathrate, or polymorph thereof) is administered about 5 mg BID.

In certain embodiments, the compound provided herein (e.g., a compound of Formula I (e.g., Compound 292), or an enantiomer or a mixture of enantiomers thereof, or a pharmaceutically acceptable salt, solvate, hydrate, co-crystal, clathrate, or polymorph thereof) is administered once per day from one day to six months, from one week to three months, from one week to four weeks, from one week to three weeks, or from one week to two weeks. In certain embodiments, the compound provided herein is administered once per day for one week, two weeks, three weeks, or four weeks. In one embodiment, the compound provided herein is administered once per day for one week. In another embodiment, the compound provided herein is administered once per day for two weeks. In yet another embodiment, the compound provided herein is administered once per day for three weeks. In still another embodiment, the compound provided herein is administered once per day for four weeks.

The compound provided herein (e.g., a compound of Formula I (e.g., Compound 292), or an enantiomer or a mixture of enantiomers thereof, or a pharmaceutically acceptable salt, solvate, hydrate, co-crystal, clathrate, or polymorph thereof) can be delivered as a single dose such as, e.g., a single bolus injection, or oral tablets or pills; or over time, such as, e.g., continuous infusion over time or divided bolus doses over time. The compound can be administered repeatedly if necessary, for example, until the patient experiences stable disease or regression, or until the patient experiences disease progression or unacceptable toxicity. For example, in one embodiment, stable disease for rheumatoid arthritis means that the joint diameter (e.g., ankle diameter) of a subject having rheumatoid arthritis has not increased by 25% or more from the last measurement.

In one embodiment, the regression for rheumatoid arthritis means that the subject achieves at least 20% improvement in the American College of Rheumatology Criteria (ACR20) from baseline. In one embodiment, the regression for rheumatoid arthritis means that the subject achieves at least 50% improvement in the American College of Rheumatology Criteria (ACR50) from baseline. In one embodiment, the regression for rheumatoid arthritis means that the subject achieves at least 70% improvement in the American College of Rheumatology Criteria (ACR70) from baseline.

In one embodiment, the regression for rheumatoid arthritis is reduction (e.g., at least 20% from baseline) in number of tender or painful joints. In another embodiment, the regression for rheumatoid arthritis is reduction (e.g., at least 20% from baseline) in number of swollen joints.

In one embodiment, the regression for rheumatoid arthritis is a reduction in classification of global functional status in rheumatoid arthritis (e.g., changes from Class IV to Class III, from Class III to Class II, and from Class II to Class I).

In one embodiment, the regression for rheumatoid arthritis is improvement (e.g., at least 20% from baseline) in one or more of the subject's assessment of pain on the VAS scale, the subject's global assessment of disease activity, physician's global assessment of disease activity, the Health Assessment Questionnaire-Disability Index, and the C-reactive protein level.

### Treatments for Asthma

In one embodiment, provided herein is a method of reducing an asthma associated symptom in a biological sample, comprising contacting the biological sample with a compound provided herein (e.g., a compound of Formula I (e.g., Compound 292), or an enantiomer or a mixture of enantiomers thereof, or a pharmaceutically acceptable salt, solvate, hydrate, co-crystal, clathrate, or polymorph thereof), in an amount sufficient to reduce the asthma associated symptom. In one embodiment, the method is carried out in vivo, for example, in a mammalian subject, e.g., an animal model or as part of therapeutic protocol. In one embodiment, the compound is used as a single agent or in combination with another agent or therapeutic modality.

As used herein, and unless otherwise specified, “contacting” can be direct (e.g., by direct application of the compound provided herein to a biological sample, e.g., in vitro) or indirect (e.g., by administering the compound provided herein to a subject (e.g., by any known administration route, e.g., orally), such that the compound provided herein reaches an affected biological sample within the body.

As used herein, and unless otherwise specified, a “biological sample” includes, for example, a cell or group of cells (e.g., PBMCs, or plasmacytoid dendritic cell(s)), a tissue, or a fluid (e.g., whole blood or serum) that comes into contact with the PI3K inhibitor, thereby resulting in a decrease or inhibition of asthma or asthma associated symptoms. In some embodiments, the biological sample is present within or derived from a subject who has asthma, or from a subject at risk for developing asthma. In some embodiments, the biological sample can be contacted with the compound provided herein outside the body and then introduced into the body of a subject (e.g., into the body of the subject from whom the biological sample was derived or into the body of a different subject). In some embodiments, the biological sample includes cells that express Toll-like receptor 7 (TLR7) and/or Toll-like receptor 9 (TLR9).

In one embodiment, provided herein is a method of treating, preventing, and/or managing asthma in a subject, comprising administering an effective amount of a compound provided herein (e.g., a compound of Formula I (e.g., Compound 292), or an enantiomer or a mixture of enantiomers thereof, or a pharmaceutically acceptable salt, solvate, hydrate, co-crystal, clathrate, or polymorph thereof) to a subject in need thereof. In one embodiment, the compound is administered as a single agent. In another embodiment, the compound is administered in combination with another agent or therapeutic modality.

As used herein, and unless otherwise specified, “asthma” or a “symptom” associated with asthma encompasses all types of manifestation of asthma as disclosed herein or as known in the art. Examples of asthma include, but are not limited to, severe and/or refractory asthma, atopic (extrinsic) asthma, non-atopic (intrinsic) asthma, type 1 brittle asthma, type 2 brittle asthma, asthma attack, status asthmaticus, exercise-induced asthma, or occupational asthma. In one embodiment, the asthma is severe or refractory asthma. Examples of symptom of asthma include, but are not limited to, wheezing, coughing, chest tightness, shortness of breath, and use of accessory muscle. Symptoms are often worse at night or in the early morning, or in response to exercise or cold air. Asthma is clinically classified according to the frequency of symptoms, forced expiratory volume in 1 second (FEV₁), and peak expiratory flow rate. In one embodiment, the symptom of asthma is wheezing or chest tightness.

As used herein, and unless otherwise specified, “asthma” or a “symptom” associated with asthma also encompasses biological concomitants of asthma as disclosed herein or as known in the art. Examples include, but are not limited to, immune complexes, elevated levels of cytokines (e.g., interferons (e.g., Type I interferons, e.g., IFN-α and/or IFN-β); interleukins (e.g., IL-6, IL-8, IL-1, and IL-18) and TNF-α), elevated levels of anti-dsDNA autoantibodies, overexpression of IFN-α and/or IFN-β inducible genes, elevated levels of IP-10, elevated levels of sCD40L, reduced levels of C3-derived C3b, reduced peripheral iNKT cell frequencies, defective B cell-mediated stimulation of iNKT cells, altered CD1d expression on B cells, reduced numbers of natural regulatory T cells (Treg), altered level of C-reactive protein, overexpression of mRNA for IL-4, overexpression of mRNA for IL-21, and elevated serium anti-collagen level. In some embodiments, the symptom is overexpression of IFN-α, TNF-α, IL-6, IL-8, or IL-1. In one embodiment, the symptom is overexpression of IFN-α. In one embodiment, the symptom is overexpression of IL-6. In some embodiments, the symptom is overexpression of mRNA for IL-4 or overexpression of mRNA for IL-21. In some embodiments, the symptom is elevated serium anti-collagen level.

As used herein, and unless otherwise specified, to “decrease,” “ameliorate,” “reduce,” “inhibit,” “treat” (or the like) asthma or a symptom associated with asthma includes reducing (or preventing an increase in) the severity and/or frequency of one or more symptoms of asthma, as well as preventing asthma and/or one or more symptoms of asthma (e.g., by reducing (or preventing an increase in) the severity and/or frequency of flares of symptoms). In the context of biological molecules, to “decrease”, “ameliorate,” “reduce,” “inhibit,” or the like, includes decreasing the level (e.g., the level, e.g., of mRNA or protein, that can be measured in a biological sample) or the activity (e.g., the function) of the molecule.

In some embodiments, the symptom is reduced by at least about 2%, at least about 5%, at least about 10%, at least about 15%, at least about 20%, at least about 25%, at least about 30%, at least about 40%, at least about 50%, at least about 60%, at least about 70%, at least about 80%, at least about 90%, or at least about 95% relative to a control level. The control level includes any appropriate control as known in the art. For example, the control level can be the pre-treatment level in the sample or subject treated, or it can be the level in a control population (e.g., the level in subjects who do not have asthma or the level in samples derived from subjects who do not have asthma). In some embodiments, the decrease is statistically significant, for example, as assessed using an appropriate parametric or non-parametric statistical comparison.

In some embodiments, the subject is a mammal. In some embodiments, the subject is a human.

In certain embodiments, the subject is an animal model of asthma, a human with asthma, or a subject (e.g., a human) at risk for developing asthma. In some embodiments, the subject is a human who has a family history of asthma, who carries a gene associated with asthma, who is positive for a biomarker associated with asthma, or a combination thereof. In some embodiments, the subject has been diagnosed with asthma. In some embodiments, the subject has one or more signs or symptoms associated with asthma. In some embodiments, the subject is at risk for developing asthma (e.g., the subject carries a gene that, individually, or in combination with other genes or environmental factors, is associated with development of asthma).

In one embodiment, the subject has been previously diagnosed of asthma or has episodic symptoms of airflow obstruction (e.g., wheezing and/or chest tightness) for at least 1 week, 2 weeks, 1 month, 2 months, 3 months, 6 months, 9 months, 12 months before a compound provided herein (e.g., a compound of Formula I (e.g., Compound 292), or an enantiomer or a mixture of enantiomers thereof, or a pharmaceutically acceptable salt, solvate, hydrate, co-crystal, clathrate, or polymorph thereof) is administered. In one embodiment, the subject has been previously diagnosed of asthma or has episodic symptoms of airflow obstruction (e.g., wheezing and/or chest tightness) for at least 6 months before a compound provided herein (e.g., a compound of Formula I (e.g., Compound 292), or an enantiomer or a mixture of enantiomers thereof, or a pharmaceutically acceptable salt, solvate, hydrate, co-crystal, clathrate, or polymorph thereof) is administered.

In one embodiment, the subject has a forced expiratory volume in one second (FEV₁) value of at least 95%, 90%, 85%, 80%, 75%, 70%, 65%, 60%, 55%, or 50% of a control value. In one embodiment, the subject has a forced expiratory volume in one second (FEV₁) value of at least 70% of a control value. In one embodiment, the control value may be calculated based on American Thoracic Society (ATS)/European Respiratory Society (ERS) standards.

In one embodiment, the subject has a positive response to a skin prick test to an allergen. In one embodiment, the positive response means that the induration of skin test wheal is larger in diameter (e.g., at least 2 mm larger) than the diameter of the control wheal. The allergen can be any allergen provided herein or known in the art that can be used in the diagnosis or determining status of asthma.

In one embodiment, the subject has an early-phase asthmatic response (EAR) of at least 5%, 10%, 15%, 20%, 25%, 30%, 35%, 40%, 45%, or 50% to an inhaled allergen challenge. In one embodiment, the subject has an early-phase asthmatic response of at least 20% to an inhaled allergen challenge. In one embodiment, the EAR response is a decrease from pre-challenge in FEV₁ on 2 consecutive occasions within 0 to <3 hours of last allergen challenge.

In one embodiment, the subject has a late-phase asthmatic response (LAR) of at least 5%, 10%, 15%, 20%, 25%, 30%, 35%, 40%, 45%, or 50% to an inhaled allergen challenge. In one embodiment, the subject has a late-phase asthmatic response of at least 15% to an inhaled allergen challenge. In one embodiment, the LAR response is a decrease from pre-challenge in FEV₁ on 2 consecutive occasions within 3 to 10 hours of last allergen challenge.

In one embodiment, the subject has an early-phase asthmatic response of at least 20% and a late-phase asthmatic response of at least 15% to an inhaled allergen challenge. The inhaled allergen can be any inhaled allergen provided herein or known in the art that can be used in the diagnosis or determining status of asthma.

In one embodiment, the subject exhibits an elevated level of C-reactive protein. In one embodiment, the subject exhibits an elevated level of C-reactive protein of at least 1.0 mg/L. In one embodiment, the subject exhibits an elevated level of C-reactive protein of at least 7 mg/L.

In some embodiments, the subject exhibits elevated levels of antinuclear antibodies (e.g., anti-Smith antibodies, anti-double stranded DNA (dsDNA) antibodies, anti-U1 RNP, SS-a (or anti-Ro), SS-b (or anti-La)), antiphospholipid antibodies, anti-ss DNA antibodies, anti-histone antibodies, or anticardiolipin antibodies. In some embodiments, the subject exhibits elevated levels of anti-dsDNA antibodies. In some embodiments, the subject exhibits elevated levels of anti-Sm antibodies.

In some embodiments, the subject exhibits autoantibodies against one or more antigens that are known to be associated with asthma or with asthma subtypes. In some embodiments, the subject exhibits autoantibodies against Sm/anti-RNP or Ro/La autoantigens.

The levels of antibodies associated with asthma can be assessed using methods known in the art, e.g., indirect immunofluorescence. In some embodiments, the methods disclosed herein reduce or prevent an increase in the levels of one or more of the foregoing antibodies.

In some embodiments, the subject exhibits elevated levels of IFN-α, TNF-α, IL-6, IL-8, or IL-1. In one embodiment, the subject exhibits an elevated level of IFN-α. In another embodiment, the subject exhibits an elevated level of IL-6. In another embodiment, the subject exhibits an elevated level of mRNA for IL-4 or IL-21.

In some embodiments, the subject has a mutation (e.g., an SNP) in a gene associated with asthma. In one embodiment, the gene is selected from STAT4, IRF5, BANK1, ITGAM, PD1, FAM167A-BLK, IRF5-TNP03, KIAA1542, TNFAIP3, XKR6, 1q25.1, PXK, ATG5, ICA1, XKR6, LYN and SCUB2 or a combination thereof. In some embodiments, the subject carries the DR3 and DQ2 variants, or the DR2 and DQ6 variants of HLA class II genes. In some embodiments, the subject has a deficiency in one or more complement proteins, e.g. a deficiency of a complement protein coded by the C4A or C2 genes on chromosome 6, or the C1r and C1s genes on chromosome 12.

In some embodiments, the subject exhibits excessive PI3K activity or abnormal activity (e.g., excessive or reduced activity) of one or more components of the PI3K signaling pathway (e.g., Akt (PKB), mTOR, a Tec kinase (e.g., Btk, Itk, Tec), phospholipase C, PDK1, PKCs, NFκB, Rac GEF (e.g., Vav-1), or Rac).

In some embodiments, the subject is an animal model of asthma provided herein or known in the art. Examples include, but are not limited to, the murine lipopolysaccharide (LPS) induced pulmonary inflammation model, and the murine ovalbumin-induced allergic airway inflammation model.

In some embodiments, the subject has been previously treated for asthma. In some embodiments, the subject has been previously treated for asthma but are non-responsive to standard therapies. Thus, in one embodiment, provided herein is a method of treating, preventing, and/or managing asthma in a subject, comprising administering an effective amount of a compound provided herein (e.g., a compound of Formula I (e.g., Compound 292), or an enantiomer or a mixture of enantiomers thereof, or a pharmaceutically acceptable salt, solvate, hydrate, co-crystal, clathrate, or polymorph thereof) to a subject in need thereof, wherein the subject has been previously administered a therapy for asthma.

In some embodiments, the subject has not been previously treated for asthma.

In one embodiment, provided herein is a method of treating, preventing, and/or managing asthma in a subject, comprising administering an effective amount of a compound provided herein (e.g., a compound of Formula I (e.g., Compound 292), or an enantiomer or a mixture of enantiomers thereof, or a pharmaceutically acceptable salt, solvate, hydrate, co-crystal, clathrate, or polymorph thereof) to a subject in need thereof.

In one embodiment, without being limited by any particular theory, administering an effective amount of a compound provided herein (e.g., a compound of Formula I (e.g., Compound 292), or an enantiomer or a mixture of enantiomers thereof, or a pharmaceutically acceptable salt, solvate, hydrate, co-crystal, clathrate, or polymorph thereof) does not result in, or results in reduced, one or more common side effects of asthma treatment. The common side effects of asthma treatment include, but are not limited to, oral candidiasis, thrush, dysphonia (hoarseness), reflex cough, bronchospasm, poor growth, decreased bone density, disseminated varicella infection (chickenpox that spreads to organs), easy bruising, cataracts, glaucoma, adrenal gland suppression, stomach upset, headache, liver test abnormalities, skin rashes, Churg Strauss syndrome, bad taste in month, cough, itching, sore throat, sneezing, stuffy nose, shortness of breath, wheezing, viral illness, upper respiratory tract infections, sinusitis, feeling dizzy or faint, hives, changes in voice, swelling of the tougue, or difficulty in swallowing.

In some embodiments, the side effect is reduced by at least about 2%, at least about 5%, at least about 10%, at least about 15%, at least about 20%, at least about 25%, at least about 30%, at least about 40%, at least about 50%, at least about 60%, at least about 70%, at least about 80%, at least about 90%, or at least about 95% relative to a control level. The control level includes any appropriate control as known in the art. For example, the control level can be the side effect level in the subject treated with other asthma therapies (e.g., Xolair, Cromolyn Sodium, Nedocromil, Montelukast, and prednisone). In some embodiments, the decrease is statistically significant, for example, as assessed using an appropriate parametric or non-parametric statistical comparison.

In certain embodiments, a therapeutically or prophylactically effective amount of a compound provided herein (e.g., a compound of Formula I (e.g., Compound 292), or an enantiomer or a mixture of enantiomers thereof, or a pharmaceutically acceptable salt, solvate, hydrate, co-crystal, clathrate, or polymorph thereof) is from about 0.005 to about 1,000 mg per day, from about 0.01 to about 500 mg per day, from about 0.01 to about 250 mg per day, from about 0.01 to about 100 mg per day, from about 0.1 to about 100 mg per day, from about 0.5 to about 100 mg per day, from about 1 to about 100 mg per day, from about 0.01 to about 50 mg per day, from about 0.1 to about 50 mg per day, from about 0.5 to about 50 mg per day, from about 1 to about 50 mg per day, from about 2 to about 25 mg per day, or from about 5 to about 10 mg per day.

In certain embodiments, the therapeutically or prophylactically effective amount is about 0.1, about 0.2, about 0.5, about 1, about 2, about 5, about 10, about 15, about 20, about 25, about 30, about 40, about 45, about 50, about 60, about 70, about 80, about 90, about 100, or about 150 mg per day.

In one embodiment, the recommended daily dose range of a compound of Formula I (e.g., Compound 292), or an enantiomer or a mixture of enantiomers thereof, or a pharmaceutically acceptable salt, solvate, hydrate, co-crystal, clathrate, or polymorph thereof, for the conditions described herein lie within the range of from about 0.5 mg to about 50 mg per day, in a single once-a-day dose or in divided doses throughout a day. In some embodiments, the dosage ranges from about 1 mg to about 50 mg per day. In other embodiments, the dosage ranges from about 0.5 to about 5 mg per day. Specific doses per day include 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 mg per day.

In one embodiment, a compound provided herein (e.g., a compound of Formula I (e.g., Compound 292), or an enantiomer or a mixture of enantiomers thereof, or a pharmaceutically acceptable salt, solvate, hydrate, co-crystal, clathrate, or polymorph thereof) is administered at a dose of less than 0.1, about 0.1, less than 0.5, about 0.5, between about 0.1 and about 1.0, between about 0.5 and about 1.0, about 1, or about 2 mg per day.

In another embodiment, a compound provided herein (e.g., a compound of Formula I (e.g., Compound 292), or an enantiomer or a mixture of enantiomers thereof, or a pharmaceutically acceptable salt, solvate, hydrate, co-crystal, clathrate, or polymorph thereof) is administered at a dose of less than 0.2, about 0.2, less than 1.0, about 1.0, between about 0.2 and about 2.0, between about 1.0 and about 2.0, about 2, or about 4 mg per day.

In one embodiment, the dose is less than 0.1 mg per day.

In another embodiment, the dose is about 0.1 mg per day.

In another embodiment, the dose is less than 0.5 mg per day.

In another embodiment, the dose is about 0.5 mg per day.

In another embodiment, the dose is between about 0.1 and about 1.0 mg per day.

In another embodiment, the dose is between about 0.5 and about 1.0 mg per day.

In another embodiment, the dose is about 1 mg per day.

In another embodiment, the dose is about 2 mg per day.

In another embodiment, the dose is less than 0.2 mg per day.

In another embodiment, the dose is about 0.2 mg per day.

In another embodiment, the dose is less than 1.0 mg per day.

In another embodiment, the dose is about 1.0 mg per day.

In another embodiment, the dose is between about 0.2 and about 2.0 mg per day.

In another embodiment, the dose is between about 1.0 and about 2.0 mg per day.

In another embodiment, the dose is about 2 mg per day.

In another embodiment, the dose is about 4 mg per day.

In a specific embodiment, the recommended starting dosage may be 0.5, 1, 2, 3, 4, 5, 10, 15, 20, 25 or 50 mg per day. In another embodiment, the recommended starting dosage may be 0.5, 1, 2, 3, 4, or 5 mg per day. The dose may be escalated to 15, 20, 25, 30, 35, 40, 45 and 50 mg/day.

In certain embodiments, the therapeutically or prophylactically effective amount is from about 0.001 to about 100 mg/kg/day, from about 0.01 to about 50 mg/kg/day, from about 0.01 to about 25 mg/kg/day, from about 0.01 to about 10 mg/kg/day, from about 0.01 to about 9 mg/kg/day, 0.01 to about 8 mg/kg/day, from about 0.01 to about 7 mg/kg/day, from about 0.01 to about 6 mg/kg/day, from about 0.01 to about 5 mg/kg/day, from about 0.01 to about 4 mg/kg/day, from about 0.01 to about 3 mg/kg/day, from about 0.01 to about 2 mg/kg/day, or from about 0.01 to about 1 mg/kg/day.

The administered dose can also be expressed in units other than mg/kg/day. For example, doses for parenteral administration can be expressed as mg/m²/day. One of ordinary skill in the art would readily know how to convert doses from mg/kg/day to mg/m²/day to given either the height or weight of a subject or both (see, www.fda.gov/cder/cancer/animalframe.htm). For example, a dose of 1 mg/kg/day for a 65 kg human is approximately equal to 38 mg/m²/day.

A compound provided herein (e.g., a compound of Formula I (e.g., Compound 292), or an enantiomer or a mixture of enantiomers thereof, or a pharmaceutically acceptable salt, solvate, hydrate, co-crystal, clathrate, or polymorph thereof) can be administered once daily (QD), or divided into multiple daily doses such as twice daily (BID), three times daily (TID), and four times daily (QID). In addition, the administration can be continuous (i.e., daily for consecutive days or every day), intermittent, e.g., in cycles (i.e., including days, weeks, or months of rest without drug). As used herein, the term “daily” is intended to mean that a therapeutic compound, such as a compound of Formula I, is administered once or more than once each day, for example, for a period of time. The term “continuous” is intended to mean that a therapeutic compound, such as a compound of Formula I, is administered daily for an uninterrupted period of at least 10 days to 52 weeks. The term “intermittent” or “intermittently” as used herein is intended to mean stopping and starting at either regular or irregular intervals. For example, intermittent administration of a compound of Formula I is administration for one to six days per week, administration in cycles (e.g., daily administration for two to eight consecutive weeks, then a rest period with no administration for up to one week), or administration on alternate days. The term “cycling” as used herein is intended to mean that a therapeutic compound, such as a compound of Formula I, is administered daily or continuously but with a rest period.

In some embodiments, the frequency of administration is in the range of about a daily dose to about a monthly dose. In certain embodiments, administration is once a day, twice a day, three times a day, four times a day, once every other day, twice a week, once every week, once every two weeks, once every three weeks, or once every four weeks. In one embodiment, the compound provided herein is administered once a day. In another embodiment, the compound provided herein is administered twice a day. In yet another embodiment, the compound provided herein is administered three times a day. In still another embodiment, the compound provided herein is administered four times a day.

In one embodiment, a compound provided herein (e.g., a compound of Formula I (e.g., Compound 292), or an enantiomer or a mixture of enantiomers thereof, or a pharmaceutically acceptable salt, solvate, hydrate, co-crystal, clathrate, or polymorph thereof) is administered twice per day (BID). In one embodiment, the dose is about 0.1, 0.2, 0.25, 0.5, 1, 2, 2.5, 5, 10, 15, 20, 25, or 50 mg BID.

In one embodiment, a compound provided herein (e.g., a compound of Formula I (e.g., Compound 292), or an enantiomer or a mixture of enantiomers thereof, or a pharmaceutically acceptable salt, solvate, hydrate, co-crystal, clathrate, or polymorph thereof) is administered at a dose of less than 0.1, about 0.1, less than 0.5, about 0.5, between about 0.1 and about 1.0, between about 0.5 and about 1.0, about 1, or about 2 mg BID.

In another embodiment, a compound provided herein (e.g., a compound of Formula I (e.g., Compound 292), or an enantiomer or a mixture of enantiomers thereof, or a pharmaceutically acceptable salt, solvate, hydrate, co-crystal, clathrate, or polymorph thereof) is administered at a dose of less than 0.2, about 0.2, less than 1.0, about 1.0, between about 0.2 and about 2.0, between about 1.0 and about 2.0, about 2, or about 4 mg BID.

In one embodiment, the dose is less than 0.1 mg BID.

In another embodiment, the dose is about 0.1 mg BID.

In another embodiment, the dose is less than 0.5 mg BID.

In another embodiment, the dose is about 0.5 mg BID.

In another embodiment, the dose is between about 0.1 and about 1.0 mg BID.

In another embodiment, the dose is between about 0.5 and about 1.0 mg BID.

In another embodiment, the dose is about 1 mg BID.

In another embodiment, the dose is about 2 mg BID.

In another embodiment, the dose is less than 0.2 mg BID.

In another embodiment, the dose is about 0.2 mg BID.

In another embodiment, the dose is less than 1.0 mg BID.

In another embodiment, the dose is about 1.0 mg BID.

In another embodiment, the dose is between about 0.2 and about 2.0 mg BID.

In another embodiment, the dose is between about 1.0 and about 2.0 mg BID.

In another embodiment, the dose is about 2 mg BID.

In another embodiment, the dose is about 4 mg BID.

In one embodiment, a compound provided herein (e.g., a compound of Formula I (e.g., Compound 292), or an enantiomer or a mixture of enantiomers thereof, or a pharmaceutically acceptable salt, solvate, hydrate, co-crystal, clathrate, or polymorph thereof) is administered once daily (QD). In one embodiment, the dose is about 0.1, 0.2, 0.25, 0.5, 1, 2, 2.5, 5, 10, 15, 20, 25, or 50 mg QD.

In one embodiment, a compound provided herein (e.g., a compound of Formula I (e.g., Compound 292), or an enantiomer or a mixture of enantiomers thereof, or a pharmaceutically acceptable salt, solvate, hydrate, co-crystal, clathrate, or polymorph thereof) is administered at a dose of less than 0.1, about 0.1, less than 0.5, about 0.5, between about 0.1 and about 1.0, between about 0.5 and about 1.0, about 1, or about 2 mg QD.

In another embodiment, a compound provided herein (e.g., a compound of Formula I (e.g., Compound 292), or an enantiomer or a mixture of enantiomers thereof, or a pharmaceutically acceptable salt, solvate, hydrate, co-crystal, clathrate, or polymorph thereof) is administered at a dose of less than 0.2, about 0.2, less than 1.0, about 1.0, between about 0.2 and about 2.0, between about 1.0 and about 2.0, about 2, or about 4 mg QD.

In one embodiment, the dose is less than 0.1 mg QD.

In another embodiment, the dose is about 0.1 mg QD.

In another embodiment, the dose is less than 0.5 mg QD.

In another embodiment, the dose is about 0.5 mg QD.

In another embodiment, the dose is between about 0.1 and about 1.0 mg QD.

In another embodiment, the dose is between about 0.5 and about 1.0 mg QD.

In another embodiment, the dose is about 1 mg QD.

In another embodiment, the dose is about 2 mg QD.

In another embodiment, the dose is less than 0.2 mg QD.

In another embodiment, the dose is about 0.2 mg QD.

In another embodiment, the dose is less than 1.0 mg QD.

In another embodiment, the dose is about 1.0 mg QD.

In another embodiment, the dose is between about 0.2 and about 2.0 mg QD.

In another embodiment, the dose is between about 1.0 and about 2.0 mg QD.

In another embodiment, the dose is about 2 mg QD.

In another embodiment, the dose is about 4 mg QD.

In one embodiment, the amount of the compound administered is sufficient to provide a plasma concentration of the compound at steady state, ranging from about 0.005 to about 100 μM, from about 0.005 to about 10 μM, from about 0.01 to about 10 μM, from about 0.01 to about 5 μM, from about 0.005 to about 1 μM, from about 0.005 to about 0.5 μM, from about 0.005 to about 0.5 μM, from about 0.01 to about 0.2 μM, or from about 0.01 to about 0.1 μM. In one embodiment, the amount of the compound administered is sufficient to provide a plasma concentration at steady state, of about 0.005 to about 100 μM. In another embodiment, the amount of the compound administered is sufficient to provide a plasma concentration at steady state, of about 0.005 to about 10 μM. In yet another embodiment, the amount of the compound administered is sufficient to provide a plasma concentration at steady state, of about 0.01 to about 10 μM. In yet another embodiment, the amount of the compound administered is sufficient to provide a plasma concentration at steady state, of about 0.01 to about 5 μM. In yet another embodiment, the amount of the compound administered is sufficient to provide a plasma concentration at steady state, of about 0.005 to about 1 μM. In yet another embodiment, the amount of the compound administered is sufficient to provide a plasma concentration at steady state, of about 0.005 to about 0.5 μM. In yet another embodiment, the amount of the compound administered is sufficient to provide a plasma concentration of the compound at steady state, of about 0.01 to about 0.2 μM. In still another embodiment, the amount of the compound administered is sufficient to provide a plasma concentration of the compound at steady state, of about 0.01 to about 0.1 μM. As used herein, the term “plasma concentration at steady state” is the concentration reached after a period of administration of a compound. Once steady state is reached, there are minor peaks and troughs on the time dependent curve of the plasma concentration of the compound.

In one embodiment, the amount administered is sufficient to provide a maximum plasma concentration (peak concentration) of the compound, ranging from about 0.005 to about 100 μM, from about 0.005 to about 10 μM, from about 0.01 to about 10 μM, from about 0.01 to about 5 μM, from about 0.005 to about 1 μM, from about 0.005 to about 0.5 μM, from about 0.01 to about 0.2 μM, or from about 0.01 to about 0.1 μM. In one embodiment, the amount of the compound administered is sufficient to provide a maximum plasma concentration of the compound of about 0.005 to about 100 μM. In another embodiment, the amount of the compound administered is sufficient to provide a maximum plasma concentration of the compound of about 0.005 to about 10 μM. In yet another embodiment, the amount of the compound administered is sufficient to provide a maximum plasma concentration of the compound of about 0.01 to about 10 μM. In yet another embodiment, the amount of the compound administered is sufficient to provide a maximum plasma concentration of the compound of about 0.01 to about 5 μM. In yet another embodiment, the amount of the compound administered is sufficient to provide a maximum plasma concentration of the compound of about 0.005 to about 1 μM. In yet another embodiment, the amount of the compound administered is sufficient to provide a maximum plasma concentration of the compound of about 0.005 to about 0.5 μM. In yet another embodiment, the amount of the compound administered is sufficient to provide a maximum plasma concentration of the compound of about 0.01 to about 0.2 μM. In still another embodiment, the amount of the compound administered is sufficient to provide a maximum plasma concentration of the compound of about 0.01 to about 0.1 μM.

In one embodiment, the amount administered is sufficient to provide a minimum plasma concentration (trough concentration) of the compound, ranging from about 0.005 to about 100 μM, from about 0.005 to about 10 μM, from about 0.01 to about 10 μM, from about 0.01 to about 5 μM, from about 0.005 to about 1 μM, about 0.005 to about 0.5 μM, from about 0.01 to about 0.2 μM, or from about 0.01 to about 0.1 μM, when more than one doses are administered. In one embodiment, the amount of the compound administered is sufficient to provide a minimum plasma concentration of the compound of about 0.005 to about 100 μM. In another embodiment, the amount of the compound administered is sufficient to provide a minimum plasma concentration of the compound of about 0.005 to about 10 μM. In yet another embodiment, the amount of the compound administered is sufficient to provide a minimum plasma concentration of the compound of about 0.01 to about 10 μM. In yet another embodiment, the amount of the compound administered is sufficient to provide a minimum plasma concentration of the compound of about 0.01 to about 5 μM. In yet another embodiment, the amount of the compound administered is sufficient to provide a minimum plasma concentration of the compound of about 0.005 to about 1 μM. In yet another embodiment, the amount of the compound administered is sufficient to provide a minimum plasma concentration of the compound of about 0.005 to about 0.5 μM. In yet another embodiment, the amount of the compound administered is sufficient to provide a minimum plasma concentration of the compound of about 0.01 to about 0.2 μM. In still another embodiment, the amount of the compound administered is sufficient to provide a minimum plasma concentration of the compound of about 0.01 to about 0.1 μM.

In one embodiment, the amount administered is sufficient to provide an area under the curve (AUC) of the compound, ranging from about 50 to about 10,000 ng*hr/mL, about 100 to about 50,000 ng*hr/mL, from about 100 to 25,000 ng*hr/mL, or from about 10,000 to 25,000 ng*hr/mL.

The compound provided herein (e.g., a compound of Formula I (e.g., Compound 292), or an enantiomer or a mixture of enantiomers thereof, or a pharmaceutically acceptable salt, solvate, hydrate, co-crystal, clathrate, or polymorph thereof) may be administered by oral, parenteral (e.g., intramuscular, intraperitoneal, intravenous, CIV, intracistemal injection or infusion, subcutaneous injection, or implant), inhalation, nasal, vaginal, rectal, sublingual, or topical (e.g., transdermal or local) routes of administration. In one embodiment, the compound is administered orally. In another embodiment, the compound is administered parenterally. In yet another embodiment, the compound is administered intravenously.

In certain embodiments, a compound provided herein (e.g., a compound of Formula I (e.g., Compound 292), or an enantiomer or a mixture of enantiomers thereof, or a pharmaceutically acceptable salt, solvate, hydrate, co-crystal, clathrate, or polymorph thereof) is administered once per day from one day to six months, from one week to three months, from one week to four weeks, from one week to three weeks, or from one week to two weeks. In certain embodiments, the compound provided herein is administered once per day for one week, two weeks, three weeks, or four weeks. In one embodiment, the compound provided herein is administered once per day for one week. In another embodiment, the compound provided herein is administered once per day for two weeks. In yet another embodiment, the compound provided herein is administered once per day for three weeks. In still another embodiment, the compound provided herein is administered once per day for four weeks.

In certain embodiments, a compound provided herein (e.g., a compound of Formula I (e.g., Compound 292), or an enantiomer or a mixture of enantiomers thereof, or a pharmaceutically acceptable salt, solvate, hydrate, co-crystal, clathrate, or polymorph thereof) is administered twice per day from one day to six months, from one week to three months, from one week to four weeks, from one week to three weeks, or from one week to two weeks. In certain embodiments, the compound provided herein is administered twice per day for one week, two weeks, three weeks, or four weeks. In one embodiment, the compound provided herein is administered twice per day for one week. In another embodiment, the compound provided herein is administered twice per day for two weeks. In yet another embodiment, the compound provided herein is administered twice per day for three weeks. In still another embodiment, the compound provided herein is administered twice per day for four weeks.

The compound provided herein (e.g., a compound of Formula I (e.g., Compound 292), or an enantiomer or a mixture of enantiomers thereof, or a pharmaceutically acceptable salt, solvate, hydrate, co-crystal, clathrate, or polymorph thereof) can be delivered as a single dose such as, e.g., a single bolus injection, or oral tablets or pills; or over time, such as, e.g., continuous infusion over time or divided bolus doses over time. The compound can be administered repeatedly if necessary, for example, until the patient experiences stable disease or regression, or until the patient experiences disease progression or unacceptable toxicity.

In one embodiment, the regression of asthma is a decrease (e.g., at least 5%, 10%, 15%, 20%, 25%, 30%, 35%, 40%, 45%, 50% decrease) in the level of maximal decrease from pre-allergen challenge in FEV₁ following allergen challenge. The level of maximal decrease from pre-allergen challenge in FEV₁ following allergen challenge can be measured in EAR or LAR.

In one embodiment, the regression of asthma is a decrease (e.g., at least 5%, 10%, 15%, 20%, 25%, 30%, 35%, 40%, 45%, 50% decrease) in area under the curve (AUC) of FEV₁ following allergen challenge.

In one embodiment, the regression of asthma is an increase (e.g., at least 5%, 10%, 15%, 20%, 25%, 30%, 35%, 40%, 45%, 50% increase) in the amount of methacholine that is required to induce a 20% fall in FEV₁ (PC₂₀) following allergen challenge.

In one embodiment, the regression of asthma is a decrease (e.g., at least 5%, 10%, 15%, 20%, 25%, 30%, 35%, 40%, 45%, 50% decrease) in exhaled nitric oxide level of the subject.

In one embodiment, the regression of asthma is a decrease (e.g., at least 5%, 10%, 15%, 20%, 25%, 30%, 35%, 40%, 45%, 50% decrease) in the C-reactive protein (CRP) level of the subject.

In one embodiment, the regression of asthma is a decrease (e.g., at least 5%, 10%, 15%, 20%, 25%, 30%, 35%, 40%, 45%, 50% decrease) in white blood cell count and/or differential cell count in induced sputum of the subject after allergen challenge.

### Combination Treatments

In some embodiments, the compound provided herein is administered in combination with one or more other therapies. Such therapies include therapeutic agents as well as other medical interventions, behavioral therapies (e.g., avoidance of sunlight), and the like.

By “in combination with,” it is not intended to imply that the other therapy and the compound provided herein must be administered at the same time and/or formulated for delivery together, although these methods of delivery are within the scope of the invention. The compound provided herein can be administered concurrently with, prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks before), or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks after), one or more other therapies (e.g., one or more other additional agents). In general, each therapeutic agent will be administered at a dose and/or on a time schedule determined for that particular agent. The other therapeutic agent can be administered with the compound provided herein in a single composition or separately in a different composition. Triple therapy is also contemplated herein.

In general, it is expected that additional therapeutic agents employed in combination be utilized at levels that do not exceed the levels at which they are utilized individually. In some embodiments, the levels utilized in combination will be lower than those utilized individually.

In some embodiments, the compound provided herein is a first line treatment for rheumatoid arthritis or asthma, i.e., it is used in a subject who has not been previously administered another drug intended to treat rheumatoid arthritis, one or more symptoms of rheumatoid arthritis, asthma, or one or more symptoms of asthma.

In other embodiments, the compound provided herein is a second line treatment for rheumatoid arthritis or asthma, i.e., it is used in a subject who has been previously administered another drug intended to treat rheumatoid arthritis, one or more symptoms of rheumatoid arthritis, asthma, or one or more symptoms of asthma.

In other embodiments, the compound provided herein is a third or fourth line treatment for rheumatoid arthritis or asthma, i.e., it is used in a subject who has been previously administered two or three other drugs intended to treat rheumatoid arthritis, one or more symptoms of rheumatoid arthritis, asthma, or one or more symptoms of asthma.

In embodiments where two agents are administered, the agents can be administered in any order. For example, the two agents can be administered concurrently (i.e., essentially at the same time, or within the same treatment) or sequentially (i.e., one immediately following the other, or alternatively, with a gap in between administration of the two). In some embodiments, the compound provided herein is administered sequentially (i.e., after the first therapeutic).

In some embodiments, the compound provided herein and the second agent are administered as separate compositions, e.g., pharmaceutical compositions. In some embodiments, the compound provided herein and the agent are administered separately, but via the same route (e.g., both orally or both intravenously). In other embodiments, the PI3K inhibitor and the agent are administered in the same composition, e.g., pharmaceutical composition.

In some embodiments, the compound provided herein (e.g., PI3Kδ inhibitor) is administered in combination with an agent that inhibits IgE production or activity. In some embodiments, the compound provided herein (e.g., PI3Kδ inhibitor) is administered in combination with an inhibitor of mTOR. Agents that inhibit IgE production are known in the art and they include but are not limited to one or more of TEI-9874, 24446-cyclohexyloxy-2-naphtyloxy)phenylacetamide)benzoic acid, rapamycin, rapamycin analogs (i.e., rapalogs), TORC1 inhibitors, TORC2 inhibitors, and any other compounds that inhibit mTORC1 and mTORC2. Agents that inhibit IgE activity include, for example, anti-IgE antibodies such as for example Omalizumab and TNX-901.

In some embodiments, a compound provided herein can be used in combination with commonly prescribed drugs for the treatment of autoimmune disease, including, but not limited to Enbrel®, Remicade®, Humira®, Avonex®, and Rebif®.

In certain embodiments, wherein inflammation (e.g., arthritis, asthma) is treated, prevented and/or managed, a compound provided herein can be combined with, for example: PI3K inhibitors such as GS-1101, XL 499, GDC-0941, and AMG-319; BTK inhibitors such as ibrutinib and AVL-292; JAK inhibitors such as tofacitinib, fostamatinib, and GLPG0636.

In certain embodiments wherein arthritis is treated, prevented and/or managed, a compound provided herein can be combined with, for example: TNF antagonist (e.g., a TNF antibody or fragment, a soluble TNF receptor or fragment, fusion proteins thereof, or a small molecule TNF antagonist); other biologic antirhheumatics (e.g., IL-6 antagonists, IL-1 antagonists, costimulatory modulators); an antirheumatic (e.g., methotrexate, auranofin, aurothioglucose, azathioprine, etanercept, gold sodium thiomalate, chrloroquine, hydroxychloroquine sulfate, leflunomide, sulfasalzine, penicillamine); a muscle relaxant; a narcotic; a non-steroid anti-inflammatory drug (NSAID); an analgesic; an anesthetic; a sedative; a local anesthetic; a neuromuscular blocker; an antimicrobial (e.g., an aminoglycoside, an antifungal, an antiparasitic, an antiviral, a carbapenem, cephalosporin, a fluoroquinolone, a macrolide, a penicillin, a sulfonamide, a tetracycline, another antimicrobial); an antipsoriatic; a corticosteroid; an anabolic steroid; a cytokine or a cytokine antagonist; a calcineurin inhibitor (e.g., cyclosporine, tacrolimus).

In some embodiments, a compound provided herein (e.g., a compound of Formula I (e.g., Compound 292), or an enantiomer or a mixture of enantiomers thereof, or a pharmaceutically acceptable salt, solvate, hydrate, co-crystal, clathrate, or polymorph thereof) is administered in combination with an agent for the treatment of rheumatoid arthritis. Examples of agents for the treatment of rheumatoid arthritis include, but are not limited to, various NSAIDs, corticosteroids, sulfasalazine, auranofin, methotrexate, azathioprine, penicillamine, cyclosporine, Arava (leflunomide), TNF inhibitors (e.g., Enbrel (etanercept), Remicade (infliximab), Humira (adalimumab), Simponi (golimumab), and Cimzia (certolizumab)), IL-1 inhibitors (e.g., Kineret (anakinra)), T-cell costimulatory modulators (e.g., Orencia (abatacept)), Anti-CD20 (e.g., Rituxan (rituximab)), and IL-6 inhibitors (e.g., Actemra (tocilizumab)). In one embodiment, the agent is Cimzia (certolizumab). In another embodiment, the agent is Actemra (tocilizumab).

In some embodiments, a compound provided herein (e.g., a compound of Formula I (e.g., Compound 292), or an enantiomer or a mixture of enantiomers thereof, or a pharmaceutically acceptable salt, solvate, hydrate, co-crystal, clathrate, or polymorph thereof) is administered in combination with an agent for rheumatology. Examples of agents for rheumatology include, but are not limited to, Rayos (prednisone), Stendra (avanafil), Actemra (tocilizumab), Duexis (ibuprofen and famotidine), Actemra (tocilizumab), Krystexxa (pegloticase), Vimovo (naproxen+esomeprazole), Cimzia (certolizumab pegol), Colcrys (colchicine), Pennsaid (diclofenac sodium topical solution), Simponi (golimumab), Uloric (febuxostat), Orencia (abatacept), Elaprase (idursulfase), Orencia (abatacept), Vioxx (rofecoxib), Enbrel (etanercept), Humira (adalimumab), Remicade (infliximab), Bextra, Kineret, Remicade (infliximab), Supartz, Mobic (meloxicam), Vivelle (estradiol transdermal system), Lodine XL (etodolac), Arava, Salagen, Arthrotec, Etodolac, Ketoprofen, Synvisc, Tolmetin Sodium, Azulfidine EN-tabs Tablets (sulfasalazine delayed release tablets, USP), and Naprelan (naproxen sodium).

In some embodiments, the second agent is selected from belimumab, AGS-009, rontalizumab, vitamin D3, sifalimumab, AMG 811, IFNα Kinoid, CEP33457, epratuzumab, LY2127399, Ocrelizumab, Atacicept, A-623, SBI-087, AMG557, laquinimod, rapamycin, cyclophosphamide, azathioprine, mycophenolate, leflunomide, methotrexate, CNTO 136, tamibarotene, N-acetylcysteine, CDP7657, hydroxychloroquine, rituximab, carfilzomib, bortezomib, ONX 0914, IMO-3100, DV 1179, sulfasalazine, and chloroquine. In one embodiment, the second agent is methotrexate, sulfasalazine, chloroquine, or hydroxychloroquine. In one embodiment, the second agent is methotrexate.

In some embodiments, a compound provided herein (e.g., a compound of Formula I (e.g., Compound 292), or an enantiomer or a mixture of enantiomers thereof, or a pharmaceutically acceptable salt, solvate, hydrate, co-crystal, clathrate, or polymorph thereof) is administered in combination of methotrexate (MTX). In one embodiment, MTX is administered to the subject at least 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks before a compound provided herein (e.g., a compound of Formula I (e.g., Compound 292), or an enantiomer or a mixture of enantiomers thereof, or a pharmaceutically acceptable salt, solvate, hydrate, co-crystal, clathrate, or polymorph thereof) is administered. In another embodiment, MTX is administered concurrently with a compound provided herein (e.g., a compound of Formula I (e.g., Compound 292), or an enantiomer or a mixture of enantiomers thereof, or a pharmaceutically acceptable salt, solvate, hydrate, co-crystal, clathrate, or polymorph thereof). In yet another embodiment, MTX is administered to the subject at least 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks after a compound provided herein (e.g., a compound of Formula I (e.g., Compound 292), or an enantiomer or a mixture of enantiomers thereof, or a pharmaceutically acceptable salt, solvate, hydrate, co-crystal, clathrate, or polymorph thereof) is administered. In one embodiment, the compound is Compound 292.

In one embodiment, MTX is administered at least 3 months before Compound 292 is administered. In one embodiment, MTX is administered on a stable dose before Compound 292 is administered. In one embodiment, MTX is administered on a stable dose for at least 6 weeks before Compound 292 is administered. In one embodiment, MTX is administered on a stable dose of about 7.5 to about 25.0 mg once per week (split doses are permitted) for at least 6 weeks before Compound 292 is administered.

In some embodiments, a compound provided herein can be combined with other agents that act to relieve the symptoms of inflammatory conditions, such as encephalomyelitis, asthma, and the other diseases described herein. These agents include, but are not limited to, non-steroidal anti-inflammatory drugs (NSAIDs), e.g., acetylsalicylic acid; ibuprofen; naproxen; indomethacin; nabumetone; and tolmetin. In some embodiments, corticosteroids are used to reduce inflammation and suppress activity of the immune system.

In some embodiments, a compound provided herein (e.g., a compound of Formula I (e.g., Compound 292), or an enantiomer or a mixture of enantiomers thereof, or a pharmaceutically acceptable salt, solvate, hydrate, co-crystal, clathrate, or polymorph thereof) is administered in combination with an agent for pulmonary or respiratory diseases. Examples of agents for pulmonary or respiratory diseases include, but are not limited to, Dymista (azelastine hydrochloride and fluticasone propionate), Kalydeco (ivacaftor), Qnas1 (beclomethasone dipropionate) nasal aerosol, Rayos (prednisone) delayed-release tablets, Surfaxin (lucinactant), Tudorza Pressair (aclidinium bromide inhalation powder), Arcapta (indacaterol maleate inhalation powder), Daliresp (roflumilast), Xalkori (crizotinib), Cayston (aztreonam for inhalation solution), Dulera (mometasone furoate+formoterol fumarate dihydrate), Teflaro (ceftaroline fosamil), Adcirca (tadalafil), Tyvaso (treprostinil), Alvesco (ciclesonide), Patanase (olopatadine hydrochloride), Letairis (ambrisentan), Xyzal (levocetirizine dihydrochloride), Brovana (arformoterol tartrate), Tygacil (tigecycline), Ketek (telithromycin), Spiriva HandiHaler (tiotropium bromide), Aldurazyme (laronidase), Iressa (gefitinib), Xolair (omalizumab), Zemaira (alphal-proteinase inhibitor), Clarinex, Qvar (beclomethasone dipropionate), Remodulin (treprostinil), Xopenex, Avelox I.V. (moxifloxacin hydrochloride), DuoNeb (albuterol sulfate and ipratropium bromide), Foradil Aerolizer (formoterol fumarate inhalation powder), Invanz, NasalCrom Nasal Spray, Tavist (clemastine fumarate), Tracleer (bosentan), Ventolin HFA (albuterol sulfate inhalation aerosol), Biaxin XL (clarithromycin extended-release tablets), Cefazolin and Dextrose USP, Tri-Nasal Spray (triamcinolone acetonide spray), Accolate, Cafcit Injection, Proventil HFA Inhalation Aerosol, Rhinocort Aqua Nasal Spray, Tequin, Tikosyn Capsules, Allegra-D, Clemastine fumarate syrup, Curosurf, Dynabac, Infasurf, Priftin, Pulmozyme (dornase alfa), Sclerosol Intrapleural Aerosol, Singulair, Synagis, Ceftin (cefuroxime axetil), Cipro (ciprofloxacin HCl), Claritin RediTabs (10 mg loratadine rapidly-disintegrating tablet), Flonase Nasal Spray, Flovent Rotadisk, Metaprotereol Sulfate Inhalation Solution (5%), Nasacort AQ (triamcinolone acetonide) Nasal Spray, Omnicef, Raxar (grepafloxacin), Serevent, Tilade (nedocromil sodium), Tobi, Vanceril 84 mcg Double Strength (beclomethasone dipropionate, 84 mcg) Inhalation Aerosol, Zagam (sparfloxacin) tablets, Zyflo (Zileuton), Accolate, Allegra (fexofenadine hydrochloride), Astelin nasal spray, Atrovent (ipratropium bromide), Augmentin (amoxicillin/clavulanate), Azmacort (triamcinolone acetonide) Inhalation Aerosol, Breathe Right, Claritin Syrup (loratadine), Claritin-D 24 Hour Extended Release Tablets (10 mg loratadine, 240 mg pseudoephedrine sulfate), Covera-HS (verapamil), Nasacort AQ (triamcinolone acetonide) Nasal Spray, OcuHist, Pulmozyme (dornase alfa), RespiGam (Respiratory Syncitial Virus Immune Globulin Intravenous), Tavist (clemastine fumarate), Tripedia (Diptheria and Tetanus Toxoids and Acellular Pertussis Vaccine Absorbed), Vancenase AQ 84 mcg Double Strength, Visipaque (iodixanol), Zosyn (sterile piperacillin sodium/tazobactam sodium), Cedax (ceftibuten), and Zyrtec (cetirizine HCl). In one embodiment, the agent for pulmonary or respiratory diseases is Arcapta, Daliresp, Dulera, Alvesco, Brovana, Spiriva HandiHaler, Xolair, Qvar, Xopenex, DuoNeb, Foradil Aerolizer, Accolate, Singulair, Flovent Rotadisk, Tilade, Vanceril, Zyflo, or Azmacort Inhalation Aerosol. In one embodiment, the agent for pulmonary or respiratory diseases is Spiriva HandiHaler.

In some embodiments, a compound provided herein (e.g., a compound of Formula I (e.g., Compound 292), or an enantiomer or a mixture of enantiomers thereof, or a pharmaceutically acceptable salt, solvate, hydrate, co-crystal, clathrate, or polymorph thereof) is administered in combination with an agent for immunology or infectious diseases. Examples of agents for immunology or infectious diseases include, but are not limited to, Horizant (gabapentin enacarbil), Qnasl (beclomethasone dipropionate) nasal aerosol, Rayos (prednisone) delayed-release tablets, Stribild (elvitegravir, cobicistat, emtricitabine, tenofovir disoproxil fumarate), Tudorza Pressair (aclidinium bromide inhalation powder), Arcapta (indacaterol maleate inhalation powder), Benlysta (belimumab), Complera (emtricitabine/rilpivirine/tenofovir disoproxil fumarate), Daliresp (roflumilast), Dificid (fidaxomicin), Edurant (rilpivirine), Firazyr (icatibant), Gralise (gabapentin), Incivek (telaprevir), Nulojix (belatacept), Victrelis (boceprevir), Cayston (aztreonam for inhalation solution), Egrifta (tesamorelin for injection), Menveo (meningitis vaccine), Oravig (miconazole), Prevnar 13 (Pneumococcal 13-valent Conjugate Vaccine), Teflaro (ceftaroline fosamil), Zortress (everolimus), Zymaxid (gatifloxacin ophthalmic solution), Bepreve (bepotastine besilate ophthalmic solution), Berinert (C1 Esterase Inhibitor (Human)), Besivance (besifloxacin ophthalmic suspension), Cervarix [Human Papillomavirus Bivalent (Types 16 and 18) Vaccine, Recombinant], Coartem (artemether/lumefantrine), Hiberix (Haemophilus b Conjugate Vaccine; Tetanus Toxoid Conjugate), Ilaris (canakinumab), Ixiaro (Japanese Encephalitis Vaccine, Inactivated, Adsorbed), Kalbitor (ecallantide), Qutenza (capsaicin), Vibativ (telavancin), Zirgan (ganciclovir ophthalmic gel), Aptivus (tipranavir), Astepro (azelastine hydrochloride nasal spray), Cinryze (C1 Inhibitor (Human)), Intelence (etravirine), Moxatag (amoxicillin), Rotarix (Rotavirus Vaccine, Live, Oral), Tysabri (natalizumab), Viread (tenofovir disoproxil fumarate), Altabax (retapamulin), AzaSite (azithromycin), Doribax (doripenem), Extina (ketoconazole), Isentress (raltegravir), Selzentry (maraviroc), Veramyst (fluticasone furoate), Xyzal (levocetirizine dihydrochloride), Eraxis (anidulafungin), Gardasil (quadrivalent human papillomavirus (types 6, 11, 16, 18) recombinant vaccine), Noxafil (posaconazole), Prezista (darunavir), Rotateq (rotavirus vaccine, live oral pentavalent), Tyzeka (telbivudine), Veregen (kunecatechins), Aptivus (tipranavir), Baraclude (entecavir), Tygacil (tigecycline), Ketek (telithromycin), Tindamax, tinidazole, Xifaxan (rifaximin), Amevive (alefacept), FluMist (Influenza Virus Vaccine), Fuzeon (enfuvirtide), Lexiva (fosamprenavir calcium), Reyataz (atazanavir sulfate), Alinia (nitazoxanide), Clarinex, Daptacel, Fluzone Preservative-free, Hepsera (adefovir dipivoxil), Pediarix Vaccine, Pegasys (peginterferon alfa-2a), Restasis (cyclosporine ophthalmic emulsion), Sustiva, Vfend (voriconazole), Avelox I.V. (moxifloxacin hydrochloride), Cancidas, Peg-Intron (peginterferon alfa-2b), Rebetol (ribavirin), Spectracef, Twinrix, Valcyte (valganciclovir HCl), Viread (tenofovir disoproxil fumarate), Xigris (drotrecogin alfa [activated]), ABREVA (docosanol), Biaxin XL (clarithromycin extended-release tablets), Cefazolin and Dextrose USP, Children's Motrin Cold, Evoxac, Kaletra Capsules and Oral Solution, Lamisil (terbinafine hydrochloride) Solution (1%), Lotrisone (clotrimazole/betamethasone diproprionate) lotion, Malarone (atovaquone; proguanil hydrochloride) Tablet, Rapamune (sirolimus) Tablets, Rid Mousse, Tri-Nasal Spray (triamcinolone acetonide spray), Trivagizole 3 (clotrimazole) Vaginal Cream, Trizivir (abacavir sulfate; lamivudine; zidovudine AZT) Tablet, Agenerase (amprenavir), Cleocin (clindamycin phosphate), Famvir (famciclovir), Norvir (ritonavir), Panretin Gel, Rapamune (sirolimus) oral solution, Relenza, Synercid I.V., Tamiflu capsule, Vistide (cidofovir), Allegra-D, CellCept, Clemastine fumarate syrup, Cleocin (clindamycin phosphate), Dynabac, REBETRON™ Combination Therapy, Simulect, Timentin, Viroptic, INFANRIX (Diphtheria and Tetanus Toxoids and Acellular Pertussis Vaccine Adsorbed), Acyclovir Capsules, Aldara (imiquimod), Aphthasol, Combivir, Condylox Gel 0.5% (pokofilox), Famvir (famciclovir), Flagyl ER, Flonase Nasal Spray, Fortovase, INFERGEN (interferon alfacon-1), Intron A (interferon alfa-2b, recombinant), Norvir (ritonavir), Rescriptor Tablets (delavirdine mesylate tablets), SPORANOX (itraconazole), Stromectol (ivermectin), Taxol, Trovan, VIRACEPT (nelfinavir mesylate), Zerit (stavudine), Albenza (albendazole), Apthasol (Amlexanox), Carrington patch, Confide, Crixivan (Indinavir sulfate), Gastrocrom Oral Concentrate (cromolyn sodium), Havrix, Lamisil (terbinafine hydrochloride) Tablets, Leukine (sargramostim), Oral Cytovene, RespiGam (Respiratory Syncitial Virus Immune Globulin Intravenous), Videx (didanosine), Viramune (nevirapine), Vistide (cidofovir), Vitrasert Implant, Zithromax (azithromycin), Cedax (ceftibuten), Clarithromycin (Biaxin), Epivir (lamivudine), Intron A (Interferon alfa-2b, recombinant), Invirase (saquinavir), Valtrex (valacyclovir HCl), Western blot confirmatory device, Zerit (stavudine), and Zyrtec (cetirizine HCl).

### Biomarkers

Provided herein are methods relating to the use of mRNAs or proteins as biomarkers to ascertain the effectiveness of autoimmune arthritis (e.g., rheumatoid arthritis or asthma) therapy. mRNA or protein levels can be used to determine whether a particular agent is likely to be successful in the treatment of autoimmune arthritis (e.g., rheumatoid arthritis or asthma). The level of a biomarker (e.g., mRNA) provided herein can be measured by the methods provided herein or known in the art. Examples include, but are not limited to, quantitative RT-PCR (qRT-PCR), and gene expression chip (e.g., Mammaprint assay by Agendia, Inc.). In one embodiment, the level of an mRNA biomarker provided herein is measured by quantitative RT-PCR (qRT-PCR).

As used herein, and unless otherwise specified, a biological marker or “biomarker” is a substance whose detection indicates a particular biological state, such as, for example, the presence of autoimmune arthritis (e.g., rheumatoid arthritis) or asthma. In some embodiments, biomarkers can either be determined individually, or several biomarkers can be measured simultaneously.

In some embodiments, a “biomarker” indicates a change in the level of mRNA expression that may correlate with the risk or progression of a disease, or with the susceptibility of the disease to a given treatment. In some embodiments, the biomarker is a nucleic acid, such as a mRNA or cDNA.

In additional embodiments, a “biomarker” indicates a change in the level of polypeptide or protein expression that may correlate with the risk, susceptibility to treatment, or progression of a disease. In some embodiments, the biomarker can be a polypeptide or protein, or a fragment thereof. The relative level of specific proteins can be determined by methods known in the art. For example, antibody based methods, such as an immunoblot, enzyme-linked immunosorbent assay (ELISA), bead-based immunoassay, or other methods can be used.

The methods provided herein encompass methods for screening or identifying autoimmune arthritis (e.g., rheumatoid arthritis) or asthma patients for treatment with a compound provided herein (e.g., a compound of Formula I (e.g., Compound 292), or an enantiomer or a mixture of enantiomers thereof, or a pharmaceutically acceptable salt, solvate, hydrate, co-crystal, clathrate, or polymorph thereof). The method comprises obtaining a biological sample from the subject, and measuring the level of a biomarker in the biological sample, where an abnormal baseline level (e.g., higher or lower than the level in a control group) of the biomarker indicates a higher likelihood that the subject has autoimmune arthritis (e.g., rheumatoid arthritis) or asthma that can be treated with a compound provided herein (e.g., a compound of Formula I (e.g., Compound 292), or an enantiomer or a mixture of enantiomers thereof, or a pharmaceutically acceptable salt, solvate, hydrate, co-crystal, clathrate, or polymorph thereof). In one embodiment, the method optionally comprises isolating or purifying mRNA from the biological sample, amplifying the mRNA transcripts (e.g., by RT-PCR). In one embodiment, the level of a biomarker is the level of an mRNA or a protein.

In some embodiments, provided herein are methods of predicting the sensitivity to treatment with a compound provided herein (e.g., a compound of Formula I (e.g., Compound 292), or an enantiomer or a mixture of enantiomers thereof, or a pharmaceutically acceptable salt, solvate, hydrate, co-crystal, clathrate, or polymorph thereof) in a autoimmune arthritis (e.g., rheumatoid arthritis) or asthma patient. The method comprises obtaining a biological sample from the patient, and measuring the level of a biomarker in the biological sample, where an abnormal baseline level (e.g., higher or lower than the level in a control group) of the biomarker indicates a higher likelihood that the autoimmune arthritis (e.g., rheumatoid arthritis) or asthma will be sensitive to treatment with a compound provided herein (e.g., a compound of Formula I (e.g., Compound 292), or an enantiomer or a mixture of enantiomers thereof, or a pharmaceutically acceptable salt, solvate, hydrate, co-crystal, clathrate, or polymorph thereof).). In one embodiment, the method optionally comprises isolating or purifying mRNA from the biological sample, amplifying the mRNA transcripts (e.g., by RT-PCR). In one embodiment, the level of a biomarker is the level of an mRNA or a protein.

In one embodiment, provided herein is a method for treating or managing autoimmune arthritis (e.g., rheumatoid arthritis) or asthma in a patient, comprising: (i) obtaining a biological sample from the patient and measuring the level of a biomarker in the biological sample; and (ii) administering to the patient with an abnormal baseline level (e.g., higher or lower than the level in a control group) of at least one biomarker a therapeutically effective amount of a compound provided herein (e.g., a compound of Formula I (e.g., Compound 292), or an enantiomer or a mixture of enantiomers thereof, or a pharmaceutically acceptable salt, solvate, hydrate, co-crystal, clathrate, or polymorph thereof). In one embodiment, step (i) optionally comprises isolating or purifying mRNA from the biological sample, amplifying the mRNA transcripts (e.g., by RT-PCR). In one embodiment, the level of a biomarker is the level of an mRNA or a protein.

In another embodiment, provided herein is a method of monitoring response to treatment with a compound provided herein (e.g., a compound of Formula I (e.g., Compound 292), or an enantiomer or a mixture of enantiomers thereof, or a pharmaceutically acceptable salt, solvate, hydrate, co-crystal, clathrate, or polymorph thereof) in a autoimmune arthritis (e.g., rheumatoid arthritis) or asthma patient. The method comprises obtaining a biological sample from the patient, measuring the level of a biomarker in the biological sample, administering a compound provided herein (e.g., a compound of Formula I (e.g., Compound 292), or an enantiomer or a mixture of enantiomers thereof, or a pharmaceutically acceptable salt, solvate, hydrate, co-crystal, clathrate, or polymorph thereof) to the patient, thereafter obtaining a second biological sample from the patient, measuring the level of the biomarker in the second biological sample, and comparing the two levels of the biomarker, where an altered (e.g., increased or decreased) level of the biomarker after treatment indicates the likelihood of rheumatoid arthritis or asthma improvement. In one embodiment, a decreased level of biomarker after treatment indicates the likelihood of rheumatoid arthritis or asthma improvement. In another embodiment, an increased level of biomarker after treatment indicates the likelihood of rheumatoid arthritis or asthma improvement. The level of biomarker can be, for example, the level of an mRNA or a protein. The expression in the treated sample can increase, for example, by about 1.5×, 2.0×, 3×, 5×, or more.

In yet another embodiment, a method for monitoring patient compliance with a drug treatment protocol is provided. The method comprises obtaining a biological sample from the patient, measuring the level of at least one biomarker in the sample, and determining if the level is increased or decreased in the patient sample compared to the level in a control untreated sample, wherein an increased or decreased level indicates patient compliance with the drug treatment protocol. In one embodiment, the level of at least one biomarker is increased. The biomarker level monitored can be, for example, mRNA level or protein level. The expression in the treated sample can increase, for example, by about 1.5×, 2.0×, 3×, 5×, or more.

A subset of TLRs (e.g., TLR7, TLR8, and TLR9) induce an immune response characterized by induction of IFN-α. PI3K inhibition by Compound 292 inhibits induction of IFN-α via TLR9. TLR9 is a nucleotide-sensing TLR; and functions as a receptor for viral and bacterial nucleic acids, as well as cellular danger or stress signals, e.g., acute phase reactants. In addition to the recognition of foreign nucleic acids, TLR9 has been shown to recognize self nucleic acid complexes in inflammatory conditions, such as rheumatoid arthritis or asthma. As stated above, biological concomitants of inflammatory conditions (e.g., rheumatoid arthritis or asthma) can include increased levels of TLR 9 signaling induced cytokines such as IFN-α. The potent inhibition of the TLR9-induced IFN-α signaling pathway by Compound 292 indicates Compound 292 can be used to prevent or treat disorders where the IFN-α or a TLR (e.g., TLR9) signaling pathway is altered, (e.g., increased or decreased). Examples of such disorders include, but are not limited to, inflammatory conditions, lupus, cutaneous lupus, rheumatoid arthritis, scleroderma, and dermatomyositis.

In other embodiments, an altered level (e.g., increased or decreased) of TLR 9-induced cytokines, such as IFN-α, can be used as a biomarker to select patients for treatment with Compound 292. For example, a subject, e.g., a patient suffering from an inflammatory condition, e.g., lupus, cutaneous lupus, rheumatoid arthritis, scleroderma, systemic scleroderma, or dermatomyositis, can be screened for expression of TLR 9 induced cytokine expression, and/or IFN-α; based on the cytokine expression profile, the subject selected or not selected for treatment with Compound 292. Other embodiments include, screening a subject, e.g., a patient diagnosed with autoimmune arthritis (e.g., rheumatoid arthritis) or asthma, for expression of IFN-α, if the subject expresses an increased level of IFN-α as compared to a reference value (e.g., a reference standard), the subject is then selected for treatment with Compound 292.

A gene signature characteristic of a type I interferon response commonly activated in rheumatic diseases can also be evaluated. Rheumatic diseases that can be evaluated can include, but are not limited to, systemic lupus erythematosus, dermatomyositis, polymyositis, rheumatoid arthritis, and systemic scleroderma (e.g., as described in Higgs et al. Ann Rheum Dis (2011) 70: 2029-2036). The gene signature can include analysis of the level (e.g., expression) of one or more genes involved in a type I interferon induced response, e.g., IF16, RSAD2, STAT2, IF144, LIPA, IF144L and IF127 (e.g., as described in Higgs et al 2011, supra).

In an embodiment, the gene signature can include analysis of the level (e.g., expression) of one or more of: type I IFNs, TNF-α, IL-1β, IL-10, IL-13, IL-17, or GM-CSF (e.g., as described in Higgs et al. International Journal of Rheumatic Diseases (2012) 15: 25-35). In one embodiment, the gene signature can include analysis of the level (e.g., expression) of one or more of the following: IFN-α serum levels of high-mobility group box protein 1 (HMGB1), C3a, or dsDNA (e.g., as described in Ruan et al. The Journal of Immunology (2010) 185: 4213-4222). In an embodiment, the gene signature can include analysis of the level (e.g., expression) of one or more of: inflammatory cytokines, e.g., type I IFNs, type II IFNs, IL-6, IL-1, TNF-α; immunomodulatory cytokines, e.g., IL-10 and TGF-β; IL-21, IL-17, or IL-2 (e.g., as described in Ohl et al. Journal of Biomedicine and Biotechnology (2011) Article ID: 432595).

Any combination of the aforementioned genes can be used to evaluate a subject. In one embodiment, the the levels, e.g., expression, of one, two, three, four, five, six, seven, eight, nine, ten, eleven, twelve, thirteen, fourteen, fifteen or more than fifteen of: IFN-α, type I IFNs, type II IFNs, TNF-α, IL-1β, IL-4, IL-6, IL-1, IL-2, IL-8, IL-10, IL-13, IL-17, IL-21, GM-CSF, TGF-13, IF16, RSAD2, STAT2, IF144, LIPA, IF144L or IF127 can be evaluated. In another embodiment, the levels, e.g., expression, of one or more of: IFN-α, TNF-α, IL-6, IL-8, or IL-21. In one embodiment, the gene signature can include analysis of level (e.g., expression) of IL-17.

In some embodiments, the methods provided herein result in inhibition of immune complexes, cytokines (e.g., interferons (e.g., Type I interferons, e.g., IFN-α and/or IFN-β); interleukins (e.g., IL-6, IL-8, and IL-1) and TNF-α), anti-dsDNA autoantibodies, IFN-α and/or IFN-β inducible genes, IP-10, or sCD40L. In some embodiments, the methods provided herein result in inhibition of a Type I IFN (e.g., IFN-α).

In some embodiments, the methods provided herein result in modulation (e.g., inhibition) of a cytokine (e.g., a Type I IFN (e.g., IFN-α)) released as a result of TLR activation. In some embodiments, the TLR is TLR9. In some embodiments, the methods result in inhibition of IFN-α released as a result of TLR9 activation.

In some embodiments, the methods provided herein result in decreases in antinuclear antibodies (e.g., anti-Smith antibodies, anti-double stranded DNA (dsDNA) antibodies, or anti-histone antibodies. In some embodiments, the method provided herein result in decreases in anticardiolipin antibodies.

In one embodiment, the biomarker used in the methods provided herein is the expression level of IL-6. In one embodiment, the expression level of IL-6 is determined from a serum or plasma sample from the subject. In one embodiment, the expression level of IL-6 is determined by techniques known in the art (e.g., ELISA).

In one embodiment, the biomarker used in the methods provided herein is the expression level of mRNA for IL-4 or IL-21. In one embodiment, the expression level of mRNA for IL-4 or IL-21 is determined from a whole blood sample from the subject. In one embodiment, the expression level of mRNA for IL-4 or IL-21 is determined by techniques known in the art (e.g., RNA expression).

In one embodiment, the biomarker used in the methods provided herein is a germline SNP that has been previously linked to autoimmune disease susceptibility (e.g., PTPN22) or to pathways of drug metabolism or transport (e.g., CYP3A family and/or other drug metabolizing enzymes that have been associated with metabolism of a compound provided herein).

In one embodiment, the biomarker used in the methods provided herein is an immunophenotyping biomarker. In one embodiment, the biomarker is the absolute count or percentage of mature human T lymphocytes (CD3+), natural killer cells (CD56+), B lymphocytes (CD19+), suppressor/cytotoxic (CD3+CD8+) T-lymphocyte subsets, or helper/inducer (CD3+CD4+) T-lymphocyte subsets.

In one embodiment, the biomarker used in a method provided herein is the level of one or more of EGF, Eotaxin (CCL11), FGF-2, Flt-3 ligand, Fractalkine, G-CSF, GM-CSF, GRO, IFNα2, IFNα, IL-α, IL-β, IL-1ra, IL-2, sIL-2Rα, IL-3, IL-4, IL-5, IL-6, IL-7, IL-8, IL-9, IL-10, IL-12 (p40), IL-12 (p70), IL-13, IL-15, IL-17, IL-1ra, IL-1α, IL-143, MCP-1, MCP-3, MDC (CCL22), MIP-1α (CCL3), MIP-1β (CCL4), PDGF-AA, PDGF-AB/BB, RANTES (CCL5), sCD40L, sIL-2Rα, TGFα, TNFα, TNFβ, VEGF, 6Ckine, BCA-1 (CXCL13), CTACK, ENA-78, Eotaxin-2 (CCL24), Eotaxin-3 (CCL26), 1-309, IL-16, IL-20, IL-21, IL-23, IL-28, IL-33, LIF, MCP-2, MCP-4 (CCL13), MIP-1d, SCF, SDF-1α+β (CXCL12), TARC (CCL17), TPO, TRAIL, or TSLP. In one embodiment, the biomarker used in a method provided herein is the level of one or more of Eotaxin (CCL11), IL-4, IL-5, IL-9, IL-13, MDC (CCL22), RANTES (CCL5), Eotaxin-2 (CCL24), Eotaxin-3 (CCL26), MCP-4 (CCL13), SCF, TARC (CCL17), or TSLP. In another embodiment, the biomarker used in a method provided herein is the level of one or more of MIP-1α (CCL3), MIP-1β (CCL4), TNFα, BCA-1 (CXCL13), or SDF- 1α+β (CXCL12).

In one embodiment, the biomarker used in a method provided herein is the level of one or more of IL-13, IL-5, KC/GRO, or TNF-α. In one embodiment, the biomarker used in a method provided herein is the level of one or more of IL-13, IL-5, KC/GRO, or TNF-α in the bronchoalveolar lavage (BAL) fluid in a rodent (e.g., mouse or rat) ovalbumin allergic asthma model. In one embodiment, a decrease in the level of one or more of IL-13, IL-5, KC/GRO, or TNF-α is indicative of inhibition of lung inflammation or asthma.

In one embodiment, the biomarker used in a method provided herein is the level of one or more of periostin, exhaled NO, sputum eosinophils, or serum cytokines.

In one embodiment, the biomarker used in the methods provided herein is an altered level (e.g., increased or decreased) of a protein in a biological sample. In another embodiment, the biomarker used in the methods provided herein is the level of a modification of a protein (e.g., phosphorylation of a protein) in a biological sample. Examples of the biological sample include, but are not limited to, blood, serum, plasma, urine, CSF, semen, tissue, and feces. The protein can be any protein known in the art or provided herein that is associated with diagnosis and determining status of autoimmune arthritis (e.g., rheumatoid arthritis) or asthma.

In one embodiment, the biomarker used in the methods provided herein is an altered level (e.g., increased or decreased) of a metabolomic, lipid, autoantibody (ACPA), acute phase protein, danger associated molecular patterns (DAMPS), or RF, wherein the said metabolomic, lipid, autoantibody (ACPA), acute phase protein, danger associated molecular patterns (DAMPS), or RF is associated with diagnosis and determining status of autoimmune arthritis (e.g., rheumatoid arthritis) or asthma. In one embodiment, the biomarker used in the methods provided herein is an altered level (e.g., increased or decreased) of CRp, ACPA, Vetrix DA, 14-3-3 protein, or DAMPS.

In one embodiment, provided herein is a method for screening for anti-inflammatory or anti-asthma compound in an animal (e.g., rat) pouch assay. In one embodiment, provided herein is a method for screening for anti-inflammatory or anti-asthma compound comprising (a) creating a pouch at the back of an animal (e.g., rat) and introducing a stimuli into the pouch; (b) administrating a compound to the animal; (c) measuring the influx of leukocyte (e.g., neutrophil and/or eosinophil) into the pouch; and (d) comparing the influx of leukocyte to that of a control vehicle; wherein a reduction in the influx of leukocyte indicates the compound is an anti-inflammatory or anti-asthma compound. In one embodiment, provided herein is a method for screening for PI3K-γ selective inhibitors comprising (a) creating a pouch at the back of an animal (e.g., rat) and introducing a PI3K-γ specific stimuli (e.g., IL-8) into the pouch; (b) administrating a compound to the animal; (c) measuring the influx of leukocyte (e.g., neutrophil and/or eosinophil) into the pouch; and (d) comparing the influx of leukocyte to that of a control vehicle; wherein a reduction in the influx of leukocyte indicates the compound is a PI3Kγ selective inhibitor. In one embodiment, the influx of leukocyte is determined by counting the number of cells present in the pouch.

PI3K-isoform specific activity of a compound can also be determined by other techniques known in the art or provided herein. In one embodiment, PI3K-isoform-specific activity of a compound is determined by the inhibition of a biological signal in cells stimulated by a PI3K-isoform-specific stimulus. In one embodiment, the cells are whole blood cells. In one embodiment, PI3K-δ-specific activity of a compound is determined by the inhibition of degranulation of basophils in cells stimulated by a PI3K-δ-specific stimulus (e.g., anti-FcεR1 antibody). In another embodiment, PI3K-γ-specific activity of a compound is determined by the inhibition of degranulation of basophils in cells stimulated by a PI3K-γ-specific stimulus (e.g., formyl-Methionyl-Leucyl-Phenylalanine (fMLP)). In yet another embodiment, PI3K-β-specific activity of a compound is determined by the inhibition of the activation of GPIIb/IIIa in cells stimulated by a PI3K-β-specific stimulus (e.g., a thrombin peptide stimulus).

### Kits

Kits are also provided herein. The kits include a compound provided herein (e.g., a compound of Formula I (e.g., Compound 292), or an enantiomer or a mixture of enantiomers thereof, or a pharmaceutically acceptable salt, solvate, hydrate, co-crystal, clathrate, or polymorph thereof), or a composition thereof, in suitable packaging, and written material. The written material can include any of the following information: instructions for use, discussion of clinical studies, listing of side effects, scientific literature references, package insert materials, clinical trial results, and/or summaries of these and the like. The written material can indicate or establish the activities and/or advantages of the composition, and/or describe dosing, administration, side effects, drug interactions, or other information useful to the health care provider. Such information can be based on the results of various studies, for example, studies using experimental animals involving in vivo models and/or studies based on human clinical trials. The kit can further contain another therapy (e.g., another agent) and/or written material such as that described above that serves to provide information regarding the other therapy (e.g., the other agent). In some embodiments, the compound provided herein (e.g., a compound of Formula I (e.g., Compound 292), or an enantiomer or a mixture of enantiomers thereof, or a pharmaceutically acceptable salt, solvate, hydrate, co-crystal, clathrate, or polymorph thereof) and the agent are provided as separate compositions in separate containers within the kit. In some embodiments, the compound of the present invention and the agent are provided as a single composition within a container in the kit. Suitable packaging and additional articles for use (e.g., measuring cup for liquid preparations, foil wrapping to minimize exposure to air, and the like) are known in the art and can be included in the kit. Kits described herein can be provided, marketed and/or promoted to health providers, including physicians, nurses, pharmacists, formulary officials, and the like. Kits can also, in some embodiments, be marketed directly to the consumer.

Also provided herein are kits useful for predicting the likelihood of an effective autoimmune arthritis (e.g., rheumatoid arthritis) or asthma treatment or for monitoring the effectiveness of a treatment with a compound provided herein (e.g., a compound of Formula I (e.g., Compound 292), or an enantiomer or a mixture of enantiomers thereof, or a pharmaceutically acceptable salt, solvate, hydrate, co-crystal, clathrate, or polymorph thereof).

In one embodiment, the kit comprises a solid support, and a means for detecting the protein expression of at least one biomarker in a biological sample. Such a kit may employ, for example, a dipstick, a membrane, a chip, a disk, a test strip, a filter, a microsphere, a slide, a multiwell plate, or an optical fiber. The solid support of the kit can be, for example, a plastic, silicon, a metal, a resin, glass, a membrane, a particle, a precipitate, a gel, a polymer, a sheet, a sphere, a polysaccharide, a capillary, a film, a plate, or a slide. The biological sample can be, for example, a cell culture, a cell line, a tissue, an oral tissue, gastrointestinal tissue, an organ, an organelle, a biological fluid, a blood sample, a urine sample, or a skin sample. The biological sample can be, for example, a lymph node biopsy, a bone marrow biopsy, or a sample of peripheral blood tumor cells.

In one embodiment, the kit comprises a solid support, at least one nucleic acid contacting the support, where the nucleic acids are complementary to at least 20, 50, 100, 200, 350, or more bases of mRNA of the biomarker, and a means for detecting the expression of the mRNA in a biological sample.

In certain embodiments, the kits provided herein employ means for detecting the expression of a biomarker by quantitative real-time PCR (QRT-PCR), microarray, flow cytometry or immunofluorescence. In other embodiments, the expression of the biomarker is measured by ELISA-based methodologies or other similar methods known in the art.

### EXAMPLES

### Example 1

### C50 Values for Selected PI3K Inhibitors

The IC₅₀ values for selected compounds were determined and are provided in Table 3. These data demonstrate that these compounds can serve as PI3K δ inhibitors.

TABLE 3
In Vitro IC₅₀ data for selected compounds.

| | + (greater than 10 | ++ (less than 10 | +++ (less than 1 | |
|---|---|---|---|---|
| | microMolar) | microMolar) | microMolar | ++++ (less than 100 nM) |
| IC50 (nM) | Compound No. | Compound No. | Compound No. | Compound No. |
| PI3K δ | 197, 199, 241, 259, | 1, 5, 22, 27, 38, 39, | 4, 14, 15, 17, 18, 21, | 2, 3, 6, 7, 8, 9, 10, 11, |
| | 261, 263, 280, 282, | 40, 41, 46, 92, 117, | 26, 29, 31, 32, 34, 35, | 12, 13, 16, 19, 20, 23, |
| | 283, 314, 315, 318, | 118, 120, 129, 132, | 36, 42, 43, 44, 45, 47, | 24, 25, 28, 30, 33, 37, |
| | 321, 322 | 164, 165, 172, 188, | 49, 57, 69, 71, 85, 87, | 48, 50, 51, 52, 53, 54, |
| | | 186, 193, 194, 195, | 94, 106, 107, 143, | 55, 56, 58, 59, 60, 61, |
| | | 217, 242, 246, 281, | 175, 179, 181, 182, | 62, 63, 64, 65, 66, 67, |
| | | 284, 305, 317, 325 | 183, 187, 189, 192, | 68, 70, 72, 73, 74, 75, |
| | | | 225, 226, 228, 235, | 76, 77, 78, 79, 80, 81, |
| | | | 236, 239, 248, 250, | 82, 83, 84, 86, 88, 89, |
| | | | 258, 269, 274, 275, | 90, 91, 93, 95, 96, 97, |
| | | | 285, 286, 297, 298, | 98, 99, 100, 101, 102, |
| | | | 299, 300, 307, 309, | 103, 104, 105, 108, |
| | | | 313, 319, | 109, 110, 111, 112, |
| | | | | 113, 114, 115, 119, |
| | | | | 123, 124, 125, 126, |
| | | | | 128, 134, 135, 136, |
| | | | | 137, 138, 139, 141, |
| | | | | 142, 144, 145, 146, |
| | | | | 147, 148, 149, 150, |
| | | | | 151. 152, 153, 154, |
| | | | | 155, 156, 157, 158, |
| | | | | 159, 160, 161, 162, |
| | | | | 166, 167, 168, 169, |
| | | | | 170, 171, 173, 174, |
| | | | | 176, 177, 178, 180, |
| | | | | 185, 188, 190, 191, |
| | | | | 196, 198, 200, 201, |
| | | | | 202, 203, 204, 205, |
| | | | | 206, 207, 208, 209, |
| | | | | 210, 211, 212, 213, |
| | | | | 214, 215, 216, 218, |
| | | | | 219, 220, 221, 222, |
| | | | | 223, 224, 227, 229, |
| | | | | 230, 231, 232, 233, |
| | | | | 234, 237, 238, 240, |
| | | | | 243, 244, 245, 247, |
| | | | | 249, 251, 252, 253, |
| | | | | 254, 255, 256, 257, |
| | | | | 260, 262, 264, 265, |
| | | | | 266, 267, 268, 270, |
| | | | | 271, 272, 273, 276, |
| | | | | 277, 278, 279, 287, |
| | | | | 288, 289, 290, 291, |
| | | | | 292, 293, 294, 295, |
| | | | | 296, 301, 302, 303, |
| | | | | 306, 308, 310, 311, |
| | | | | 312, 316, 320, 323, |
| | | | | 324 |
| PI3K γ | 1, 4, 5, 18, 38, 43, 60, | 17, 34, 35, 37, 38, 40, | 2, 8, 9, 10, 11, 14, 15, | 3, 6, 7, 12, 13, 16, 19, |
| | 69, 169, 172, 192, | 42, 57, 61, 65, 91, 92, | 20, 22, 27, 28, 39, 41, | 21, 23, 24, 25, 26, 29, |
| | 193, 194, 199, 227, | 94, 105, 107, 164, | 46, 47, 49, 51, 55, 58, | 30, 31, 33, 36, 44, 45, |
| | 228, 233, 259, 263, | 170, 175, 179, 181, | 66, 70, 71, 73, 76, 78, | 48, 50, 52, 53, 54, 56, |
| | 280, 281, 282, 283, | 183, 184, 186, 187, | 80, 93, 98, 99, 100, | 59, 62, 63, 64, 67, 68, |
| | 314, 315, 317, 318, | 189, 195, 197, 219, | 103, 104, 106, 108, | 72, 74, 75, 77, 79, 81, |
| | 321, 322, 325 | 221, 224, 232, 239, | 109, 161, 162, 163, | 82, 83, 84, 86, 87, 88, |
| | | 241, 242, 246, 248, | 165, 166, 180, 188, | 89, 90, 95, 96, 97, |
| | | 258, 261, 274, 284, | 202, 206, 209, 212, | 101, 102, 142, 145, |
| | | 285, 294, 299, 303, | 214, 216, 218, 220, | 146, 147, 148, 149, |
| | | 305, 307, 309, 312, | 222, 229, 234, 236, | 150, 151, 152, 160, |
| | | 313, 319 | 238, 250, 267, 268, | 167, 168, 171, 173, |
| | | | 269, 271, 275, 279, | 174, 176, 177, 178. |
| | | | 286, 293, 298, 300, | 182, 185, 190, 191, |
| | | | 301, 308, 316 | 196, 198, 200, 201, |
| | | | | 203, 204, 205, 207, |
| | | | | 208, 210, 211, 213, |
| | | | | 215, 223, 230, 231, |
| | | | | 235, 237, 240, 243, |
| | | | | 244, 245, 247, 249, |
| | | | | 251, 252, 253, 254, |
| | | | | 255, 256, 257, 260, |
| | | | | 262, 264, 265, 266, |
| | | | | 270, 272, 273, 276, |
| | | | | 277, 278, 287, 288, |
| | | | | 289, 290, 291, 292, |
| | | | | 295, 296, 302, 304, |
| | | | | 306, 310, 311, 320, |
| | | | | 323, 324 |
| PI3K α | 6, 8, 9, 10, 11, 12, 13, | 3, 7, 63, 66, 84, 86, | 53, 95, 101, 102, 145, | 142, 148, 150, 153, |
| | 14, 15, 16, 17, 18, 19, | 89, 90, 97, 108, 113, | 147, 149, 151, 177, | 154, 155, 156, 157, |
| | 20, 21, 22, 23, 24, 25, | 115, 152, 168, 171, | 208, 257, 260, 262, | 158, 159, 176, 201, |
| | 26, 27, 28, 29, 30, 31, | 173, 185, 190, 198, | 264, 270, 272, 276, | 252 |
| | 32, 33, 34, 35, 36, 37, | 203, 204, 205, 206, | 277, 278, 287, 288, | |
| | 39, 40, 41, 42, 43, 44, | 207, 209, 210, 213, | 289, 320, 323 | |
| | 45, 46, 47, 48, 49, 50, | 223, 235, 237, 240, | | |
| | 51, 52, 54, 55, 56, 57, | 243, 244, 245, 251, | | |
| | 58, 59, 60, 61, 62, 64, | 253, 254, 255, 256, | | |
| | 65, 67, 68, 69, 70, 71, | 269, 273, 279, 291, | | |
| | 72, 73, 74, 79, 80, 81, | 292, 295, 296 | | |
| | 82, 83, 85, 87, 88, 91, | | | |
| | 93, 96, 98, 99, 100, | | | |
| | 103, 104, 105, 106, | | | |
| | 107, 109, 110, 111, | | | |
| | 112, 114, 146, 160, | | | |
| | 161, 162, 163, 164, | | | |
| | 165, 166, 167, 169, | | | |
| | 170, 172, 174, 175, | | | |
| | 179, 180, 181, 182, | | | |
| | 183, 184, 186, 187, | | | |
| | 188, 189, 191, 192, | | | |
| | 193, 194, 197, 202, | | | |
| | 211, 212, 214, 215, | | | |
| | 216, 218, 219, 220, | | | |
| | 221, 222, 224, 227, | | | |
| | 228, 238, 239, 241, | | | |
| | 242, 246, 247, 248, | | | |
| | 249, 250, 258, 259, | | | |
| | 261, 263, 265, 266, | | | |
| | 267, 268, 271, 274, | | | |
| | 275, 280, 281, 282, | | | |
| | 283, 284, 285, 286, | | | |
| | 290, 293, 294, 298, | | | |
| | 299, 300, 304, 308, | | | |
| | 309, 313, 314, 315, | | | |
| | 316, 317, 318, 319, | | | |
| | 321, 322, 324, 325 | | | |
| PI3K β | 8, 9, 10, 11, 14, 21, | 3, 12, 13, 23, 25, 53, | 7, 62, 66, 82, 89, 90, | 101, 142, 155, 156, |
| | 22, 24, 26, 27, 28, 29, | 55, 58, 61, 63, 65, 67, | 95, 97, 100, 102, 150, | 157, 200, 253, 254, |
| | 34, 35, 36, 37, 38, 39, | 71, 72, 74, 75, 77, 81, | 153, 159, 176, 185, | 255, 256, 257, 260, |
| | 40, 41, 42, 43, 44, 46, | 82, 83, 84, 85, 86, 96, | 201, 204, 208, 213, | 262, 264, 268, 270, |
| | 52, 54, 56, 57, 59, 60, | 99, 106, 108, 110, | 227, 237, 251, 252, | 272, 273, 278, 279, |
| | 64, 68, 69, 70, 73, 76, | 111, 113, 114, 115, | 267, 276, 277, 290, | 287, 288, 289, 291, |
| | 78, 79, 80, 87, 88, 91, | 145, 147, 149, 151, | 292, 293 | 320, 323, |
| | 93, 98, 103, 104, 105, | 154, 158, 160, 161, | | |
| | 107, 109, 112, 146, | 167, 168, 171, 173, | | |
| | 152, 162, 163, 164, | 174, 177, 178, 190, | | |
| | 165, 166, 169, 170, | 191, 198, 202, 203, | | |
| | 172, 175, 179, 180, | 205, 206, 207, 209, | | |
| | 181, 182, 183, 184, | 210, 211, 212, 214, | | |
| | 186, 187, 188, 189, | 215, 219, 220, 223, | | |
| | 192, 193, 194, 197, | 228, 235, 240, 243, | | |
| | 216, 217, 218, 221, | 244, 247, 249, 265, | | |
| | 222, 224, 238, 248, | 269, 274, 281, 295, | | |
| | 259, 261, 263, 266, | 296, 298, 300, 308, | | |
| | 271, 275, 280, 282, | 316, 324 | | |
| | 283, 284, 285, 286, | | | |
| | 294, 299, 304, 310, | | | |
| | 311, 312, 315, 317, | | | |
| | 321, 322, 325 | | | |
| B cell proliferation | 38, 162, 199 | 1, 2, 5, 22, 26, 27, 39, | 4, 8, 9, 10, 11, 14, 15, | 3, 6, 7, 12, 13, 16, 17, |
| EC₅₀ (nM) | | 40, 43, 49, 57, 71, 87, | 18, 19, 20, 21, 24, 25, | 23, 33, 37, 44, 48, 53, |
| | | 112, 197, 207, 235 | 28, 29, 30, 31, 32, 34, | 54, 55, 62, 63, 66, 67, |
| | | | 35, 36, 41, 42, 45, 46, | 68, 72, 73, 74, 75, 81, |
| | | | 47, 50, 51, 61, 69, 70, | 82, 83, 84, 88, 89, 90, |
| | | | 76, 77, 78, 79, 80, 85, | 93, 95, 96, 97, 99, |
| | | | 86, 91, 98, 100, 103, | 101, 102, 108, 109, |
| | | | 104, 105, 106, 107, | 113, 115, 123, 125, |
| | | | 110, 111, 114, 119, | 126, 128, 134, 136, |
| | | | 124, 133, 135, 145, | 137, 138, 139, 141, |
| | | | 152, 161, 162, 163, | 142, 144, 146, 147, |
| | | | 169, 195, 212, 243, | 148, 149, 150, 151, |
| | | | 294, 312 | 153, 154, 155, 156, |
| | | | | 157, 158, 159, 160, |
| | | | | 166, 167, 168, 170, |
| | | | | 171, 173, 174, 176, |
| | | | | 177, 178, 180, 187, |
| | | | | 185, 188, 190, 191. |
| | | | | 196, 198, 200, 201, |
| | | | | 202, 203, 204, 205, |
| | | | | 206, 208, 209, 210, |
| | | | | 211, 213, 214, 215, |
| | | | | 216, 219, 220, 221, |
| | | | | 222, 223, 224, 227, |
| | | | | 228, 229, 230, 231, |
| | | | | 232, 233, 234, 237, |
| | | | | 244, 245, 247, 248, |
| | | | | 249, 251, 252, 253, |
| | | | | 254, 255, 256, 257, |
| | | | | 270, 276, 277, 278, |
| | | | | 289, 290, 292, 295, |
| | | | | 296, 298, 300, 301, |
| | | | | 302, 303, 306, 308, |
| | | | | 310, 311 |

TABLE 4
Structures of the Compounds for the IC50 results described in Table 3.
Structure

| | |
|---|---|
| 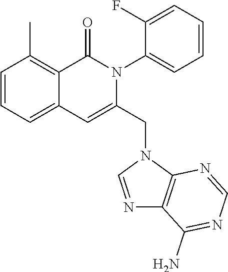 [SMILES: Cc1cccc2cc(Cn3cnc4c(N)ncnc43)n(-c3ccccc3F)c(=O)c12] | Compound 1 |
| 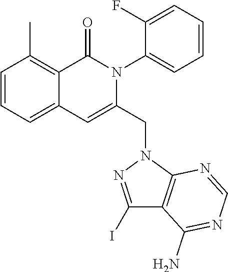 [SMILES: Cc1cccc2cc(Cn3nc(I)c4c(N)ncnc43)n(-c3ccccc3F)c(=O)c12] | Compound 2 |
| 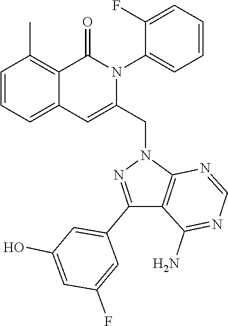 [SMILES: Cc1cccc2cc(Cn3nc(-c4cc(O)cc(F)c4)c4c(N)ncnc43)n(-c3ccccc3F)c(=O)c12] | Compound 3 |
| 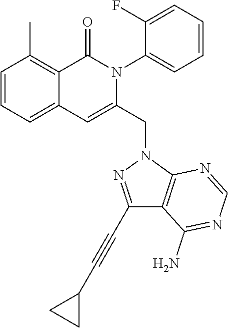 [SMILES: Cc1cccc2cc(Cn3nc(C#CC4CC4)c4c(N)ncnc43)n(-c3ccccc3F)c(=O)c12] | Compound 4 |
| 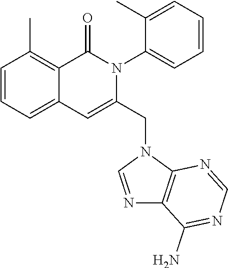 [SMILES: Cc1ccccc1-n1c(Cn2cnc3c(N)ncnc32)cc2cccc(C)c2c1=O] | Compound 5 |
| 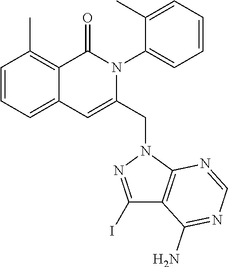 [SMILES: Cc1ccccc1-n1c(Cn2nc(I)c3c(N)ncnc32)cc2cccc(C)c2c1=O] | Compound 6 |
| 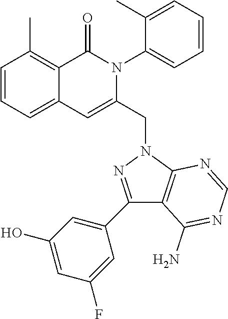 [SMILES: Cc1ccccc1-n1c(Cn2nc(-c3cc(O)cc(F)c3)c3c(N)ncnc32)cc2cccc(C)c2c1=O] | Compound 7 |
| 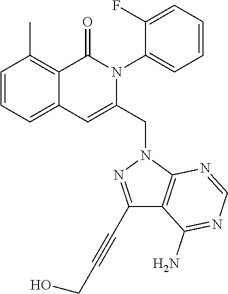 [SMILES: Cc1cccc2cc(Cn3nc(C#CCO)c4c(N)ncnc43)n(-c3ccccc3F)c(=O)c12] | Compound 8 |
| 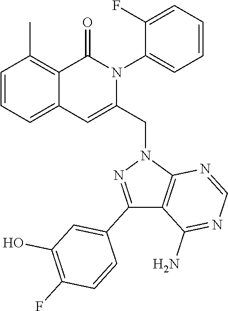 [SMILES: Cc1cccc2cc(Cn3nc(-c4ccc(F)c(O)c4)c4c(N)ncnc43)n(-c3ccccc3F)c(=O)c12] | Compound 9 |
| 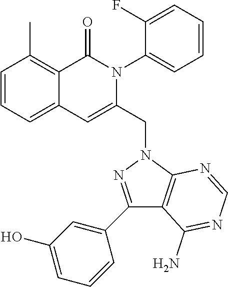 [SMILES: Cc1cccc2cc(Cn3nc(-c4cccc(O)c4)c4c(N)ncnc43)n(-c3ccccc3F)c(=O)c12] | Compound 10 |
| 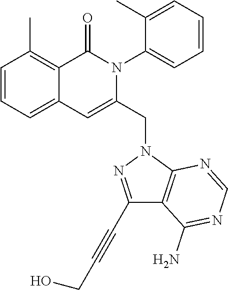 [SMILES: Cc1ccccc1-n1c(Cn2nc(C#CCO)c3c(N)ncnc32)cc2cccc(C)c2c1=O] | Compound 11 |
| 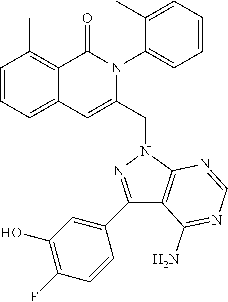 [SMILES: Cc1ccccc1-n1c(Cn2nc(-c3ccc(F)c(O)c3)c3c(N)ncnc32)cc2cccc(C)c2c1=O] | Compound 12 |
| 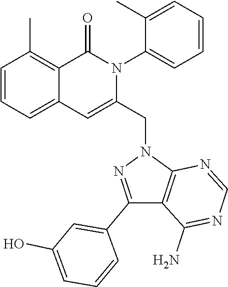 [SMILES: Cc1ccccc1-n1c(Cn2nc(-c3cccc(O)c3)c3c(N)ncnc32)cc2cccc(C)c2c1=O] | Compound 13 |
| 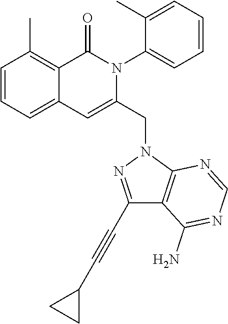 [SMILES: Cc1ccccc1-n1c(Cn2nc(C#CC3CC3)c3c(N)ncnc32)cc2cccc(C)c2c1=O] | Compound 14 |
| 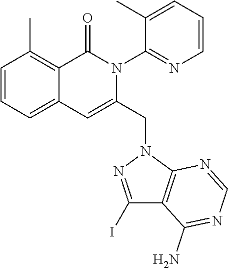 [SMILES: Cc1cccnc1-n1c(Cn2nc(I)c3c(N)ncnc32)cc2cccc(C)c2c1=O] | Compound 15 |
| 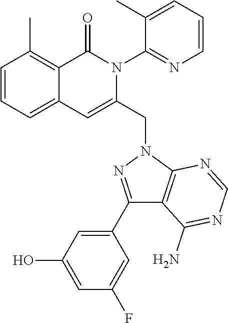 [SMILES: Cc1cccnc1-n1c(Cn2nc(-c3cc(O)cc(F)c3)c3c(N)ncnc32)cc2cccc(C)c2c1=O] | Compound 16 |
| 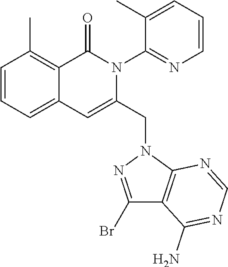 [SMILES: Cc1cccnc1-n1c(Cn2nc(Br)c3c(N)ncnc32)cc2cccc(C)c2c1=O] | Compound 17 |
| 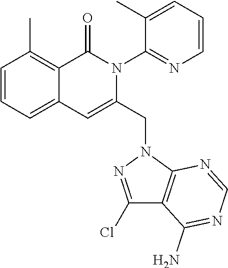 [SMILES: Cc1cccnc1-n1c(Cn2nc(Cl)c3c(N)ncnc32)cc2cccc(C)c2c1=O] | Compound 18 |
| 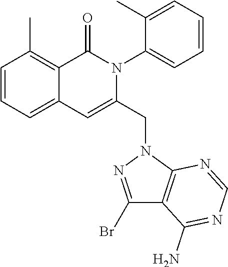 [SMILES: Cc1ccccc1-n1c(Cn2nc(Br)c3c(N)ncnc32)cc2cccc(C)c2c1=O] | Compound 19 |
| 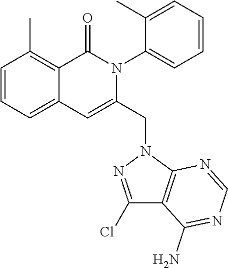 [SMILES: Cc1ccccc1-n1c(Cn2nc(Cl)c3c(N)ncnc32)cc2cccc(C)c2c1=O] | Compound 20 |
| 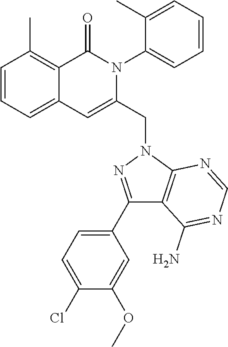 [SMILES: COc1cc(-c2nn(Cc3cc4cccc(C)c4c(=O)n3-c3ccccc3C)c3ncnc(N)c23)ccc1Cl] | Compound 21 |
| 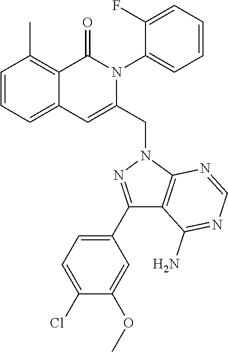 [SMILES: COc1cc(-c2nn(Cc3cc4cccc(C)c4c(=O)n3-c3ccccc3F)c3ncnc(N)c23)ccc1Cl] | Compound 22 |
| 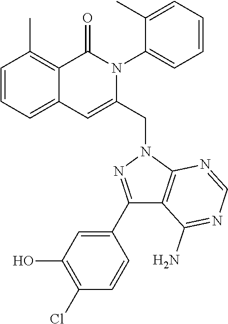 [SMILES: Cc1ccccc1-n1c(Cn2nc(-c3ccc(Cl)c(O)c3)c3c(N)ncnc32)cc2cccc(C)c2c1=O] | Compound 23 |
| 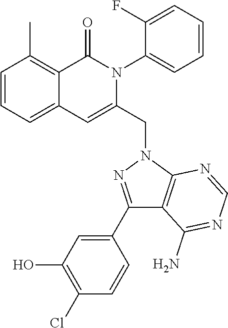 [SMILES: Cc1cccc2cc(Cn3nc(-c4ccc(Cl)c(O)c4)c4c(N)ncnc43)n(-c3ccccc3F)c(=O)c12] | Compound 24 |
| 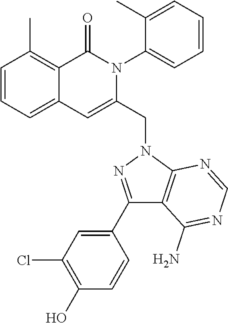 [SMILES: Cc1ccccc1-n1c(Cn2nc(-c3ccc(O)c(Cl)c3)c3c(N)ncnc32)cc2cccc(C)c2c1=O] | Compound 25 |
| 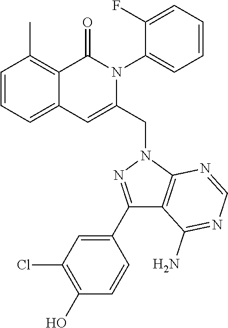 [SMILES: Cc1cccc2cc(Cn3nc(-c4ccc(O)c(Cl)c4)c4c(N)ncnc43)n(-c3ccccc3F)c(=O)c12] | Compound 26 |
| 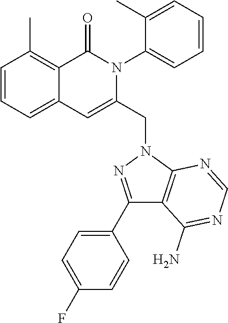 [SMILES: Cc1ccccc1-n1c(Cn2nc(-c3ccc(F)cc3)c3c(N)ncnc32)cc2cccc(C)c2c1=O] | Compound 27 |
| 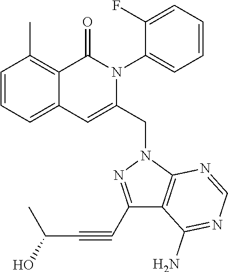 [SMILES: Cc1cccc2cc(Cn3nc(C#C[C@@H](C)O)c4c(N)ncnc43)n(-c3ccccc3F)c(=O)c12] | Compound 28 |
| 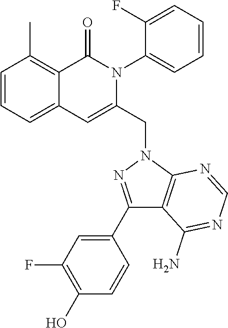 [SMILES: Cc1cccc2cc(Cn3nc(-c4ccc(O)c(F)c4)c4c(N)ncnc43)n(-c3ccccc3F)c(=O)c12] | Compound 29 |
| 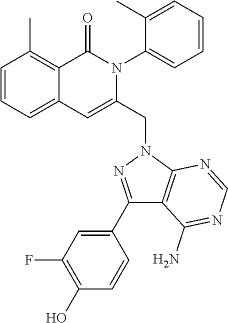 [SMILES: Cc1ccccc1-n1c(Cn2nc(-c3ccc(O)c(F)c3)c3c(N)ncnc32)cc2cccc(C)c2c1=O] | Compound 30 |
| 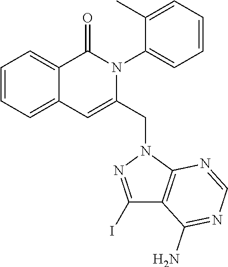 [SMILES: Cc1ccccc1-n1c(Cn2nc(I)c3c(N)ncnc32)cc2ccccc2c1=O] | Compound 31 |
| 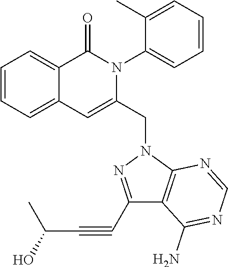 [SMILES: Cc1ccccc1-n1c(Cn2nc(C#C[C@@H](C)O)c3c(N)ncnc32)cc2ccccc2c1=O] | Compound 32 |
| 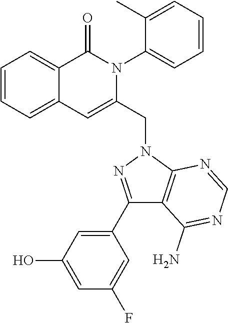 [SMILES: Cc1ccccc1-n1c(Cn2nc(-c3cc(O)cc(F)c3)c3c(N)ncnc32)cc2ccccc2c1=O] | Compound 33 |
| 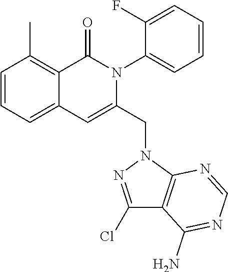 [SMILES: Cc1cccc2cc(Cn3nc(Cl)c4c(N)ncnc43)n(-c3ccccc3F)c(=O)c12] | Compound 34 |
| 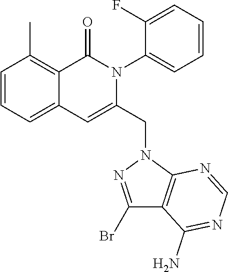 [SMILES: Cc1cccc2cc(Cn3nc(Br)c4c(N)ncnc43)n(-c3ccccc3F)c(=O)c12] | Compound 35 |
| 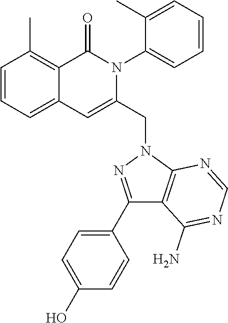 [SMILES: Cc1ccccc1-n1c(Cn2nc(-c3ccc(O)cc3)c3c(N)ncnc32)cc2cccc(C)c2c1=O] | Compound 36 |
| 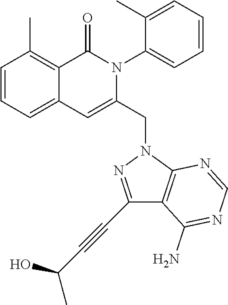 [SMILES: Cc1ccccc1-n1c(Cn2nc(C#C[C@@H](C)O)c3c(N)ncnc32)cc2cccc(C)c2c1=O] | Compound 37 |
| 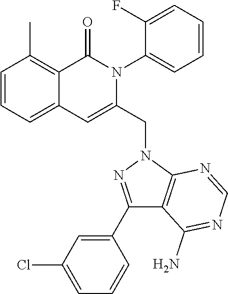 [SMILES: Cc1cccc2cc(Cn3nc(-c4cccc(Cl)c4)c4c(N)ncnc43)n(-c3ccccc3F)c(=O)c12] | Compound 38 |
| 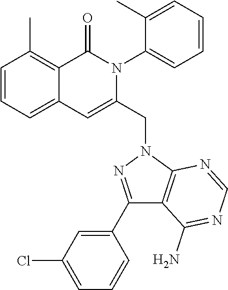 [SMILES: Cc1ccccc1-n1c(Cn2nc(-c3cccc(Cl)c3)c3c(N)ncnc32)cc2cccc(C)c2c1=O] | Compound 39 |
| 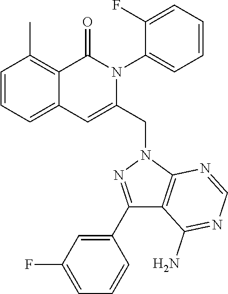 [SMILES: Cc1cccc2cc(Cn3nc(-c4cccc(F)c4)c4c(N)ncnc43)n(-c3ccccc3F)c(=O)c12] | Compound 40 |
| 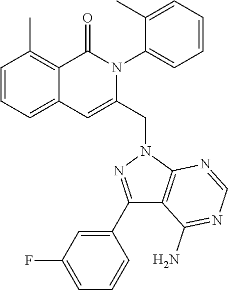 [SMILES: Cc1ccccc1-n1c(Cn2nc(-c3cccc(F)c3)c3c(N)ncnc32)cc2cccc(C)c2c1=O] | Compound 41 |
| 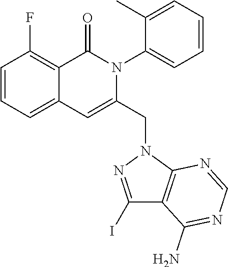 [SMILES: Cc1ccccc1-n1c(Cn2nc(I)c3c(N)ncnc32)cc2cccc(F)c2c1=O] | Compound 42 |
| 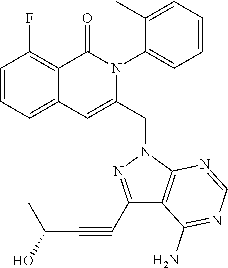 [SMILES: Cc1ccccc1-n1c(Cn2nc(C#C[C@@H](C)O)c3c(N)ncnc32)cc2cccc(F)c2c1=O] | Compound 43 |
| 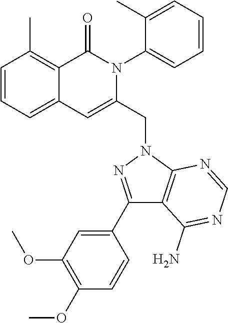 [SMILES: COc1ccc(-c2nn(Cc3cc4cccc(C)c4c(=O)n3-c3ccccc3C)c3ncnc(N)c23)cc1OC] | Compound 44 |
| 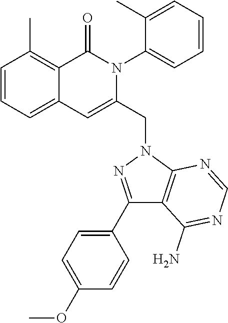 [SMILES: COc1ccc(-c2nn(Cc3cc4cccc(C)c4c(=O)n3-c3ccccc3C)c3ncnc(N)c23)cc1] | Compound 45 |
| 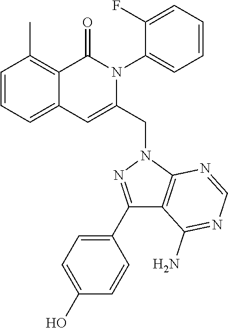 [SMILES: Cc1cccc2cc(Cn3nc(-c4ccc(O)cc4)c4c(N)ncnc43)n(-c3ccccc3F)c(=O)c12] | Compound 46 |
| 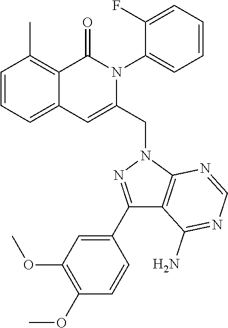 [SMILES: COc1ccc(-c2nn(Cc3cc4cccc(C)c4c(=O)n3-c3ccccc3F)c3ncnc(N)c23)cc1OC] | Compound 47 |
| 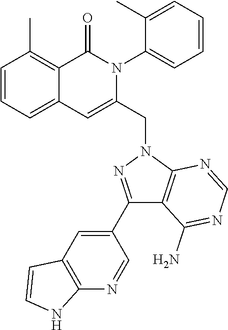 [SMILES: Cc1ccccc1-n1c(Cn2nc(-c3cnc4[nH]ccc4c3)c3c(N)ncnc32)cc2cccc(C)c2c1=O] | Compound 48 |
| 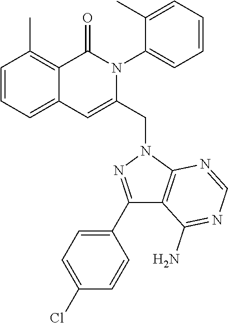 [SMILES: Cc1ccccc1-n1c(Cn2nc(-c3ccc(Cl)cc3)c3c(N)ncnc32)cc2cccc(C)c2c1=O] | Compound 49 |
| 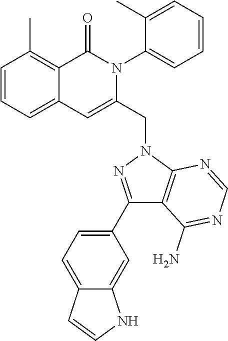 [SMILES: Cc1ccccc1-n1c(Cn2nc(-c3ccc4cc[nH]c4c3)c3c(N)ncnc32)cc2cccc(C)c2c1=O] | Compound 50 |
| 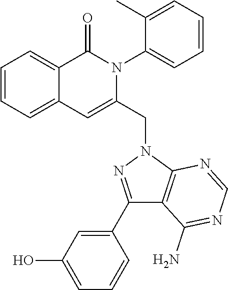 [SMILES: Cc1ccccc1-n1c(Cn2nc(-c3cccc(O)c3)c3c(N)ncnc32)cc2ccccc2c1=O] | Compound 51 |
| 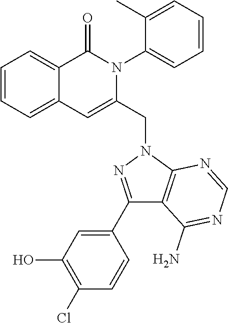 [SMILES: Cc1ccccc1-n1c(Cn2nc(-c3ccc(Cl)c(O)c3)c3c(N)ncnc32)cc2ccccc2c1=O] | Compound 52 |
| 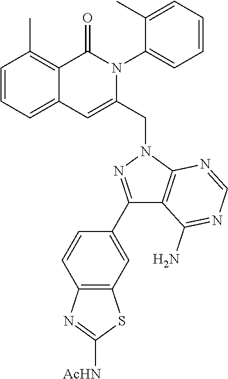 [SMILES: CC(=O)Nc1nc2ccc(-c3nn(Cc4cc5cccc(C)c5c(=O)n4-c4ccccc4C)c4ncnc(N)c34)cc2s1] | Compound 53 |
| 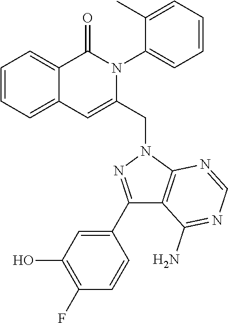 [SMILES: Cc1ccccc1-n1c(Cn2nc(-c3ccc(F)c(O)c3)c3c(N)ncnc32)cc2ccccc2c1=O] | Compound 54 |
| 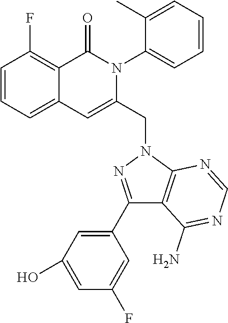 [SMILES: Cc1ccccc1-n1c(Cn2nc(-c3cc(O)cc(F)c3)c3c(N)ncnc32)cc2cccc(F)c2c1=O] | Compound 55 |
| 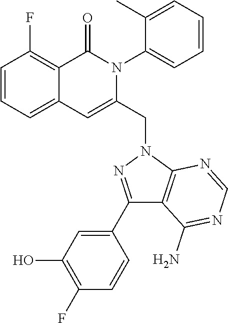 [SMILES: Cc1ccccc1-n1c(Cn2nc(-c3ccc(F)c(O)c3)c3c(N)ncnc32)cc2cccc(F)c2c1=O] | Compound 56 |
| 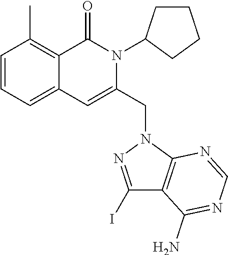 [SMILES: Cc1cccc2cc(Cn3nc(I)c4c(N)ncnc43)n(C3CCCC3)c(=O)c12] | Compound 57 |
| 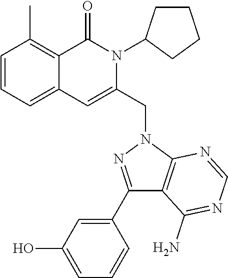 [SMILES: Cc1cccc2cc(Cn3nc(-c4cccc(O)c4)c4c(N)ncnc43)n(C3CCCC3)c(=O)c12] | Compound 58 |
| 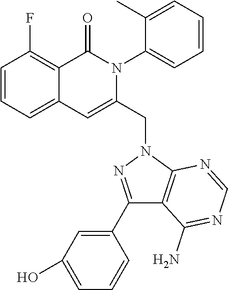 [SMILES: Cc1ccccc1-n1c(Cn2nc(-c3cccc(O)c3)c3c(N)ncnc32)cc2cccc(F)c2c1=O] | Compound 59 |
| 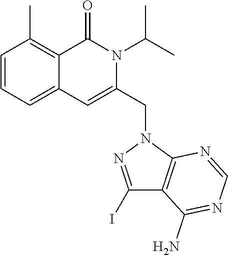 [SMILES: Cc1cccc2cc(Cn3nc(I)c4c(N)ncnc43)n(C(C)C)c(=O)c12] | Compound 60 |
| 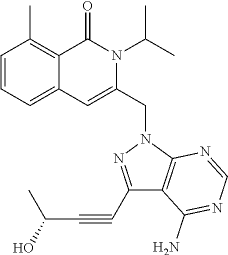 [SMILES: Cc1cccc2cc(Cn3nc(C#C[C@@H](C)O)c4c(N)ncnc43)n(C(C)C)c(=O)c12] | Compound 61 |
| 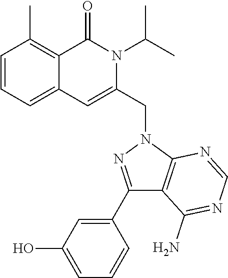 [SMILES: Cc1cccc2cc(Cn3nc(-c4cccc(O)c4)c4c(N)ncnc43)n(C(C)C)c(=O)c12] | Compound 62 |
| 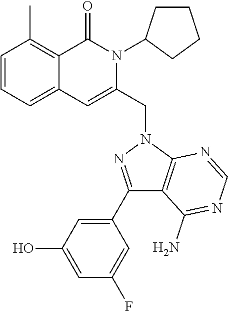 [SMILES: Cc1cccc2cc(Cn3nc(-c4cc(O)cc(F)c4)c4c(N)ncnc43)n(C3CCCC3)c(=O)c12] | Compound 63 |
| 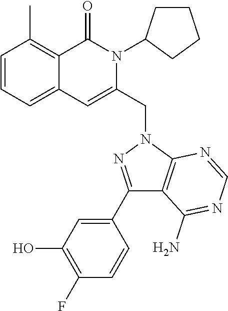 [SMILES: Cc1cccc2cc(Cn3nc(-c4ccc(F)c(O)c4)c4c(N)ncnc43)n(C3CCCC3)c(=O)c12] | Compound 64 |
| 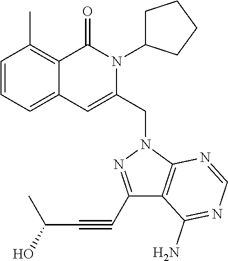 [SMILES: Cc1cccc2cc(Cn3nc(C#C[C@@H](C)O)c4c(N)ncnc43)n(C3CCCC3)c(=O)c12] | Compound 65 |
| 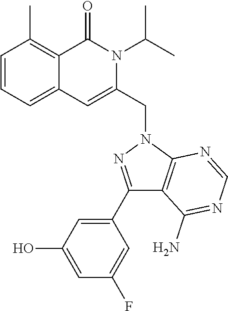 [SMILES: Cc1cccc2cc(Cn3nc(-c4cc(O)cc(F)c4)c4c(N)ncnc43)n(C(C)C)c(=O)c12] | Compound 66 |
| 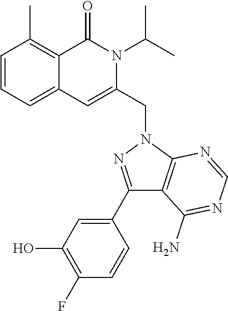 [SMILES: Cc1cccc2cc(Cn3nc(-c4ccc(F)c(O)c4)c4c(N)ncnc43)n(C(C)C)c(=O)c12] | Compound 67 |
| 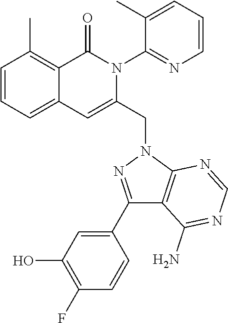 [SMILES: Cc1cccnc1-n1c(Cn2nc(-c3ccc(F)c(O)c3)c3c(N)ncnc32)cc2cccc(C)c2c1=O] | Compound 68 |
| 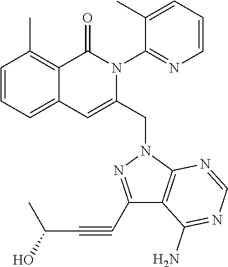 [SMILES: Cc1cccnc1-n1c(Cn2nc(C#C[C@@H](C)O)c3c(N)ncnc32)cc2cccc(C)c2c1=O] | Compound 69 |
| 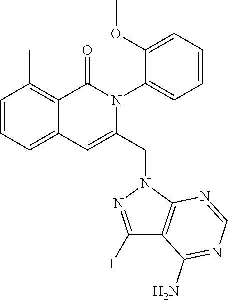 [SMILES: COc1ccccc1-n1c(Cn2nc(I)c3c(N)ncnc32)cc2cccc(C)c2c1=O] | Compound 70 |
| 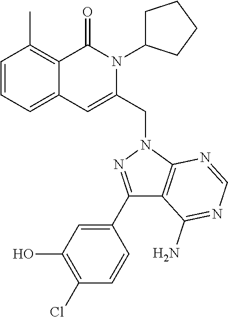 [SMILES: Cc1cccc2cc(Cn3nc(-c4ccc(Cl)c(O)c4)c4c(N)ncnc43)n(C3CCCC3)c(=O)c12] | Compound 71 |
| 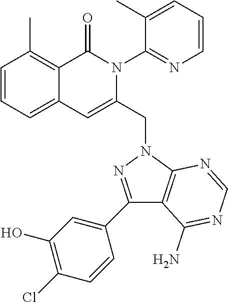 [SMILES: Cc1cccnc1-n1c(Cn2nc(-c3ccc(Cl)c(O)c3)c3c(N)ncnc32)cc2cccc(C)c2c1=O] | Compound 72 |
| 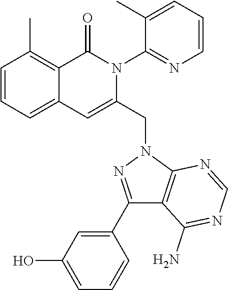 [SMILES: Cc1cccnc1-n1c(Cn2nc(-c3cccc(O)c3)c3c(N)ncnc32)cc2cccc(C)c2c1=O] | Compound 73 |
| 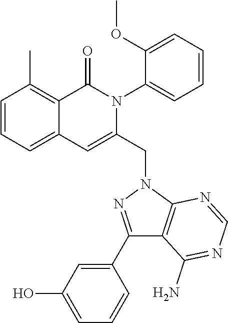 [SMILES: COc1ccccc1-n1c(Cn2nc(-c3cccc(O)c3)c3c(N)ncnc32)cc2cccc(C)c2c1=O] | Compound 74 |
| 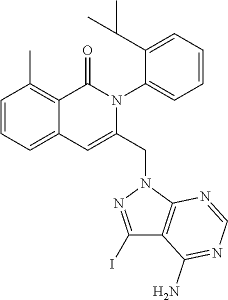 [SMILES: Cc1cccc2cc(Cn3nc(I)c4c(N)ncnc43)n(-c3ccccc3C(C)C)c(=O)c12] | Compound 75 |
| 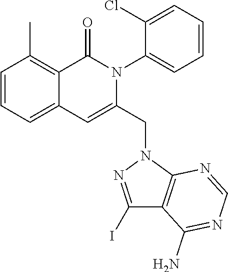 [SMILES: Cc1cccc2cc(Cn3nc(I)c4c(N)ncnc43)n(-c3ccccc3Cl)c(=O)c12] | Compound 76 |
| 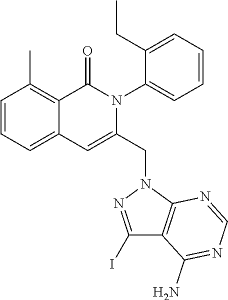 [SMILES: CCc1ccccc1-n1c(Cn2nc(I)c3c(N)ncnc32)cc2cccc(C)c2c1=O] | Compound 77 |
| 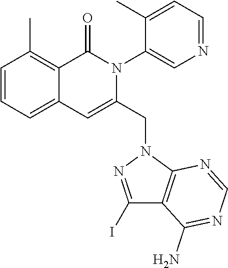 [SMILES: Cc1ccncc1-n1c(Cn2nc(I)c3c(N)ncnc32)cc2cccc(C)c2c1=O] | Compound 78 |
| 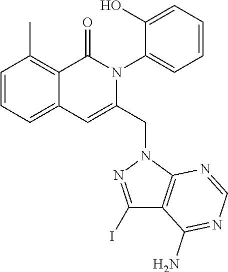 [SMILES: Cc1cccc2cc(Cn3nc(I)c4c(N)ncnc43)n(-c3ccccc3O)c(=O)c12] | Compound 79 |
| 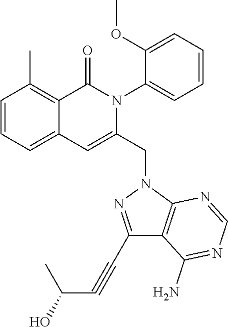 [SMILES: COc1ccccc1-n1c(Cn2nc(C#C[C@@H](C)O)c3c(N)ncnc32)cc2cccc(C)c2c1=O] | Compound 80 |
| 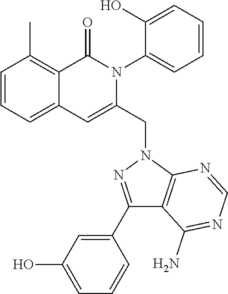 [SMILES: Cc1cccc2cc(Cn3nc(-c4cccc(O)c4)c4c(N)ncnc43)n(-c3ccccc3O)c(=O)c12] | Compound 81 |
| 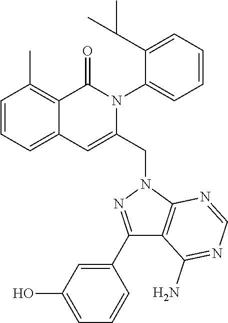 [SMILES: Cc1cccc2cc(Cn3nc(-c4cccc(O)c4)c4c(N)ncnc43)n(-c3ccccc3C(C)C)c(=O)c12] | Compound 82 |
| 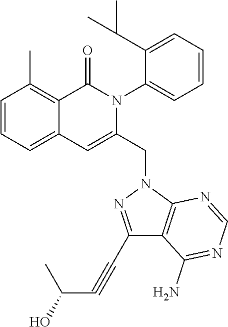 [SMILES: Cc1cccc2cc(Cn3nc(C#C[C@@H](C)O)c4c(N)ncnc43)n(-c3ccccc3C(C)C)c(=O)c12] | Compound 83 |
| 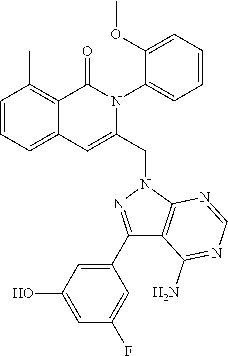 [SMILES: COc1ccccc1-n1c(Cn2nc(-c3cc(O)cc(F)c3)c3c(N)ncnc32)cc2cccc(C)c2c1=O] | Compound 84 |
| 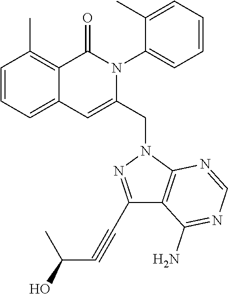 [SMILES: Cc1ccccc1-n1c(Cn2nc(C#C[C@H](C)O)c3c(N)ncnc32)cc2cccc(C)c2c1=O] | Compound 85 |
| 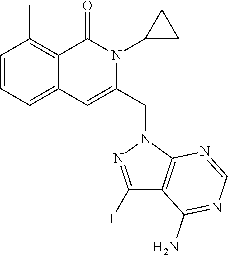 [SMILES: Cc1cccc2cc(Cn3nc(I)c4c(N)ncnc43)n(C3CC3)c(=O)c12] | Compound 86 |
| 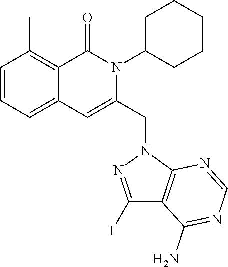 [SMILES: Cc1cccc2cc(Cn3nc(I)c4c(N)ncnc43)n(C3CCCCC3)c(=O)c12] | Comopund 87 |
| 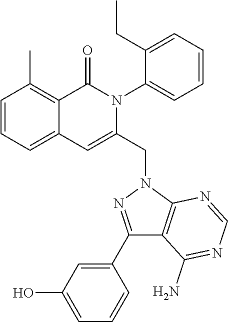 [SMILES: CCc1ccccc1-n1c(Cn2nc(-c3cccc(O)c3)c3c(N)ncnc32)cc2cccc(C)c2c1=O] | Compound 88 |
| 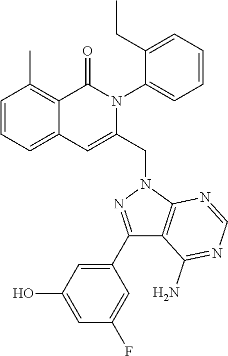 [SMILES: CCc1ccccc1-n1c(Cn2nc(-c3cc(O)cc(F)c3)c3c(N)ncnc32)cc2cccc(C)c2c1=O] | Compound 89 |
| 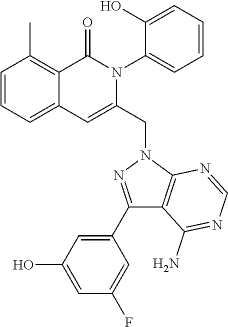 [SMILES: Cc1cccc2cc(Cn3nc(-c4cc(O)cc(F)c4)c4c(N)ncnc43)n(-c3ccccc3O)c(=O)c12] | Compound 90 |
| 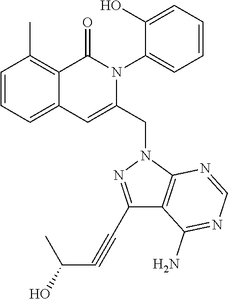 [SMILES: Cc1cccc2cc(Cn3nc(C#C[C@@H](C)O)c4c(N)ncnc43)n(-c3ccccc3O)c(=O)c12] | Compound 91 |
| 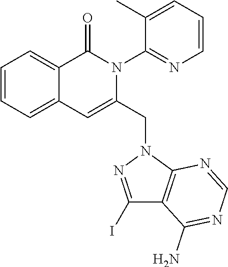 [SMILES: Cc1cccnc1-n1c(Cn2nc(I)c3c(N)ncnc32)cc2ccccc2c1=O] | Compound 92 |
| 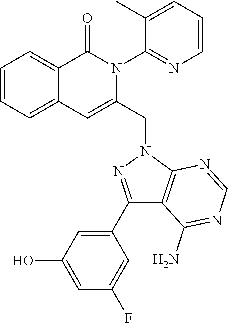 [SMILES: Cc1cccnc1-n1c(Cn2nc(-c3cc(O)cc(F)c3)c3c(N)ncnc32)cc2ccccc2c1=O] | Compound 93 |
| 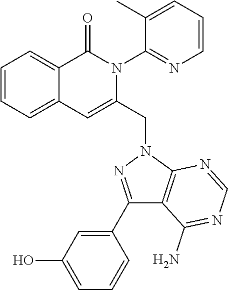 [SMILES: Cc1cccnc1-n1c(Cn2nc(-c3cccc(O)c3)c3c(N)ncnc32)cc2ccccc2c1=O] | Compound 94 |
| 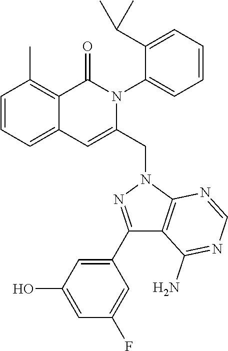 [SMILES: Cc1cccc2cc(Cn3nc(-c4cc(O)cc(F)c4)c4c(N)ncnc43)n(-c3ccccc3C(C)C)c(=O)c12] | Compound 95 |
| 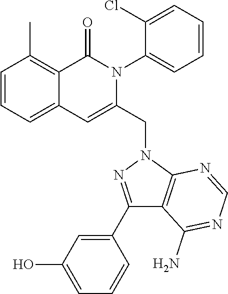 [SMILES: Cc1cccc2cc(Cn3nc(-c4cccc(O)c4)c4c(N)ncnc43)n(-c3ccccc3Cl)c(=O)c12] | Compound 96 |
| 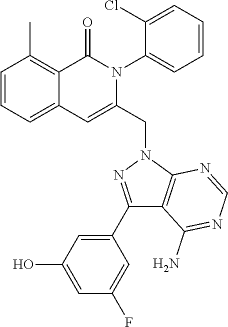 [SMILES: Cc1cccc2cc(Cn3nc(-c4cc(O)cc(F)c4)c4c(N)ncnc43)n(-c3ccccc3Cl)c(=O)c12] | Compound 97 |
| 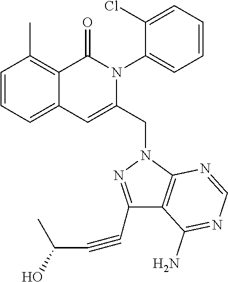 [SMILES: Cc1cccc2cc(Cn3nc(C#C[C@@H](C)O)c4c(N)ncnc43)n(-c3ccccc3Cl)c(=O)c12] | Compound 98 |
| 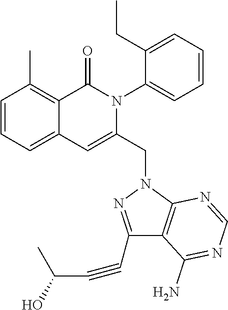 [SMILES: CCc1ccccc1-n1c(Cn2nc(C#C[C@@H](C)O)c3c(N)ncnc32)cc2cccc(C)c2c1=O] | Compound 99 |
| 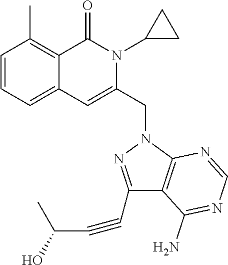 [SMILES: Cc1cccc2cc(Cn3nc(C#C[C@@H](C)O)c4c(N)ncnc43)n(C3CC3)c(=O)c12] | Compound 100 |
| 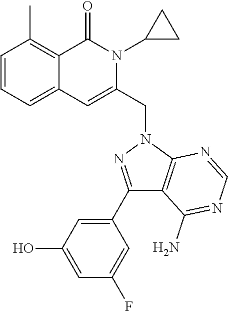 [SMILES: Cc1cccc2cc(Cn3nc(-c4cc(O)cc(F)c4)c4c(N)ncnc43)n(C3CC3)c(=O)c12] | Compound 101 |
| 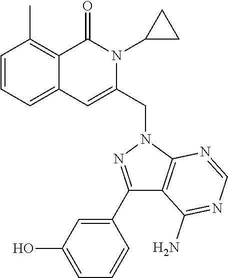 [SMILES: Cc1cccc2cc(Cn3nc(-c4cccc(O)c4)c4c(N)ncnc43)n(C3CC3)c(=O)c12] | Compound 102 |
| 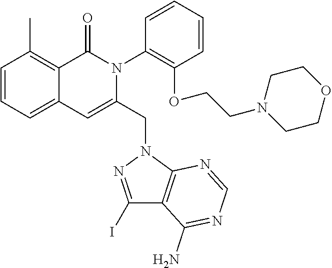 [SMILES: Cc1cccc2cc(Cn3nc(I)c4c(N)ncnc43)n(-c3ccccc3OCCN3CCOCC3)c(=O)c12] | Compound 103 |
| 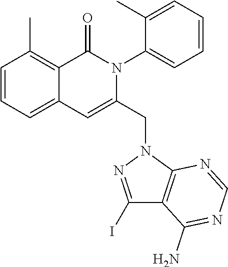 [SMILES: Cc1ccccc1-n1c(Cn2nc(I)c3c(N)ncnc32)cc2cccc(C)c2c1=O] | Compound 104 |
| 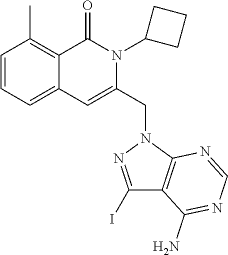 [SMILES: Cc1cccc2cc(Cn3nc(I)c4c(N)ncnc43)n(C3CCC3)c(=O)c12] | Compound 105 |
| 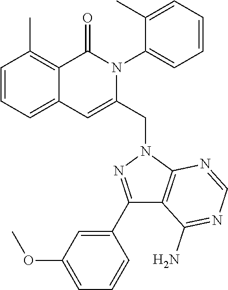 [SMILES: COc1cccc(-c2nn(Cc3cc4cccc(C)c4c(=O)n3-c3ccccc3C)c3ncnc(N)c23)c1] | Compound 106 |
| 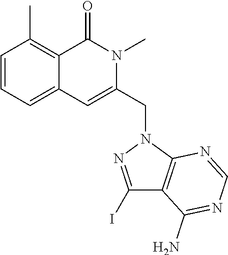 [SMILES: Cc1cccc2cc(Cn3nc(I)c4c(N)ncnc43)n(C)c(=O)c12] | Compound 107 |
| 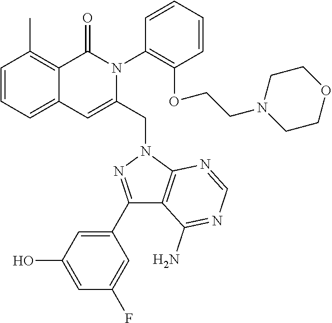 [SMILES: Cc1cccc2cc(Cn3nc(-c4cc(O)cc(F)c4)c4c(N)ncnc43)n(-c3ccccc3OCCN3CCOCC3)c(=O)c12] | Compound 108 |
| 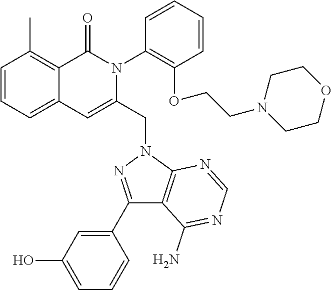 [SMILES: Cc1cccc2cc(Cn3nc(-c4cccc(O)c4)c4c(N)ncnc43)n(-c3ccccc3OCCN3CCOCC3)c(=O)c12] | Compound 109 |
| 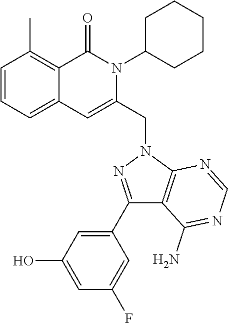 [SMILES: Cc1cccc2cc(Cn3nc(-c4cc(O)cc(F)c4)c4c(N)ncnc43)n(C3CCCCC3)c(=O)c12] | Compound 110 |
| 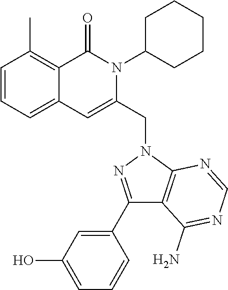 [SMILES: Cc1cccc2cc(Cn3nc(-c4cccc(O)c4)c4c(N)ncnc43)n(C3CCCCC3)c(=O)c12] | Compound 111 |
| 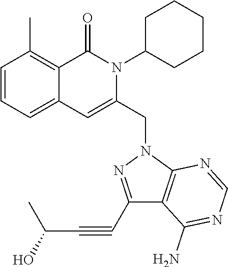 [SMILES: Cc1cccc2cc(Cn3nc(C#C[C@@H](C)O)c4c(N)ncnc43)n(C3CCCCC3)c(=O)c12] | Compound 112 |
| 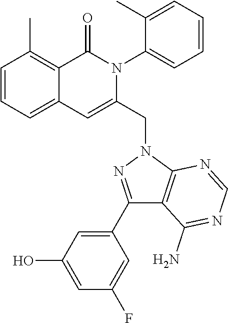 [SMILES: Cc1ccccc1-n1c(Cn2nc(-c3cc(O)cc(F)c3)c3c(N)ncnc32)cc2cccc(C)c2c1=O] | Compound 113 |
| 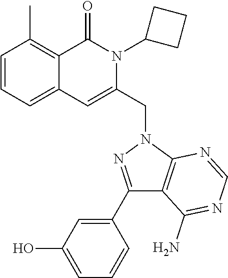 [SMILES: Cc1cccc2cc(Cn3nc(-c4cccc(O)c4)c4c(N)ncnc43)n(C3CCC3)c(=O)c12] | Compound 114 |
| 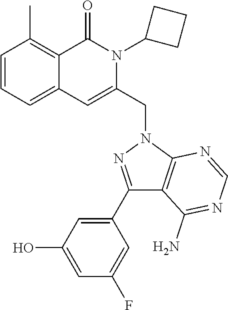 [SMILES: Cc1cccc2cc(Cn3nc(-c4cc(O)cc(F)c4)c4c(N)ncnc43)n(C3CCC3)c(=O)c12] | Compound 115 |
| 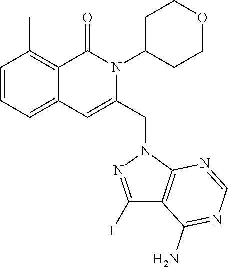 [SMILES: Cc1cccc2cc(Cn3nc(I)c4c(N)ncnc43)n(C3CCOCC3)c(=O)c12] | Compound 116 |
| 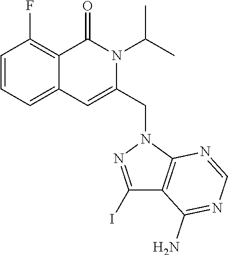 [SMILES: CC(C)n1c(Cn2nc(I)c3c(N)ncnc32)cc2cccc(F)c2c1=O] | Compound 117 |
| 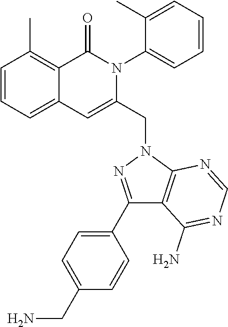 [SMILES: Cc1ccccc1-n1c(Cn2nc(-c3ccc(CN)cc3)c3c(N)ncnc32)cc2cccc(C)c2c1=O] | Compound 118 |
| 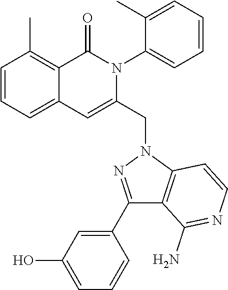 [SMILES: Cc1ccccc1-n1c(Cn2nc(-c3cccc(O)c3)c3c(N)nccc32)cc2cccc(C)c2c1=O] | Compound 119 |
| 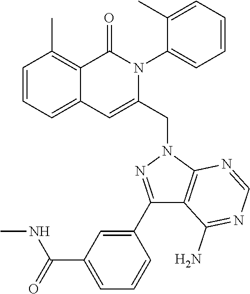 [SMILES: CNC(=O)c1cccc(-c2nn(Cc3cc4cccc(C)c4c(=O)n3-c3ccccc3C)c3ncnc(N)c23)c1] | Compound 120 |
| 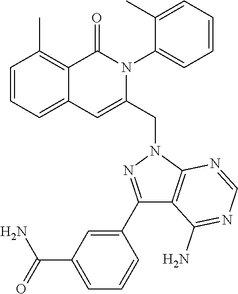 [SMILES: Cc1ccccc1-n1c(Cn2nc(-c3cccc(C(N)=O)c3)c3c(N)ncnc32)cc2cccc(C)c2c1=O] | Compound 121 |
| 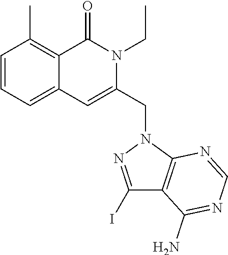 [SMILES: CCn1c(Cn2nc(I)c3c(N)ncnc32)cc2cccc(C)c2c1=O] | Compound 122 |
| 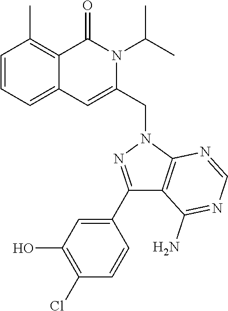 [SMILES: Cc1cccc2cc(Cn3nc(-c4ccc(Cl)c(O)c4)c4c(N)ncnc43)n(C(C)C)c(=O)c12] | Compound 123 |
| 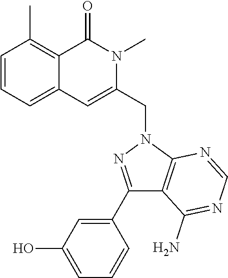 [SMILES: Cc1cccc2cc(Cn3nc(-c4cccc(O)c4)c4c(N)ncnc43)n(C)c(=O)c12] | Compound 124 |
| 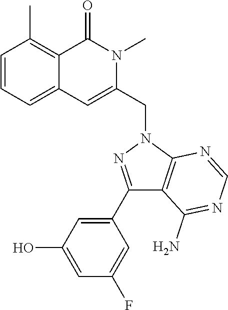 [SMILES: Cc1cccc2cc(Cn3nc(-c4cc(O)cc(F)c4)c4c(N)ncnc43)n(C)c(=O)c12] | Compound 125 |
| 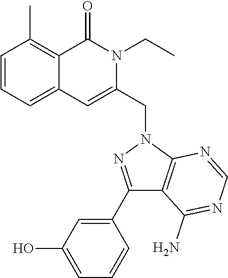 [SMILES: CCn1c(Cn2nc(-c3cccc(O)c3)c3c(N)ncnc32)cc2cccc(C)c2c1=O] | Compound 126 |
| 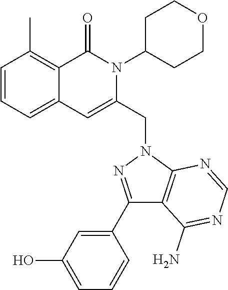 [SMILES: Cc1cccc2cc(Cn3nc(-c4cccc(O)c4)c4c(N)ncnc43)n(C3CCOCC3)c(=O)c12] | Compound 127 |
| 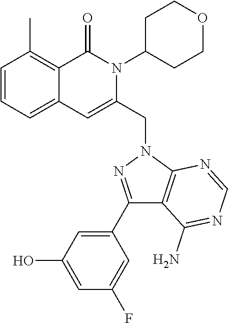 [SMILES: Cc1cccc2cc(Cn3nc(-c4cc(O)cc(F)c4)c4c(N)ncnc43)n(C3CCOCC3)c(=O)c12] | Compound 128 |
| 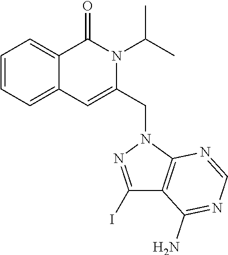 [SMILES: CC(C)n1c(Cn2nc(I)c3c(N)ncnc32)cc2ccccc2c1=O] | Compound 129 |
| 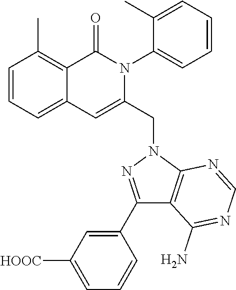 [SMILES: Cc1ccccc1-n1c(Cn2nc(-c3cccc(C(=O)O)c3)c3c(N)ncnc32)cc2cccc(C)c2c1=O] | Compound 130 |
| 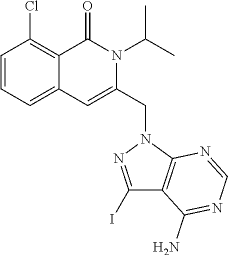 [SMILES: CC(C)n1c(Cn2nc(I)c3c(N)ncnc32)cc2cccc(Cl)c2c1=O] | Compound 131 |
| 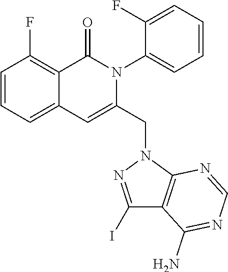 [SMILES: Nc1ncnc2c1c(I)nn2Cc1cc2cccc(F)c2c(=O)n1-c1ccccc1F] | Compound 132 |
| 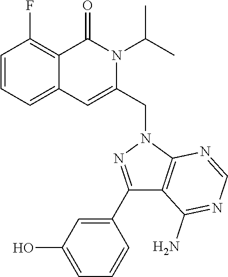 [SMILES: CC(C)n1c(Cn2nc(-c3cccc(O)c3)c3c(N)ncnc32)cc2cccc(F)c2c1=O] | Compound 133 |
| 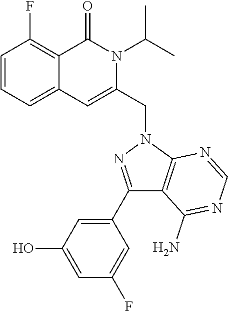 [SMILES: CC(C)n1c(Cn2nc(-c3cc(O)cc(F)c3)c3c(N)ncnc32)cc2cccc(F)c2c1=O] | Compound 134 |
| 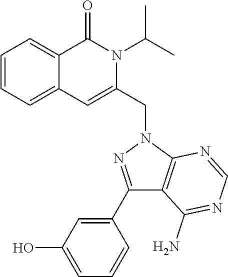 [SMILES: CC(C)n1c(Cn2nc(-c3cccc(O)c3)c3c(N)ncnc32)cc2ccccc2c1=O] | Compound 135 |
| 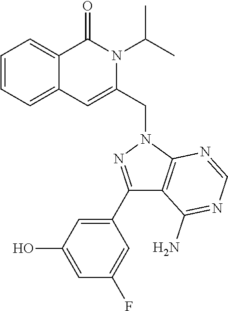 [SMILES: CC(C)n1c(Cn2nc(-c3cc(O)cc(F)c3)c3c(N)ncnc32)cc2ccccc2c1=O] | Compound 136 |
| 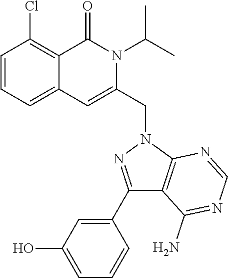 [SMILES: CC(C)n1c(Cn2nc(-c3cccc(O)c3)c3c(N)ncnc32)cc2cccc(Cl)c2c1=O] | Compound 137 |
| 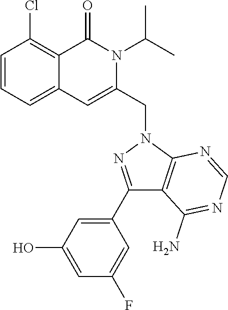 [SMILES: CC(C)n1c(Cn2nc(-c3cc(O)cc(F)c3)c3c(N)ncnc32)cc2cccc(Cl)c2c1=O] | Compound 138 |
| 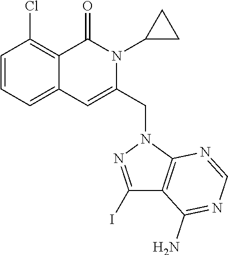 [SMILES: Nc1ncnc2c1c(I)nn2Cc1cc2cccc(Cl)c2c(=O)n1C1CC1] | Compound 139 |
| 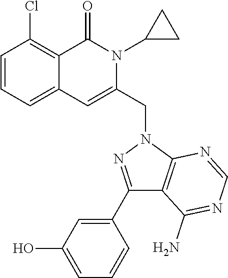 [SMILES: Nc1ncnc2c1c(-c1cccc(O)c1)nn2Cc1cc2cccc(Cl)c2c(=O)n1C1CC1] | Compound 141 |
| 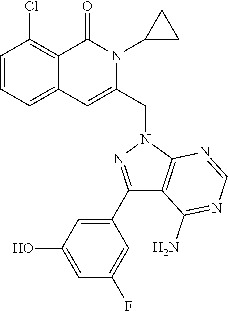 [SMILES: Nc1ncnc2c1c(-c1cc(O)cc(F)c1)nn2Cc1cc2cccc(Cl)c2c(=O)n1C1CC1] | Compound 142 |
| 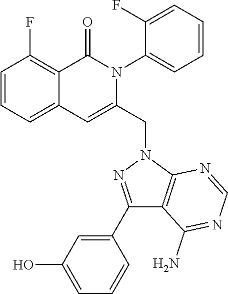 [SMILES: Nc1ncnc2c1c(-c1cccc(O)c1)nn2Cc1cc2cccc(F)c2c(=O)n1-c1ccccc1F] | Compound 143 |
| 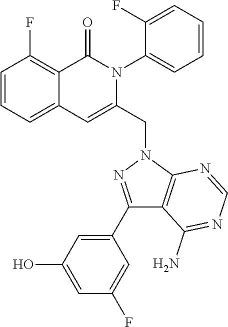 [SMILES: Nc1ncnc2c1c(-c1cc(O)cc(F)c1)nn2Cc1cc2cccc(F)c2c(=O)n1-c1ccccc1F] | Compound 144 |
| 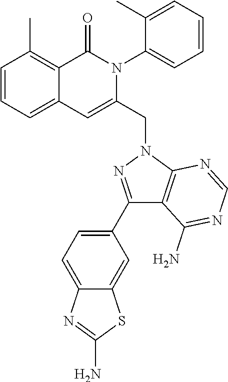 [SMILES: Cc1ccccc1-n1c(Cn2nc(-c3ccc4nc(N)sc4c3)c3c(N)ncnc32)cc2cccc(C)c2c1=O] | Compound 145 |
| 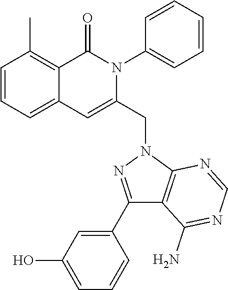 [SMILES: Cc1cccc2cc(Cn3nc(-c4cccc(O)c4)c4c(N)ncnc43)n(-c3ccccc3)c(=O)c12] | Compound 146 |
| 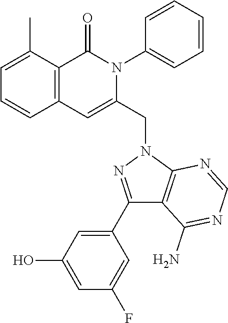 [SMILES: Cc1cccc2cc(Cn3nc(-c4cc(O)cc(F)c4)c4c(N)ncnc43)n(-c3ccccc3)c(=O)c12] | Compound 147 |
| 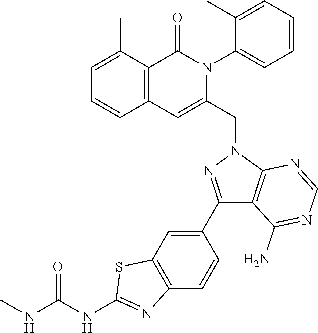 [SMILES: CNC(=O)Nc1nc2ccc(-c3nn(Cc4cc5cccc(C)c5c(=O)n4-c4ccccc4C)c4ncnc(N)c34)cc2s1] | Compound 148 |
| 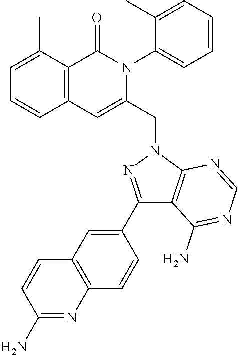 [SMILES: Cc1ccccc1-n1c(Cn2nc(-c3ccc4nc(N)ccc4c3)c3c(N)ncnc32)cc2cccc(C)c2c1=O] | Compound 149 |
| 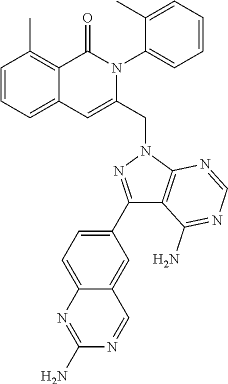 [SMILES: Cc1ccccc1-n1c(Cn2nc(-c3ccc4nc(N)ncc4c3)c3c(N)ncnc32)cc2cccc(C)c2c1=O] | Compound 150 |
| 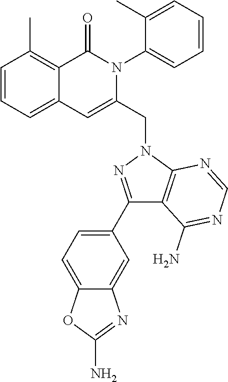 [SMILES: Cc1ccccc1-n1c(Cn2nc(-c3ccc4oc(N)nc4c3)c3c(N)ncnc32)cc2cccc(C)c2c1=O] | Compound 151 |
| 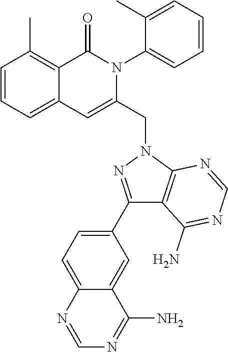 [SMILES: Cc1ccccc1-n1c(Cn2nc(-c3ccc4ncnc(N)c4c3)c3c(N)ncnc32)cc2cccc(C)c2c1=O] | Compound 152 |
| 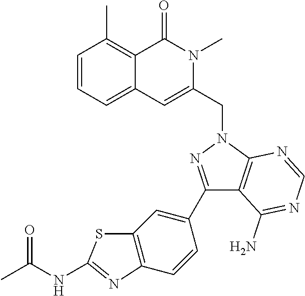 [SMILES: CC(=O)Nc1nc2ccc(-c3nn(Cc4cc5cccc(C)c5c(=O)n4C)c4ncnc(N)c34)cc2s1] | Compound 153 |
| 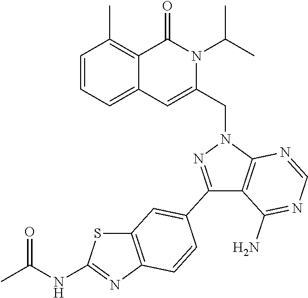 [SMILES: CC(=O)Nc1nc2ccc(-c3nn(Cc4cc5cccc(C)c5c(=O)n4C(C)C)c4ncnc(N)c34)cc2s1] | Compound 154 |
| 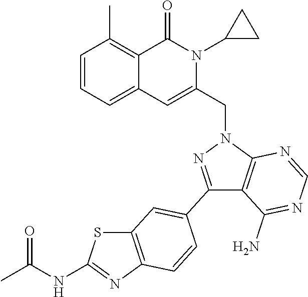 [SMILES: CC(=O)Nc1nc2ccc(-c3nn(Cc4cc5cccc(C)c5c(=O)n4C4CC4)c4ncnc(N)c34)cc2s1] | Compound 155 |
| 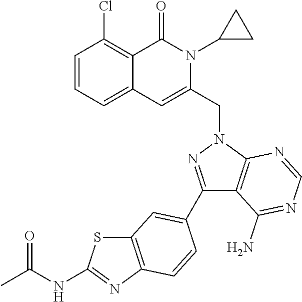 [SMILES: CC(=O)Nc1nc2ccc(-c3nn(Cc4cc5cccc(Cl)c5c(=O)n4C4CC4)c4ncnc(N)c34)cc2s1] | Compound 156 |
| 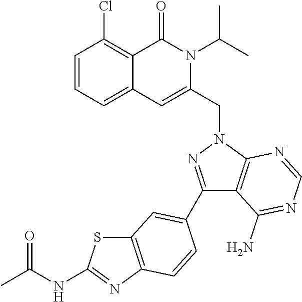 [SMILES: CC(=O)Nc1nc2ccc(-c3nn(Cc4cc5cccc(Cl)c5c(=O)n4C(C)C)c4ncnc(N)c34)cc2s1] | Compound 157 |
| 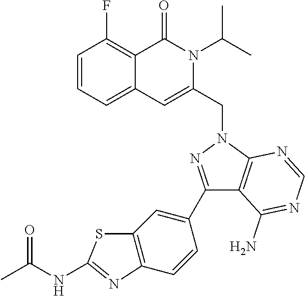 [SMILES: CC(=O)Nc1nc2ccc(-c3nn(Cc4cc5cccc(F)c5c(=O)n4C(C)C)c4ncnc(N)c34)cc2s1] | Compound 158 |
| 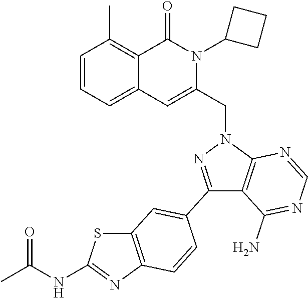 [SMILES: CC(=O)Nc1nc2ccc(-c3nn(Cc4cc5cccc(C)c5c(=O)n4C4CCC4)c4ncnc(N)c34)cc2s1] | Compound 159 |
| 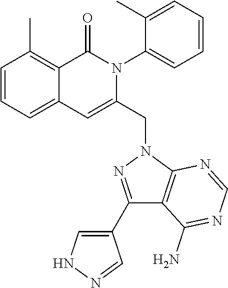 [SMILES: Cc1ccccc1-n1c(Cn2nc(-c3cn[nH]c3)c3c(N)ncnc32)cc2cccc(C)c2c1=O] | Compound 160 |
| 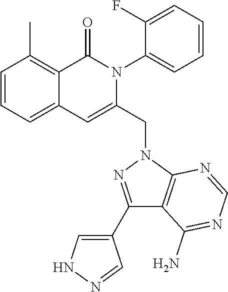 [SMILES: Cc1cccc2cc(Cn3nc(-c4cn[nH]c4)c4c(N)ncnc43)n(-c3ccccc3F)c(=O)c12] | Compound 161 |
| 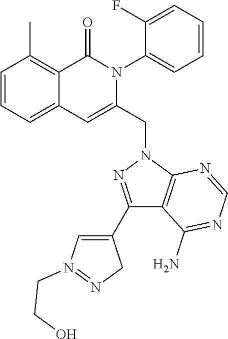 | Compound 162 |
| 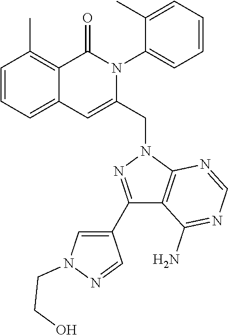 [SMILES: Cc1ccccc1-n1c(Cn2nc(-c3cnn(CCO)c3)c3c(N)ncnc32)cc2cccc(C)c2c1=O] | Compound 163 |
| 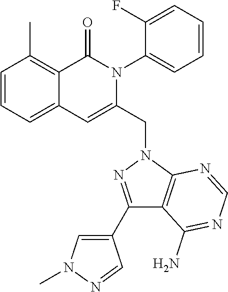 [SMILES: Cc1cccc2cc(Cn3nc(-c4cnn(C)c4)c4c(N)ncnc43)n(-c3ccccc3F)c(=O)c12] | Compound 164 |
| 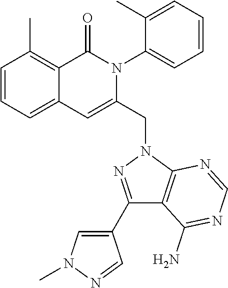 [SMILES: Cc1ccccc1-n1c(Cn2nc(-c3cnn(C)c3)c3c(N)ncnc32)cc2cccc(C)c2c1=O] | Compound 165 |
| 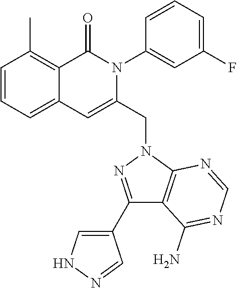 [SMILES: Cc1cccc2cc(Cn3nc(-c4cn[nH]c4)c4c(N)ncnc43)n(-c3cccc(F)c3)c(=O)c12] | Compound 166 |
| 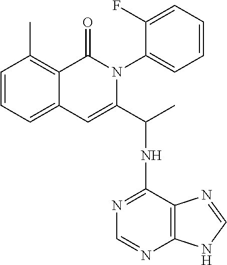 [SMILES: Cc1cccc2cc(C(C)Nc3ncnc4[nH]cnc34)n(-c3ccccc3F)c(=O)c12] | Compound 167 |
| 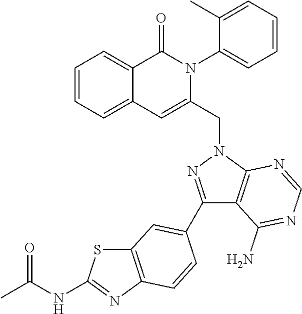 [SMILES: CC(=O)Nc1nc2ccc(-c3nn(Cc4cc5ccccc5c(=O)n4-c4ccccc4C)c4ncnc(N)c34)cc2s1] | Compound 168 |
| 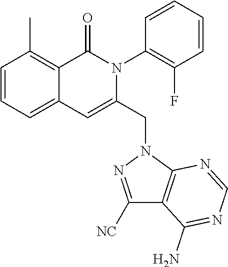 [SMILES: Cc1cccc2cc(Cn3nc(C#N)c4c(N)ncnc43)n(-c3ccccc3F)c(=O)c12] | Compound 169 |
| 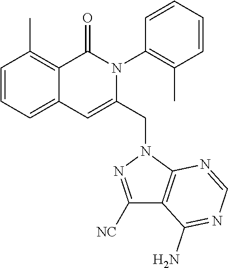 [SMILES: Cc1ccccc1-n1c(Cn2nc(C#N)c3c(N)ncnc32)cc2cccc(C)c2c1=O] | Compound 170 |
| 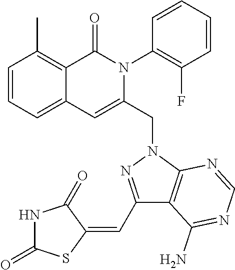 [SMILES: Cc1cccc2cc(Cn3nc(/C=C4/SC(=O)NC4=O)c4c(N)ncnc43)n(-c3ccccc3F)c(=O)c12] | Compound 171 |
| 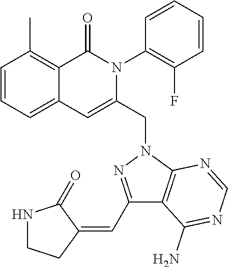 [SMILES: Cc1cccc2cc(Cn3nc(/C=C4/CCNC4=O)c4c(N)ncnc43)n(-c3ccccc3F)c(=O)c12] | Compound 172 |
| 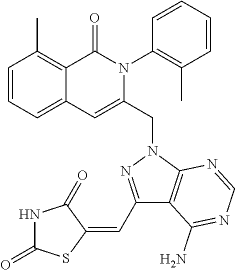 [SMILES: Cc1ccccc1-n1c(Cn2nc(/C=C3/SC(=O)NC3=O)c3c(N)ncnc32)cc2cccc(C)c2c1=O] | Compound 173 |
| 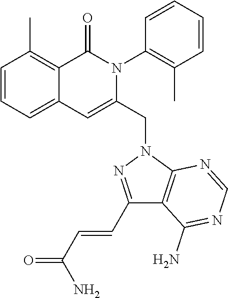 [SMILES: Cc1ccccc1-n1c(Cn2nc(/C=C/C(N)=O)c3c(N)ncnc32)cc2cccc(C)c2c1=O] | Compound 174 |
| 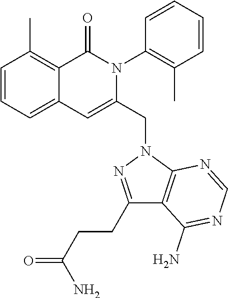 [SMILES: Cc1ccccc1-n1c(Cn2nc(CCC(N)=O)c3c(N)ncnc32)cc2cccc(C)c2c1=O] | Compound 175 |
| 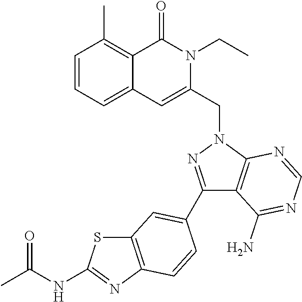 [SMILES: CCn1c(Cn2nc(-c3ccc4nc(NC(C)=O)sc4c3)c3c(N)ncnc32)cc2cccc(C)c2c1=O] | Compound 176 |
| 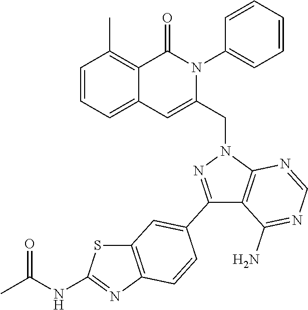 [SMILES: CC(=O)Nc1nc2ccc(-c3nn(Cc4cc5cccc(C)c5c(=O)n4-c4ccccc4)c4ncnc(N)c34)cc2s1] | Compound 177 |
| 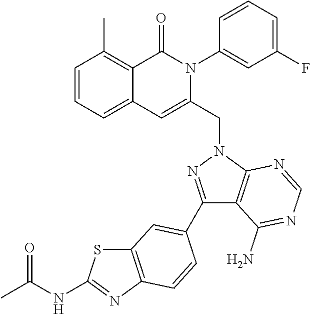 [SMILES: CC(=O)Nc1nc2ccc(-c3nn(Cc4cc5cccc(C)c5c(=O)n4-c4cccc(F)c4)c4ncnc(N)c34)cc2s1] | Compound 178 |
| 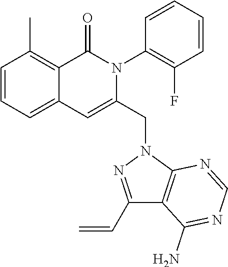 [SMILES: C=Cc1nn(Cc2cc3cccc(C)c3c(=O)n2-c2ccccc2F)c2ncnc(N)c12] | Compound 179 |
| 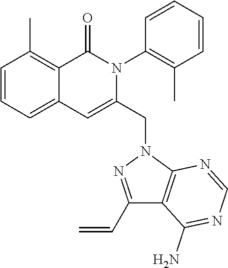 [SMILES: C=Cc1nn(Cc2cc3cccc(C)c3c(=O)n2-c2ccccc2C)c2ncnc(N)c12] | Compound 180 |
| 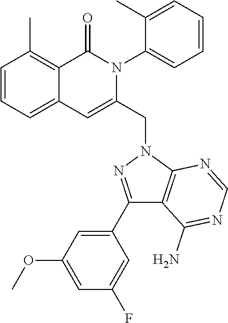 [SMILES: COc1cc(F)cc(-c2nn(Cc3cc4cccc(C)c4c(=O)n3-c3ccccc3C)c3ncnc(N)c23)c1] | Compound 181 |
| 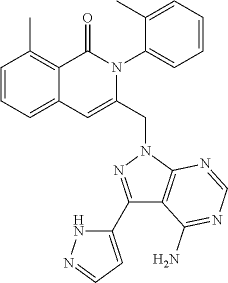 [SMILES: Cc1ccccc1-n1c(Cn2nc(-c3ccn[nH]3)c3c(N)ncnc32)cc2cccc(C)c2c1=O] | Compound 182 |
| 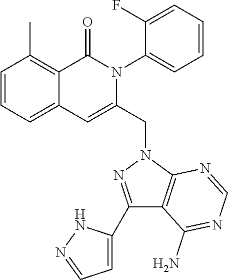 [SMILES: Cc1cccc2cc(Cn3nc(-c4ccn[nH]4)c4c(N)ncnc43)n(-c3ccccc3F)c(=O)c12] | Compound 183 |
| 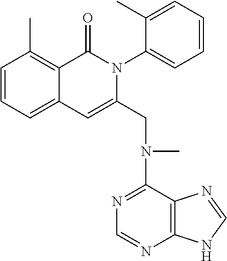 [SMILES: Cc1ccccc1-n1c(CN(C)c2ncnc3[nH]cnc23)cc2cccc(C)c2c1=O] | Compound 184 |
| 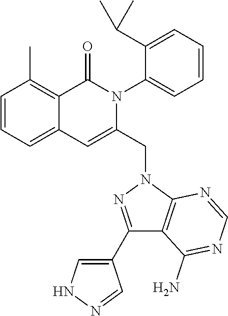 [SMILES: Cc1cccc2cc(Cn3nc(-c4cn[nH]c4)c4c(N)ncnc43)n(-c3ccccc3C(C)C)c(=O)c12] | Compound 185 |
| 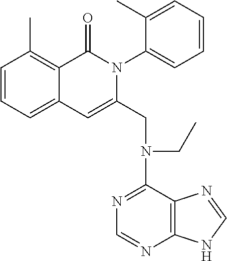 [SMILES: CCN(Cc1cc2cccc(C)c2c(=O)n1-c1ccccc1C)c1ncnc2[nH]cnc12] | Compound 186 |
| 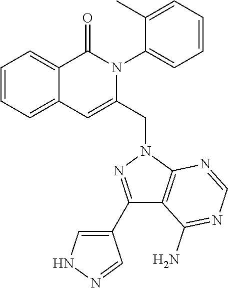 [SMILES: Cc1ccccc1-n1c(Cn2nc(-c3cn[nH]c3)c3c(N)ncnc32)cc2ccccc2c1=O] | Compound 187 |
| 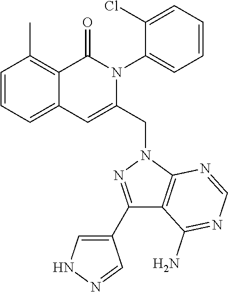 [SMILES: Cc1cccc2cc(Cn3nc(-c4cn[nH]c4)c4c(N)ncnc43)n(-c3ccccc3Cl)c(=O)c12] | Compound 188 |
| 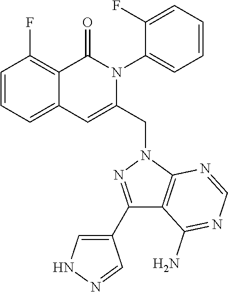 [SMILES: Nc1ncnc2c1c(-c1cn[nH]c1)nn2Cc1cc2cccc(F)c2c(=O)n1-c1ccccc1F] | Compound 189 |
| 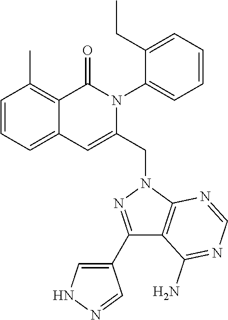 [SMILES: CCc1ccccc1-n1c(Cn2nc(-c3cn[nH]c3)c3c(N)ncnc32)cc2cccc(C)c2c1=O] | Compound 190 |
| 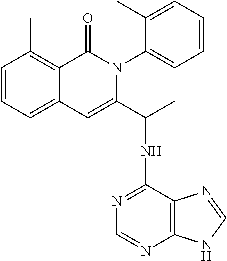 [SMILES: Cc1ccccc1-n1c(C(C)Nc2ncnc3[nH]cnc23)cc2cccc(C)c2c1=O] | Compound 191 |
| 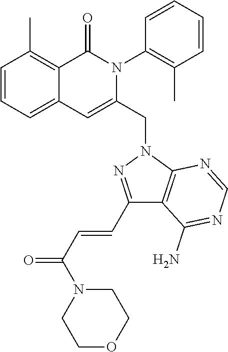 [SMILES: Cc1ccccc1-n1c(Cn2nc(/C=C/C(=O)N3CCOCC3)c3c(N)ncnc32)cc2cccc(C)c2c1=O] | Compound 192 |
| 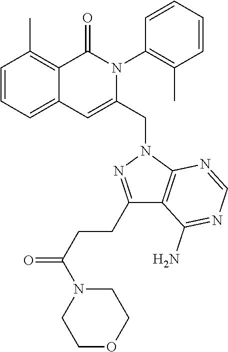 [SMILES: Cc1ccccc1-n1c(Cn2nc(CCC(=O)N3CCOCC3)c3c(N)ncnc32)cc2cccc(C)c2c1=O] | Compound 193 |
| 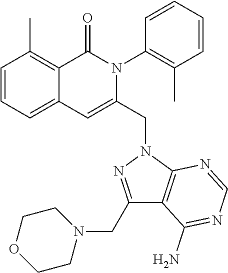 [SMILES: Cc1ccccc1-n1c(Cn2nc(CN3CCOCC3)c3c(N)ncnc32)cc2cccc(C)c2c1=O] | Compound 194 |
| 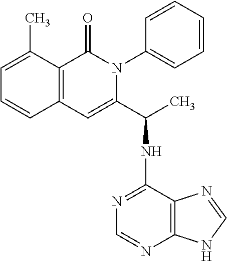 [SMILES: Cc1cccc2cc([C@@H](C)Nc3ncnc4[nH]cnc34)n(-c3ccccc3)c(=O)c12] | Compound 195 |
| 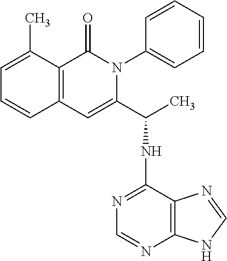 [SMILES: Cc1cccc2cc([C@H](C)Nc3ncnc4[nH]cnc34)n(-c3ccccc3)c(=O)c12] | Compound 196 |
| 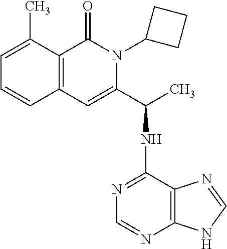 [SMILES: Cc1cccc2cc([C@@H](C)Nc3ncnc4[nH]cnc34)n(C3CCC3)c(=O)c12] | Compound 197 |
| 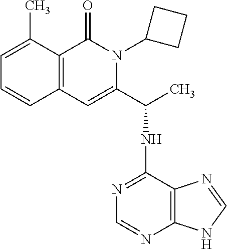 [SMILES: Cc1cccc2cc([C@H](C)Nc3ncnc4[nH]cnc34)n(C3CCC3)c(=O)c12] | Compound 198 |
| 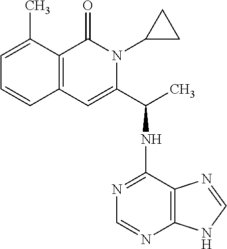 [SMILES: Cc1cccc2cc([C@@H](C)Nc3ncnc4[nH]cnc34)n(C3CC3)c(=O)c12] | Compound 199 |
| 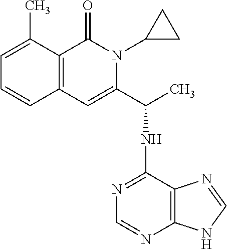 [SMILES: Cc1cccc2cc([C@H](C)Nc3ncnc4[nH]cnc34)n(C3CC3)c(=O)c12] | Compound 200 |
| 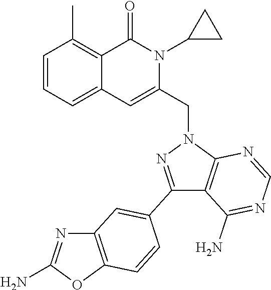 [SMILES: Cc1cccc2cc(Cn3nc(-c4ccc5oc(N)nc5c4)c4c(N)ncnc43)n(C3CC3)c(=O)c12] | Compound 201 |
| 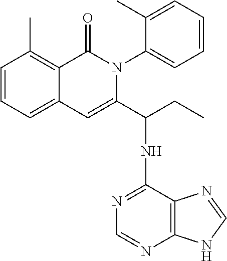 [SMILES: CCC(Nc1ncnc2[nH]cnc12)c1cc2cccc(C)c2c(=O)n1-c1ccccc1C] | Compound 202 |
| 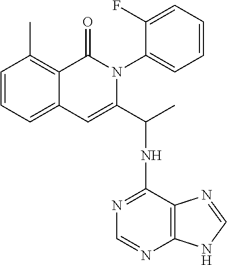 [SMILES: Cc1cccc2cc(C(C)Nc3ncnc4[nH]cnc34)n(-c3ccccc3F)c(=O)c12] | Compound 203 |
| 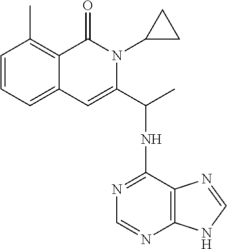 [SMILES: Cc1cccc2cc(C(C)Nc3ncnc4[nH]cnc34)n(C3CC3)c(=O)c12] | Compound 204 |
| 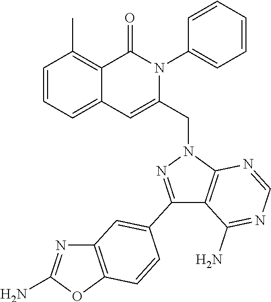 [SMILES: Cc1cccc2cc(Cn3nc(-c4ccc5oc(N)nc5c4)c4c(N)ncnc43)n(-c3ccccc3)c(=O)c12] | Compound 205 |
| 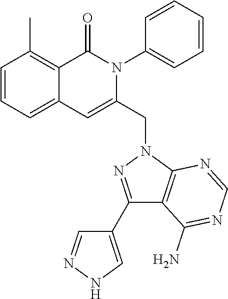 [SMILES: Cc1cccc2cc(Cn3nc(-c4cn[nH]c4)c4c(N)ncnc43)n(-c3ccccc3)c(=O)c12] | Compound 206 |
| 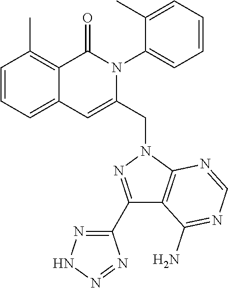 [SMILES: Cc1ccccc1-n1c(Cn2nc(-c3nn[nH]n3)c3c(N)ncnc32)cc2cccc(C)c2c1=O] | Compound 207 |
| 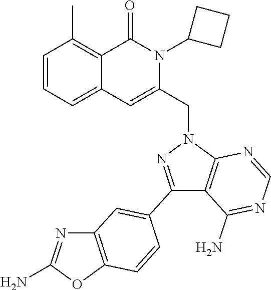 [SMILES: Cc1cccc2cc(Cn3nc(-c4ccc5oc(N)nc5c4)c4c(N)ncnc43)n(C3CCC3)c(=O)c12] | Compound 208 |
| 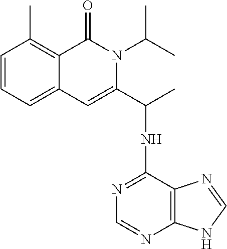 [SMILES: Cc1cccc2cc(C(C)Nc3ncnc4[nH]cnc34)n(C(C)C)c(=O)c12] | Compound 209 |
| 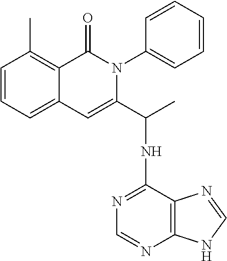 [SMILES: Cc1cccc2cc(C(C)Nc3ncnc4[nH]cnc34)n(-c3ccccc3)c(=O)c12] | Compound 210 |
| 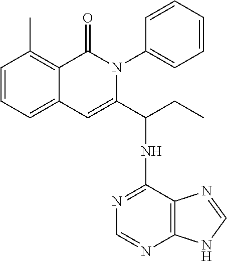 [SMILES: CCC(Nc1ncnc2[nH]cnc12)c1cc2cccc(C)c2c(=O)n1-c1ccccc1] | Compound 211 |
| 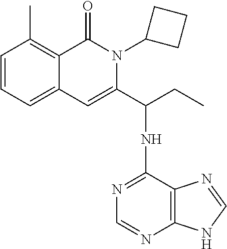 [SMILES: CCC(Nc1ncnc2[nH]cnc12)c1cc2cccc(C)c2c(=O)n1C1CCC1] | Compound 212 |
| 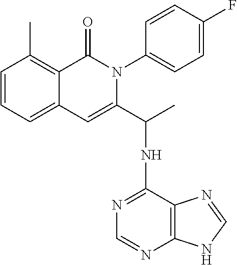 [SMILES: Cc1cccc2cc(C(C)Nc3ncnc4[nH]cnc34)n(-c3ccc(F)cc3)c(=O)c12] | Compound 213 |
| 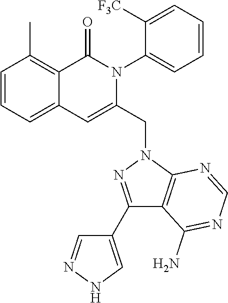 [SMILES: Cc1cccc2cc(Cn3nc(-c4cn[nH]c4)c4c(N)ncnc43)n(-c3ccccc3C(F)(F)F)c(=O)c12] | Compound 214 |
| 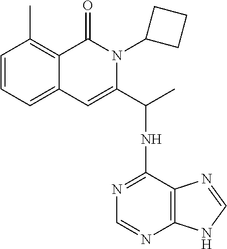 [SMILES: Cc1cccc2cc(C(C)Nc3ncnc4[nH]cnc34)n(C3CCC3)c(=O)c12] | Compound 215 |
| 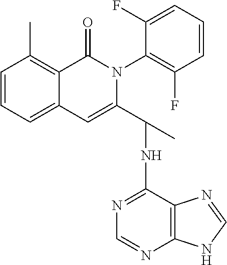 [SMILES: Cc1cccc2cc(C(C)Nc3ncnc4[nH]cnc34)n(-c3c(F)cccc3F)c(=O)c12] | Compound 216 |
| 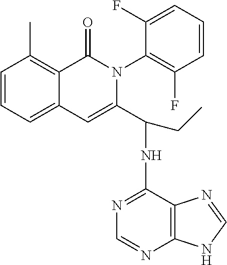 [SMILES: CCC(Nc1ncnc2[nH]cnc12)c1cc2cccc(C)c2c(=O)n1-c1c(F)cccc1F] | Compound 217 |
| 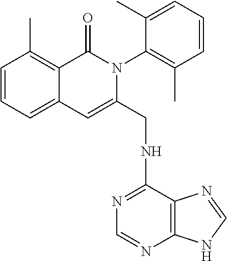 [SMILES: Cc1cccc(C)c1-n1c(CNc2ncnc3[nH]cnc23)cc2cccc(C)c2c1=O] | Compound 218 |
| 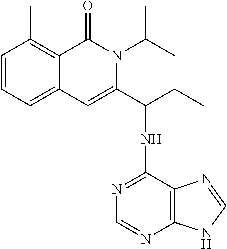 [SMILES: CCC(Nc1ncnc2[nH]cnc12)c1cc2cccc(C)c2c(=O)n1C(C)C] | Compound 219 |
| 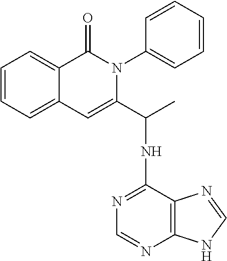 [SMILES: CC(Nc1ncnc2[nH]cnc12)c1cc2ccccc2c(=O)n1-c1ccccc1] | Compound 220 |
| 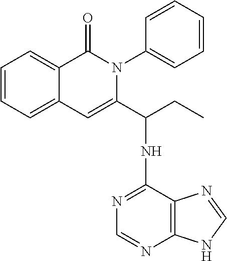 [SMILES: CCC(Nc1ncnc2[nH]cnc12)c1cc2ccccc2c(=O)n1-c1ccccc1] | Compound 221 |
| 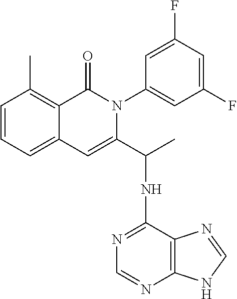 [SMILES: Cc1cccc2cc(C(C)Nc3ncnc4[nH]cnc34)n(-c3cc(F)cc(F)c3)c(=O)c12] | Compound 222 |
| 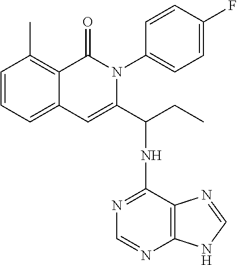 [SMILES: CCC(Nc1ncnc2[nH]cnc12)c1cc2cccc(C)c2c(=O)n1-c1ccc(F)cc1] | Compound 223 |
| 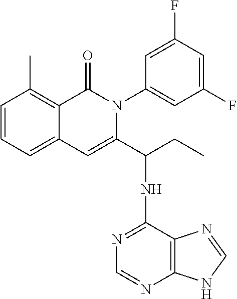 [SMILES: CCC(Nc1ncnc2[nH]cnc12)c1cc2cccc(C)c2c(=O)n1-c1cc(F)cc(F)c1] | Compound 224 |
| 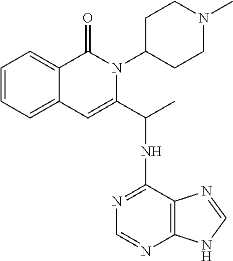 [SMILES: CC(Nc1ncnc2[nH]cnc12)c1cc2ccccc2c(=O)n1C1CCN(C)CC1] | Compound 225 |
| 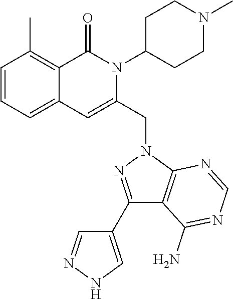 [SMILES: Cc1cccc2cc(Cn3nc(-c4cn[nH]c4)c4c(N)ncnc43)n(C3CCN(C)CC3)c(=O)c12] | Compound 226 |
| 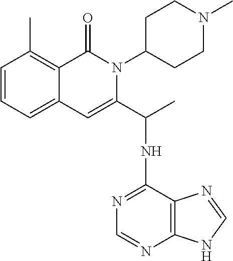 [SMILES: Cc1cccc2cc(C(C)Nc3ncnc4[nH]cnc34)n(C3CCN(C)CC3)c(=O)c12] | Compound 227 |
| 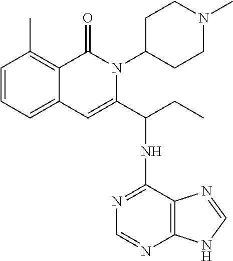 [SMILES: CCC(Nc1ncnc2[nH]cnc12)c1cc2cccc(C)c2c(=O)n1C1CCN(C)CC1] | Compound 228 |
| 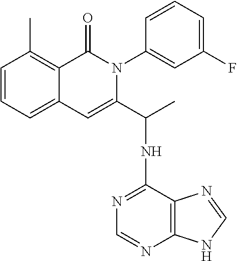 [SMILES: Cc1cccc2cc(C(C)Nc3ncnc4[nH]cnc34)n(-c3cccc(F)c3)c(=O)c12] | Compound 229 |
| 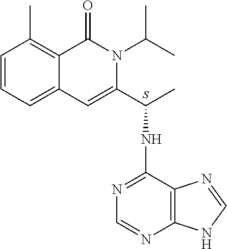 | Compound 230 |
| 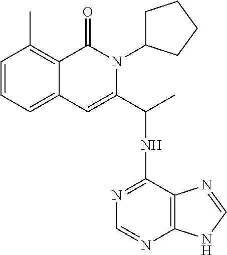 [SMILES: Cc1cccc2cc(C(C)Nc3ncnc4[nH]cnc34)n(C3CCCC3)c(=O)c12] | Compound 231 |
| 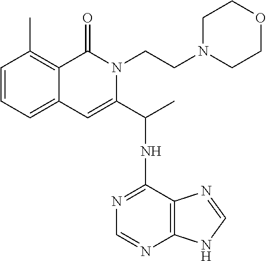 [SMILES: Cc1cccc2cc(C(C)Nc3ncnc4[nH]cnc34)n(CCN3CCOCC3)c(=O)c12] | Compound 232 |
| 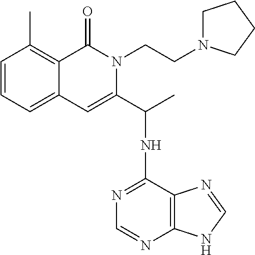 [SMILES: Cc1cccc2cc(C(C)Nc3ncnc4[nH]cnc34)n(CCN3CCCC3)c(=O)c12] | Compound 233 |
| 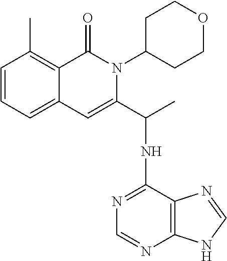 [SMILES: Cc1cccc2cc(C(C)Nc3ncnc4[nH]cnc34)n(C3CCOCC3)c(=O)c12] | Compound 234 |
| 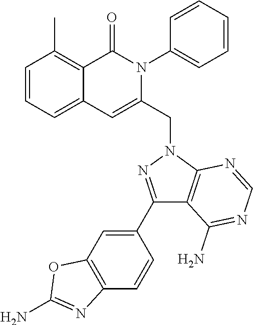 [SMILES: Cc1cccc2cc(Cn3nc(-c4ccc5nc(N)oc5c4)c4c(N)ncnc43)n(-c3ccccc3)c(=O)c12] | Compound 235 |
| 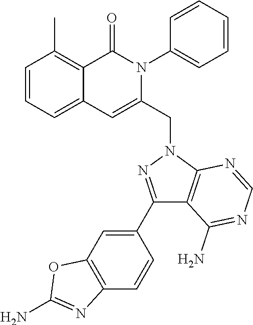 [SMILES: Cc1cccc2cc(Cn3nc(-c4ccc5nc(N)oc5c4)c4c(N)ncnc43)n(-c3ccccc3)c(=O)c12] | Compound 236 |
| 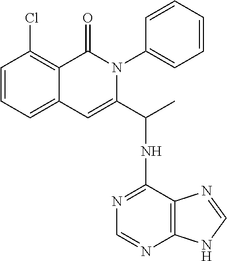 [SMILES: CC(Nc1ncnc2[nH]cnc12)c1cc2cccc(Cl)c2c(=O)n1-c1ccccc1] | Compound 237 |
| 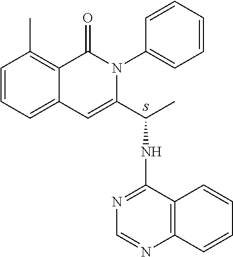 | Compound 238 |
| 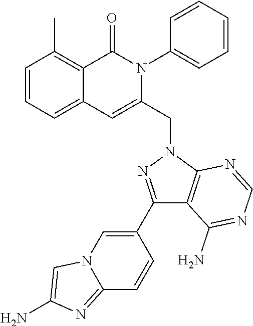 [SMILES: Cc1cccc2cc(Cn3nc(-c4ccc5nc(N)cn5c4)c4c(N)ncnc43)n(-c3ccccc3)c(=O)c12] | Compound 239 |
| 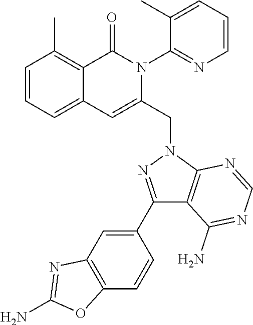 [SMILES: Cc1cccnc1-n1c(Cn2nc(-c3ccc4oc(N)nc4c3)c3c(N)ncnc32)cc2cccc(C)c2c1=O] | Compound 240 |
| 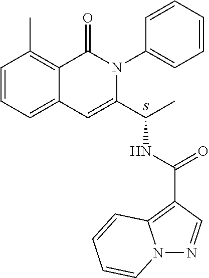 | Compound 241 |
| 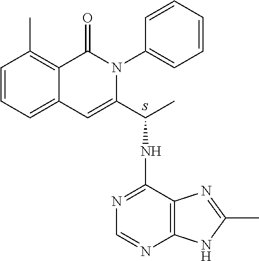 | Compound 242 |
| 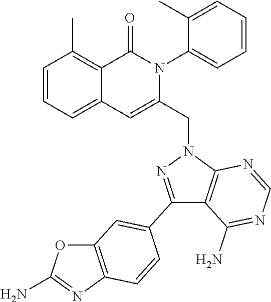 [SMILES: Cc1ccccc1-n1c(Cn2nc(-c3ccc4nc(N)oc4c3)c3c(N)ncnc32)cc2cccc(C)c2c1=O] | Compound 243 |
| 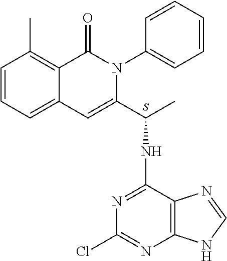 | Compound 244 |
| 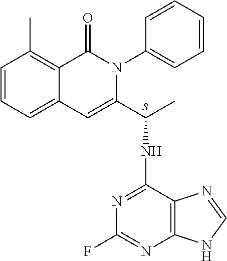 | Compound 245 |
| 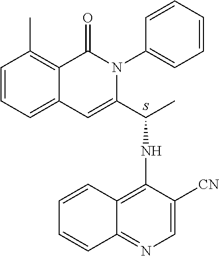 | Compound 246 |
| 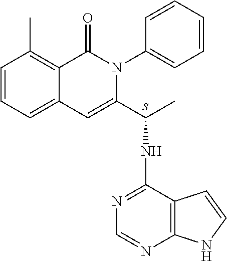 | Compound 247 |
| 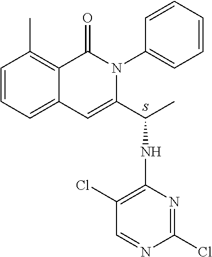 | Compound 248 |
| 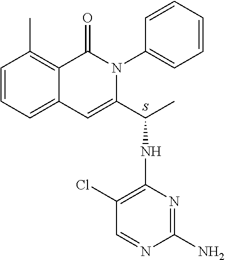 | Compound 249 |
| 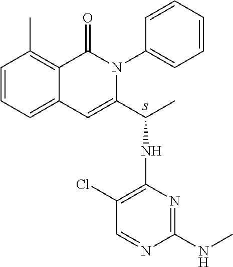 | Compound 250 |
| 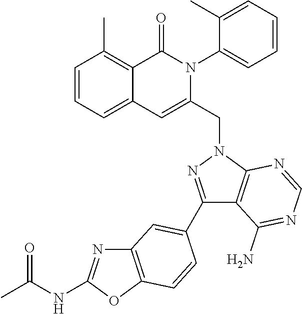 [SMILES: CC(=O)Nc1nc2cc(-c3nn(Cc4cc5cccc(C)c5c(=O)n4-c4ccccc4C)c4ncnc(N)c34)ccc2o1] | Compound 251 |
| 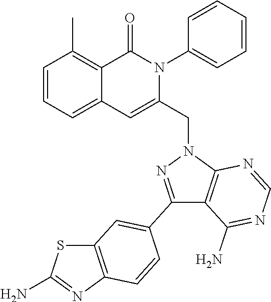 [SMILES: Cc1cccc2cc(Cn3nc(-c4ccc5nc(N)sc5c4)c4c(N)ncnc43)n(-c3ccccc3)c(=O)c12] | Compound 252 |
| 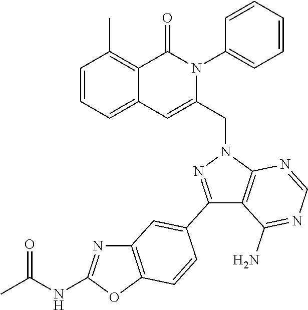 [SMILES: CC(=O)Nc1nc2cc(-c3nn(Cc4cc5cccc(C)c5c(=O)n4-c4ccccc4)c4ncnc(N)c34)ccc2o1] | Compound 253 |
| 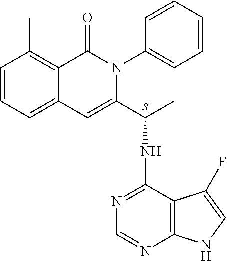 | Compound 254 |
| 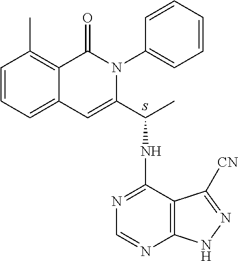 | Compound 255 |
| 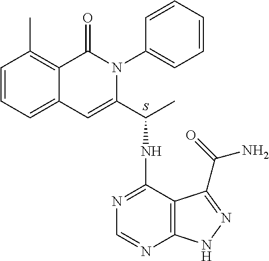 | Compound 256 |
| 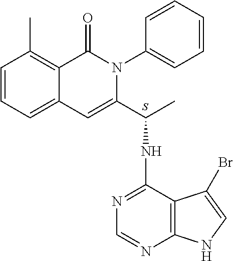 | Compound 257 |
| 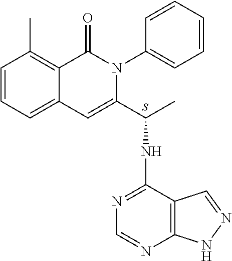 | Compound 258 |
| 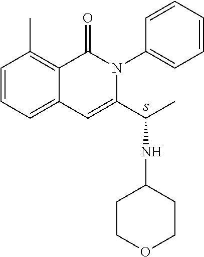 | Compound 259 |
| 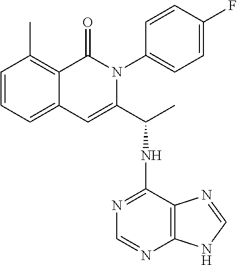 [SMILES: Cc1cccc2cc([C@H](C)Nc3ncnc4[nH]cnc34)n(-c3ccc(F)cc3)c(=O)c12] | Compound 260 |
| 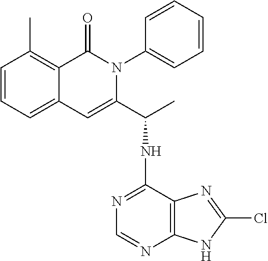 [SMILES: Cc1cccc2cc([C@H](C)Nc3ncnc4[nH]c(Cl)nc34)n(-c3ccccc3)c(=O)c12] | Compound 261 |
| 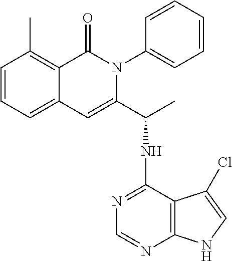 [SMILES: Cc1cccc2cc([C@H](C)Nc3ncnc4[nH]cc(Cl)c34)n(-c3ccccc3)c(=O)c12] | Compound 262 |
| 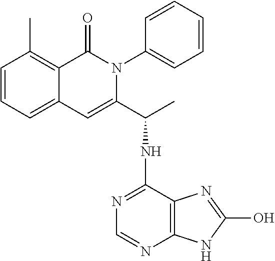 [SMILES: Cc1cccc2cc([C@H](C)Nc3ncnc4[nH]c(O)nc34)n(-c3ccccc3)c(=O)c12] | Compound 263 |
| 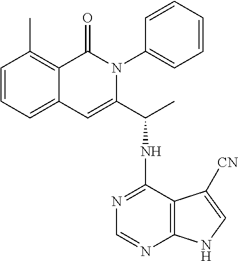 [SMILES: Cc1cccc2cc([C@H](C)Nc3ncnc4[nH]cc(C#N)c34)n(-c3ccccc3)c(=O)c12] | Compound 264 |
| 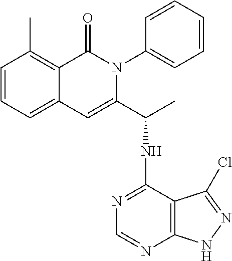 [SMILES: Cc1cccc2cc([C@H](C)Nc3ncnc4[nH]nc(Cl)c34)n(-c3ccccc3)c(=O)c12] | Compound 265 |
| 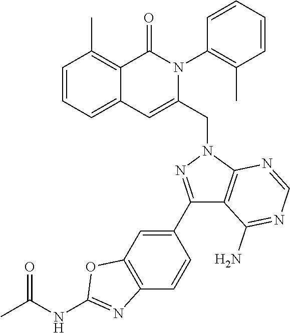 [SMILES: CC(=O)Nc1nc2ccc(-c3nn(Cc4cc5cccc(C)c5c(=O)n4-c4ccccc4C)c4ncnc(N)c34)cc2o1] | Compound 266 |
| 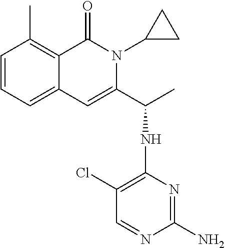 [SMILES: Cc1cccc2cc([C@H](C)Nc3nc(N)ncc3Cl)n(C3CC3)c(=O)c12] | Compound 267 |
| 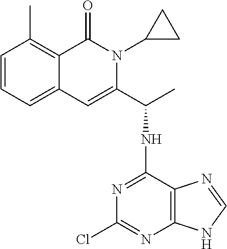 [SMILES: Cc1cccc2cc([C@H](C)Nc3nc(Cl)nc4[nH]cnc34)n(C3CC3)c(=O)c12] | Compound 268 |
| 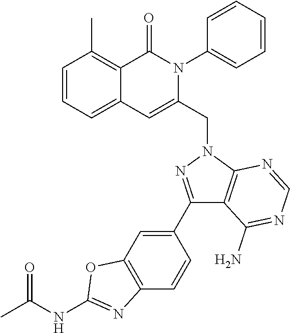 [SMILES: CC(=O)Nc1nc2ccc(-c3nn(Cc4cc5cccc(C)c5c(=O)n4-c4ccccc4)c4ncnc(N)c34)cc2o1] | Compound 269 |
| 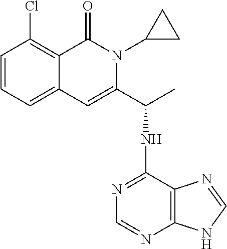 [SMILES: C[C@H](Nc1ncnc2[nH]cnc12)c1cc2cccc(Cl)c2c(=O)n1C1CC1] | Compound 270 |
| 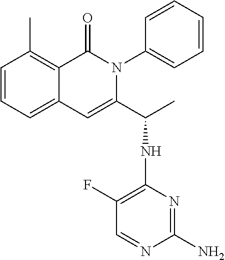 [SMILES: Cc1cccc2cc([C@H](C)Nc3nc(N)ncc3F)n(-c3ccccc3)c(=O)c12] | Compound 271 |
| 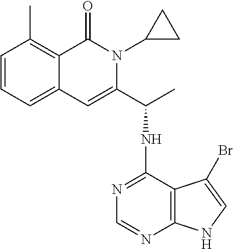 [SMILES: Cc1cccc2cc([C@H](C)Nc3ncnc4[nH]cc(Br)c34)n(C3CC3)c(=O)c12] | Compound 272 |
| 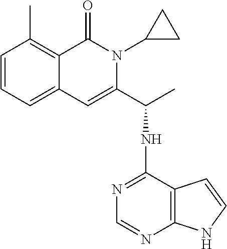 [SMILES: Cc1cccc2cc([C@H](C)Nc3ncnc4[nH]ccc34)n(C3CC3)c(=O)c12] | Compound 273 |
| 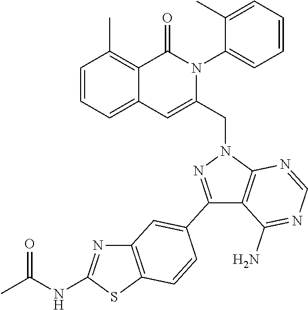 [SMILES: CC(=O)Nc1nc2cc(-c3nn(Cc4cc5cccc(C)c5c(=O)n4-c4ccccc4C)c4ncnc(N)c34)ccc2s1] | Compound 274 |
| 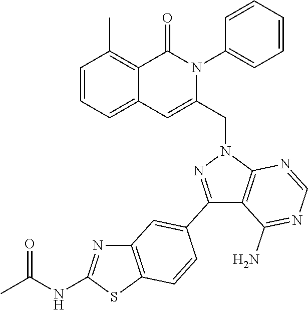 [SMILES: CC(=O)Nc1nc2cc(-c3nn(Cc4cc5cccc(C)c5c(=O)n4-c4ccccc4)c4ncnc(N)c34)ccc2s1] | Compound 275 |
| 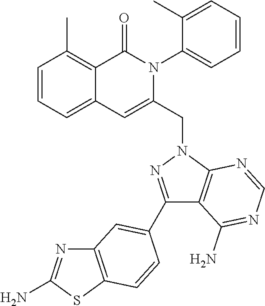 [SMILES: Cc1ccccc1-n1c(Cn2nc(-c3ccc4sc(N)nc4c3)c3c(N)ncnc32)cc2cccc(C)c2c1=O] | Compound 276 |
| 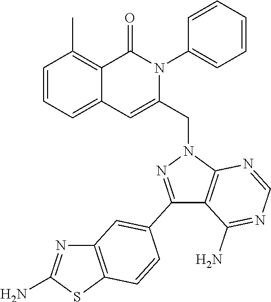 [SMILES: Cc1cccc2cc(Cn3nc(-c4ccc5sc(N)nc5c4)c4c(N)ncnc43)n(-c3ccccc3)c(=O)c12] | Compound 277 |
| 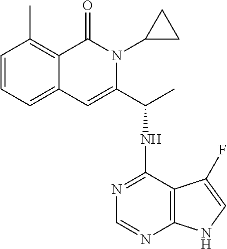 [SMILES: Cc1cccc2cc([C@H](C)Nc3ncnc4[nH]cc(F)c34)n(C3CC3)c(=O)c12] | Compound 278 |
| 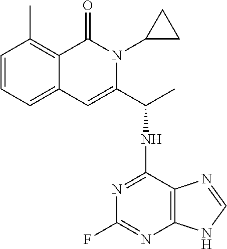 [SMILES: Cc1cccc2cc([C@H](C)Nc3nc(F)nc4[nH]cnc34)n(C3CC3)c(=O)c12] | Compound 279 |
| 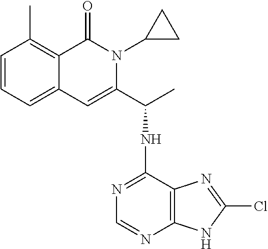 [SMILES: Cc1cccc2cc([C@H](C)Nc3ncnc4[nH]c(Cl)nc34)n(C3CC3)c(=O)c12] | Compound 280 |
| 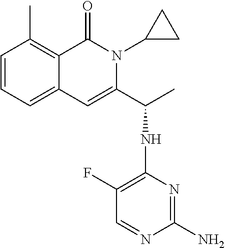 [SMILES: Cc1cccc2cc([C@H](C)Nc3nc(N)ncc3F)n(C3CC3)c(=O)c12] | Compound 281 |
| 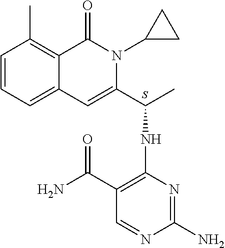 | Compound 282 |
| 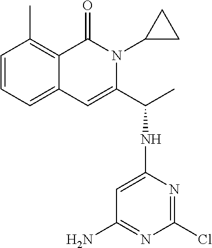 [SMILES: Cc1cccc2cc([C@H](C)Nc3cc(N)nc(Cl)n3)n(C3CC3)c(=O)c12] | Compound 283 |
| 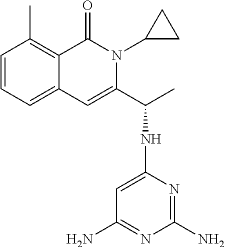 [SMILES: Cc1cccc2cc([C@H](C)Nc3cc(N)nc(N)n3)n(C3CC3)c(=O)c12] | Compound 284 |
| 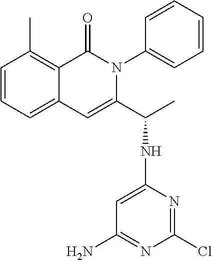 [SMILES: Cc1cccc2cc([C@H](C)Nc3cc(N)nc(Cl)n3)n(-c3ccccc3)c(=O)c12] | Compound 285 |
| 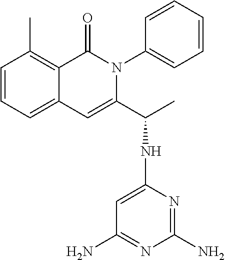 [SMILES: Cc1cccc2cc([C@H](C)Nc3cc(N)nc(N)n3)n(-c3ccccc3)c(=O)c12] | Compound 286 |
| 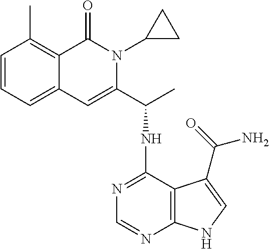 [SMILES: Cc1cccc2cc([C@H](C)Nc3ncnc4[nH]cc(C(N)=O)c34)n(C3CC3)c(=O)c12] | Compound 287 |
| 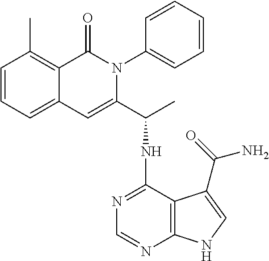 [SMILES: Cc1cccc2cc([C@H](C)Nc3ncnc4[nH]cc(C(N)=O)c34)n(-c3ccccc3)c(=O)c12] | Compound 288 |
| 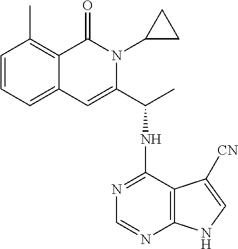 [SMILES: Cc1cccc2cc([C@H](C)Nc3ncnc4[nH]cc(C#N)c34)n(C3CC3)c(=O)c12] | Compound 289 |
| 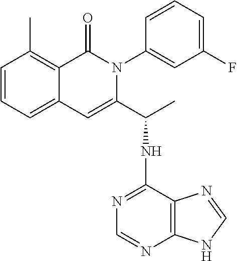 [SMILES: Cc1cccc2cc([C@H](C)Nc3ncnc4[nH]cnc34)n(-c3cccc(F)c3)c(=O)c12] | Compound 290 |
| 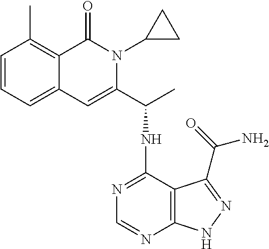 [SMILES: Cc1cccc2cc([C@H](C)Nc3ncnc4[nH]nc(C(N)=O)c34)n(C3CC3)c(=O)c12] | Compound 291 |
| 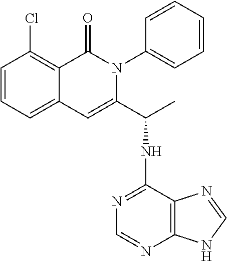 [SMILES: C[C@H](Nc1ncnc2[nH]cnc12)c1cc2cccc(Cl)c2c(=O)n1-c1ccccc1] | Compound 292 |
| 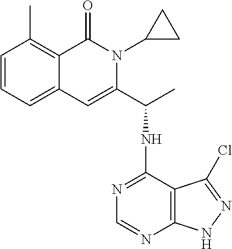 [SMILES: Cc1cccc2cc([C@H](C)Nc3ncnc4[nH]nc(Cl)c34)n(C3CC3)c(=O)c12] | Compound 293 |
| 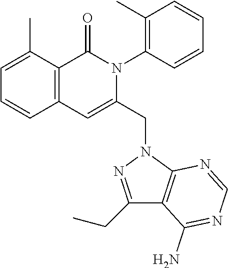 [SMILES: CCc1nn(Cc2cc3cccc(C)c3c(=O)n2-c2ccccc2C)c2ncnc(N)c12] | Compound 294 |
| 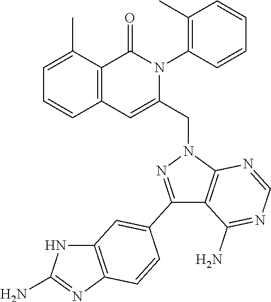 [SMILES: Cc1ccccc1-n1c(Cn2nc(-c3ccc4nc(N)[nH]c4c3)c3c(N)ncnc32)cc2cccc(C)c2c1=O] | Compound 295 |
| 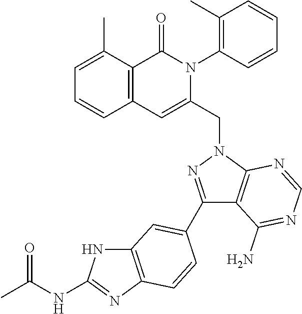 [SMILES: CC(=O)Nc1nc2ccc(-c3nn(Cc4cc5cccc(C)c5c(=O)n4-c4ccccc4C)c4ncnc(N)c34)cc2[nH]1] | Compound 296 |
| 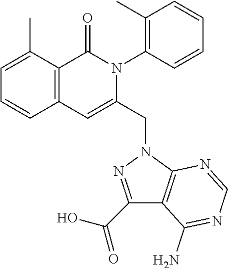 [SMILES: Cc1ccccc1-n1c(Cn2nc(C(=O)O)c3c(N)ncnc32)cc2cccc(C)c2c1=O] | Compound 297 |
| 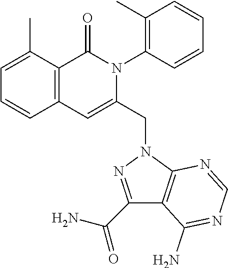 [SMILES: Cc1ccccc1-n1c(Cn2nc(C(N)=O)c3c(N)ncnc32)cc2cccc(C)c2c1=O] | Compound 298 |
| 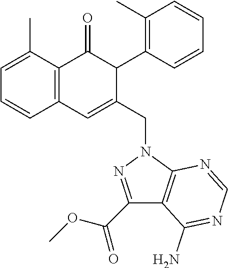 [SMILES: COC(=O)c1nn(CC2=Cc3cccc(C)c3C(=O)C2c2ccccc2C)c2ncnc(N)c12] | Compound 299 |
| 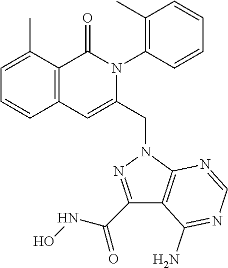 [SMILES: Cc1ccccc1-n1c(Cn2nc(C(=O)NO)c3c(N)ncnc32)cc2cccc(C)c2c1=O] | Compound 300 |
| 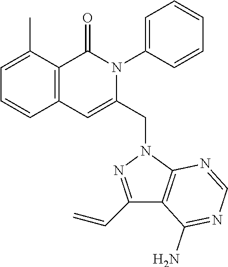 [SMILES: C=Cc1nn(Cc2cc3cccc(C)c3c(=O)n2-c2ccccc2)c2ncnc(N)c12] | Compound 301 |
| 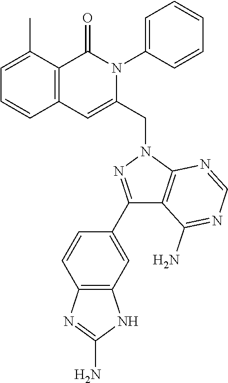 [SMILES: Cc1cccc2cc(Cn3nc(-c4ccc5nc(N)[nH]c5c4)c4c(N)ncnc43)n(-c3ccccc3)c(=O)c12] | Compound 302 |
| 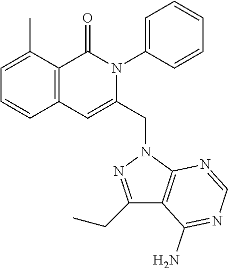 [SMILES: CCc1nn(Cc2cc3cccc(C)c3c(=O)n2-c2ccccc2)c2ncnc(N)c12] | Compound 303 |
| 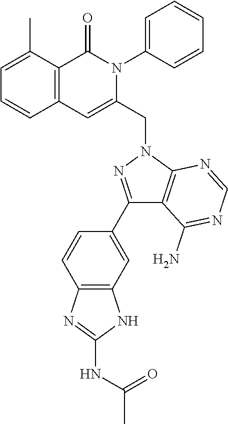 [SMILES: CC(=O)Nc1nc2ccc(-c3nn(Cc4cc5cccc(C)c5c(=O)n4-c4ccccc4)c4ncnc(N)c34)cc2[nH]1] | Compound 304 |
| 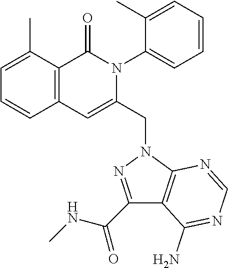 [SMILES: CNC(=O)c1nn(Cc2cc3cccc(C)c3c(=O)n2-c2ccccc2C)c2ncnc(N)c12] | Compound 305 |
| 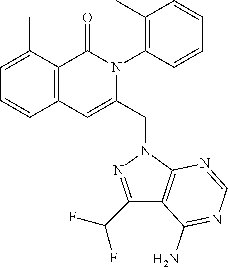 [SMILES: Cc1ccccc1-n1c(Cn2nc(C(F)F)c3c(N)ncnc32)cc2cccc(C)c2c1=O] | Compound 306 |
| 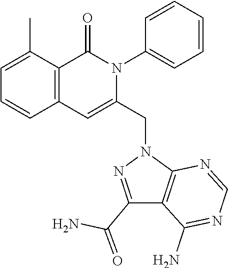 [SMILES: Cc1cccc2cc(Cn3nc(C(N)=O)c4c(N)ncnc43)n(-c3ccccc3)c(=O)c12] | Compound 307 |
| 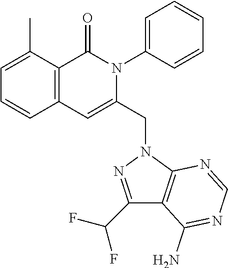 [SMILES: Cc1cccc2cc(Cn3nc(C(F)F)c4c(N)ncnc43)n(-c3ccccc3)c(=O)c12] | Compound 308 |
| 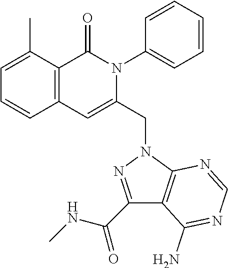 [SMILES: CNC(=O)c1nn(Cc2cc3cccc(C)c3c(=O)n2-c2ccccc2)c2ncnc(N)c12] | Compound 309 |
| 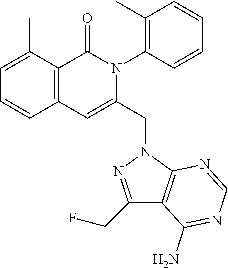 [SMILES: Cc1ccccc1-n1c(Cn2nc(CF)c3c(N)ncnc32)cc2cccc(C)c2c1=O] | Compound 310 |
| 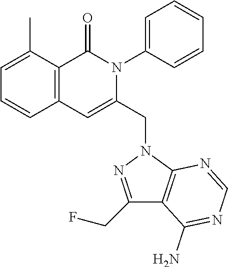 [SMILES: Cc1cccc2cc(Cn3nc(CF)c4c(N)ncnc43)n(-c3ccccc3)c(=O)c12] | Compound 311 |
| 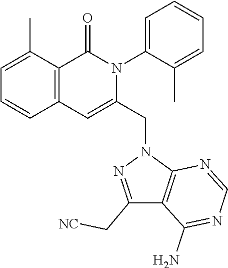 [SMILES: Cc1ccccc1-n1c(Cn2nc(CC#N)c3c(N)ncnc32)cc2cccc(C)c2c1=O] | Compound 312 |
| 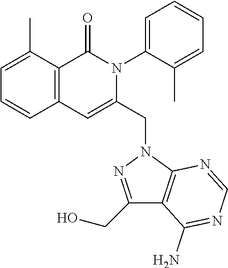 [SMILES: Cc1ccccc1-n1c(Cn2nc(CO)c3c(N)ncnc32)cc2cccc(C)c2c1=O] | Compound 313 |
| 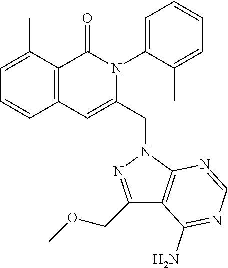 [SMILES: COCc1nn(Cc2cc3cccc(C)c3c(=O)n2-c2ccccc2C)c2ncnc(N)c12] | Compound 314 |
| 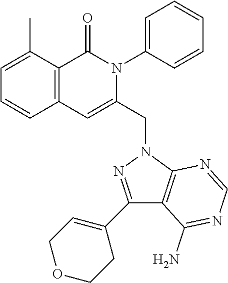 [SMILES: Cc1cccc2cc(Cn3nc(C4=CCOCC4)c4c(N)ncnc43)n(-c3ccccc3)c(=O)c12] | Compound 315 |
| 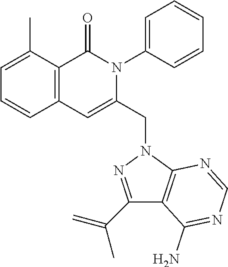 [SMILES: C=C(C)c1nn(Cc2cc3cccc(C)c3c(=O)n2-c2ccccc2)c2ncnc(N)c12] | Compound 316 |
| 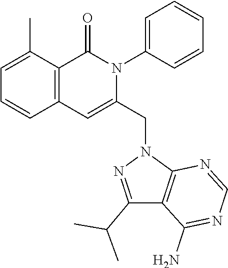 [SMILES: Cc1cccc2cc(Cn3nc(C(C)C)c4c(N)ncnc43)n(-c3ccccc3)c(=O)c12] | Compound 317 |
| 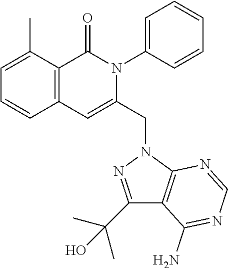 [SMILES: Cc1cccc2cc(Cn3nc(C(C)(C)O)c4c(N)ncnc43)n(-c3ccccc3)c(=O)c12] | Compound 318 |
| 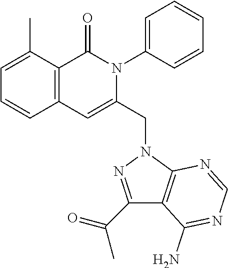 [SMILES: CC(=O)c1nn(Cc2cc3cccc(C)c3c(=O)n2-c2ccccc2)c2ncnc(N)c12] | Compound 319 |
| 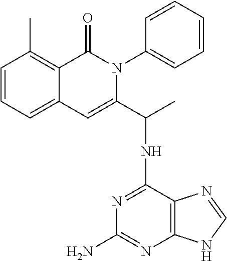 [SMILES: Cc1cccc2cc(C(C)Nc3nc(N)nc4[nH]cnc34)n(-c3ccccc3)c(=O)c12] | Compound 320 |
| 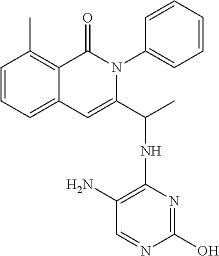 [SMILES: Cc1cccc2cc(C(C)Nc3nc(O)ncc3N)n(-c3ccccc3)c(=O)c12] | Compound 321 |
| 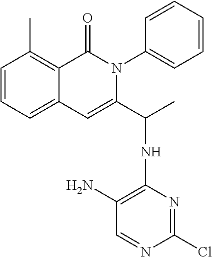 [SMILES: Cc1cccc2cc(C(C)Nc3nc(Cl)ncc3N)n(-c3ccccc3)c(=O)c12] | Compound 322 |
| 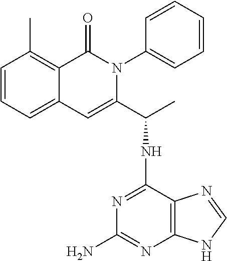 [SMILES: Cc1cccc2cc([C@H](C)Nc3nc(N)nc4[nH]cnc34)n(-c3ccccc3)c(=O)c12] | Compound 323 |
| 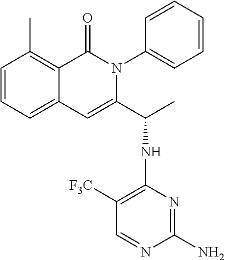 [SMILES: Cc1cccc2cc([C@H](C)Nc3nc(N)ncc3C(F)(F)F)n(-c3ccccc3)c(=O)c12] | Compound 324 |
| 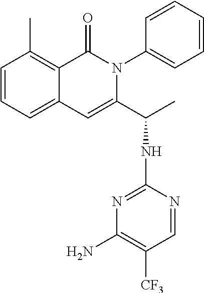 [SMILES: Cc1cccc2cc([C@H](C)Nc3ncc(C(F)(F)F)c(N)n3)n(-c3ccccc3)c(=O)c12] | Compound 325 |

### Example 2

### Expression and Inhibition Assays of p110α/p85α, p110β/p85α, p110δ/p85α, and p110γ

Class I PI3-Ks can be either purchased (p110α/p85α, p110β/p85α, p110δ/p85α from Upstate, and p110γ from Sigma) or expressed as previously described (Knight et al., 2004). IC50 values are measured using either a standard TLC assay for lipid kinase activity (described below) or a high-throughput membrane capture assay. Kinase reactions are performed by preparing a reaction mixture containing kinase, inhibitor (2% DMSO final concentration), buffer (25 mM HEPES, pH 7.4, 10 mM MgCl2), and freshly sonicated phosphatidylinositol (100 μg/ml). Reactions are initiated by the addition of ATP containing 10 μCi of γ-32P-ATP to a final concentration 10 or 100 μM and allowed to proceed for 5 minutes at room temperature. For TLC analysis, reactions are then terminated by the addition of 105 μl 1N HCl followed by 160 μl CHCl₃:MeOH (1:1). The biphasic mixture is vortexed, briefly centrifuged, and the organic phase is transferred to a new tube using a gel loading pipette tip precoated with CHCl₃. This extract is spotted on TLC plates and developed for 3-4 hours in a 65:35 solution of n-propanol:1M acetic acid. The TLC plates are then dried, exposed to a phosphorimager screen (Storm, Amersham), and quantitated. For each compound, kinase activity is measured at 10-12 inhibitor concentrations representing two-fold dilutions from the highest concentration tested (typically, 200 μM). For compounds showing significant activity, IC50 determinations are repeated two to four times, and the reported value is the average of these independent measurements.

Other commercial kits or systems for assaying PI3-K activities are available. The commercially available kits or systems can be used to screen for inhibitors and/or agonists of PI3-Ks including but not limited to PI 3-Kinase α, β, δ, and γ. An exemplary system is PI 3-Kinase (human) HTRF™ Assay from Upstate. The assay can be carried out according to the procedures suggested by the manufacturer. Briefly, the assay is a time resolved FRET assay that indirectly measures PIP3 product formed by the activity of a PI3-K. The kinase reaction is performed in a microtitre plate (e.g., a 384 well microtitre plate). The total reaction volume is approximately 20 ul per well. In the first step, each well receives 2 ul of test compound in 20% dimethylsulphoxide resulting in a 2% DMSO final concentration. Next, approximately 14.5 ul of a kinase/PIP2 mixture (diluted in 1× reaction buffer) is added per well for a final concentration of 0.25-0.3 ug/ml kinase and 10 uM PIP2. The plate is sealed and incubated for 15 minutes at room temperature. To start the reaction, 3.5 ul of ATP (diluted in 1× reaction buffer) is added per well for a final concentration of 10 uM ATP. The plate is sealed and incubated for 1 hour at room temperature. The reaction is stopped by adding 5 ul of Stop Solution per well and then 5 ul of Detection Mix is added per well. The plate is sealed, incubated for 1 hour at room temperature, and then read on an appropriate plate reader. Data is analyzed and IC50s are generated using GraphPad Prism® 5.

### Example 3

### Compound 292 Inhibits PI3K-δ, PI3K-γ, PI3K-β, and PI3K-α

The PI3K inhibitory activity of Compound 292 was tested in several assays described herein. The results are shown in Table 5 below, indicating that Compound 292 is a potent inhibitor of PI3K-δ and PI3K-γ. In these assays, Compound 292 inhibits PI3K-δ activity at lower doses compared to other PI3Ks (e.g., at least 10-fold lower dose compared to PI3K-γ, PI3K-β or PI3K-α).

TABLE 5
Biochemical and Cellular Comparison of Compound 292

| Compound 292 | PI3K-α | PI3K-β | PI3K-δ | PI3K-γ |
|---|---|---|---|---|
| Kᵢ | 25,900 pM | 1,564 pM | 23.2 pM | 243 pM |
| TLC IC₅₀ | 1602 nM | 85 nM | 2.5 nM | 27.4 nM |
| Cellular IC₅₀ | 1547 nM | 171 nM | 1 nM | 43 nM |

### Example 4

### Functional Cellular Activity of Compound 292

The functional cellular activities of Compound 292 were assessed. The results are shown in Table 6 below. Compound 292 suppressed murine B-cell proliferation and human B-cell proliferation at subnanomolar concentrations, with an EC₅₀ of 0.5 nM. Compound 292 suppressed human T-cell proliferation at nanomolar concentrations, with an EC₅₀ of 9.5 nM.

To determine PI3K-δ,γ isoform activity in vitro, Compound 292 was assessed in PI3K-δ and PI3K-γ selective cell-based assays. To assess the ability to inhibit the PI3K-δ isoform, AKT phosphorylation (T308) was measured by enzyme-linked immunosorbent assay (ELISA) in anti-IgM antibody-stimulated RAJI cells, a human Burkitt's lymphoma cell line, in the presence or absence of Compound 292. Compound 292 potently inhibited AKT phosphorylation with an IC₅₀ value of 2.0 nM. To assess the ability to inhibit the PI3K-γ isoform, the murine macrophage-like cell line, RAW 264.7, was stimulated with C5a, and the level of AKT phosphorylation (T308) was measured by ELISA. Compound 292 inhibited PI3K-γ in C5a activated RAW 264.7 cells with an IC₅₀ value of 44.0 nM. Compound 292 is a potent inhibitor of both PI3K-δ and PI3K-γ in isoform-selective cell-based assays.

TABLE 6
Compound 292 Functional Cellular Activity

| Functional Cellular Activity | EC₅₀ |
|---|---|
| Murine B-cell proliferation | 0.5 nM |
| Human B-cell proliferation | 0.5 nM |
| Human T-cell proliferation | 9.5 nM |
| PI3K-δ selective assay (RAJI cells, human | 2 nM |
| lymphoma cell line) | |
| PI3K-γ selective assay (RAW 264.7, murine | 44 nM |
| macrophage-like cell line) | |
| Anti-fCER1 BAT (delta) | 78 nM |

In one exemplary assay tested, Compound 292 potently inhibited PI3K-δ specific basophil activation in human whole blood with an IC₅₀ of 78 nM.

### Example 5

### Compound 292 Potently Inhibits Induction of IFN-α in Primary Human PBMCs

The role of PI3Ks in TLR signaling is not clear from the literature. For TLR-9 induced cytokines, PI3K inhibition has been called neutral, suppressive, and positive. Thus, the literature suggests that PI3K inhibition may not be effective in inhibiting IFN-α via TLR9. The present example demonstrates that PI3K inhibition by Compound 292 inhibits induction of IFN-α via TLR9.

Human PBMCs were stimulated with CPG-A. CPG-A selectively activates PDCs, inducing IFN-α production via TLR9. IFN-α is a PDC cell selective readout. Primary human PBMCs from two normal human donors were used as a source of PDC cells. 200 K cells per well were used. Cells were pretreated with the specified concentration of Compound 292 for 30 minutes. Then the cells were treated with the specified concentration of CPG-A for 16 hours. RPMI-5% serum was employed.

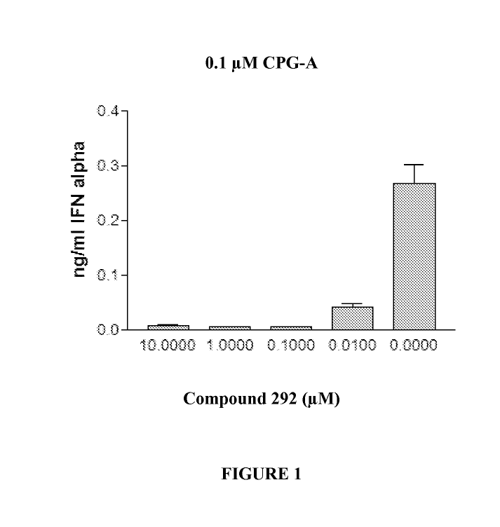

FIG. 1 shows that the extent of inhibition by Compound 292 depends on the extent of IFN-α induction, reflecting the positive feedback of IFN-α on itself. Compound 292 potently inhibited induction of IFN-α induction via TLR9, as demonstrated with 0.1 μM CPG-A induction and a 30 minute pre-incubation period and 0.2 μM CPG-A induction. These data show that PI3K delta and/or gamma are important in the induction of IFN-α in PDCs. Furthermore, PI3K inhibition with Compound 292 blocked the induction IFN-α in PDCs and can thus have therapeutic benefit in the treatment of rheumatoid arthritis or asthma (e.g., severe/refractory asthma).

### Example 6

### Effects of Compound 292 on Induced Cytokine Release

Using methods analogous to those presented in Example 5, the effects of Compound 292 were investigated using CPG-A and other TLR ligands, including LPS, PAM2CSK4, and R848 to induce release of cytokines, including IFN-α, IL-1, IL-6, IL-8, and TNF. These experiments were conducted using PBMCs.

CPGA induced Cytokines

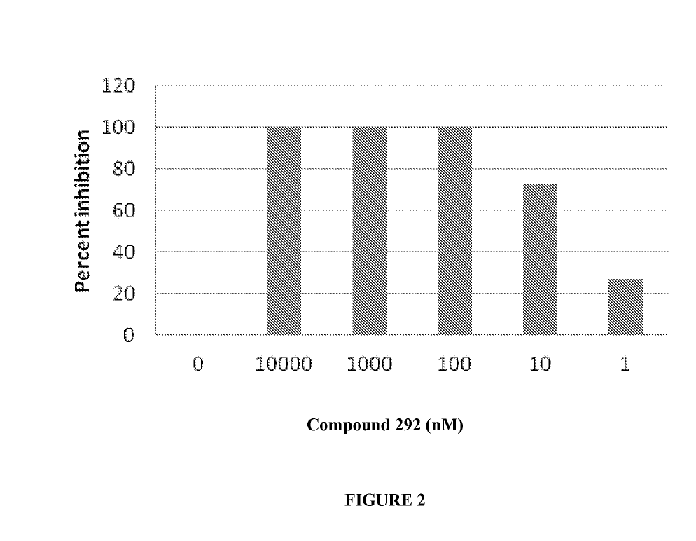

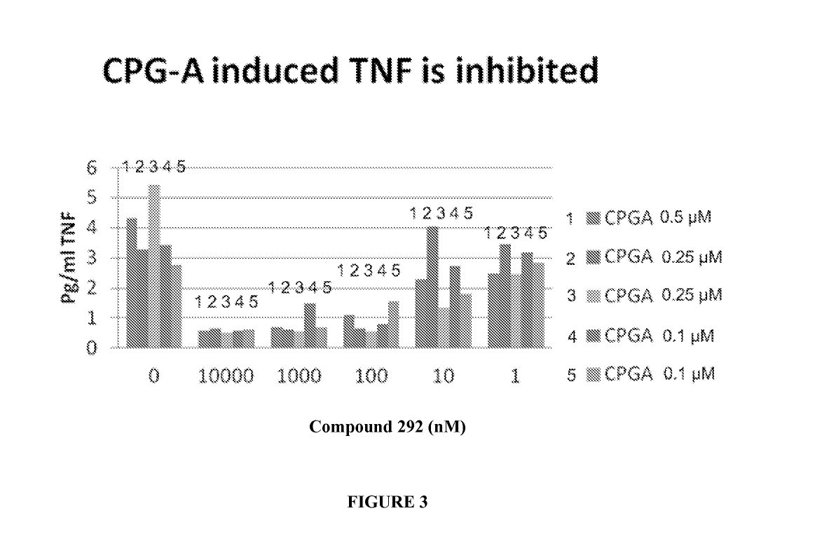

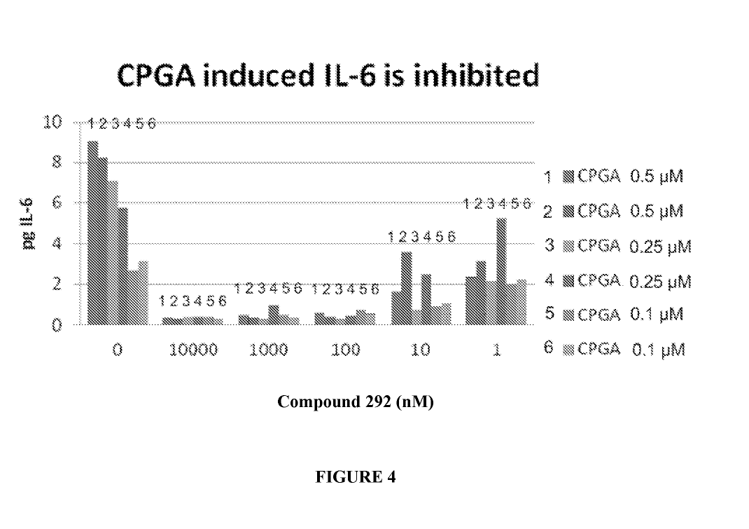

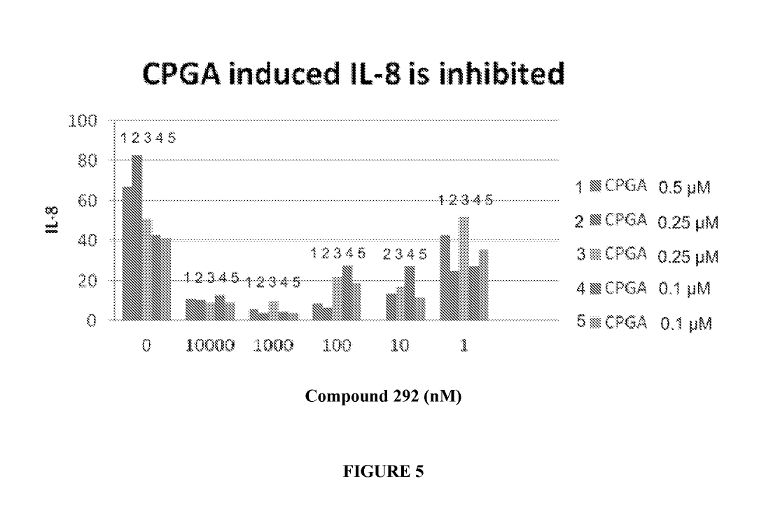

Consistent with the results provided in the previous example, Compound 292 blocked CPG-A induced IFN-α. See FIG. 2, which shows the results as percent inhibition (all samples combined). Compound 292 also inhibited CPG-A induced TNF-α (see FIG. 3), IL-6 (see FIG. 4), and IL-8 (see FIG. 5). CPG-A did not detectably induce IL-1.

### PAM2CSK4 Induced Cytokines

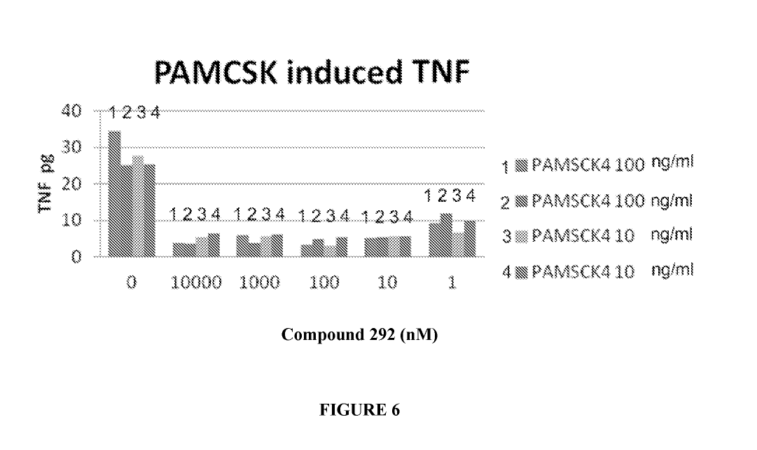

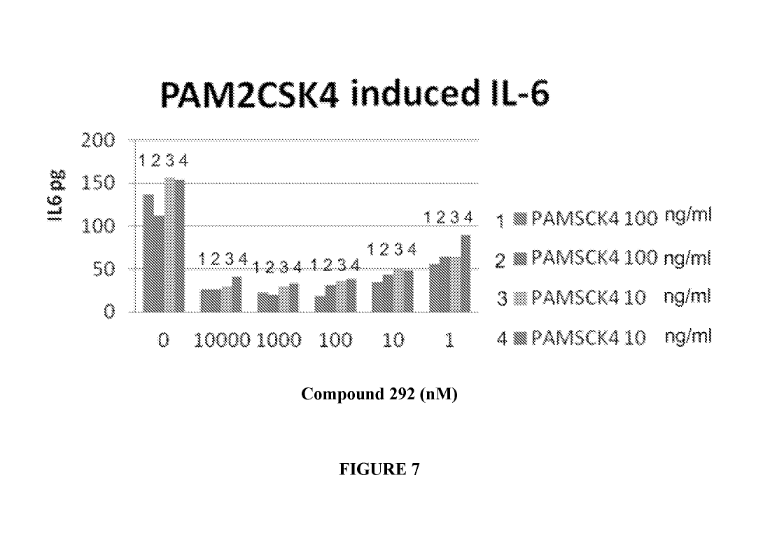

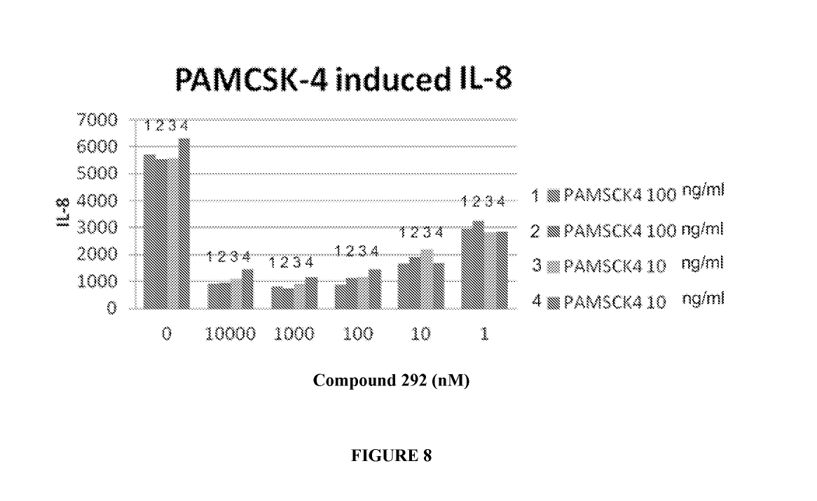

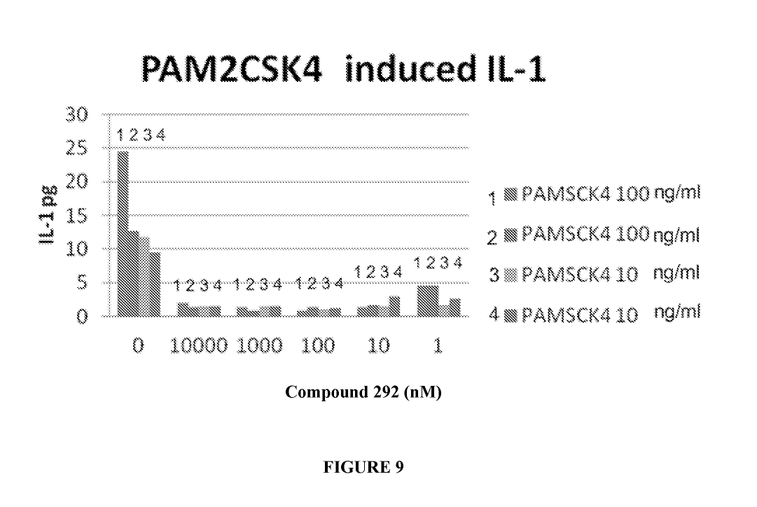

PAM2CSK4 signals through TL2/TLR6, which plays a role in atherosclerosis, colitis, ischemic injury, e.g., cardiac events and stroke. Compound 292 inhibited PAM2CSK4 induced TNF-α (see FIG. 6), IL-6 (see FIG. 7), IL-8 (see FIG. 8), and IL-1 (see FIG. 9). PAM2CSK4 treatment did not detectably induce IFN-α production (data not shown).

### R848 Induced Cytokines

Compound 292 did not detectably affect R848 induced IFN-α, TNF-α, IL-6, or IL-8. Compound 292 slightly inhibited R848 induced IL-1.

### LPS Induced Cytokines

Compound 292 enhanced the production of LPS induced TNF-α, IL-6, and IL-1. Compound 292 did not detectably affect the production of LPS induced IL-8. LPS treatment did not detectably induce IFN-α production (data not shown).

The results described above are summarized in Table 7. These results show that CPGA induced cytokines, including IFN-α, IL-6, IL-8, and TNF-α were inhibited by Compound 292. Similarly, PAM2CSK4 induced cytokines, including TNF-α, IL-6, IL-8, and ILL were inhibited by Compound 292. R848 induced cytokines were not affected by Compound 292 to the extent that CPGA or PAM2CSK4 induced cytokines were. LPS induced IL-1, IL6, and TNF-α were increased by Compound 292, whereas LPS-induced IL-8 was not affected.

TABLE 7
Summary of observed effects of Compound 292 on induced
cytokine production

| TLR ligand | | | | | |
|---|---|---|---|---|---|
| inducer | IFN-α | TNF-α | IL-6 | IL-8 | IL-1 |
| CPG-A | Inhibited; | Inhibited | Inhibited | Inhibited | N/A: no |
| | FIG. 2 | FIG. 3 | FIG. 4 | FIG. 5 | induction |
| PAMCSK | N/A: no | Inhibited | Inhibited | Inhibited | Inhibited |
| | induction | FIG. 6 | FIG. 7 | FIG. 8 | FIG. 9 |

### Example 7

### Effects of Compound 292 in the Rat Collagen-Induced Arthritis Model

Rat collagen induced arthritis is an experimental model of polyarthritis that has been widely used for nonclinical testing of numerous anti-arthritic agents that are either under nonclinical or clinical investigation or are currently used as therapeutics in this disease. The hallmarks of this model are reliable onset and progression of robust, easily measurable, polyarticular inflammation, marked cartilage destruction in association with pannus formation and mild to moderate bone resorption and periosteal bone proliferation. Therapeutic agents that inhibit interleukin-1 (IL-1) production or activity are especially active in this test system but other types of anti-inflammatory agents have good to excellent activity.

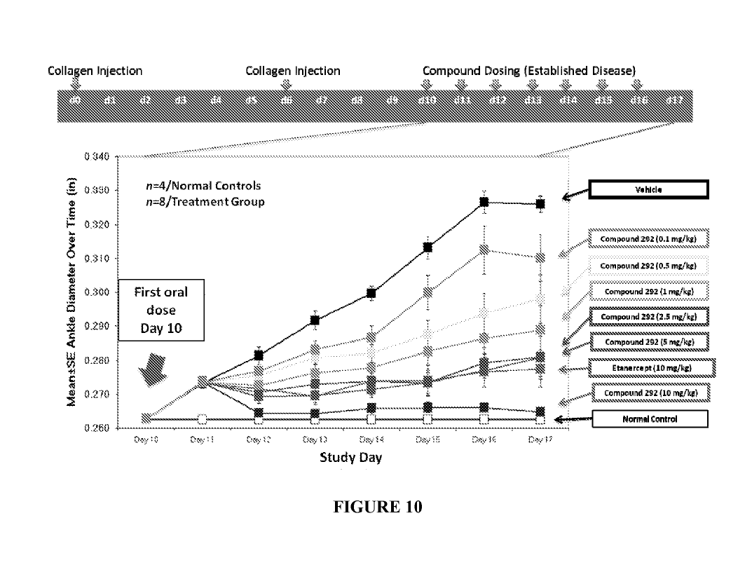

To assess the anti-inflammatory action of Compound 292, female Lewis rats with established type II collagen-induced arthritis were treated orally (PO) with Compound 292 (0.1, 0.5, 1, 2.5, 5, or 10 mg/kg) or vehicle once daily (QD) for 7 days (days 10-16) after induction with type II collagen. Etanercept (10 mg/kg) was administered subcutaneously on days 10 and 13 as a positive control. The study was terminated on day 17. The results of the study are shown in FIG. 10.

Ankle diameter for vehicle treated rats peaked on day 16 and 17. At the end of the treatment period, the mean ankle diameter was significantly decreased for all active treatment groups compared to vehicle-treated diseased animals, except at the lowest dose of Compound 292 (0.1 mg/kg).

The area under the curve (AUC) from the mean ankle diameter over time profile was used as a parameter to evaluate the effect of Compound 292 treatment on ankle diameter over several days of dosing. For each dose group, the percent reduction in the AUC relative to vehicle-treated, diseased animals was determined Across the 0.1 to 10 mg/kg dose range evaluated, reductions in the ankle diameter AUC ranged from 25% to 89% relative to vehicle controls. In the same study, treatment with etanercept (10 mg/kg) reduced ankle diameter AUC by 70% relative to animals treated with vehicle.

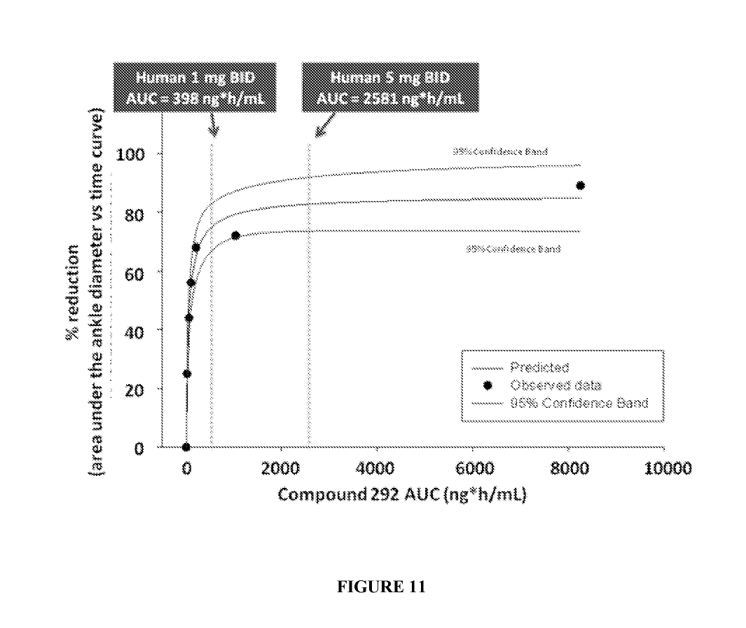

The correlation between the AUC of Compound 292 and the reductions in the ankle diameter AUC is shown in FIG. 11, which demonstrates that Compound 292 can achieve high percentage of reduction in the ankle diameter AUC (e.g., about 70%) at an AUC level of Compound 292 below the AUC level of Compound 292 after human 1 mg BID dosing and human 5 mg BID dosing.

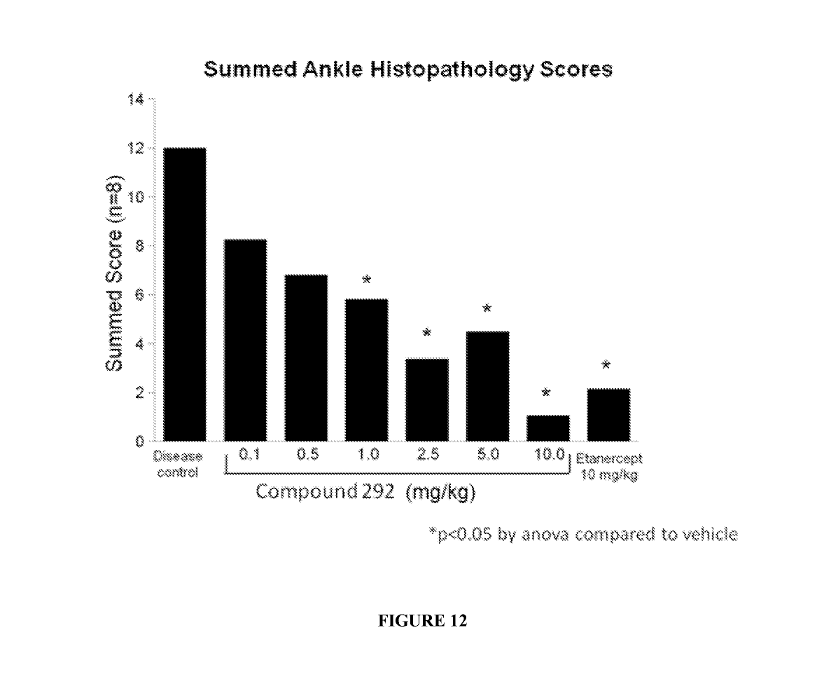

The summed ankle histological scores of the study are shown in FIG. 12, which demonstrates that Compound 292 prevented inflammation and protected joint bone and cartilage in the rat CIA model.

In rodents, it is expected that significant PI3K-δ inhibition would increase immunoglobulin (Ig) class switching to IgE resulting in increased plasma levels of IgE. Significant increases in serum IgE levels were observed in rats treated with etanercept and Compound 292, as compared to vehicle controls.

In conclusion, daily oral treatment with Compound 292 displayed dose-dependent beneficial effects on the parameters associated with established type II collagen-induced arthritis in rats.

### Example 8

### Effects of Compound 292, in Combination with Methotrexate or Etanercept, in the Rat Collagen-Induced Arthritis Model

The purpose of this study was to determine the efficacy of oral Compound 292 administered daily, alone or in combination with methotrexate (MTX) (QD, 0.06 mg/kg days 0-20) or etanercept (subcutaneously (SC), 1 mg/kg days 10, 13, 16 and 19) for inhibition of the inflammation (paw swelling), cartilage destruction and bone resorption that occurs in developing or established type II collagen arthritis in rats.

Female Lewis rats with type II collagen arthritis were treated PO with vehicle 1 (5% NMP, 10% Solutol, 85% PEG400) or QD with Compound 292 (2.5, 5 or 50 mg/kg) on days 0-20 (developing disease) or days 10-20 (established disease). Treatment was administered either alone or in combination with the reference compounds MTX, or etanercept. All rats were administered MTX, vehicle 2 (1% CMC in water), or vehicle 1 on days 0-20 in order to keep dose manipulations consistent for all groups. Animals were terminated on day 21. Efficacy evaluation was based on daily ankle caliper measurements, ankle diameter expressed as area under the curve (AUC), and terminal hind paw weights.

Daily ankle diameter measurements for vehicle treated rats peaked on day 16, and then declined slightly through day 18 before increasing again through day 20, as is commonly seen when this model is extended beyond 17 days. Daily ankle diameter measurements were significantly decreased toward normal for rats in all treatment groups as compared to vehicle treated control rats. When compared to MTX or etanercept treated rats, daily ankle diameter measurements were significantly reduced by all combinations tested.

In conclusion, results of this study indicated that daily oral treatment with Compound 292 (2.5, 5 or 50 mg/kg) administered alone or in combination with MTX or etanercept was well tolerated and had beneficial effects on the parameters associated with developing and established type II collagen arthritis in rats.

### Example 9

### Effects of Compound 292 in Freund's Complete Adjuvant Induced Rat Model of Arthritis

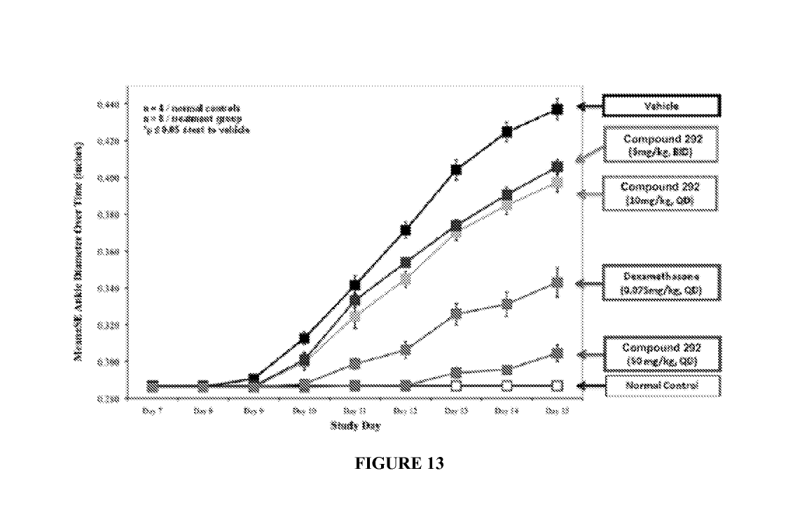

Freund's complete adjuvant induced arthritis in the rat is an experimental model of polyarthritis that has been widely used for nonclinical testing of numerous anti-arthritic agents that are either under nonclinical or clinical investigation, or are currently used as therapeutics in this disease. Therefore, a study was conducted to determine the dose responsive efficacy of Compound 292 administered orally either daily (10 or 50 mg/kg) or twice daily (5 mg/kg) for 10 days (days 5-14) in inhibiting the periarticular inflammation and bone resorption of developing adjuvant arthritis. The results of the study are shown in FIG. 13.

Daily ankle diameter measurements were significantly reduced towards normal for rats in all treatment groups, with 50 mg/kg Compound 292 more effective then dexamethasone at reducing ankle diameter over time. Significant and dose-responsive inhibition of ankle diameter AUC (d7-14) increase was seen in rats treated with 50 mg/kg Compound 292 QD (95% inhibition), 10 mg/kg Compound 292 QD (31%), 5 mg/kg Compound 292 twice a day (BID, 24%) or dexamethasone (71%) as compared to vehicle treated controls.

Histopathologic bone resorption was significantly and dose-responsively reduced toward normal for rats treated with Compound 292 50 mg/kg QD (100% reduction), 10 mg/kg QD (67%), 5 mg/kg BID (72%), or dexamethasone (97%) as compared to vehicle treated controls. Histopathologic inflammation was significantly and dose-responsively reduced toward normal for rats treated with 50 mg/kg Compound 292 QD (88% reduction), 10 mg/kg Compound 292 QD (15%), 5 mg/kg Compound 292 BID (18%), or dexamethasone (56%) as compared to vehicle treated controls. Finally, histopathologic summed scores and ankle measurements were significantly and dose-responsively reduced toward normal for rats treated with Compound 292 or dexamethasone as compared to vehicle treated controls.

Final paw weights were significantly and dose-responsively reduced toward normal for rats treated with 50 mg/kg Compound 292 QD (98% reduction), 10 mg/kg Compound 292 QD (23%), 5 mg/kg Compound 292 BID (30%), or dexamethasone as compared to vehicle treated controls.

Results of this study indicate that once or twice daily oral treatment with Compound 292 (10 or 50 mg/kg QD, or 5 mg/kg BID) effectively and dose-responsively inhibited the parameters associated with developing adjuvant arthritis. Results of QD treatment with 10 mg/kg Compound 292, and BID treatment with 5 mg/kg Compound 292 were similar.

### Example 10

### Effects of Compound 292 in Rat PG-PS Model

### Systemic Arthritis Model

All injections are performed under anesthesia. 60 female Lewis rats (150-170) are anesthetized by inhalation isoflurane using a small animal anesthesia machine. The animals are placed in the induction chamber until anesthetized by delivery of 4-5% isoflurane in 02 and then held in that state using a nose cone on the procedure table. Maintenance level of isoflurane is at 1-2%. Animals are injected intraperitoneally (i.p.) with a single injection of purified PG-PS 105 Group A, D58 strain (concentration 25 μg/g of bodyweight) suspended in sterile 0.85% saline. Each animal receives a total volume of 500 microliters administered in the lower left quadrant of the abdomen using a 1 milliliter syringe with a 23 gauge needle. Placement of the needle is critical to avoid injecting the PG-PS 105 into either the stomach or caecum. Animals are under continuous observation until fully recovered from anesthesia and moving about the cage. An acute response of a sharp increase in ankle measurement, typically 20% above baseline measurement can peak in 3-5 days post injection. Treatment with test compounds can be PO, SC, IV or IP. Rats are dosed no more than two times in a 24 hour time span. Treatment can begin on day 0 or any day after that through day 30. The animals are weighed on days 0, 1, 2, 3, 4, 5, 6, 7 and beginning again on day 12-30 or until the study is terminated. Paw/ankle diameter is measured with a digital caliper on the left and right side on day 0 prior to injection and again on day 1, 2, 3, 4, 5, 6 and 7. On day 12, measurements begin again and continue on through day 30. At this time, animals can be anesthetized with isoflurane, as described above, and terminal blood samples can be obtained by tail vein draws for the evaluation of the compound blood levels, clinical chemistry or hematology parameters. Animals are them euthanized with carbon dioxide overdose. A thoracotomy can be conducted as a means of death verification.

### Monoarticular Arthritis Model

Animals (5-10/group), housed 5/cage, are anesthetized with Isoflurane and injected with 20 μl of PGPS (0.125 mg/ml, total) in the right ankle on day-14 (initiation of priming), after at least 3 days of acclimation. Animals are initially randomized into groups based on body weight but any with no ankle swelling following priming are eliminated from the groups to be reactivated. 10/group are retained into the primary treatment groups. The remaining animals are the baseline controls (Group 1).

Rats that have ankle inflammation are allowed to recover for two weeks, and then the swelling is reactivated (day 0) by a tail vein injection of 0.5 ml of 0.4 mg/ml PG-PS. Treatment with the test compounds is given QD the day prior to reactivation on day 0 and then daily.

Pain testing occurs at 3 times, prior to reactivation to establish baseline (day −3), on day 1 (24 hrs post reactivation), day 2 (48 hrs post-reactivation) and on day 4 (prior to termination).

Pain response testing using the Incapacitance (IC) test is done (after a training event) on d(−)3 (prior to dosing and reactivation-day 0) to determine what amount of residual discomfort is present prior to reactivation. Pain response testing using the Incapacitance meter occurs on day 1 (approximately 24 hrs post-reactivation on day 0), day 2 (48 hrs post-reactivation) and 4 with the test occurring 2 hrs post dose of the test articles Animals are placed in the plexiglass housing of the incapacitance meter and allowed to acclimate for approximately 2-5 minutes or until the rat appears to be calmly standing with both feet on the force plates. The position of the animal is such that each hind paw rests on a separate force plate. The force exerted by each hind paw is averaged over a 1 second interval, and the mean of three readings constitutes 1 data point. The change in hind paw weight distribution is determined by the difference in the amount of weight between the right and left limbs (expressed as a percent of the combined total). See, S. E. Bove et al., MIA-induced changes in weight bearing, Osteoarthritis and Cartilage Vol. 11, No. 11.

Gait analysis is done on days −3, 1, 2 and 4 according to the following methods: Rear feet of rats are placed in colored ink and black ink is applied to the dorsal side of the foot on the suspected painful leg. Rats are placed on paper and allowed to walk the full length then removed. Abnormalities are scored and then group means compared using some appropriate statistical method with p≦0.05 as significant.
0=Normal, equal ink staining on both feet
1=Slight limp, toe staining evident and some heel staining for all steps, no carrying or dragging. If left has very little heel staining (rat walks mainly on toes) then slightly less toe staining in right leg for most steps. (or approximately 25% less staining on right vs left)
2=Limping, toes only staining for all steps, no carrying or dragging. If left has very little heel staining (rat walks mainly on toes) then slightly less toe staining in right leg for most steps. (or approximately 50% less staining on right vs left)
3=Dragging and carrying leg, black drag marks from dorsal side of foot present or some attempt to use right as evidenced by minimal toe staining in at least one print, may pause and place right foot down for a single step. (or approximately 75% less staining on right vs left)
4=Carrying leg entire time, no staining from painful leg or only minor black drag marks, no toe staining in any print. (100% less staining on right vs left)

Rats may be re-randomized based on pre-reactivation pain response data in order to insure that groups are similar prior to reactivation.

Rats are dosed QD with compounds (vehicle or drug) on days 0-4. Reactivation will occur on day 0 and pain testing will be 2 hrs post-dose on day 1, 2 and 4.

Animals have caliper measures and paw volumes taken of ankles on day −3 (prior to reactivation on day 0 in the AM) and then on days 1, 2, 3, 4 (2 hrs post-dose) to determine if any anti-inflammatory effects are present in treated rats.

Animals are then anesthetized for serum collection and then euthanized and right and left hind paws transected at the medial and lateral malleolus and weighed for group comparisons of final paw weights. Paws are collected for potential histopathology.

Processing of Joints (Extent to be determined after live phase is completed): Following 5-7 days in 5% formic acid decalcifier, ankles or knees are cut in the sagittal (ankles) or frontal (knees) plane into 2 approximately equal halves and processed for paraffin embedding, then section and stained with T. Blue.

Scoring of Joints: PGPS ankles or knees are given scores of 0-5 for inflammation and bone resorption according to the following criteria:

### Bone Resorption:

0=Normal
0.5=Score reserved for those that are normal on low magnification but have the earliest hint of small areas of resorption in the metaphysis with no resorption in the tarsal bones
1=Minimal=small definite areas of resorption in distal tibial trabecular or cortical bone, or in the tarsal bones, not readily apparent on low magnification, rare osteoclasts
2=Mild=more numerous areas (≦25% loss of bone in growth plate area) of resorption in distal tibial trabecular or cortical bone and tarsals apparent on low magnification, osteoclasts more numerous
3=Moderate=obvious resorption of medullary trabecular and cortical bone without full thickness defects in both distal tibial cortices, loss of some medullary trabeculae with 26-50% loss across growth plate and cortices, some loss in tarsal bones, lesion apparent on low magnification, osteoclasts more numerous
4=Marked=Full or near full thickness defects in both distal tibial cortices, often with distortion of profile of remaining cortical surface, marked loss of medullary bone of distal tibia (50-100% loss across growth plate area and cortices and up to 50% loss in small tarsals if minor in tibia), numerous osteoclasts, minor to mild resorption in smaller tarsal bones
5=Severe=Full thickness defects in both distal tibial cortices with >75% loss across growth plate and both cortices and >50% loss in tarsals, often with distortion of profile of remaining cortical surface, marked loss of medullary bone of distal tibia, numerous osteoclasts

### Inflammation:

0=Normal (dorsal to ventral skin measures approx. 80-100 units on 16×, 5040-6300 μm)
0.5=Minimal generally focal infiltration of inflammatory cells in periarticular tissues (dorsal to ventral skin measures approx. 80-100 units on 16×, 5040-6300 m)
1=Minimal infiltration of inflammatory cells in periarticular tissue (dorsal to ventral skin measures approx. 80-100 units on 16×, 5040-6300 m)
2=Mild infiltration (dorsal to ventral skin measures approx. 100-120 units on 16×, 6300-7560 μm)
3=Moderate infiltration with moderate edema (dorsal to ventral skin measures approx. 121-130 units on 16×, 7623-8190 m)
4=Marked infiltration with marked edema (dorsal to ventral skin measures approx. 131-140 units on 16×, 8253-8820 μm)
5=Severe infiltration with severe edema, (dorsal to ventral skin measures approx. 141 or greater units on 16×, 8883- or more μm)

Measurements are taken from the dorsal skin surface (in flexion angle) to ventral skin surface (across the tarsal joints) in an attempt to semiquantitate the inflammatory edema.

Pannus and cartilage damage are scored as follows:

### Pannus:

0=Normal
0.5=Minimal, focal or only few marginal zones affected
1=Minimal infiltration of pannus in cartilage and subchondral bone, primarily affects marginal zones
2=Mild infiltration (<¼ of tibia or tarsals at marginal zones)
3=Moderate infiltration (¼ to ⅓ of tibia or small tarsals affected at marginal zones)
4=Marked infiltration (½-¾ of tibia or tarsals affected at marginal zones)
5=Severe infiltration (>¾ of tibia or tarsals affected at marginal zones, severe distortion of overall architecture)

### Cartilage Damage (Emphasis on Small Tarsals):

0=Normal
0.5=Minimal, focal or only few marginal zones affected
1=Minimal to mild loss of toluidine blue staining with no obvious chondrocyte loss or collagen disruption
2=Mild loss of toluidine blue staining with focal mild (superficial) chondrocyte loss and/or collagen disruption
3=Moderate loss of toluidine blue staining with multifocal moderate (depth to middle zone) chondrocyte loss and/or collagen disruption, smaller tarsals affected to ½-¾ depth with rare areas of full thickness loss
4=Marked loss of toluidine blue staining with multifocal marked (depth to deep zone) chondrocyte loss and/or collagen disruption, 1 or 2 small tarsals surfaces have full thickness loss of cartilage
5=Severe diffuse loss of toluidine blue staining with multifocal severe (depth to tide mark) chondrocyte loss and/or collagen disruption affecting more than 2 cartilage surfaces

Periosteal new bone formation may be scored/measured using criteria based on overall extent of this lesion as well as measurements taken from original periosteum to outer border of new bone on the ventral surface of the calcaneous in an area of non-tangential section thought to best represent the greatest area of increase.

0=None

1=100-300 μm

2=301-500 μm

3=501-700 μm

4=701-900 μm

5>1000 μm

Statistical Analysis: Data are analyzed using a Student's t-test or Mann-Whitney U test (non-parametric). If applicable, data are further analyzed across all groups using a one-way analysis of variance (1-way ANOVA) or Kruskal-Wallis test (non-parametric), along with the appropriate multiple comparison post-test. Unless indicated, Bolder BioPATH, Inc. performs statistical analysis on raw (untransformed) data only. Statistical tests make certain assumptions regarding the data's normality and homogeneity of variance, and further analysis may be required if testing resulted in violations of these assumptions. Significance for all tests is set at p≦0.05.

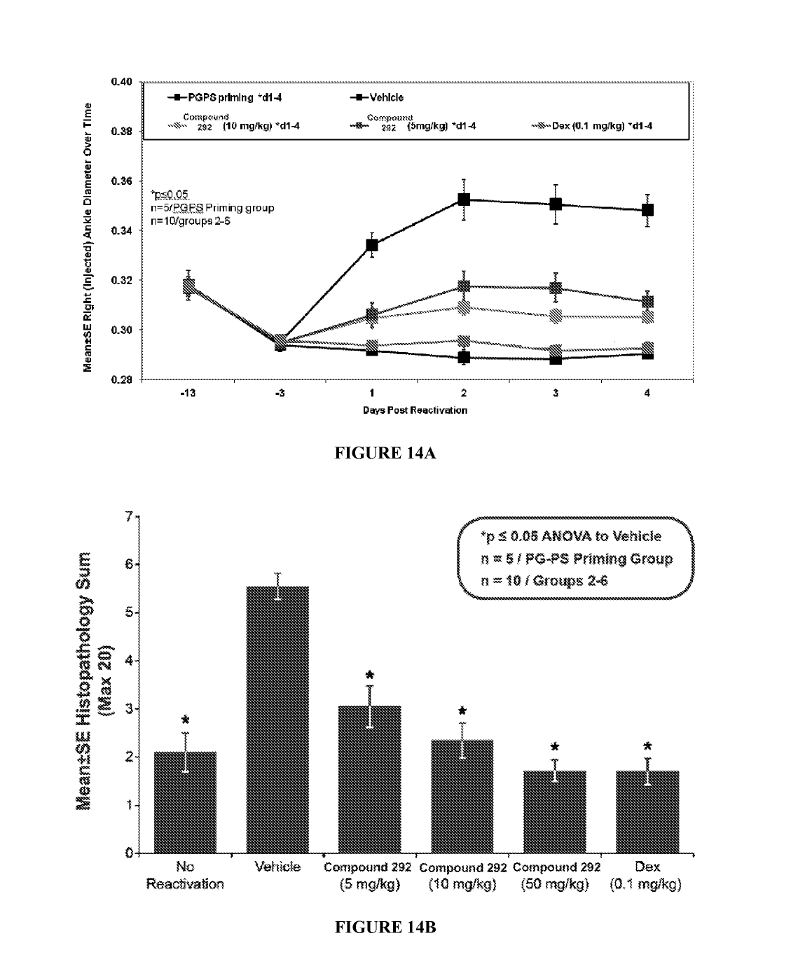

A study was conducted to determine the dose responsive efficacy of Compound 292 in rat mono-articular PG-PS model, as provided above. The results of the study are shown in FIG. 14A and FIG. 14B. FIG. 14A demonstrates that Compound 292 dose-dependently controlled inflammation when reactivated with PG-PS. FIG. 14B demonstrates that Compound 292 dose-dependently improved joint pathology in PG-PS model of arthritis.

### Example 11

### Effects of Compound 292 in Murine Model of Delayed Type Hypersensitivity

The anti-inflammatory properties of Compound 292 were examined in a murine model of DTH, a commonly used model of allergic contact dermatitis that is associated in large part with T-cell mediated immune responses Animals were sensitized on the plantar surface of the hind paw with 2,4-dinitrofluorobenzene (DNFB) on days 0 and 1. The animals were subsequently challenged with DNFB on the pinna of the ear on day 5 to initiate an inflammatory reaction that was measured 24 hours later. Compound 292 was administered orally at 0.3, 1, 3, and 10 mg/kg on day 5, 30 minutes prior to the challenge with DNFB Animals dosed with vehicle only or 5 mg/kg dexamethasone (Dex) served as negative and positive controls, respectively. Dose dependent inhibition of ear swelling by Compound 292 was observed. The highest dose group exhibited approximately 50% inhibition when compared to the vehicle control animals, and the effect was similar to that observed with dexamethasone.

### Example 12

### Effects of Compound 292 in Murine Model of LPS-Induced Lung Inflammation

The ability of Compound 292 to reduce LPS-induced pulmonary inflammation was evaluated in mice. Compound 292 was administered orally at 3, 10, 30, and 60 mg/kg, 60 minutes prior to intranasal instillation of LPS. Animals dosed with vehicle only or 5 mg/kg dexamethasone served as study controls. Multiple parameters of pulmonary inflammation were determined 6 h after LPS challenge. Instillation of LPS induced significant influx of leukocytes, mostly neutrophils, into the airways, as assessed by counting the number of cells in the bronchoalveolar lavage (BAL) fluid. Treatment with 3 mg/kg Compound 292 inhibited LPS-induced total leukocyte and neutrophil influx in BAL fluid, as effectively as dexamethasone at 5 mg/kg. These data demonstrate that LPS-induced neutrophil influx in BAL fluid is strongly inhibited by Compound 292.

### Example 13

### Effects of Compound 292 in Ovalbumin-Induced Murine Model of Asthma

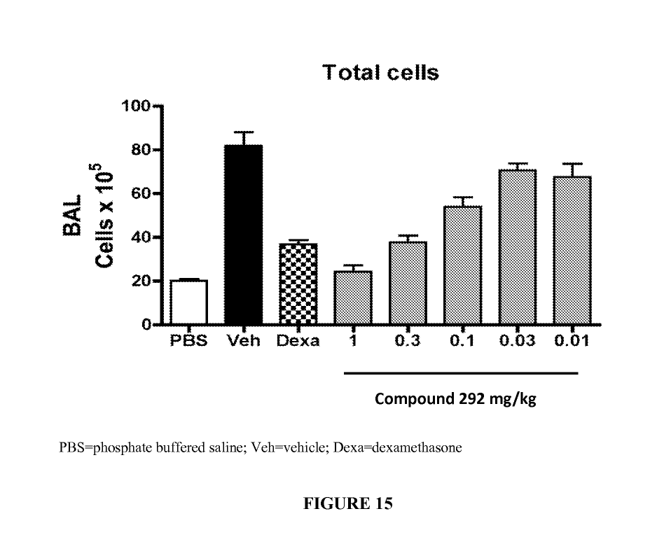

The ability of Compound 292 to prevent lung inflammation was examined in a murine model of allergic asthma. Pulmonary inflammation was measured 4 days after 4 consecutive daily intranasal instillations of ovalbumin in mice previously sensitized with ovalbumin. Compound 292 (0.03, 0.1, 0.3, 1, 3, and 10 mg/kg) was administered by oral gavage 30 minutes prior to each of the 4 ovalbumin challenges Animals dosed daily with vehicle only or 5 mg/kg dexamethasone served as study controls. Instillation of ovalbumin induced a significant influx of leukocytes, primarily eosinophils, into the airways, as determined by counting the number of cells present in the BAL fluid. Compound 292 significantly blocked eosinophil influx into the airway space at all doses tested (0.03 to 10 mg/kg). This reduction was mirrored by an effect on the total number of infiltrating leukocytes into the BAL fluid (FIG. 15). Compound 292 also reduced both lymphocyte and monocyte accumulation in BAL fluid induced by ovalbumin.

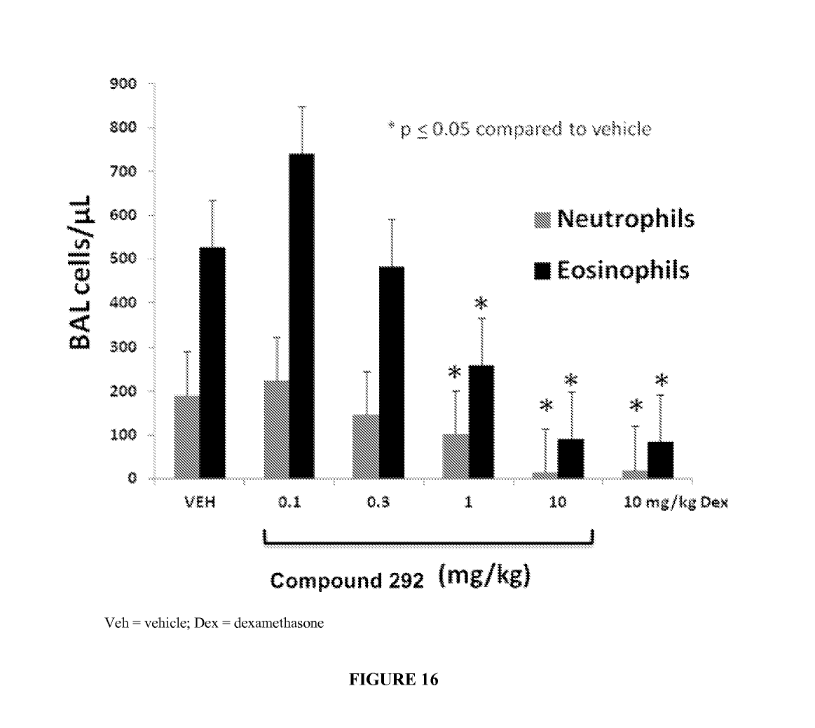

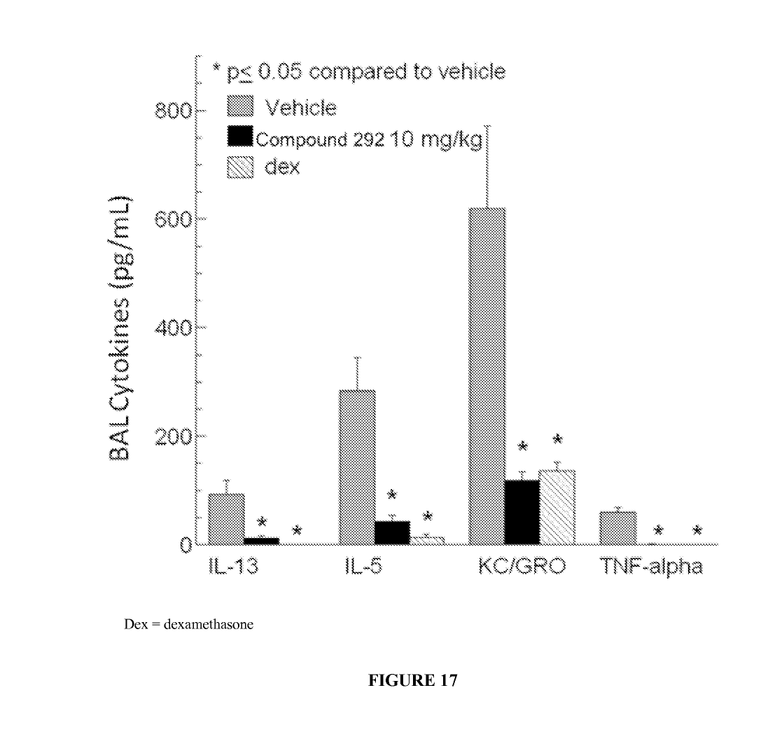

The ability of Compound 292 to suppress cellular inflammation was also examined in a rat ovalbumin allergic asthma model. Pulmonary inflammation was measured after intranasal instillations of ovalbumin in rat previously sensitized with ovalbumin. Compound 292 (0.1, 0.3, 1, and 10 mg/kg) was administered by oral gavage 60 minutes prior to re-challenge by ovalbumin, and BAL fluid analysis was performed 48 hours after the re-challenge. Animals dosed with vehicle only or 10 mg/kg dexamethasone served as study controls. Instillation of ovalbumin induced a significant influx of leukocytes, primarily neutrophils and eosinophils, into the airways, as determined by counting the number of cells present in the BAL fluid. Compound 292 significantly reduced neutrophils and eosinophils in the bronchoalveolar lavage (BAL) after 1 and 10 mg/kg doses (FIG. 16). At 10 mg/kg of Compound 292, the inhibition of neutrophil and eosinophil infiltration was equivalent to the positive control, 10 mg/kg of dexamethasone (FIG. 16). Levels of TNF-α, KC/GRO, IL-13, and IL-5 in the BAL were significantly suppressed by 10 mg/kg of Compound 292 treatment, suggesting that reductions in these inflammatory mediators could be related to the efficacy observed in this model (FIG. 17).

### Example 14

### Effects of PI3K-γ and/or PI3K-δ Inhibitors on Neutrophil Migration into Rat Air Pouches

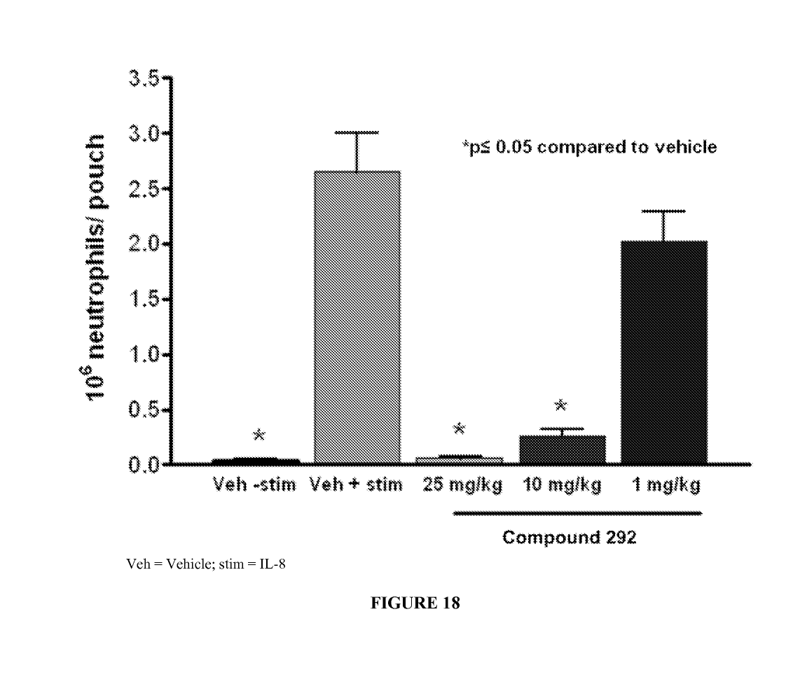

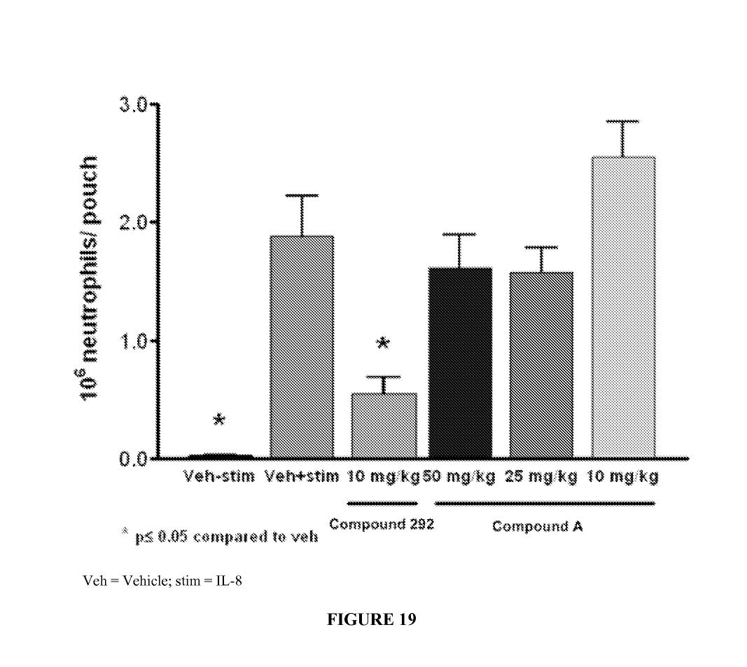

The ability of PI3K-δ,γ inhibitors to block leukocyte (e.g., neutrophil) migration was examined in a rat air pouch model. Air pouches were created by subcutaneous air injections into the back of the rats on Days 0 and 3. On Day 6, Compound 292 (1, 10, and 25 mg/kg) was administered orally to the animals at time 0 hour. IL-8 was injected into the pouch at 1 hour after the administration of Compound 292, and a PK sample was also collected. Lavage fluid was collected from the pouch at 5 hours after administration of Compound 292, and PK sample was collected again. Animals dosed with vehicle only, with or without IL-8 stimulation, served as study controls. Stimulation with IL-8 induced a significant influx of neutrophils into the air pouches, as determined by counting the number of cells present in the pouch. Compound 292 significantly blocked neutrophil influx into the pouches at doses of 10 and 25 mg/kg (FIG. 18), but only slightly at dose of 1 mg/kg. Based on the PK data (not shown) and Kᵢ values (see Table 5), Compound 292 at doses of 10 and 25 mg/kg inhibited both the PI3K-γ and PI3K-δ isoforms, while Compound 292 at dose of 1 mg/kg inhibited PI3K-δ, but not PI3K-γ. Compound A, a PI3K-δ selective inhibitor (PI3K-α Kᵢ=17700 pM, PI3K-β Kᵢ=18046 pM, PI3K-δ Kᵢ=2.1 pM, and PI3K-γ Kᵢ=1908 pM) was also tested in the rat air pouch model described above. It was found that Compound A did not inhibit neutrophil migration into air pouches stimulated with IL-8 at any of the doses tested (10, 25, and 50 mg/kg) (FIG. 19). These data indicate that the inhibition of neutrophil migration into rat air pouches stimulated with IL-8 is PI3K-γ dependent, but may be independent of PI3K-δ. These data also indicate that the assay described above is useful for screening compounds for anti-inflammatory or anti-asthma agent such as PI3K-γ selective inhibitor.

### Example 15

### Compound 292 Activity in PI3K-δ and PI3K-γ Mediated Human Whole Blood Assays

The PI3K pathway plays a critical role in the activation of basophils by relaying signals from cell surface receptors to downstream mediators. Whereas stimulation via the IgE Fc receptor by addition of anti-FcεR1 antibody occurs through PI3K-δ, stimulation with formyl-Methionyl-Leucyl-Phenylalanine (fMLP) occurs primarily through PI3K-γ. Using these two basophil stimuli in whole blood, Compound 292 inhibited PI3K-δ-specific degranulation of basophils with an average IC₅₀ of 96.1 nM, and PI3K-γ-specific degranulation with an average IC₅₀ of 1028 nM. The higher IC₅₀ in these whole blood assays compared to the isozyme-specific cellular assays (plasma protein-free), is consistent with protein binding determinations for Compound 292, which indicates it is 95% protein bound in human plasma. In addition, the effect of Compound 292 on PI3K-β function was determined in platelets using a thrombin peptide stimulus and measuring the inhibition of activated GPIIb/IIIa. The average IC₅₀ for Compound 292 in this PI3K-β specific assay was 4700 nM, indicating an about 4 fold window between PI3K-γ and PI3K-β inhibition in whole blood.

### Example 16

### Safety Pharmacology Studies of Compound 292

In Vitro hERG Assay

The in vitro effects of Compound 292 on the hERG channel current were examined as a surrogate for I_{Kr}, the rapidly activating, delayed rectifier cardiac potassium current. Compound 292 inhibited hERG current by 11.9% at 10 μM, 33.2% at 30 μM, 71.1% at 100 μM, and 92.8% at 300 μM compared to 0.9% in the vehicle control. The IC₅₀ value for the inhibitory effect of Compound 292 on hERG potassium current was 49.8 μM (Hill coefficient=1.3).

Compound 292 was highly bound in vitro to components of plasma of all species tested, including the rat, monkey, and human. In rat, monkey, and human plasma, Compound 292 was 85.8, 76.8, and 85.9% protein bound, respectively, at 100 μM (41700 ng/mL). The hERG assay was performed in a protein-free solution. Therefore, based on the free fractions, the IC₅₀ value of 49.8 μM (20800 ng/mL) for unbound Compound 292 would equate to total plasma concentrations of 351 μM (146200 ng/mL), 215 μM (89500 ng/mL), and 353 μM (147200 ng/mL) in rat, monkey, and human, respectively. These high concentrations suggest a very low potential for QT prolongation in humans.

### Neurofunctional Study in Sprague-Dawley Rat

This study was conducted to evaluate the potential effects of Compound 292 on the central nervous system following a single oral administration in male rats. During this study, a Functional Observation Battery (FOB) test and motor activity evaluation were performed pre-dose and at 2, 6, and 24 h following Compound 292 administration.

Compound 292, administered to male rats as a single oral dose up to 350 mg/kg, caused no changes in qualitative or quantitative FOB parameters up to 24 h post-dose. Significant decreases in locomotor activity were observed in animals tested 2 h after a 350 mg/kg dose. However, given that no concurrent effects on locomotor activity or arousal were noted in the FOB arena at the same time period, a definitive effect of Compound 292 could not be confirmed at these assessment intervals. No effects on the central nervous system were observed at dose levels ≦50 mg/kg.

### Respiratory Study in Sprague-Dawley Rat

This study was conducted to evaluate the potential effects of Compound 292 on the respiratory system following a single oral administration in the male rat. During this study, animals were placed in “head out” plethysmographs and respiratory parameters (tidal volume, respiratory rate, and derived minute volume) were measured for a period of approximately 30 minutes pre-dose, continuously from 1 to 3 h post-dose, and for 30-minute intervals at 6 and 24 h post-dose.

A single oral administration of Compound 292 at dose levels up to 350 mg/kg resulted in no Compound 292-related effects on respiratory parameters, including respiratory rate, tidal volume, and minute volume.

### Cardiovascular Study in Instrumented Cynomolgus Monkey

This study was conducted to evaluate the potential effects of Compound 292 on the hemodynamic and electrocardiographic parameters following a single oral administration to cynomolgus monkeys via telemetry. Four non-naive, male monkeys implanted with radiotelemetry transmitters were utilized during the conduct of this study.

No Compound 292-related effects were observed on hemodynamic or electrocardiographic parameters (arterial blood pressures (systolic, diastolic, mean and pulse pressure), heart rate, and quantitative electrocardiographic intervals (PR, QRS, QT and QTc)) following a single oral dose of 5, 30, and 150 mg/kg in male cynomolgus monkeys. In addition, no waveform abnormalities or arrhythmias related to the administration of Compound 292 up to 150 mg/kg were noted.

### Example 17

### Pharmacokinetics of Compound 292 in Animals

The absorption and pharmacokinetics of Compound 292 were investigated in absolute bioavailability studies in mice, rats, dogs, and monkeys. The results of these bioavailability studies are summarized in Table 8. The data demonstrate that Compound 292 was readily absorbed in a majority of the nonclinical test species when administered as a suspension formulation with oral bioavailability values of 57%, 40%, 40% and 7% in rats, monkeys, dogs and mice, respectively. The half-life of Compound 292 was 5 hrs in monkeys, 2 hrs in the dog, and less than 2 hrs in the rat and mouse. Compound 292 achieved a high volume of distribution and showed low to moderate clearance in monkey and rat. Binding of Compound 292 to plasma proteins was concentration and species dependent. Percent Compound 292 free in rat and monkey plasma was consistently higher than in human plasma at all concentrations tested. Distribution of Compound 292 into rat tissues was rapid and extensive based on the blood to tissue ratio being greater than 1 for a majority of tissues. Elimination of radiolabelled Compound 292 from tissues was also rapid with a majority of tissues without quantifiable levels of radioactivity at 24 hr.

TABLE 8
Compound 292 Pharmacokinetic Parameters in BALB/c Mice, Sprague-Dawley Rats, Beagle
Dogs and Cynomolgus Monkeys Following Intravenous and Oral Administration

| Species | # | | | | | | | | | | |
|---|---|---|---|---|---|---|---|---|---|---|---|
| (Report | animals/ | | Dose | Cₘₐₓ | Tₘₐₓ | AUC₀₋ₗₐₛₜ | AUC_{0-inf} | T_{1/2} | Cl | Vₛₛ | Fₒᵣₐₗ |
| Number) | gender | Route | (mg/kg) | (ng/mL) | (h) | (ng * h/mL) | (ng * h/mL) | (h) | (L/h/kg) | (L/kg) | (%) |
| Mouse | 27/M | IV^{c} | 10 | 5563 | 0.083 | 1900 | 1903 | 0.22 | 5.25 | 1.14 | — |
| | 27/M | PO^{d} | 10 | 390 | 0.083 | 136.8 | NC | NC | — | — | 7ⁱ |
| Rat | 3/M | IV^{c} | 2 | 1519 | 0.083 | 1153 | 1157 | 0.73 | 1.83 | 1.66 | — |
| | 3/M | PO^{d} | 10 | 785 | 1.2 | 2929 | 3298 | 2.4 | — | — | 57 |
| Dog | 3/M | IV^{e} | 0.5 | 4413^{a} | NC | 11738^{b} | 11921 | 2 | 0.051 | 0.13 | — |
| | 3/M | PO^{f} | 5 | 9597 | 3.00 | 105068^{b} | 107062 | 3.9 | — | — | 97^{g,i} |
| Dog | 3/M | IV^{e} | 1 | 1804^{a} | NC | 5875^{b} | 6268 | 1.83 | 0.194 | 0.493 | — |
| | 3/M | PO^{f} | 5 | 2367 | 1.33 | 10942^{b} | 13805 | 3.15 | — | — | 40^{h,i} |
| Monkey | 4/(2M, | IV^{c} | 1 | 1545 | 0.083 | 2357 | 2379 | 5.0 | 0.43 | 1.27 | — |
| | 2F) | | | | | | | | | | |
| | 4/(2M, | PO^{d} | 5 | 1327 | 1.5 | 4596 | 4685 | 5.4 | — | — | 40 |
| | 2F) | | | | | | | | | | |

— = not applicable
NC = not calculated
^{a}Reported value is C₀
^{b}AUC₀₋₂₄
^{c}IV formulation (mouse, rat, monkey) = 5% NMP, 10% Solutol ® HS 15, 30% PEG400, 55% water with 3% dextrose
^{d}PO formulation (mouse, rat, monkey) = 0.5% (w/v) low viscosity CMC and 0.05% (v/v) TWEEN ® 80 in ultra pure water
^{e}IV formulation (dog) = 5% 0.1N HCl, 5% PEG400 in 10% (2-hydroxypropyl)-β-cyclodextrin or 2.5% 1N HCl, 20% PEG400 in PBS
^{f}PO formulation (dog) = 5% NMP, 60% PEG400 and 35% water solution (ADME-11-008) or 5% NMP and 95% water suspension (ADME-11-009)
^{g}Fₒᵣₐₗ was calculated using 0.5 mg/kg IV dose as reference
^{h}Fₒᵣₐₗ was calculated using 1 mg/kg IV dose as reference
ⁱFₒᵣₐₗ was calculated using AUC₀₋ₗₐₛₜ

Membrane permeability and interaction of Compound 292 with human P-glycoprotein was assessed in vitro using Caco-2 cell monolayers. It was determined that Compound 292 has moderate cell membrane permeability, is a P-gp substrate and has the potential to inhibit the active transport of other P-gp substrates.

### Example 18

### Toxicology of Compound 292 in Animals

Single-dose toxicity study was conducted to determine the maximum tolerated dose (MTD) following a single oral dose and potential toxicity following 7-day repeat oral doses of Compound 292 in monkeys. It was determined that the MTD following a single oral administration of Compound 292 in monkeys was 500 mg/kg.

4- and 13-Week repeat-dose nonclinical safety studies were conducted in which rats and cynomolgus monkeys received daily Compound 292 doses by oral gavage. The no observed adverse effect level (NOAEL) in the 13-week rat study was 25 mg/kg/day (150 mg/m²/day) and the NOAEL in the 13-week monkey study was 5 mg/kg/day (60 mg/m²/day). On Day 91, the mean AUC₀₋₂₄ hr values for combined sexes at the NOAELs were 14150 ng*h/mL in the rat, and 4015 ng*h/mL in the monkey. Based on PK data from the clinical study in healthy subjects, exposure in humans following repeated oral doses of 5 mg BID Compound 292 (mean AUC₀₋₂₄ hr=2582 ng-h/mL following 14 days of oral dosing) is less than exposure at either the rat or monkey NOAEL.

There was no genetic toxicity associated with Compound 292 in the in vitro genetic toxicity studies, and Compound 292 had no direct adverse effect in the in vivo rat micronucleus assay. Reproductive toxicity of Compound 292 was assessed in embryo/fetal developmental toxicity studies in rats and rabbits. The maternal and fetal NOAELs of Compound 292 in the rat and rabbit were 35 mg/kg/day (210 mg/m²/day) and 75 mg/kg/day (900 mg/m²/day), respectively. On the last day of dosing, the mean AUC₀₋₂₄ hr values at the NOAELs were 62200 ng*h/mL and 66200 ng*h/mL for pregnant rats and rabbits, respectively.

### Example 19

### Clinical Safety Studies

A randomized, double-blind, placebo-controlled, clinical study in healthy adult subjects was conducted with Compound 292. One-hundred and six (106) subjects were enrolled overall, which included 36 subjects in the single ascending dose (SAD) portion (24 active treatment; 12 placebo), 48 subjects in the multiple ascending dose (MAD) portion (36 active treatment; 12 placebo), 6 subjects in the food effect (FE) effect portion (consisting of Compound 292 dosing with sequential fed and fasting portions), and 16 subjects in the DDI portion (consisting of Compound 292 dosing periods with and without ketoconazole). The total subject exposure to Compound 292 is summarized in Table 9.

TABLE 9
Subject Exposure of Compound 292 in Clinical Safety Studies

| | | | Total | Total No. of |
|---|---|---|---|---|
| | | Duration of | Exposure per | Subjects |
| PART | Treatment Exposure | Treatment | Subject (mg) | Exposed |
| SAD | Placebo SD | 1 day | 0 | 12 |
| | 1 mg Compound 292 SD | 1 day | 1 | 4 |
| | 2 mg Compound 292 SD | 1 day | 2 | 4 |
| | 5 mg Compound 292 SD | 1 day | 5 | 4 |
| | 10 mg Compound 292 SD | 1 day | 10 | 4 |
| | 20 mg Compound 292 SD | 1 day | 20 | 4 |
| | 30 mg Compound 292 SD | 1 day | 30 | 4 |
| MAD | Placebo Q12h or Q24h | 14 days | 0 | 12 |
| | 1 mg Compound 292 Q12h* | 14 days | 26 | 9 |
| | 2 mg Compound 292 Q12h* | 14 days | 52 | 9 |
| | 5 mg Compound 292 Q12h* | 14 days | 130 | 9 |
| | 10 mg Compound 292 Q24h | 14 days | 140 | 9 |
| FE | 25 mg Compound 292 Fasted-Fed | 2 days | 50 | 3 |
| | 25 mg Compound 292 Fed-Fasted | 2 days | 50 | 3 |
| DDI | 10 mg Compound 292 SD | 2 days | 20 | 16 |

SD = single dose;
Q12h = once every 12 hrs;
Q24h = once every 24 hrs;
SAD = single ascending dose;
MAD = multiple ascending dose;
FE = food effect;
DDI = drug-drug interaction.
*includes QD dosing on Days 1 and 14.

Compound 292 was well tolerated at the doses evaluated. There were no deaths and no serious adverse events (SAEs). There did not appear to be a dose-related increase in AEs across the single dose range of 1 to 30 mg or the multiple dose range of 2 to 10 mg daily of Compound 292. No clinically significant safety laboratory or electrocardiogram (ECG) abnormalities were observed during any portion of the study.

Pharmacokinetic assessments demonstrated that Compound 292 was rapidly absorbed following single and multiple dose oral administration, with the maximum plasma concentration observed typically 1 hr after dosing. Across the dose ranges evaluated, Compound 292 exposure increased proportionally to dose. The mean elimination half-life ranged from 6.5 to 11.7 hrs after repeat dosing and did not depend on the dose level administered. Compound 292 accumulation was less than 2-fold following 14 days of Q12 h oral administration. A summary of Compound 292 PK parameters from the single dose portion is provided in Table 10 below. A summary of Compound 292 PK parameters from the multiple dose portion is provided in Table 11 below.

TABLE 10
Summary of Compound 292 PK Parameters Following Single Dose Administration (Mean, % CV)

| Compound | Cₘₐₓ | Tₘₐₓ | AUC₍₀₋ₜ₎ | AUC₍₀₋₂₄₎ | AUC_{(0-inf)} | CL/F | Vz/F | T_{1/2} |
|---|---|---|---|---|---|---|---|---|
| 292 Dose | (ng/mL) | (hr)* | (ng * hr/mL) | (ng * hr/mL) | (ng * hr/mL) | (L/h) | (L) | (hr) |
| 1 mg | 43.4 (31) | 1.00 (1.00-1.00) | 148 (68) | 149 (67) | 151 (68) | 8.39 (42) | 38.8 (28) | 3.52 (29) |
| 2 mg | 78.8 (16) | 1.00 (0.50-2.00) | 291 (45) | 289 (43) | 296 (44) | 7.69 (37) | 57.9 (38) | 5.43 (25) |
| 5 mg | 246 (16) | 1.00 (0.50-1.50) | 735 (5) | 733 (5) | 743 (5) | 6.74 (5) | 53.0 (15) | 5.43 (10) |
| 10 mg | 454 (40) | 0.50 (0.50-1.50) | 905 (15) | 891 (14) | 914 (14) | 11.1 (15) | 147 (29) | 9.47 (38) |
| 20 mg | 997 (32) | 1.00 (1.00-1.00) | 2243 (16) | 2193 (16) | 2250 (16) | 9.09 (18) | 99.1 (46) | 7.79 (51) |
| 30 mg | 1140 (38) | 1.00 (0.50-1.00) | 3384 (38) | 3263 (38) | 3395 (38) | 9.73 (33) | 113 (31) | 8.12 (18) |

*median (range);
h = hours

TABLE 11
Summary of Compound 292 PK Parameters Following Multiple Dose
Administration (Mean, % CV)

| Compound 292 | | | Tₘₐₓ | AUC₍₀₋ₜₐᵤ₎ | T_{1/2} | |
|---|---|---|---|---|---|---|
| Dose Regimen | Day | Cₘₐₓ (ng/mL) | (h)* | (ng * h/mL) | (h) | Racc |
| 1 mg Q12 h | 1 | 49.1 (26) | 0.52 (0.50-1.00) | 124 (40) | 3.46 (39) | — |
| | 14 | 66.8 (36) | 1.00 (0.50-1.50) | 199 (39) | 6.46 (20) | 1.65 (19) |
| 2 mg Q12 h | 1 | 101 (31) | 1.00 (0.50-2.00) | 290 (49) | 6.34 (35) | — |
| | 14 | 140 (36) | 1.00 (0.50-2.00) | 524 (47) | 9.75 (37) | 1.83 (22) |
| 5 mg Q12 h | 1 | 257 (38) | 1.00 (0.50-1.50) | 774 (41) | 5.76 (11) | — |
| | 14 | 355 (37) | 1.00 (0.50-2.02) | 1291 (38) | 8.32 (35) | 1.71 (15) |
| 10 mg Q24 h | 1 | 553 (27) | 0.52 (0.50-1.52) | 1527 (37) | 6.00 (13) | — |
| | 14 | 605 (16) | 1.00 (0.50-1.55) | 2232 (25) | 11.7 (82) | 1.54 (18) |

h = hours,
CV = coefficient of variation,
Racc = accumulation ratio,
*Median (range)

Data from the food effect portion indicate that food does not significantly alter systemic exposure to Compound 292. When administered in the presence of a high fat meal, Compound 292 concentration decreased by approximately 10% and median Tₘₐₓ was delayed from 1 hr (fasted) to 3 hrs (fed). Overall exposure, as assessed by AUC₍₀₋ₗₐₛₜ₎ and AUC_{(0-inf)}, increased by approximately 9% in the presence of a high fat meal.

Data from the DDI portion indicated that concomitant administration of 200 mg q12h ketoconazole increased exposure to Compound 292. On average, Cₘₐₓ, AUC₀₋ₗₐₛₜ and AUC_{0-inf} increased by approximately 66%, 285% and 295%, respectively, in the presence of ketoconazole compared to Compound 292 administered alone.

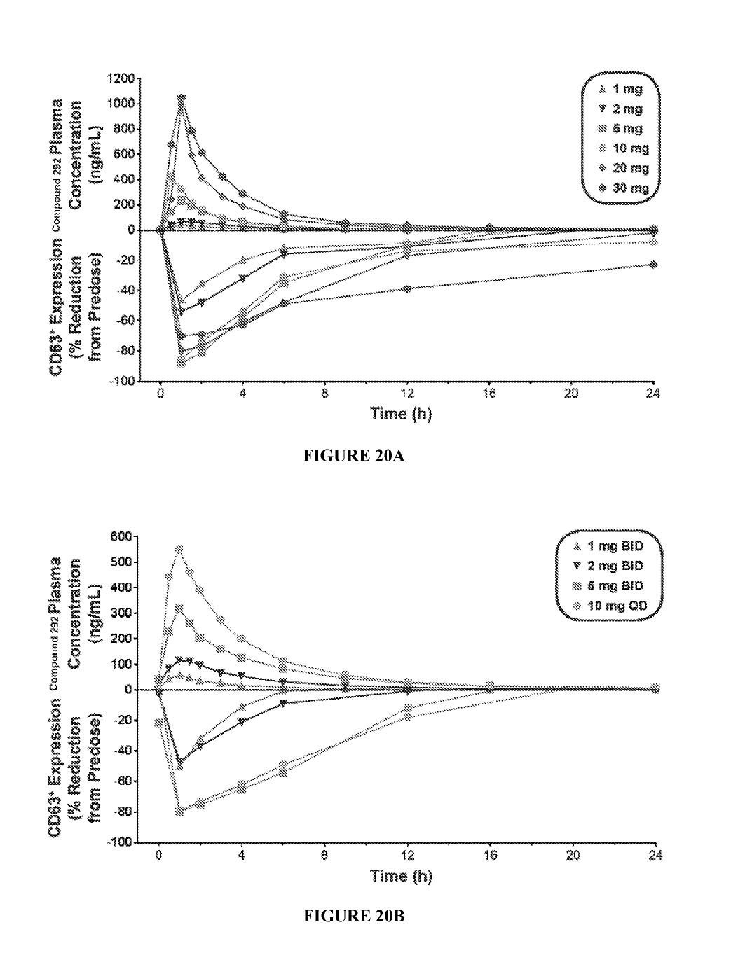

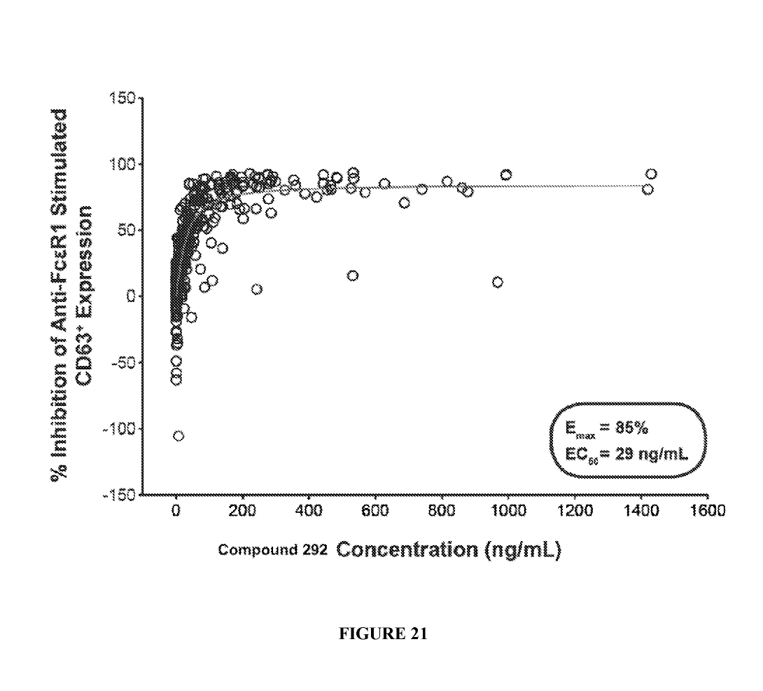

Following single and multiple Compound 292 doses, a dose-dependent reduction of basophil activation was observed at all dose levels, with a maximum reduction at 1 hr post dose; no notable change was observed following treatment with placebo. The PK/PD summary following single dose administration and multiple dose administration are shown in FIG. 20A and FIG. 20B, respectively, which demonstrate that the PD response was rapid and that maximal response was achieved at 5 mg dosing. A relationship was apparent between reduction of basophil activation and Compound 292 plasma concentrations, with saturation of the effect at higher Compound 292 plasma concentrations. The relationship between the pharmacodynamic response and the concentration of Compound 292 is depicted in FIG. 21, which demonstrates that the value of Eₘₐₓ is 85% and the value of EC₅₀ is 29 ng/mL.

Serial ECGs were performed at multiple time points after dosing in all study groups. No subject had a QTcF greater than 500 msec at any assessment, and the largest change from baseline in QTcF was 37 msec.

Overall, Compound 292 was well tolerated in healthy subjects at single doses up to 30 mg (highest dose tested) and up to 10 mg total daily dose (highest dose tested; 5 mg BID or 10 mg QD) for 14 days. In healthy subjects, the PK profile of Compound 292 is characterized by rapid absorption (peak plasma concentrations reached within 0.5-1 hour), moderately rapid elimination (half-life 3.5 to 9.5 hours following a single dose and 6.5 to 11.7 hours following repeat dosing) and dose proportional increases in systemic exposure (Cₘₐₓ and AUC). Minimal accumulation was observed after multiple dose administration (accumulation ratio 1.65-1.83 for BID dosing and 1.54 for QD dosing). Following single oral dose administration, clearance ranged from 6.7 L/h to 11.1 L/h and the volume of distribution ranged from 38.8 L to 147 L. Excretion of unchanged Compound 292 in urine was <2% of the administered dose, indicating minimal renal elimination of parent drug. CD63 expression on the surface of activated CCR3+ basophils was reduced in a dose-dependent manner at all single and multiple dose levels, with a maximum reduction at 1 hour post dose, corresponding to the time of maximum Compound 292 plasma concentrations Inhibition of basophil activation mirrored the Compound 292 concentration-time profile, with CD63 expression returning to baseline levels as plasma concentrations declined. Administration of 5 mg BID maintained PI3K-δ inhibition (EC₅₀=48 ng/mL) throughout the 12 hour dosing interval. Concomitant administration of a high-fat, high-calorie meal decreased Cₘₐₓ approximately 10%, shifted median Tₘₐₓ from 1 to 3 hours, and increased overall exposure (AUC) approximately 8-9%. These data suggest Compound 292 may be administered without regard to meals.

### Example 20

### Clinical Efficacy Studies: Rheumatoid Arthritis

A clinical study is conducted to examine the efficacy and safety of multiple dose levels of Compound 292 in subjects with active moderate-to-severe RA. The study employs a randomized, double-blind, placebo-controlled, parallel design. Approximately 316 adult subjects with moderate-to-severe RA who are on a stable dose of MTX at Screening are enrolled.

At Screening, adult male and female subjects will be evaluated for evidence of active RA. Approximately 316 subjects who meet all the eligibility criteria at Screening will be randomized at Baseline in a 1:1:1:1 ratio to one of 4 dose groups: Compound 292 0.5 mg BID, 1 mg BID or 5 mg BID, or placebo BID. After randomization, subjects will enter a 12-week Treatment Period where study drug will be self-administered as an outpatient; at the Week 2 visit, the morning dose must be taken in clinic to facilitate collection of PK samples. During the Treatment Period subjects will return to the clinic for efficacy and safety assessments at Week 2 (Day 14±2), Week 4 (Day 28±2), Week 6 (Day 42±2), Week 8 (Day 56±2), Week 10 (Day 70±2), and Week 12 (Day 84±2). Following Treatment Period completion at Week 12, subjects will enter a 3-week Follow-up Period which will include one clinic visit approximately 3 weeks after the last dose of study drug for a final assessment of safety.

### Blinding

Subjects, Sponsor study team members, vendor personnel, Investigators, and investigative site personnel are blinded to subject-level treatment assignments throughout the duration of the study. Personnel at the bioanalytical laboratory are unblinded.

The following controls are employed to maintain the double-blind status of the study at the investigational sites: (1) The capsules containing active drug and placebo are indistinguishable in appearance and taste; each subject receives one white (1 mg Compound 292 and/or placebo) and one orange (5 mg Compound 292 and/or placebo) capsule per dose, regardless of treatment arm. (2) The Investigator and other members of site staff involved with the study are blinded to the treatment randomization code throughout the study. (3) Individual subject C-reactive protein (CRP) values obtained for efficacy assessments are not available to investigational sites or the Sponsor until the study is completed and treatment assignments have been fully unblended. Although the results of additional laboratory tests are not considered likely to inadvertently unblind investigators or study staff to treatment assignment, the tender-joint and swollen-joint counts will be performed by a trained independent assessor who is to be blinded to laboratory test results, AEs, or changes in concomitant medications

### Study Population

This study enrolls approximately 316 subjects with active moderate-to-severe RA with a background of MTX.

### Efficacy Assessments

Primary and key secondary efficacy assessments in this study includes the following:

Joint Assessment (68 joints for pain/tenderness; 66 joints for swelling): The joint assessment is collected at all study visits except the EOS/Week 15 Visit, which includes Screening, Day 1, Week 2, Week 4, Week 6, Week 8, Week 10, and Week 12.

HAQ-DI: The Health Assessment Questionnaire (HAQ)-Disability Index (DI) measures self-report functional status (disability) and is a well-established instrument in arthritis. It is widely used throughout the world and has become a standard outcome measure for clinical trials in RA. The HAQ-DI evaluates the subject's ability to function in activities of daily living. The score results in values from 0 to 3 with higher scores representing greater disability. The HAQ-DI is assessed at each of the study visits during the treatment period (Day 1, Week 2, Week 4, Week 6, Week 8, Week 10, and Week 12).

Visual Analogue Scale (VAS) for subject assessment of pain is administered at each of the study visits during the Treatment Period (Day 1, Week 2, Week 4, Week 6, Week 8, Week 10, and Week 12).

VAS for Subject Global Assessment of Disease Activity is administered at each of the study visits during the Treatment Period (Day 1, Week 2, Week 4, Week 6, Week 8, Week 10, and Week 12).

VAS for Physician Global Assessment of Disease Activity is administered at each of the study visits during the Treatment Period (Day 1, Week 2, Week 4, Week 6, Week 8, Week 10, and Week 12).

C-reactive Protein, a biomarker of inflammation, is collected via a blood draw at each study visit (Screening, Day 1, Week 2, Week 4, Week 6, Week 8, Week 10, Week 12, and EOS/Week 15).

The Disease Activity Score using 28 joint counts (DAS28) has been used extensively and been validated to monitor disease activity in daily clinical practice as well as in clinical trials in subjects with RA. The 3-variable DAS28-CRP is calculated using the tender joint count (TJC), swollen joint count (SJC) and the CRP. The DAS28 is calculated at each study visit through Week 8. The following formula will be applied: DAS28-3(CRP)=[0.56*sqrt(TJC28)+0.28*sqrt(SJC28)+0.36*ln(CRP+1)]*1.10+1.15. Larger values represent greater disease activity.

Exploratory assessments of efficacy include the following:

FACIT-fatigue—This score measures fatigue while performing activities of daily living during the previous week. The score, a composite of 13-items rated on a 0-4 Likert Scale, ranges from 0 to 52. Lower scores indicate less fatigue.

SF-36—The SF-36 is a 36-item questionnaire to evaluating 8 domains: Role-Physical (RP), Bodily Pain (BP), Vitality (VT), Social Functioning (SF), Role-Emotional (RE), Mental Health (MH), Physical Functioning (PF), and General Health (GH). These scales comprise the physical and mental health summary scores. The 1-week, acute recall version of the SF-36 is evaluated.

Biomarkers of disease (e.g., rheumatoid arthritis) and pathway activity (e.g., PI3K pathway), including those known in the art and those provided herein and elsewhere, are measured and monitored.

The ACR20, ACR50, and ACR70 are composite endpoints of some of the above assessments. An ACR20 response (yes/no) will be defined as follows: At least a 20% improvement from baseline must be observed in each of the following: tender joints (68-count) and swollen joints (66-count); in addition, at least a 20% improvement from baseline must be observed in at least 3 of the following: subject assessment of pain on the VAS scale, subject global assessment of disease activity, physician global assessment of disease activity, HAQ-DI, and CRP. ACR50 and ACR70 responses will be defined using the above criteria, but showing at least 50% and 70% improvement, respectively.

### Pharmacokinetic Sampling

Blood samples for PK analysis are collected at Week 2, Week 4, Week 6, Week 8, Week 10, and Week 12. At the Week 2 visit, samples are collected pre-dose and approximately 2 hrs (±10 minutes) after administration of the morning dose of study medication which must be administered at the clinical site. At Weeks 4, 6, 8, 10, and 12 one PK sample is obtained anytime during the clinic visit. At all visits, the exact date and time of the PK sample collection will be recorded.

### Biomarkers

Biomarkers of Inflammatory Disease and/or PI3K Pathway Activity

Protein Biomarkers of Inflammatory Disease and PI3K Pathway Activity (Serum and Plasma): Serum and plasma samples are collected at Baseline, Weeks 2, 4, 8, 12 and the End of Study/Follow-up Visit to examine whether Compound 292 impacts levels of protein biomarkers of inflammatory disease and PI3K signaling pathway activity. As an example, IL-6 is a serum biomarker that is an acute phase protein and has been previously associated with severity of RA and response to treatment. See Chung et al., “The Correlation between Increased Serum Concentrations of Interleukin-6 Family Cytokines and Disease Activity in Rheumatoid Arthritis Patients,” Yonsei Medical Journal, 2011; 52(1):113.

RNA Biomarkers of Inflammatory Disease and PI3K Pathway Activity (Whole Blood): Whole blood samples are collected, where allowed, at Baseline, Weeks 2, 4, 8, 12, and End of Study/Follow-up Visit to examine whether Compound 292 impacts levels of RNA biomarkers of inflammatory disease and PI3K pathway activity. In regions where allowed, and at site where it is technically feasible, sites may collect an additional blood sample for isolation of PBMCs, also for the purpose of RNA isolation. Where possible, results from whole blood RNA and PBMC RNA are compared. As an example, expression of mRNA for IL4 and IL21 have been previously linked to the PI3K delta pathway in T follicular helper (Tfh) cells in a pre-clinical mouse model. See Rolf et al., “Phosphoinositide 3-Kinase Activity in T Cells Regulates the Magnitude of the Germinal Center Reaction,” The Journal of Immunology, 2010; 185(7):4042-52. Tfh cells have also been implicated in the pathogenesis of autoimmune diseases including RA. See Ma et al., “Increased Frequency of Circulating Follicular Helper T Cells in Patients with Rheumatoid Arthritis,” Clinical and Developmental Immunology, 2012; 2012:1-7.

### Predictors of Drug Response

The levels of serum/plasma protein biomarkers of inflammatory disease and/or PI3K pathway activity measured at Baseline are compared to measures of clinical outcome (e.g., ACR20) to determine if any biomarkers are predictive of Compound 292 activity.

Whole blood is collected on an optional basis (where allowed) at Baseline (or at any other time point if not collected at Baseline) for isolation of genomic DNA. Germline SNPs that have been previously linked to autoimmune disease susceptibility (e.g., PTPN22) or to pathways of drug metabolism or transport (e.g., CYP3A family and/or other drug metabolizing enzymes that have been associated with Compound 292 metabolism), may be examined in relation to clinical outcome (e.g., ACR20) and/or the PK of Compound 292.

### Immunophenotyping

Blood samples are taken for immunophenotyping Immunophenotyping provides additional information regarding the potential effect of Compound 292 on lymphocyte sub-populations. For immunophenotyping, the following are measured: absolute counts of mature human T lymphocytes (CD3+), natural killer cells (CD56+), and B lymphocytes (CD 19+); and percentages and absolute counts of mature human T lymphocytes (CD3+), suppressor/cytotoxic (CD3+CD8+) T-lymphocyte subsets, and helper/inducer (CD3+CD4+) T-lymphocyte subsets.

### Dosage and Administration

Compound 292 drug substance is a white to off-white crystalline powder. For this study, the Compound 292 drug product is supplied in capsule form, as size 2 white opaque hard gelatin capsules (0.5 and 1 mg) and size 2 orange opaque hard gelatin capsules (5 mg). The 0.5 mg capsule is filled with Compound 292 drug substance only. The 1 mg and 5 mg capsule strengths are formulated with Compound 292 drug substance and the excipients silicified microcrystalline cellulose, crospovidone, and magnesium stearate. All excipients used are listed in FDA's Inactive Ingredients Database for approved drug products and/or Generally Regarded as Safe (GRAS).

Compound 292 placebo is supplied in capsule form and consists of capsules filled with silicified microcrystalline cellulose. The Compound 292 placebo capsules are supplied as size 2, white opaque hard gelatin capsules to match the 0.5 and 1 mg Compound 292 drug product capsules and as size 2 orange opaque hard gelatin capsules to match the 5 mg Compound 292 drug product capsule.

Compound 292 (0.5 mg, 1 mg, and 5 mg) and placebo are self-administered as an oral capsule BID by subjects as outpatients for 12 weeks. At the Week 2 study visit, the morning dose of study drug is administered at the clinic.

The date, time, and quantity of each capsule taken are recorded by subjects. Subjects are advised to take each dose at approximately the same time of day and to record their study drug dosing on a daily basis. Missed doses should not be taken outside the BID schedule and should not be repeated. All missed doses should be recorded.

Compound 292 capsules should be swallowed whole with a glass of ambient water (approximately 8 ounces or 240 mL) at approximately the same time(s) each day, every 12±2 hrs. Intake of food and liquid is not restricted during the study other than grapefruit, grapefruit juice, and grapefruit containing products, which should be avoided for the duration of the study.

### Example 21

### Clinical Efficacy Studies

A clinical study is conducted to examine the efficacy and safety of multiple dose levels of Compound 292 in mile asthmatic subjects. The study employs a randomized, double-blind, placebo-controlled, multi-dose, 2-way cross-over design. Efficacy is evaluated based on the effect of Compound 292 on lung function and inflammatory indices following allergen challenge.

Up to 3 dosing cohorts of up to 10 subjects per cohort are enrolled sequentially. Interim data are analyzed after the completion of each cohort to determine the next dose to be studied. Baseline values for selected endpoints (e.g., the amount of allergen causing a decrease in FEV₁ of at least 20% in the EAR and at least 15% in the LAR) are established during Screening.

After fulfilling the eligibility criteria and completing the Screening assessments, subjects participate in two sequential treatment periods (TP 1 and TP 2). In TP 1 subjects receive either Compound 292 or placebo Q12±2 hours self-administered orally at home for 13 days. On the morning of Day 14 of both TP 1 (TP 1 Day 14) and TP 2 (TP 2 Day 14) subjects are admitted to the clinic prior to taking their morning dose. At this visit, subjects undergo an allergen challenge; spirometry and other efficacy endpoints are assessed, and serial blood samples are collected for PK. Subjects are confined overnight for safety observation. On Day 15 additional efficacy endpoints are evaluated in both treatment periods. Following the Day 15 assessments (TP 1 only), subjects enter a 7- to 12-day Washout Period before entering TP 2. During TP 2, subjects are dosed for 14 days with the alternate treatment from what they receive in TP 1. Following the end of TP 2 (Day 15), subjects return to the clinic 7 to 10 days after their last dose of study drug for a Safety Follow-up Visit. A final Safety Follow-up Phone Screen occurs 21 days after the last dose of study drug in TP 2.

Lung function, inflammatory indices, and other efficacy endpoints are assessed before and after each allergen challenge of each treatment period. Safety information is collected from the signing of the informed consent form at Screening through 21 days following the last dose of study drug. Pharmacokinetic (PK) sampling takes place on Day 14 of each treatment period.

### Blinding

Subjects, Sponsor study team members, vendor personnel, Investigators, and investigative site personnel are blinded to subject-level treatment assignments throughout the duration of the study. Personnel at the bioanalytical laboratory and pharmacists are unblinded.

The following controls will be employed to maintain the double-blind status of the study at the clinic: (1) The capsules and oral solution containing active drug and placebo are indistinguishable in appearance and taste. (2) The randomization code list is provided to the unblinded pharmacist for treatment assignment and dispensing purposes and kept in a secured, locked pharmacy, accessible to the pharmacist and the pharmacy assistant only. (3) The Investigator and other members of site staff involved with the study (apart from pharmacy staff and the bioanalytical laboratory) are blinded to the treatment randomization code throughout the study. (4) Interim bioanalytical data will be provided to the Sponsor in a blinded manner

### Study Population

Overall, this study enrolls between approximately 20 and 30 allergen-reactive subjects with mild asthma across either 2 or 3 dose cohorts. The study initially enrolls up to 10 subjects in Cohort 1 and up to 10 subjects in Cohort 2. If the results from Cohort 2 permit an additional dose to be tested in Cohort 3, then up to another 10 subjects are enrolled in Cohort 3.

### Efficacy Assessments

Allergen reactivity testing by skin test are performed at Screening to determine subject eligibility and to identify the allergen to be used in the allergen challenge. During the study, the following assessments are performed related to assessment of asthma or response to allergen challenge.

Screening Allergen Challenges:

Subject eligibility are also determined based on the results of two allergen challenges performed during Screening. Prior to the challenge, spirometry is measured to determine baseline lung function parameters. The first challenge is an incremental allergen challenge, where subjects are given increasing doses of a chosen inhaled allergen to achieve a target decrease in FEV₁ (forced expiratory volume in 1 second) of 25% from pre-challenge baseline. Subjects continue to undergo spirometry assessments through 10 hours after the last allergen dose to determine whether or not there is an adequate late asthmatic response (LAR) (measured between 3 and 10 hours post last allergen dose), defined here as a decrease in FEV₁ of at least 15% from their pre-challenge baseline. Following this incremental allergen challenge, there is a 21-day washout period prior to initiating the second Screening allergen challenge. A second “bolus” allergen challenge is done to confirm the results of the incremental allergen challenge. Subjects are administered one bolus of inhaled allergen equal to the total cumulative dose they received during the incremental allergen challenge. Subjects need to demonstrate at least a 20% reduction in FEV₁ during the EAR and at least a 15% reduction during the LAR from pre-challenge baseline to be eligible for the study.

Treatment Period Allergen Challenges:

On Day 1 (predose) subjects undergo a single series of spirometry assessments. On Day 14 of each treatment period, subjects undergo an allergen challenge similar in design to the bolus challenge described above. Spirometry measurements are performed through 10 hours post allergen challenge. The measurements obtained from the spirometry following the allergen challenges (FEV₁) constitute the primary evaluation of efficacy.

Other Measurements of Efficacy:

(1) Induced Sputum: sputum is collected at Screening and on Day 15 to examine WBC count and differential; cytokines and other inflammatory mediators may also be measured. (2) Exhaled nitric oxide (NO): NO is measured during both treatment periods at Day 1, Day 7, and on Days 14 and 15 (6 and 24 hours following allergen challenge) via NIOX Mino. (3) Methacholine challenge: Subjects are challenged with methacholine to determine their PC₂₀ on Day 15, approximately 24 hours after allergen challenge via 5 breath dosimeter method.

### Pharmacokinetic Sampling

Blood samples (˜3 mL each) for determination of plasma Compound 292 concentrations are collected for PK analysis on Day 14 of each treatment period at the following time points relative to the first dose of the day: pre-dose and 0.5, 1, 1.5, 2, 4, 6 (−1/+10 min), and 12 (−1/+15 min) hours post-dose. The 12-hour sample must be collected prior to administration of the second dose on Day 14.

The blood samples are collected into K2-EDTA-containing tubes via an indwelling IV catheter or by direct venipuncture. The exact times of blood sampling are recorded.

The plasma samples are analyzed to determine Compound 292 concentrations using a validated HPLC-MS/MS method.

On the days that include serial blood draws for PK analysis, food intake is not allowed for a minimum of 4 hours pre-dose to 4 hours post-dose. Water is allowed ad libitum, except for 1 hour prior to dosing and 1 hour following dosing.

### Dosage and Administration

Compound 292 drug substance is a white to off-white crystalline powder. For this study, the Compound 292 drug product may be supplied in two dosage forms: (1) Compound 292 1.0 mg capsule with the excipients silicified microcrystalline cellulose, crospovidone, and magnesium stearate, which are listed in FDA's Inactive Ingredients Database for approved drug products and/or Generally Regarded as Safe (GRAS), in size 2 white opaque hard gelatin capsules. (2) Compound 292 powder for oral solution (unformulated drug substance), 20 mg per bottle, for use in the preparation of oral solutions of Compound 292. At the clinical site, the powder for oral solution is used to prepare solutions consisting of 0.001 mg/mL and 0.01 mg/mL Compound 292 in 0.1 M citrate buffer, pH 2.5, and 0.5% ethanol. Individual unit doses are prepared in the pharmacy and dispensed to subjects for administration.

For this study, the Compound 292 placebo is supplied in capsule form and consists of capsules filled with silicified microcrystalline cellulose. The Compound 292 placebo capsules are supplied as size 2, white opaque hard gelatin capsules to match the 1.0 mg Compound 292 drug product capsule. The Compound 292 placebo for the oral solution is prepared at the clinical site and consists of 0.1 M citrate buffer, pH 2.5, and 0.5% ethanol. Individual unit doses are prepared in the pharmacy and dispensed to subjects for administration.

For Cohort 1, Compound 292 and placebo are self-administered by subjects at home Q12±2 hours as an oral capsule for 14 days, each in one of two treatment periods per the 2-way crossover study design. Future cohorts (Cohorts 2 and 3) may administer study drugs as either an oral solution or as capsules. The dose for Cohorts 2 and 3 can be determined by the following scheme:

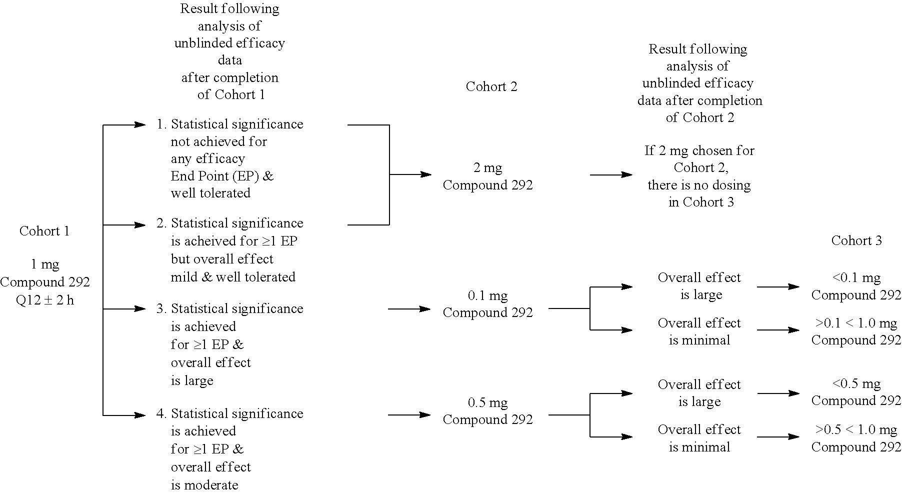

Compound 292 is administered as a fixed dose and should be administered orally, using the oral solution or minimal number of capsules, as necessary. For Cohort 1, Compound 292 1.0 mg and placebo are administered as capsules in single-dose units. If future Cohorts utilize a dose >1.0 mg, study drugs are supplied as a capsule in units of 1 mg. If future cohorts utilize doses <1.0 mg, the study drugs are administered as oral solutions in single-dose units.

The initial cohort (Cohort 1) is administered study drug Q12±2 hours. If a future cohort utilizes a once daily schedule (Q24h), doses must be taken Q24±2 hours. The date, time, and quantity of each capsule strength or bottle of oral solution taken are recorded by subjects. An attempt should be made to take each dose at approximately the same time of day.

Compound 292 capsules should be swallowed whole with a glass of ambient water (approximately 8 ounces or 240 mL) at approximately the same time(s) each day. Oral solutions are administered with 2 water rinses of the dosing bottle (approximately 20 mL per rinse for the 30 mL bottle and 50 mL per rinse for the 100 mL bottle). Intake of food and liquid is not restricted, other than on Day 14 of each treatment period. Prior to the Day 14 PK blood draws, subjects need to fast at least 4 hours prior to dose and for 4 hours postdose. Subjects must avoid grapefruit or grapefruit juice and grapefruit containing products for the duration of the study.

While exemplary embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein can be employed in practicing the invention. It is intended that the following claims define the scope of the invention and that methods and structures within the scope of these claims and their equivalents be covered thereby.

1. A method for reducing a rheumatoid arthritis-associated symptom in a subject, comprising administering to the subject a phosphoinositide 3-kinase inhibitor (PI3K) inhibitor, in an amount sufficient to decrease one or more symptoms, wherein the subject has been previously administered a therapy for rheumatoid arthritis.

2. The method of claim 1, wherein the symptom comprises one or more of an elevated level of IFN-α, TNF-α, IL-6, IL-8, IL-1, or an anti-dsDNA autoantibody.

3. The method of claim 1, wherein the symptom is selected from one or more of joint tenderness, joint swelling, and joint pain.

4. The method of claim 1, wherein the symptom is ankle inflammation or knee inflammation.

5. The method of claim 1, wherein the symptom affects one or more of the skin, kidney, heart, lung, blood, or nervous system.

6. The method of claim 1, wherein the subject has been previously administered a therapy for rheumatoid arthritis at least 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks before the PI3K inhibitor is administered.

7. The method of claim 1, wherein the subject has been previously administered a therapy for rheumatoid arthritis at least 1 week, 2 weeks, 1 month, 2 months, 3 months, or 4 months before the PI3K inhibitor is administered.

8. The method of claim 7, wherein the subject has been previously administered a therapy for rheumatoid arthritis at least 3 months before the PI3K inhibitor is administered.

9. The method of claim 1, wherein the subject has been administered a stable dose of a therapy for rheumatoid arthritis for at least 24 hours, 48 hours, 72 hours, 96 hours, 1 week, 2 weeks, 3 weeks, 4 weeks, 5 weeks, 6 weeks, 8 weeks, 12 weeks, or 16 weeks before the PI3K inhibitor is administered.

10. The method of claim 9, wherein the subject has been administered a stable dose of a therapy for rheumatoid arthritis for at least 6 weeks before the PI3K inhibitor is administered.

11. The method of claim 1, wherein the subject has been previously administered a therapy for rheumatoid arthritis at least 3 months before, and the subject has been administered a stable dose of the same therapy for rheumatoid arthritis for at least 6 weeks before, before the PI3K inhibitor is administered.

12. The method of claim 1, wherein the previously administered therapy comprises administering methotrexate to the subject.

13. The method of claim 1, wherein the PI3K inhibitor is administered from about 0.5 mg BID to about 5 mg BID.

14. The method of claim 1, wherein the PI3K inhibitor is a compound of Formula I-1: 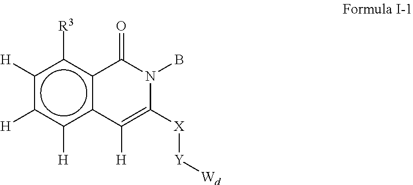
or an enantiomer or a mixture of enantiomers thereof, or a pharmaceutically acceptable salt, solvate, hydrate, co-crystal, clathrate, or polymorph thereof, wherein
B is a moiety of Formula II: 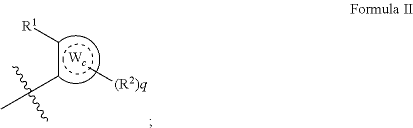
wherein W_{c} is aryl, heteroaryl, heterocycloalkyl, or cycloalkyl, and
q is an integer of 0, 1, 2, 3, or 4;
X is a bond or —(CH(R⁹))_{z}—, and z is an integer of 1;
Y is —N(R⁹)—;
W_{d} is: 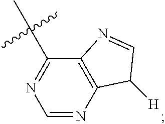 [SMILES: *c1ncnc2c1N=CC2]
R¹ is hydrogen, alkyl, alkenyl, alkynyl, alkoxy, amido, alkoxycarbonyl, sulfonamido, halo, cyano, or nitro;
R² is alkyl, alkenyl, alkynyl, cycloalkyl, heterocycloalkyl, aryl, heteroaryl, heteroarylalkyl, alkoxy, amino, halo, cyano, hydroxy or nitro;
R³ is hydrogen, alkyl, alkenyl, alkynyl, cycloalkyl, heterocycloalkyl, alkoxy, amido, amino, alkoxycarbonyl sulfonamido, halo, cyano, hydroxy or nitro; and
each instance of R⁹ is independently hydrogen, alkyl, or heterocycloalkyl.

15. The method of claim 14, wherein B is a moiety of Formula II: 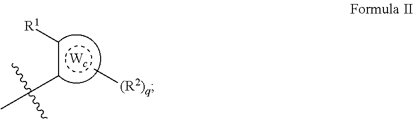
wherein W_{c} is aryl, heteroaryl, heterocycloalkyl, or cycloalkyl;
q is an integer of 0 or 1;
R¹ is hydrogen, alkyl, or halo;
R² is alkyl or halo; and
R³ is hydrogen, alkyl, or halo.

16. The method of claim 14, wherein X is —(CH(R⁹))_{z}—, and Y is —NH—.

17. The method of claim 14, wherein R³ is —H, —CH₃, —CH₂CH₃, —CF₃, —Cl or —F.

18. The method of claim 15, wherein X is —(CH(R⁹))_{z}—, wherein R⁹ is methyl and z and
W_{d} is

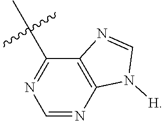

19. The method of claim 14, wherein the compound is predominately in an (S)-stereochemical configuration.

20. The method of claim 14, wherein the compound has a structure of Formula V-A2:

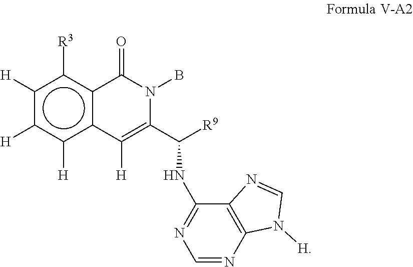

21. The method of claim 1, wherein the compound is selected from the group consisting of: 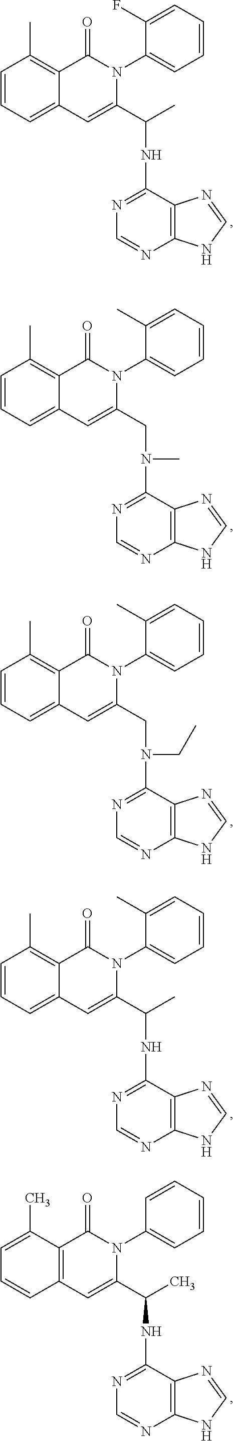 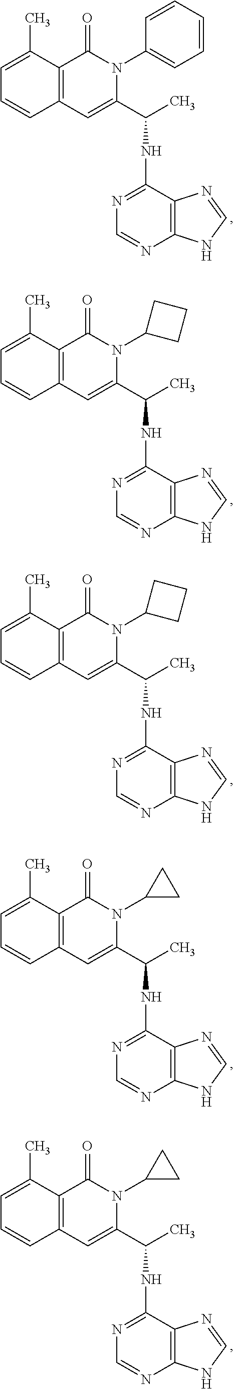 [SMILES: Cc1cccc2cc([C@H](C)Nc3ncnc4[nH]cnc34)n(-c3ccccc3)c(=O)c12 ; Cc1cccc2cc([C@@H](C)Nc3ncnc4[nH]cnc34)n(C3CCC3)c(=O)c12 ; Cc1cccc2cc([C@H](C)Nc3ncnc4[nH]cnc34)n(C3CCC3)c(=O)c12 ; Cc1cccc2cc([C@@H](C)Nc3ncnc4[nH]cnc34)n(C3CC3)c(=O)c12 ; Cc1cccc2cc([C@H](C)Nc3ncnc4[nH]cnc34)n(C3CC3)c(=O)c12] 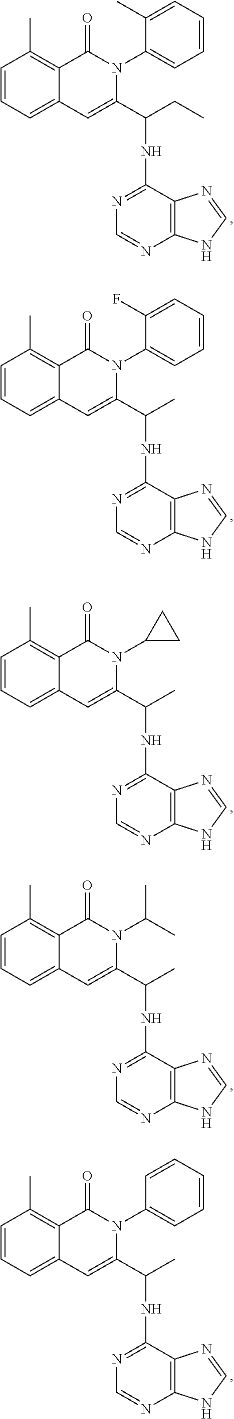 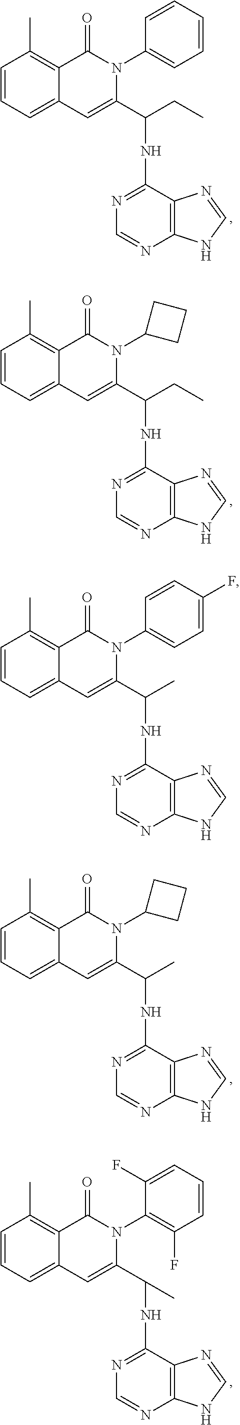 [SMILES: CCC(Nc1ncnc2[nH]cnc12)c1cc2cccc(C)c2c(=O)n1-c1ccccc1 ; CCC(Nc1ncnc2[nH]cnc12)c1cc2cccc(C)c2c(=O)n1C1CCC1 ; Cc1cccc2cc(C(C)Nc3ncnc4[nH]cnc34)n(-c3ccc(F)cc3)c(=O)c12 ; Cc1cccc2cc(C(C)Nc3ncnc4[nH]cnc34)n(C3CCC3)c(=O)c12 ; Cc1cccc2cc(C(C)Nc3ncnc4[nH]cnc34)n(-c3c(F)cccc3F)c(=O)c12] 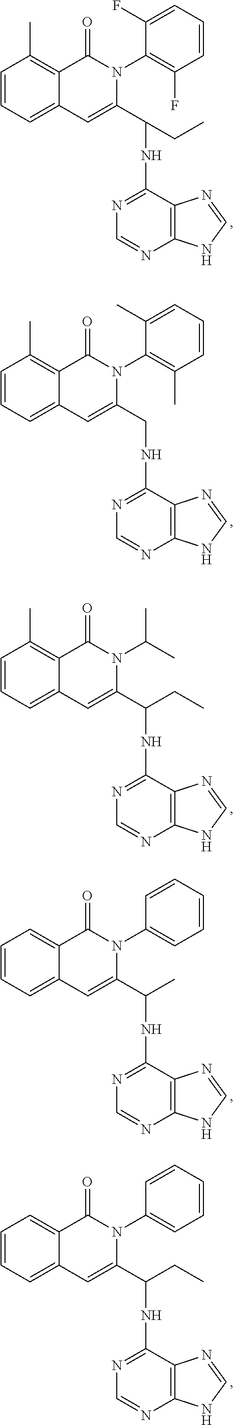 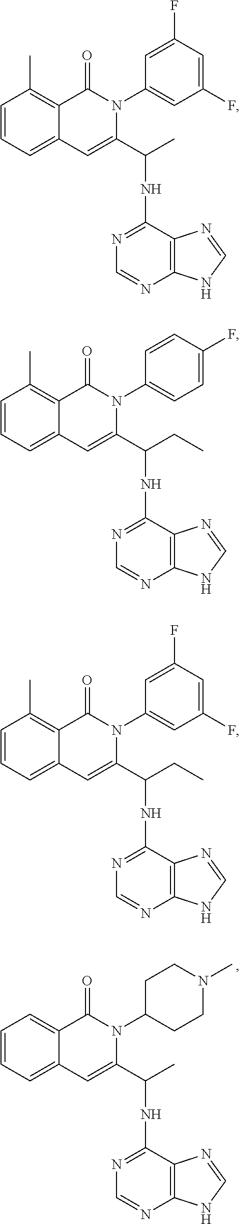 [SMILES: Cc1cccc2cc(C(C)Nc3ncnc4[nH]cnc34)n(-c3cc(F)cc(F)c3)c(=O)c12 ; CCC(Nc1ncnc2[nH]cnc12)c1cc2cccc(C)c2c(=O)n1-c1ccc(F)cc1 ; CCC(Nc1ncnc2[nH]cnc12)c1cc2cccc(C)c2c(=O)n1-c1cc(F)cc(F)c1 ; CC(Nc1ncnc2[nH]cnc12)c1cc2ccccc2c(=O)n1C1CCN(C)CC1] 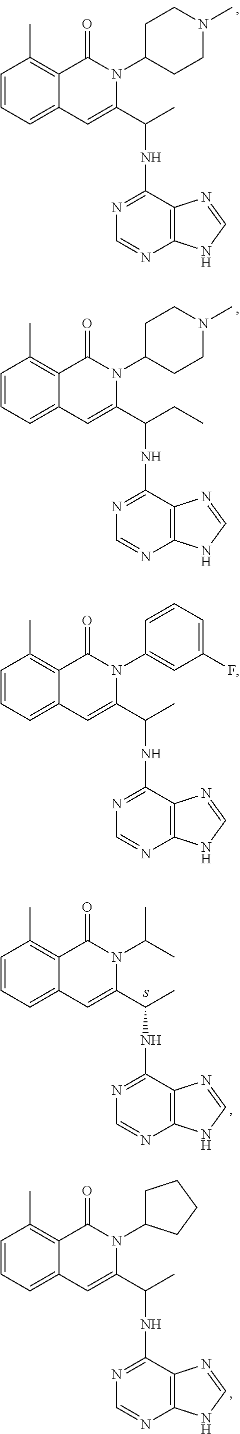 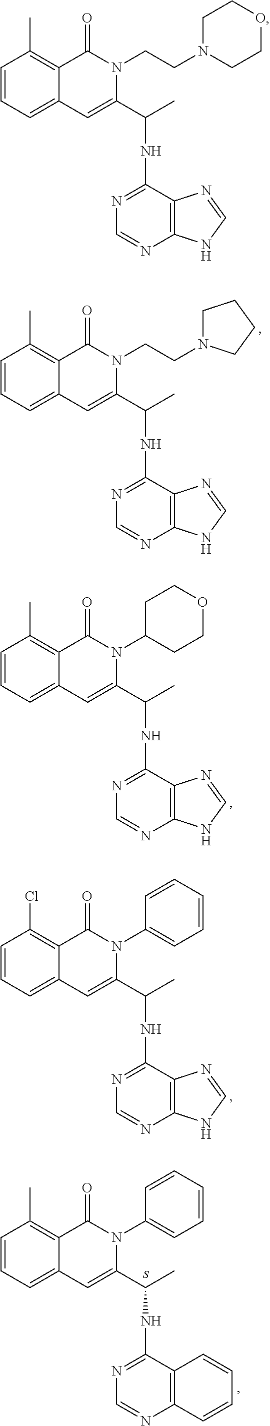 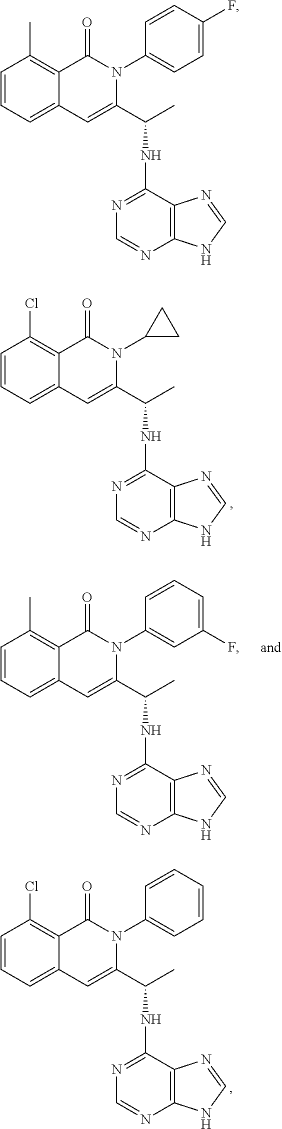 [SMILES: Cc1cccc2cc([C@H](C)Nc3ncnc4[nH]cnc34)n(-c3ccc(F)cc3)c(=O)c12 ; C[C@H](Nc1ncnc2[nH]cnc12)c1cc2cccc(Cl)c2c(=O)n1C1CC1 ; Cc1cccc2cc([C@H](C)Nc3ncnc4[nH]cnc34)n(-c3cccc(F)c3)c(=O)c12 ; C[C@H](Nc1ncnc2[nH]cnc12)c1cc2cccc(Cl)c2c(=O)n1-c1ccccc1]
or an enantiomer or a mixture of enantiomers thereof, or a pharmaceutically acceptable salt, solvate, hydrate, co-crystal, clathrate, or polymorph thereof.

22. The method of claim 21, wherein the compound is selected from the group consisting of: 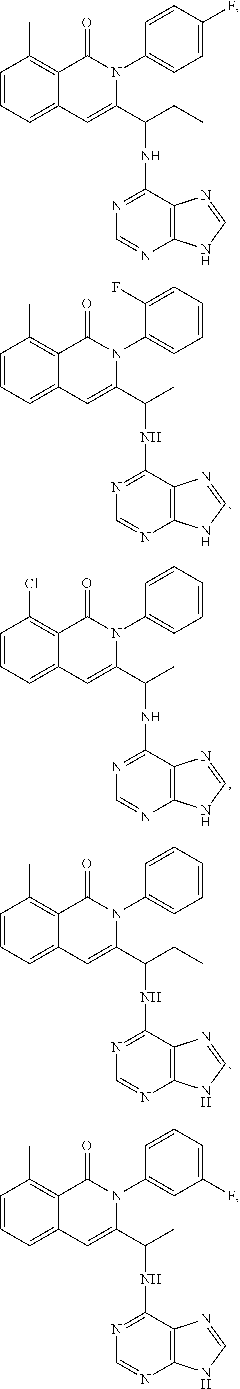 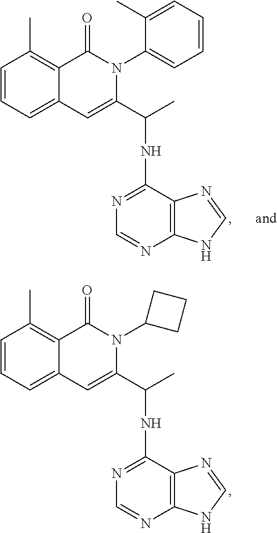
or an enantiomer or a mixture of enantiomers thereof, or a pharmaceutically acceptable salt, solvate, hydrate, co-crystal, clathrate, or polymorph thereof.

23. The method of claim 22, wherein the compound is selected from the group consisting of: 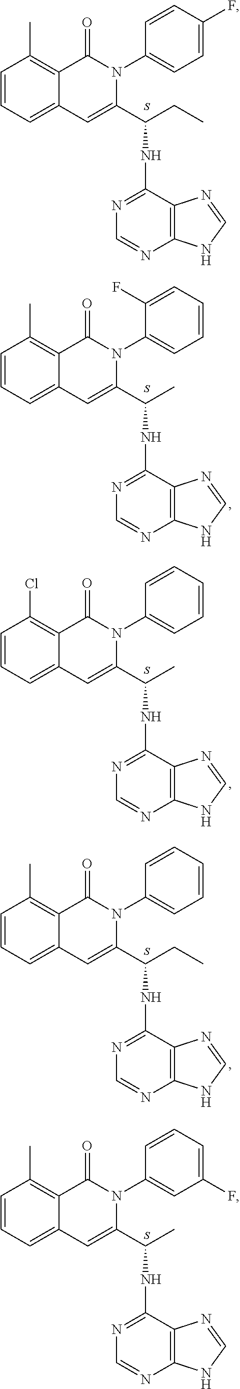 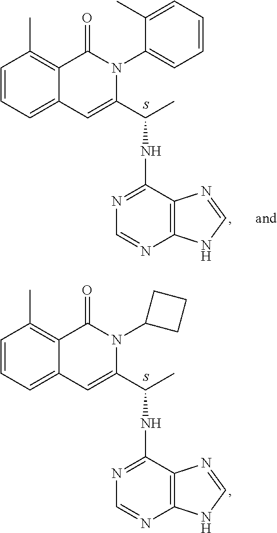
or a pharmaceutically acceptable salt, solvate, hydrate, co-crystal, clathrate, or polymorph thereof.

24. The method of claim 23, wherein the compound has the following structure: 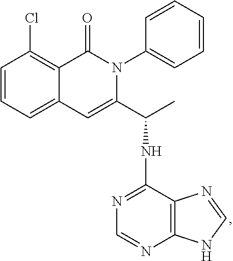 [SMILES: C[C@H](Nc1ncnc2[nH]cnc12)c1cc2cccc(Cl)c2c(=O)n1-c1ccccc1]
or a pharmaceutically acceptable salt, solvate, hydrate, co-crystal, clathrate, or polymorph thereof.

25. The method of claim 1, wherein the rheumatoid arthritis is selected from the group consisting of insidious onset rheumatoid arthritis, acute or immediate onset rheumatoid arthritis, moderate to severe rheumatoid arthritis, severe rheumatoid arthritis, early rheumatoid arthritis, seronegative rheumatoid arthritis, seropositive rheumatoid arthritis, and rheumatoid arthritis unresponsive or inadequately responsive to other disease-modifying anti-rheumatic drugs.

26. A method of treating, preventing, and/or managing rheumatoid arthritis in a subject, comprising administering an effective amount of a PI3K inhibitor to a subject in need thereof, wherein the subject has been previously administered a therapy for rheumatoid arthritis.

27. The method of claim 26, wherein the subject has been previously administered a therapy for rheumatoid arthritis at least 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks before the PI3K inhibitor is administered.

28. The method of claim 26, wherein the subject has been previously administered a therapy for rheumatoid arthritis at least 1 week, 2 weeks, 1 month, 2 months, 3 months, or 4 months before the PI3K inhibitor is administered.

29. The method of claim 26, wherein the subject has been previously administered a therapy for rheumatoid arthritis at least 3 months before the PI3K inhibitor is administered.

30. The method of claim 26, wherein the subject has been administered a stable dose of a therapy for rheumatoid arthritis for at least 24 hours, 48 hours, 72 hours, 96 hours, 1 week, 2 weeks, 3 weeks, 4 weeks, 5 weeks, 6 weeks, 8 weeks, 12 weeks, or 16 weeks before the PI3K inhibitor is administered.

31. The method of claim 26, wherein the subject has been administered a stable dose of a therapy for rheumatoid arthritis for at least 6 weeks before the PI3K inhibitor is administered.

32. The method of claim 26, wherein the subject has been previously administered a therapy for rheumatoid arthritis at least 3 months before, and the subject has been administered a stable dose of the same therapy for rheumatoid arthritis for at least 6 weeks before, before the PI3K inhibitor is administered.

33. The method of claim 26, wherein the previously administered therapy comprises administering methotrexate to the subject.

34. The method of any of claim 26, wherein the PI3K inhibitor is administered from about 0.5 mg BID to about 5 mg BID.

35. The method of claim 26, wherein the subject is a mammal.

36. The method of claim 35, wherein the subject is a human.

37. The method of claim 26, wherein the levels of one or more of IFN-α, TNF-α, IL-6, IL-8, or IL-1 are reduced.

38. The method of claim 26, wherein one or more of joint tenderness, joint swelling, or joint pain are reduced.

39. The method of claim 26, wherein the levels of immune complexes are reduced.

40. The method of claim 26, wherein the rheumatoid arthritis is selected from the group consisting of insidious onset rheumatoid arthritis, acute or immediate onset rheumatoid arthritis, moderate to severe rheumatoid arthritis, severe rheumatoid arthritis, early rheumatoid arthritis, seronegative rheumatoid arthritis, seropositive rheumatoid arthritis, and rheumatoid arthritis unresponsive or inadequately responsive to other disease-modifying anti-rheumatic drugs.

41. The method of claim 26, further comprising administration of an additional therapeutic agent.

42. The method of any one of claim 41, wherein the additional therapeutic agent is chosen from one or more of belimumab, AGS-009, rontalizumab, vitamin D3, sifalimumab, AMG 811, IFNα Kinoid, CEP33457, epratuzumab, LY2127399, Ocrelizumab, Atacicept, A-623, SBI-087, AMG557, laquinimod, rapamycin, cyclophosphamide, azathioprine, mycophenolate, leflunomide, methotrexate, CNTO 136, tamibarotene, N-acetylcysteine, CDP7657, hydroxychloroquine, rituximab, carfilzomib, bortezomib, ONX 0914, IMO-3100, DV1179, sulfasalazine, and chloroquine.

43. The method of claim 26, wherein the PI3K inhibitor is a compound of Formula I-1: 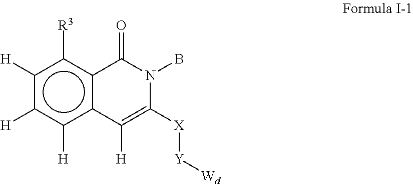
or an enantiomer or a mixture of enantiomers thereof, or a pharmaceutically acceptable salt, solvate, hydrate, co-crystal, clathrate, or polymorph thereof, wherein
B is a moiety of Formula II: 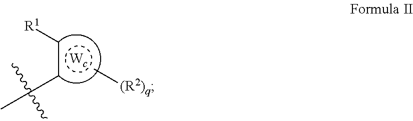
wherein W_{c} is aryl, heteroaryl, heterocycloalkyl, or cycloalkyl, and
q is an integer of 0, 1, 2, 3, or 4;
X is a bond or —(CH(R⁹))_{z}—, and z is an integer of 1;
Y is —N(R⁹)—;
W_{d} is: 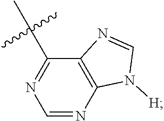
R¹ is hydrogen, alkyl, alkenyl, alkynyl, alkoxy, amido, alkoxycarbonyl, sulfonamido, halo, cyano, or nitro;
R² is alkyl, alkenyl, alkynyl, cycloalkyl, heterocycloalkyl, aryl, heteroaryl, heteroarylalkyl, alkoxy, amino, halo, cyano, hydroxy or nitro;
R³ is hydrogen, alkyl, alkenyl, alkynyl, cycloalkyl, heterocycloalkyl, alkoxy, amido, amino, alkoxycarbonyl sulfonamido, halo, cyano, hydroxy or nitro; and
each instance of R⁹ is independently hydrogen, alkyl, or heterocycloalkyl.

44. The method of claim 43, wherein B is a moiety of Formula II: 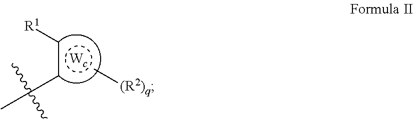
wherein W_{c} is aryl, heteroaryl, heterocycloalkyl, or cycloalkyl;
q is an integer of 0 or 1;
R¹ is hydrogen, alkyl, or halo;
R² is alkyl or halo; and
R³ is hydrogen, alkyl, or halo.

45. The method of claim 43, wherein X is —(CH(R⁹))_{z}—, and Y is —NH—.

46. The method of claim 43, wherein R³ is —H, —CH₂CH₃, —CF₃, —Cl or —F.

47. The method of claim 44, wherein X is —(CH(R⁹))_{z}—, wherein R⁹ is methyl and z=1; and
W_{d} is

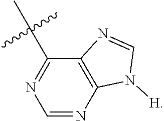

48. The method of claim 43, wherein the compound is predominately in an (S)-stereochemical configuration.

49. The method of claim 43, wherein the compound has a structure of Formula V-A2:

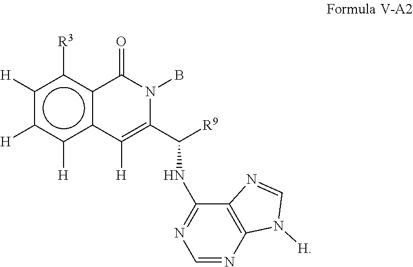

50. The method of claim 26, wherein the compound is selected from the group consisting of: 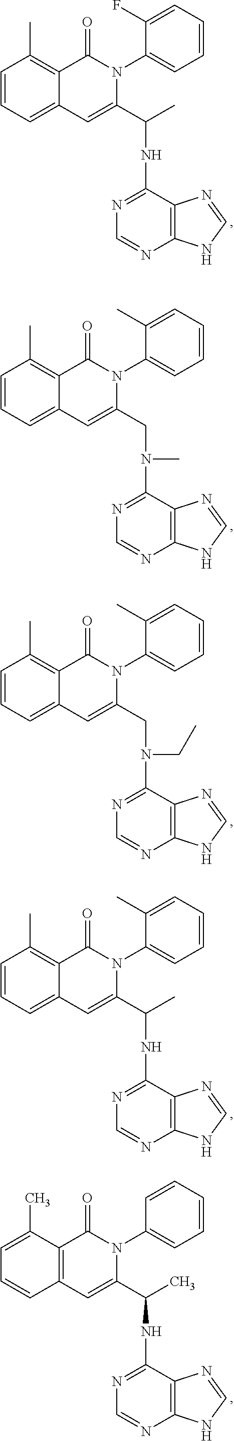 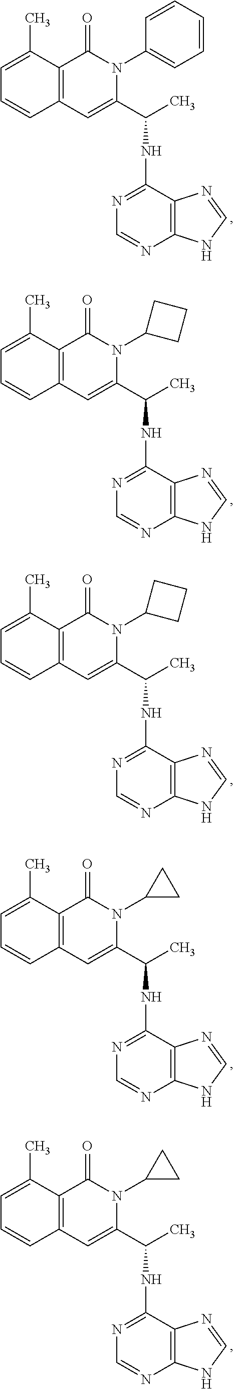 [SMILES: Cc1cccc2cc([C@H](C)Nc3ncnc4[nH]cnc34)n(-c3ccccc3)c(=O)c12 ; Cc1cccc2cc([C@@H](C)Nc3ncnc4[nH]cnc34)n(C3CCC3)c(=O)c12 ; Cc1cccc2cc([C@H](C)Nc3ncnc4[nH]cnc34)n(C3CCC3)c(=O)c12 ; Cc1cccc2cc([C@@H](C)Nc3ncnc4[nH]cnc34)n(C3CC3)c(=O)c12 ; Cc1cccc2cc([C@H](C)Nc3ncnc4[nH]cnc34)n(C3CC3)c(=O)c12] 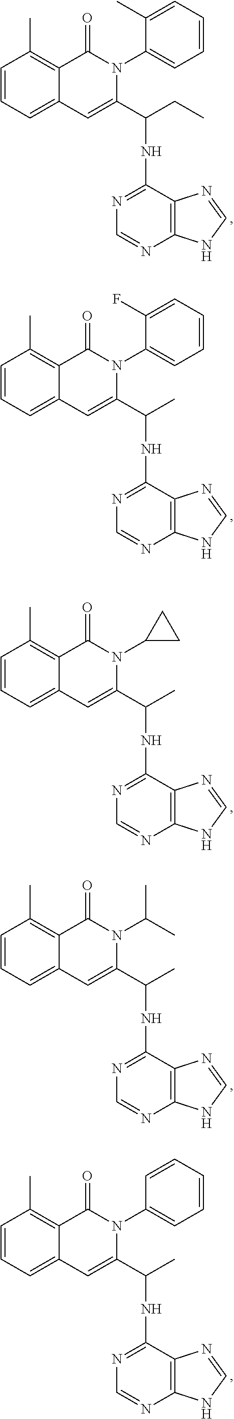 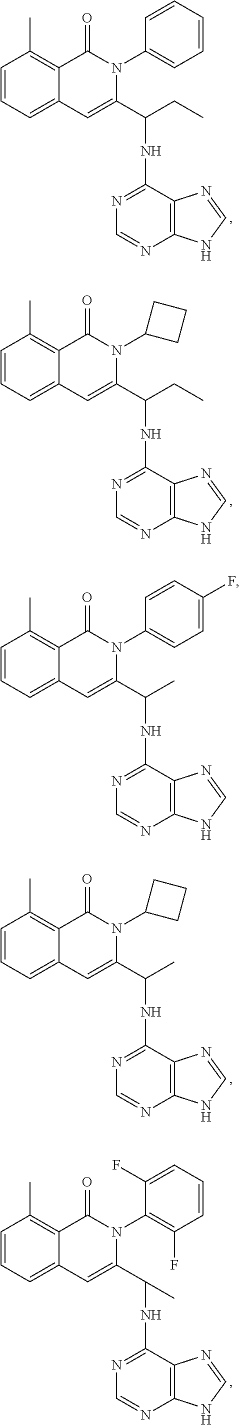 [SMILES: CCC(Nc1ncnc2[nH]cnc12)c1cc2cccc(C)c2c(=O)n1-c1ccccc1 ; CCC(Nc1ncnc2[nH]cnc12)c1cc2cccc(C)c2c(=O)n1C1CCC1 ; Cc1cccc2cc(C(C)Nc3ncnc4[nH]cnc34)n(-c3ccc(F)cc3)c(=O)c12 ; Cc1cccc2cc(C(C)Nc3ncnc4[nH]cnc34)n(C3CCC3)c(=O)c12 ; Cc1cccc2cc(C(C)Nc3ncnc4[nH]cnc34)n(-c3c(F)cccc3F)c(=O)c12] 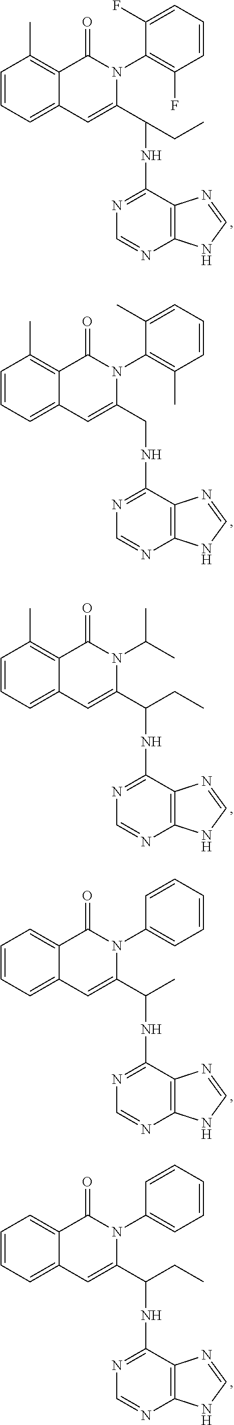 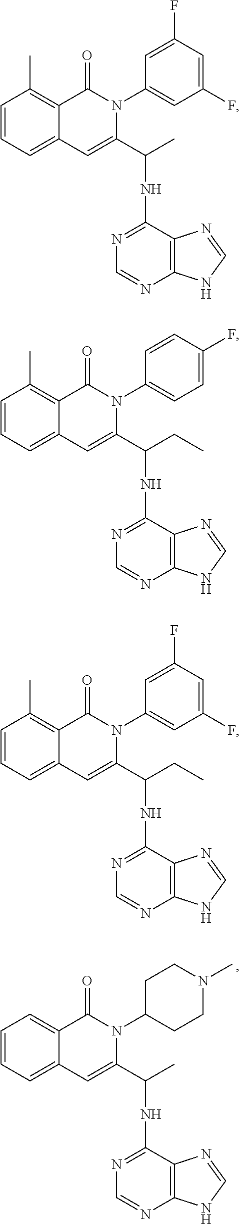 [SMILES: Cc1cccc2cc(C(C)Nc3ncnc4[nH]cnc34)n(-c3cc(F)cc(F)c3)c(=O)c12 ; CCC(Nc1ncnc2[nH]cnc12)c1cc2cccc(C)c2c(=O)n1-c1ccc(F)cc1 ; CCC(Nc1ncnc2[nH]cnc12)c1cc2cccc(C)c2c(=O)n1-c1cc(F)cc(F)c1 ; CC(Nc1ncnc2[nH]cnc12)c1cc2ccccc2c(=O)n1C1CCN(C)CC1] 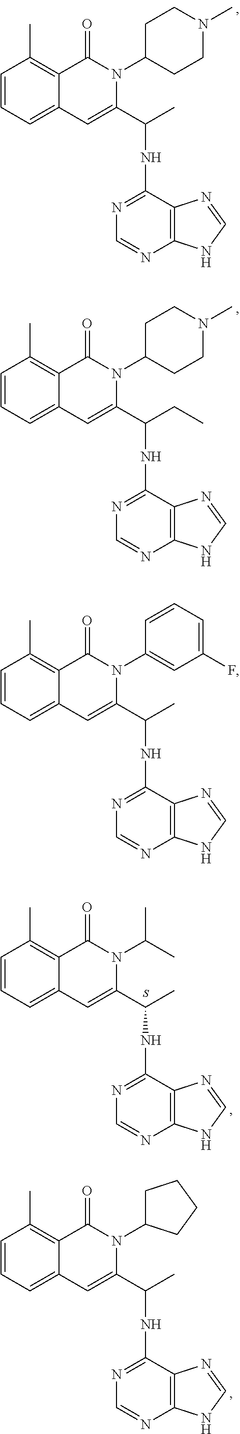 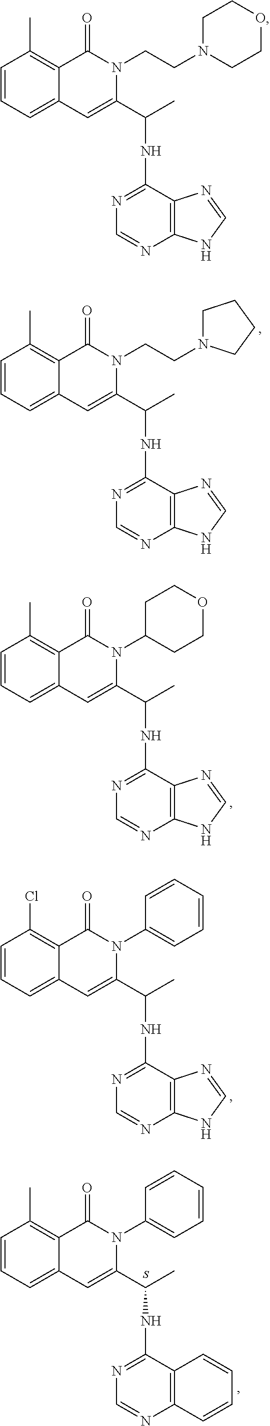 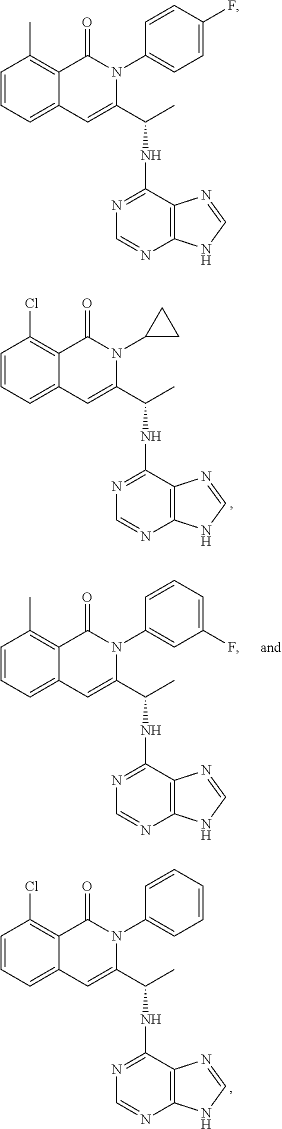 [SMILES: Cc1cccc2cc([C@H](C)Nc3ncnc4[nH]cnc34)n(-c3ccc(F)cc3)c(=O)c12 ; C[C@H](Nc1ncnc2[nH]cnc12)c1cc2cccc(Cl)c2c(=O)n1C1CC1 ; Cc1cccc2cc([C@H](C)Nc3ncnc4[nH]cnc34)n(-c3cccc(F)c3)c(=O)c12 ; C[C@H](Nc1ncnc2[nH]cnc12)c1cc2cccc(Cl)c2c(=O)n1-c1ccccc1]
or an enantiomer or a mixture of enantiomers thereof, or a pharmaceutically acceptable salt, solvate, hydrate, co-crystal, clathrate, or polymorph thereof.

51. The method of claim 50, wherein the compound is selected from the group consisting of: 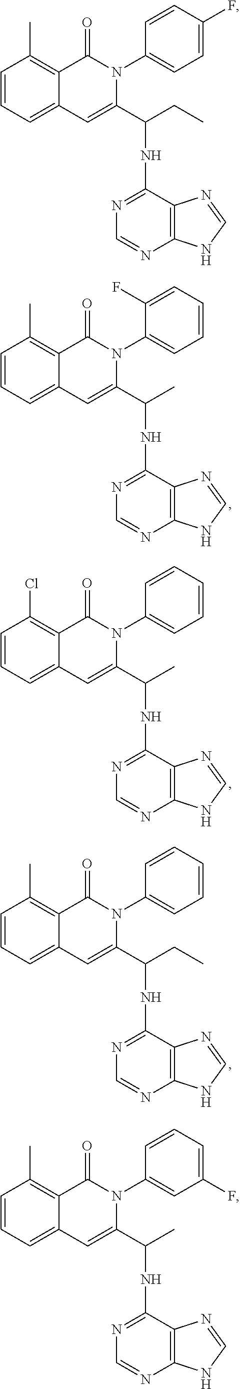 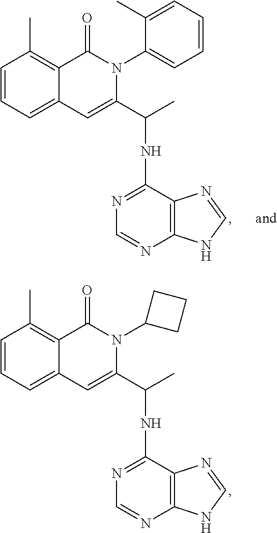
or an enantiomer or a mixture of enantiomers thereof, or a pharmaceutically acceptable salt, solvate, hydrate, co-crystal, clathrate, or polymorph thereof.

52. The method of claim 50, wherein the compound is selected from the group consisting of: 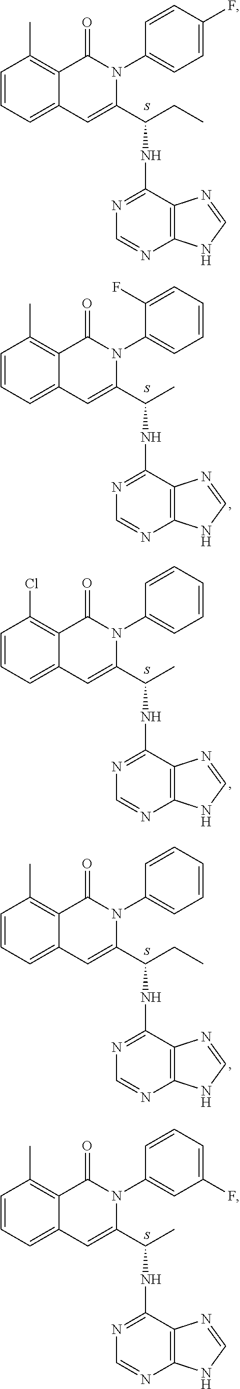 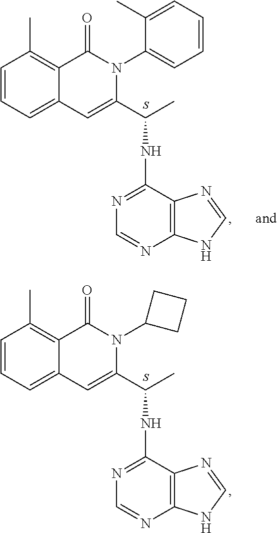
or a pharmaceutically acceptable salt, solvate, hydrate, co-crystal, clathrate, or polymorph thereof.

53. The method of claim 50, wherein the compound has the following structure: 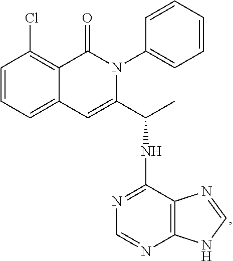 [SMILES: C[C@H](Nc1ncnc2[nH]cnc12)c1cc2cccc(Cl)c2c(=O)n1-c1ccccc1]
or a pharmaceutically acceptable salt, solvate, hydrate, co-crystal, clathrate, or polymorph thereof.

54-97. (canceled)

98. A method for screening for PI3K-γ selective inhibitors comprising (a) creating a pouch at the back of an animal and introducing a PI3K-γ specific stimuli into the pouch; (b) administrating a compound to the animal; (c) measuring the influx of leukocyte into the pouch; and (d) comparing the influx of leukocyte to that of a control vehicle; wherein a reduction in the influx of leukocyte indicates the compound is a PI3K-γ selective inhibitor.

99. The method of claim 98, wherein the animal is rat.

100. The method of claim 98, wherein the PI3K-γ specific stimuli is IL-8.

101. The method of claim 98, wherein the leukocyte is neutrophil or eosinophil.
